SEQUENCE LISTING (60 sequences)

SEQ ID NO: 1 - protein, 245 aa, Homo sapiens
MPSQQKKIIFCMAGVFSFACALGVVTALGTPLWIKATVLCKTGALLVNASGQELDKFMGEMQYGLFHGEGVRQCGLGARPFRFSFFPDLLKAIPVSIHVNVILFSAILIVLTMVGTAFFMYNAFGKPFETLHGPLGLYLLSFISVALWLPATRHQAQGSCGCLVMILFASEVKIHHLSEKIANYKEGTYVYKTQSEKYTTSFWVIFFCFFVHFLNGLLIRLAGFQFPFAKSKDAETTNVAADLMY

SEQ ID NO: 2 - DNA, 738 bp, Homo sapiens
atgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcattttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcagttgccctttggctgccagctaccaggcaccaggctcaaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctgggtcattttcttttgcttttttgttcattttctgaatgggctcctaatacgacttgctggatttcagttcccttttgcaaaatctaaagacgcagaaacaactaatgtagctgcagatctaatgtactga

SEQ ID NO: 3 - protein, 232 aa, Homo sapiens
MPSQQKKIIFCMAGVFSFACALGVVTALGTPLWIKATVLCKTGALLVNASGQELDKFMGEMQYGLFHGEGVRQCGLGARPFRFSFFPDLLKAIPVSIHVNVILFSAILIVLTMVGTAFFMYNAFGKPFETLHGPLGLYLLSFISGSCGCLVMILFASEVKIHHLSEKIANYKEGTYVYKTQSEKYTTSFWVIFFCFFVHFLNGLLIRLAGFQFPFAKSKDAETTNVAADLMY

SEQ ID NO: 4 - DNA, 699 bp, Homo sapiens
atgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcattttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctgggtcattttcttttgcttttttgttcattttctgaatgggctcctaatacgacttgctggatttcagttcccttttgcaaaatctaaagacgcagaaacaactaatgtagctgcagatctaatgtactga

SEQ ID NO: 5 - protein, 120 aa, Homo sapiens
MQALQQQPVFPDLLKAIPVSIHVNVILFSAILIVLTMVGTAFFMYNAFGKPFETLHGPLGLYLLSFISGSCGCLVMILFASEVKIHHLSEKIANYKEGTYVYKTQSEKYTTSFWLTKGHS

SEQ ID NO: 6 - DNA, 363 bp, Homo sapiens
atgcaggccctgcagcagcaaccagtttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctggctgactaaaggccacagctga

SEQ ID NO: 7 - protein, 185 aa, Homo sapiens
MPSQQKKIIFCMAGVFSFACALGVVTALGTPLWIKATVLCKTGALLVNASGQELDKFMGEMQYGLFHGEGVRQCGLGARPFRFSCYFLDPFMGLPTGVPHLLSLPCSTSCRREHTSERVQEPAGCFSAVRSKLHAGPAAATSFSRFAQSNPSEHPRQCHSLLCHPYCVNHGGDSLLHVQCFWKTF

SEQ ID NO: 8 - DNA, 558 bp, Homo sapiens
atgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcatgctattttcttgaccccttcatgggactcccaacaggggtaccccatttactcagcctgccctgctcaacctcttgcaggagggagcacacgagtgaacgagtgcaggaaccagctggctgctttagtgctgtgaggagtaaactccatgcaggccctgcagcagcaaccagtttttccagatttgctcaaagcaatcccagtgagcatccacgtcaatgtcattctcttctctgccatccttattgtgttaaccatggtggggacagccttcttcatgtacaatgcttttggaaaaccttttga

SEQ ID NO: 9 - DNA, 46837 bp, Homo sapiens
aggagatacttgaaggcagtttgaaagacttgttttacagattcttagtccaaagatttccaattagggagaagaagcagcagaaaaggagaaaagccaagtatgagtgatgatgaggccttcatctactgacatttaacctggcgagaaccgtcgatggtgaagttgccttttcagctgggagctgtccgttcagcttccgtaataaatgcagtcaaagaggcagtcccttcccattgctcacaaaggtcttgtttttgaacctcgccctcacagaagccgtttctcatcatgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcatgtaagtagcaattgcatttgagttatttaatgctttaggcagactcttcccagtgttgcgaggaattatatttgagaattttgccgtgtttactgcaggactttttaaatcggtgtgaaccatatgaaaaacctatgactctgagcaatttcttcttcctagtttttattattttatacttgctttttattataatatagagttaattcattgttacataattaaggtttttggaaatattggcaattaagatgcttaagtattaatatttatgtaaaaaattatggagtctttttaaaaaagtaaacttggggaaataggaaagctgtaaagaatgatctttatgctttttgttctttataaaaagaaccaaggtcatgggctccgtatttaaccaggttgccacctttctcatgattttgtttcctgctccccactccctcccattattcctgctaagacctttcctgctgctaaatattcagttttcatttttaactaatttggaatcatttggctatagaaatttaaaatgatctgctgtgctaactgggaaagaaatggatgcctatttagtatagaacattttaaactgattgacctgcaaatcatgtagagaatatgagagagattttcttgttgtgatttttgtgaaatggaagtgtaatccacagtatttataacctgtttatcttaagaagagaatttttaaaaattaccatgtgaataggcaactcattaaatgaaaattaataggaagtcatttgttatatctcttacaacacacattcagaagttattattatttcagaagggctggtttggaacaaccttatgaagacacagtcagtaaattactgcataaatcactcttcaggaaaggaggttaccaactgaagcatttaaaatgaattattattttgcccaggttttttttttctttctagtataggtagaaggctaaattaattgaatttattattaacatatgcagtgcctaattaaatttcagtgctggtctatttatatttctgcaacattccttatatcttcttagcagtcattggacaccaaccttcagctcacataggttactaagtgatatgaattttcatagggctccagaaaatttccaagaattggttgttagctttttaattgatgaagtggataccagttcttttcactgaatggcttttattcattaaggtaatggggctgttagagttgcttagttttcctggggaaggggaaggaagaaaacaaagcagaatgtcatgtgatatgcaactgtattaaaaaaccgaaaaggaaaaaagttgagagagatgatttaaccgtgagtcaccggcagccaaagcgtgagtaaagcttctcacagatgaatttagacaaaagcggagaaggtactggtgaattttctggagcctttacattttctacagtgaaatggagataaactttactcatgccataggacatgtttcaaaacaataataagatgttttctgaacacttactacatactaagcactttatatgctttgtctcatttaatccttacacagccacattcttctggggtttagcgaatgatttttgtggttgtgtcctatgcttgtcctgtctaaggatgaagttgttctaattgggtgcccctccttttgctttctgtgaggacttgcagaactggtggggtttaaacagtaccctcacttatctcacagaatttcattagctcccagatacccctgacattctccccctagcctagtgaagaaaatcttccatttacttgttcattctgcagtgacagctccatcaatatacaatagactatacatattaagtgtactgtatatactatacatgttaaaaatctcattcattttggtgaggcccagctaagaatacttacagtagagcttttttttttttcctaagcataaaagtatctttttcaatgcagcatgagacagagttgggaaaaccaaaataaatagatccaatggactccccaaagaggataatattcatttaaataaacacccctctcagtgttaaaactttctaatcaacatgcctttgggacacattgcaccctcaaagtttacactcccattgcaacgcagctttgtggttcacgttttttccattcagaatgtcattaccctgtcaatgatgtttcatcaacgtttgcttggatgagaatcctctgatattcttcctgatagaaatgtataagccctgttcatataaatgaataaaagatctaaccttactttctcagtagtggcttccttggagcaaaaagcagggacctccagagagctcaggtggatgactcttttctgtttcttccagagctcaacttacaattagtgcacaattcatttcccagaatgtcttctttcttattgtgcctttagaaagttattaagcaaacatttgaattcacagaatcttaccagtgtaagaggaatggaaaaggtaacttatcaaggtaacaatcacttcgtggccagttttttcggctcactgcaactacccctcctgggttcaagcgattctcctgcctcagcctcccaagtagctgggactacaggcgtgcaccaccatgcccagctaattttttttttgtatttttagtaaagacagggtttcaccatgtgggccaggctggtctcatggcaagttttctttgtgttgtcatgttattatcaattaataggaatttatatttcagttctgttaggtggataaacactattttgcatacctaaatgtttcatttatatcagcactggccaataaaaatatactataagcaggccgggtgcagtggctcacgcctgtaatcccaacttttgggaggccaacactttgggaggacacagggtcaggagatcgagaccatcctggctaacctggtgaaatcccgtctctactaaaaatacaaaaaattagccgggcgtggcgggggcgcctgtagtcccagctacttgggaggctgaggcaggagaatggcacgaacccgggaggtggagcttgcagtgagccgagatctcgccactgcactccagccttggtgacagagcaagactctgtctcaaaaaaaaaaaaaaaaaaaaaaaaaaaaaaatatatatatatatatatatattacaagccacaagccacatatgtacttttaaatgtcctagtagccatattagaaaacaaaagtaaaaaggaatagatgaaattaattttaatgatttttttaaacccaagatatccaaaatattatcattttaacatataattaaaaatttattgagatgttttacattgtttttttttttcttgacgctgtcttggaaatctagagtgtattttacatttacattacatctaattcagtctagccacatttcacatgctcatttttttgtgtgtggttttatggagcagagagtttaataggcaagaaagaaaagagaaggcagaagaaaatggctcccctgtacagagacgcgggggtggggcgctccaaagccaaaagaggaggtccctaagtatggtagacaccagccaggaatatatgcagtgtctggaggaggggatgtctgatttgcatagggtcacatgctcatttttatggctactgtattggtcagtacagatttagatggggatttgctctgaacaagttggtgattgatggtgttcatattttaattgaatttttcctgggttctgctgtacacttgtatgtgtgttagtttcatgtgcaatgcttgtgtcacttttaaaacccagatatatggcataacatgagaatgaaaaatggaccagaaaaatagtttggcaatgtagtcatgtttgttcctattaaatgttccctattgaccactctatctcttttaattataacaagaatctgccctgccagcatgcccagttacgctgggaaaacttctgcctcatttactctggctgattctcatccacttatgggtcagtggttcattttctagaggtcaccagcattcatacctagcatacaatttcattcattataatgaaggaatgttttcccttcaaagagacacaactagtgggcttaatttttcttgatatgtcacctgtaaaattttaatgatgatgtttaaactctaaatgtagccatcaagacaaaaactgcaaattttgagcctcagtgtgtgtgggtgggtttctgtttcggtaatttgaaacattgcagaatatcatcaaaatatgatacccaagaatcatatggtatcaatcattcctagatatactgatctattcattgccaagatagttcaatgagctggcaaaaacatatggaattattttcttaaaatgtgaaaaataaaatttaaccaatcatgtatcacagcttgcaactttagtcatactttgaaaagcattttaatttggacctcatgattgaaaatttataaaaagctgaacagaaattagttcacttcatattttagaaaagcagagtttctttactaaatgaggcatttgacccaaattggagagaaaatgttgaaactacttctgtgagcaagcaggtggcttctcaaacacatgttgggatgaaatggttgggcctcagggtctcagtgcctgtcactgagagttggcactctctatctccatggtctcctccaagtgtgactcttgtctcttgctgacctgacctgccccaagtgactcactggtcatgaccctgcacaccttgcgtctctcctatcaccctgccgatggcagagctacaaaggtctttgatgtagctctgtctgatatcctgtgtttccccctatggtctgtgtggaagcaggtatgggggtgtgtaaaggggaagcctatgaagttcatctgcaaagactacctggttaggggaggagaggaagaagctatatgcaccatttcaccagcaagcatgggctcttctgccttttagcttaggggtcctgttgtctagtctcactcacctattaaaacagtccagcaaatagaggttttgtttacctcccattaaaaaaaaagcaattaatggaatagaagataataatgtatgagaagcactattgtgaaagaaaaaccttcaacttctctcagccaaataattgcttcctcctctgtccttcccagaccttgatgtttgctctattatttcaaaacaactatattataaatatttgagaatgtgtatttccctgcaaggagatcttaatccccaagaaggcaggagctgtgtattattcatcccagtgctccttcaaaccagggcctcagacagtgcatggcccaaagaggtacttaataaacgtttggtaaactgacactattgaaattaagcaacctggatttgaggtgggtctctgccactcacaagagattacgctttgagaaaattccatcacttcattgattttcagttcttgcatctgtatatgggagacgatactaggtgatttctgacatctctcaccgtttaaatgctctgtgatctatacaacgaggggctcgctgttctagacaagttccttccagctttacagttgcataacccttctaatcttagtcacatgatgactccactgacagatttttggccaccatcattagacatgctgagttacgtgtgcctttgctctgatcctcaaaactcatgatttttaaagttttctgaaatatctaccatttatcaggatccagatggatttcatgaccaaagtggatgtttcttttctctcccattacaatcttttactttttgtgtgggaaattgcatgttaaagaaagggaaattgaagaatgggatgctttggaattctggcaagatggattagtgggttccagaaagtaggggcagccacaaataccgaaataaatgagatcgtattattgagaaagcacaaatggaagaaggtcaaaagcaagaagaagctgacatcctgcttcctccaatttttgctttctctgtttttccaagaaactcctcttccaagccttgctgaaaaactccactttcctaaatctaacttcttaaactgataatggcaagaagttaggaatgaccaatatgtttaacactccaagagtatttgttttgttttgttttgagactgggtctcactctgtcgtccagcctggagtgcagtggtgtgatcacagctcactgcagccttgacctctcctgcccaaggaatcctcccacctcagcctcctgagtagaagggaccacaggcatgtgccactacacctggctaacttttaaaaaatttttttgtagagatggggttgcccaggcaggtctcaaactgctgggctcaagcaatcctcctgcattggcctcccaaagtgctggaattatgggcataagccactacgcctgacctctccaagggcattctttacccagaagaggaacttggcagaacttatcctccaattggtgaggaatatggagaaaatgactttaagcaaaggaacttctggttctgcctacctaatccagaaaaagaagttttatttctcccttcccctagtaactatcttcccatattcacataaaaaagtacagaatcaacattgttcaagaattataattttacttgtaagcacatgtgcacacgcacacccatataccttccttccctttaaatcatcccacaccctaatagtagtaaaatcattgacccgagcatacctgggagaggaagaggagtctgacaggggcaggttctaagtggcactcctggaacttaaccctggtgtatatgaactttacctattgaaggatgactcctcaactgttctcacaatttgctgctctgctttcttttctaatttctgaaggtgactcatcttccccaaggactttcagacttctcagaagaaaaaaatattgggtgggtctctgccactggcaaaagattagactttgagaatcataaaagtatatcagtatatactcattaatattgaattactataattaatattatgatattgatataatgatagaatgatattgataaaagcaatattcaataatgaatattatttcagctgcccacttattgggtgcctcataggtgccaggcattttgtatgtattatctacaacccttacatgggacatattatgatgctgtttctcttgaagaaatatggaaactggaaacagagaggtcaccacaattttccaaagtcacatagctaataggtagcagacttgggattcaaattcatatgcatatggtaaatcatgctcttcctctgctacattttgcccccttagaatatgaaaaagggatacaaagagatgaagaaaatatgtaagattatccttcaatttcactatcttttttaaagttttttttattatactttaaattctgaaatacatgtgcagaacatgcaggtttgttacataggtatacacgtgccatgatggtttgttgcacccatcaacctgtcatctacattaggtatttttcctaatgctatccctcccctagtcccccacccaccgacaagccccggtgtgtgatattcccctccctgtgtccatgtgttctcattgttcaactcccacttatgagtgagaacatgcggtgtttggttttctgttcctgcgtcagtttgctgagaatgatggtttccagcttcattcatgttcctgcaaaaggcatgaactcattttatggctgcataccattctatggtatacatgtgccacattttcttaatctagtctatcattgatgggcatttggcttagttccaagtccttgctattgtgaacagtgctgcaataaacatatgtgggcatatgtctttatagtagaatgatttataatcctttgggtatagacccagtaatgggattgctgggtcaaatggcatttctggttctaggtccttcaggaattgccacactgtcttccacaatagctgaactagtttacactcccaccaacagtgtaaaagcgttcctatttctccacatcctctccaacatctgttgcttcctgactttttaatgattgccattctaattggagtgagatggtatctcagtgtggttttgattagcatttctttaatgacaagtgatgatgagctttttttcatgtttgttggccgtataaatgtcttcttttgagaagtgtccgttcatatcctttgcccactttttaacggggttttttcttgtaaatttgtttaacttccttgtagattctggatattagtcctttgtcagatgggtagattgcaaaaattttcttccattctgcaggttgcccgttcactctgataatagtttcttttgctgcgcagaagtttttttagtttaattagatcccatttgtcaattttggcttttgttgccattgcttttggtgtttagtcatgaagtctttgcccacgcctatgtcctgaatggtaatgcctaggttttcttctaggatttttatggttttaggtcttatgtttaaatctttaatccatcttgagttaatttttgtataaggtataaggaaggggtccagtttagttttctgcatatggctagccagttttcccaacaccatttattaaatagggaatcctttccccgttgcttgtttttgtcaggtttgtcaaagagcagatggttgtagatgtgtggcattatttctgaggcctctgttctgtttcattggtctctgtatctgttttgatacaagtaccatgctgttttggttactgtagacttgtagtataatttgaagtcaggtagcgtaatacctccagttttgttctttttgcttaggattgtcttgactattcaggctcttttttggttccatatgaaatttaaagtagttttttctaattctgtgaagaaagtcaatggtagcttgatgggaaaagcattgaatctataagttactttgggcagtatggccattttcatgatattaattcttcctatccgtgagcatggaatgtttttccatttgtttgtgtcctttcttatttccttgagcagtagtttgtagttctccttgaagaggtccatcacatcccttgtaagttgtattcctaggtattttgttctctttgtagcaattgtgaatggaagttcactcataagtttgctctctgtttgtctgttattggtgtataggaatgcttgtgatttttgcacattgattttgtatcctgagactttgccgaagttgcttatcagcttaaggagattttgggctgagacgacagggttttctaaatatacaatcatctcatctgcaaacagagacaatttgacttcctctcttcctatttgaatacgctttatttctttctcttgcctgtttgccctggccagaacttccaatactgtgttgaataggagtgttcaccacctattttaagaatagtattgaagcctcacaaaagctggttctcatgtaaccatctgagaatatttggccttatgacttgaattcattcattgcctttttatttcacattttagtgatcctgatgtctaaatcttaatctttgatccttgcaaggtaaaatagccaagtcaagcctgtttaataatattggttgaggaagtcacatgcttatgatcaatctttgggttatgtaattatattaccttaatgttggcagtttaggtgtaaggcagagatatctgatcacatgtgtggttagctaatttaagatcactgccaactaaaatcttcatggtatgatcttcaaagttagctactttgaccacagcaatgatttcaccacagcaattaacaaaatggcagactctttcctgaggtggcatgaacagttttaaaacaaagtcaaggaccaaaagaaaagcaggcacatggcatttgattcatttcaaaaactagtattgtattaagagccaaggggatagaattgtagcatcaattaaaatcttgtttgaaaaaaaataaaaacaaacgtccatttttatctctcaaatatattagggttttcataaagttataggtgtatttttaaaaaaacaaaactcatatacattagactgaaaaatttgcctgtcatctcatcatgcagctaaatgcaattgtctatggcgagatacactcttattagaggtatgatagcactaactaatagcaaactttgtacctggtagtctaatttatgcagggttcatatttcgctccctctcagcatgctgtaactgtggcaaagccattctcaggagttattaccccaacataatcatacccctgtggattaggagcagttaaatgggtcctgttatcagagacacatatgtgccacagccgctgtcatccctaagccaccgtgggtgattaagactcactgatgggactacctctgaataggcttgagtgaggtggatacacttcagctgagagaaattcaggtaaggggctgagaaaatcagattttgaatggttttatcataccatcaggtctccttttaagtgctggggtcatggatttcattcaacctgaccacatagcctttggaagcttggctcaatacctgagtgtgagattatgggtgatattaaaggagatgaatgcattgagctgatgtcagagaatgtcttttactggattttcataatgactggctgcagatgggctgaggggaaagtcagatcaagagcatttctgtaagaagaaagaaatcttccctcttattctctttcaagaaaatgaatagctgagcaccaagaggccaaatacttttttaaaaaacacatccttttatgtagagaaggacaggttgagacgaaaaacaggacctctgaaactgtctttatagctttaatctaggaagaaattgcggcaccattgctgacatcattatcagaggctgccttagttctgagagcttcacagatggccttttctgcattttacatctggccagatgaaggcaaaaaggttgatgaaaccaaaactattagatcagtggtccataactctggctgcactttagaatcatctggggagtccttaaaactactgatcctgaggctccatcccagaccaattgaatcaggatctctgggggtgggacctgagcattggaatgctttaaaagttcctcagggactctaatgtgcagccaaggctgagaatgactgaggtggatggtggccaagacaggtgaggccaagagttagaagcccttactgttagggaaggcagaagccacaaggaggaggggagggggaaggagcagtatttagcatttcttccacacagttggggggttttctgatcaaaattaggctgggatctttcgcttccattttcatgaaggtcttatgctttgtccacagccacctggcctcagggcaatgagcaatctgattgatgaattttcagtaagaaactgagcacacttggctctcagccccagtggcttcccctgtttccaaatctgcccaccagttacaggagcctgctcaccaactggttgggttaaagaagtcggctctgtccttggcagggaggccttcagctgtctggccctgtctgtgactgcgtgggtgaagctgcctaatttggggaacttgatggaagatctaagctatgttctctaacagttttatcagaaataaagttaacttttgacctccatctgcctgtctcctgtggaagggctctgctccttccaaagagactctcaggggttctccttagaggtgtgttatcagtccaaagatcattttagaccagccattacagaggatgtccaagaaattgcacaagggaaatagaagtaagaatgagagcaatatatcagatagtaaggaaagtatgtgactggtttcaaataagtaattaaccataaatccaaagttcctgcattagttatagcaatagtcatccacctggtcaggaaaaaaaaagtaaaagactgagctgcaagatagaaagtttgcctgaattccacgtacttcaaggactactatggaggattccttggtcccaatgcagagacatgtactctgaccacccatgggccaaatccctcttacccaccctcagtgattcctcctgggacctcactacattgagttcttacattccccactcttttcgggggaaatataacccttctgctcttctcatgatgttaaaaatatttagacaattacttaaaatttacaacaatcttccacataaatgatctcacttatgtttcttgtgagctctgtgatttatttctattatcatctatgctgatagttaaggaaactgagatatcgagaccaacatgcatagtaaatggcaaaaccagatgaattttaaactttcctaactccaaaagccacatcctgcccaacccgccatgctgccgctcagttaatgcctggctgtttgtctccccattggcccctctacccattgtgctttgatggcacactgtattccaactgcctgagactcctggttaatgccattacgtacagacttaggttgaatttactaggatttttaagatttgtaggataagagatatgactgttagactggaatcagcaatagataaaaggttaacaaagtttcagaataaaatataatagaaaaccccagcagatagaagtaaaatgaatggtaagacaactgaaatgaaagccatacatttagaaatatcaaataaaacacagattaatggcagacaataaaggaacatacttagcagttagtaaaaacactttttaccttattcttattaccctccagtaaccttttttttttttttttttgagacaaagtctcgctgtgtcgtccaggctggagggtagtgatgtgatctcggctcactgcaacttctgcctcttgggctgaagagattctcctgccccagcctcctgagtagctgggactataggggcccatcactgcacctggctaatttttgtatttttagtagagacggggtttcaccatgttggccaggctggtcttgaactcctggcctcaggtgatccacccgcctcagcctcccaaagtgctgagattacaagcgtgagccaccaagcccggctgtaacctattgaaaataacattacttgacatgtgagacaaattatttgtaagttaaagagtttatgtgccttcaatgctcccacccttccctcccctaaaaagattattgagtgcccatgatgtgcctaagccttctggtagactctgagaatgtgaagagagttagaggatacttgttcaacaacccagaatctagcagaagtgttttgaaatgaccagccacttgggagagctatgctagcatatcattcaggatgggctgggtcatgttttaataacaagtaagtttgaactttataggtttgaaacaaaaaagatgtgtttcttgctcacagttcatattttttggagattggctggagcttcattccaggatactgccaccatctggaatgttgacagttaccgtaacagagaaagagttgtggaggctgtcacactggcaattaaattcgctgacctggaactgacacatgccacctccacttatattttattggccaaggcttgttacacagccacatctaacttcagagaggtcaagatgagcaaatcctaccacgagctatagatggatgagtagcactcattattatcacaattattttatttgttataaaaactccaagaaggagaggtactctatgtgaggaataggcataggaaaatcagagggaagttcttaaagatgagctagatttcactagatggcattgaagaaacattccaagtaaatgaacagcataagcaaatgcatgaagaacttattgtggtcctagtacagacgatgcgtgagagtgtggaggagggaaaaggtaagactggagaggaaggcaggaaccagaacaggacagactgtgttcactgtcaggcagttaagtctatcttgtaggcaacatgaagccttttaaggaaggcaagcagctatgtgacaggacagaagatggattggaagaaaatcaaaagagaagtggggaccagtcataaggctcctccaatatttggggatccaaaccaaagcactggcagagaaatagaaaggaagggaaatatttccaaaatattcaaaacagaaaccaagagaacttgatgacagaagatgaacccaggtttctactgaatggacagttgttacattctctgagataaggaatacagaaggaaaaagttgagagggaatatgaaattatttttaatcatgtttaatttgatcacttgtggaacatcaaccagagatgtccatcagttacaccaatttgtaggttttgagagaggcctgaggtagaggcagagaagtgggaatcagcagattagcggcagtagttggaaccataactgaagatgagatttcccagggaggggagttgaacaggaaggtagacaaaggctgagtgcagtggctcatacctgtaatcccagcactttgggaggctgcagtgggtagatcacaaacaagatcaggagttcaagaccagcctggccaacatggtgaaaccccgtctctactaaaaaaacacaaaaattagccgggcgtggtggcacacacttgtaatcccagcctcaggaggctgaggcaggagaattgcttgaacccgggaggcagaggttgcagtgagccaagatcgtgccattgcactccagcctgggcgacagagcgagactccgtccccaccgaccccctaaaaaagaaagtagacaaagatgtgtcctagaaaacactaatatgaatgggtagagaggggagacttgtcaaggagatcgaggggagagtcagtgaggtgaaaccttagaggatagaccttcaccaaggaagtaacagtcaacaggcctaaatgccacagagatgtcaaatgagagacactggaaatggttctttgaatattacagccagaacatcacaggagaccatttccaaagcagtcttgatgaagtggtggggaagggaggtccctgaaggagctaaggagggactgggagatcatgacccagagataagtgttgcaggtataaaagggaaagactgagatcaggaaatagccacaggatccctaaggccaggcttcgggtggggtggtgtgtgtgtgtgtgtgtgtgtgtgtgtgtgtgtataaaatgggaagaccttgagtataattctgtactaagaagatagagcagtagagaggaaaaggctgaagacagatgggagggtaaatagtgaattagaaaggtctgtgcaaagatgagaaaggatgagtctgaaagcacagggagaagggccagccttggacaggagaaaacatttctttcactaagaataaagggaaggttgggtatacaccacaaagaaggtgtagatgggtggcaatggagttctgtcaagttgagggcattccatgatagcctcacctttctctgtgaaatgagagagtttagtttacaagatatttctgagcacttcataagccaggttcatagactgaaagaagtggaggtggagtgtgataggtccttaagaataggggaagtttggaatatctgacaagggacagagaggaaaaaactagaaaaggctttgcagaatgtgggcccacagatcagaggctaaggggtccccatttgtgcaggagagtaagggcagggaggcaaggctcacagcccaaaatatagagcccctcaccaaatgactgcaggagggcagctttcctatgagagcatccctatcactgttttcactccgagtcattaacttacgacttactcagctctgtttcgtaatagcagactcgagtaatgagggtatgacagcctctctctgcatgccaaggtatgcagcgtggatttcctttttcgctttctctctcctgtggcttaggtgccttctgttctgctaccaggatagagaacccagtgactagtttcttctagctctctttttctgactaggtatcttgtcagaaatttctgcttaccagacttcatggagagggaatcaagctttgaatcagggttgaaaaagtagagcttaatatatatattacaaaatgccactcacgttcttgaggttaccttgtatctataccacaactagcattcttttagaaagcaccattaccgaagtaatccctttcctgggaattcacccaaaaaggttattcccacttatcccccatctccaaaataaaaaagaaaaatgtgtgtgcttagagatgttcctggaagcatgagctgtaatactgaaccaatagaagacgaactaacagattgcagggcatccgtttggcaaaaaacttatgcagttatctaaatgatagttatgaagacaatatgtataacacattatattctgagtgaaaagaacagaaggtgatttcaaaactgcattgggataatagtaatataggaaatagtgagatgaacaaagatttcaaaggaacaaaaataaagaaaaattttgctttttatattggtaggcgtatgggtgaaattccaattttaattttaatttcatagttataaaattgttcataaaaaaaggtcccccatagacagttgggctttgggacaaactaacagaaacagagtaggaagaaaattcatcttccttcaatcccctttctctgcttaaaacaaaacaaaaaagagctttgtcatgttcaggtgtgcaacgaattctttttccaaatctggaactttacatctgctattaaacaggagtcagtttccatgtaacatgttgacaatcccccaagtgtgttggaataattttttttaatgaggagatttgaaattccatttcaattgccaacctgcctctttcaacttctaaaaacaaagtaaaacaaaacaaaaacacactgggtcctatcaccccctcttgctactactattttatctccattgccctgaattctttccaaacttctttccacccagctttgatttgttttcagtcgggtttattgaggcataatttacgtacagtaaaattcatccttcttagatttagaggtctatgcattttgactaatgcatattggcttataatgactaccacaacaaagatatagaacacacccatcactcccgggttctccctgtcccttttttggtccatctcctctcctacccccaaccccttggcaaccactgatctgttttctgtccttatcattttgctttttccaggatgttgtatataaggaatcatgcagcatgcagcctctcgagtctgacttcttccagttagcacagttatttaagatctatccgagttattgtgagtagcagcatcttttttattgctgactagtatttcatcacatggatgggccacaacttgtttatctgttcacctgtcaatggatactaagttgtttccagtttttggcaaatatgaataaagtaaacatttgcatacagatttttgtgtggacacatgttttcaattctcttaggtaaatactgagcaatgggattgctgggttatatgttaagtctatgttcagttttctaagttctgaaacaattggatatctatatgcaaaaatacaaaattaaccttgactcaaacttgtaccatacacaaaaaataacctgaaagagatcacaggtgtaaatgtaaacctagaactaaaaacttcaaggagagaaacatagcagaaaatatttgtgaccttgcattaggcaaagacttcttagatttgacatttgaaacatgatacataaaaaatcttgataaattggagttcataaaaataagaactactcttcaaaagacactgataagagaatgaaaatacaagccacagacagagaaaatatttgtgaatttcttatctgataaagggtttgtatccagcatgcataaagacttttcaaaactcaataataaacaatccaataagaaatgcacaaaagatacaaacacatcatcgaaaatgacctatgaaaggcaaataagcccacaaaaaaattctcaacatcattagacacttgggaaatgcaaattaaaacaacactgagataaactacatatctattaaatggctaccactttaaaaacctgtcaagtgccagccagaatgtggaacaagtaggactctcttacattgctagtgggaaggtgaatggtacagccactttggaaaacactccgcagcttcttatagttacacataggacctgggggtagaggatggattgactgtaaaggggcaaaacttcctggtaggggtggggaaagtttttagaagggtgaaattgttctatatcttgattattgtggtggttacacaactgtctgcatttgtcaaaactcacagaactgtacactaaaaaggtatatttttatgctctgctaattttactttaatcttaaaaataggaaggaaaaaataaaatcaatgccactgtgcgactttgggcaagttacttcacttctctgtgccttggtcttttgaaatctatacattaaggataaaataataccttcctcatactgttagaattaaatgtgctaatttatgttttatatatataaagtacttggccgggtgtggtgactcacacctgtaatcccagcactgtgggaggccgaggtgggcagatcacttgaggacaggagttcaagactagcctggtcaacatggtgaaaccctgtctctactaaaaatacaaaaattagctgggcttggtggtgcgtgcttgtaatcccagctacttgagtggctgaggtgggaggatcacttgaactccagaggtggaggctgcagtgggctccacccactgcctgggtgacagagctagactccatctcgaagaaaaaataagtacttgaaacatagcaaatgttttataattattggcttttttttcttattgttattacttgcattattgctgtttgaagaagtttggtgataatggagagaaaagggcaattaggggtctgggatggtttaagtatgaggagaccgagacacattgactcagagtgaagaaatcaaagataagagaatgaaagaaagggagggtaattctgaacgcacagacaaagttatgttactaacgtggcatcggctgtgtgttgtaataaataactccctttcacttgtcaatagctaatcaaatactttctggagaccagaagtgacttgctggatcaatgacaactcctccacagatcaaaatgttcaaatcctttttctgtgttgtaatcctaaatctaaaagaacagagagaccaagcaaatctacctcccaacatcattaaagtgacaactctcagtatttatttgaatggtctgctctcagcttcaaccaaggaaaagtcaaattagtgttggtcagaaaacagaagggtgttacgagagttctggctggtcattacagacttggggatttttgattaaaagaagaagaagaagaagaaacctgataaagtgtaaatatagcaagcagggattagtgtcctgctgggtcatgttctcacaacagtgagaatttcagagatttcataagaattaaactgctccacatgacaatttattttaccttctggcttttccaggaggcaaatcagtgcaacttctttctgcctttgtttcaatttggtaacaaccctcaattttaggacaggctaaacctagccaccctatcagagatgatgaagtagccatctttttaacaggtggggagatgaatggaatcagggtttgtttgtttgtttgtttaataactgctagtaaaaaccaagtcaatagctgactgagtgtaagggaggctccagaaggcaggttattgtagtatagatgtgactcgacttatgatgatgttacttcccgataaacccgtcataagttgaaatatcgttaaattgaaaatgcttttaatacaccgaatctaccgaacatcatagcttagctcagcctacgttaaatgtgctcaggacgcacattgcctacagctgagccaaatcacctggcaacacaggacactgtagagtatcggttgctggcccttgtgatgctgtgactgactgggagctgcgcttagtgcctctacccagcattgagagtttcttatcgcttattactagcctgggaaaagaccaaaattcaaaactcaaagtgcggtttctaccgaatgcttataactttcagaccatcatgatgttgaaaaatcgaaccatcgtaggttgggatccatcctataagacgagctacactgccggaagtgtaagactgctatgctgccggaagatggggcatagtggacaactgcaagtcctgacaacaggaggtcagcatctgcgacctttaacatccacattgacactaccacagtcttccaaacagagctgatgatatagtttggatgtcgtccctgcccaaatctcatgtcgaatcgtaatccccagtgttggaggtggggcctggtgggaggtgattgggtcatgggggcagagttcttatgaatggtttagcacggtccccccttggtactgtatagtgagtgagttctcatgcgatctggttgtttaaaagtgtgtggcacctcccctctctctctttctcctactctggccatgtgaagtgttggctcccgctttgccttccaccatgattgttaaattcccagaggtctccctagaagctaatgctgccacgtacagcctggagaactgtgagccaattaaacctcatctctttttaaattacccagtctcaggccgggcgcggtgactgacacctgtaatctcagcactttgggaggctcaggcaggaagatgatttgaggtcaggagttcgagaccagcctggccaacatggtgaaaccccatctctactgaaaatataaaaattagccaggcatggtggcgggtgcctgtaatcccagctacttgggaggctgaggcaggagaatcgcttgaacctgggagtcagaggttgcagagagccaaaatggagccactgtactccagcctgggcaatggagtgagaccctgtctcaaaaaatatatatatattacccagactcaggtatttctttacctgagactatgagagaatggactaatatagctgaagaattttattttatttttaaaaaacttttacgtttgggggtacctgtaaaagtctgttacataggtaaactcctgtcatgaggatttgttgtacagattctttcctgctcctccccctcctcccaccctccatcctcaagaagatcccagtgtctgttgtttccttctttgtgttcgtaagttctcatcatgtagctcccacgtataagtgagaacatgcagtatttggttttctgtccctgtgttagtttgctaaggatgatagcctccaactccatctatcttcctgcaaaagacatgatctcattcatttttattgctgcatagtattccatggtgtatatgtaccacattttctttatccagtctgtcattgatgggcatttaggttgattctgtgtcttcagaattgtgaatagtgctgcaacgaacattcgtgtgcttgtgtctttatagtagaatgatttctattcttctggtagtaatgggattgctgggtcaaatggtcgttctgcttttagctctttgcagaatcaccatactgctttccacagtggttgaactaatttacactcccactaacagtgtataagtgttcccttttctctgcaaccttgccagcctctgttatcttttgactttttaataaaaaccattctaattagtgtgatggtatttcattgttgttttgatttgcatttctctaatgatcagtgatgttgagctttttttcatgttcgttggctgcaggtacatcttcttttgaaaagtgtctgctcatgtcctttgcccactttttaatggggttgtttttctcttgtaaatttaagttcctcatagatgctgggtattagacctttgtcagatgtatagcttgcaaatattttctcccattctgtaggttgtctgcttactcttttgattgtttcttttaccatgcagaagctcctaagtttaattagatcccatttgtcaatttttgcttttgttgcaattgcttttggtgtctttgtcatgaaatctttgccaggtcctatgtccagaatgatattgcctaggttgtcttctagggtttttatagttttgggttttacatttaaatctttaatccatcttgagttgatttttgtgtttggtgtaaggaaggggtccagtttcaatattctgcatatggctagccagttatcccagcattatttattgagtaaggagtatctcctccgttgcttgtttttcccaggtttgttgaagatcagatggttgtaggtgtgtggccttattttggggctctctatcctgttcatttggtctatgtgcctgtttttgtaccagtaccattctgttttggttactgtagccctatagcatatttcaaagttgggtaacatgatgcctccagctttattctttttgcttagaattaccttggccatttgggctctttttggtaccatatgaagtttaaaatagttttttttctagttatgtgaagaatgtcgttggtaatttgataggaataacatgtaatgatattgattcttcctatccatgagcatgggatgtttttccatttgtttgtgtcttctctgatttcttcaagcagtgttttgtaactcatattgtagagattattcacctccttgcttagctgtattcctaggtattgtattctttctgtagtaattgtgaatgggattgcttttctgatttggccctcagcttggtattgttggtgtataggaatgctagtgattttttgtatcctgagactttgctgaagttatttatcagctgaaggagcttttgggctgagactagggggttttttagatatagaatcatgttctctgcaaacagatttagtttgacttcctctcttcctacttggatgccctttatttctttctcttgcctgatttccctggccaggacttccagtaccatgttgaataggcgtggtgagagagggcattcttgtcttgtgccagttttcagggagaatgcttccaccttttgcccattcagtacgatgtttgtggtggtttgtcatatatggctattattattttgaggtgtgttcctttaatacctagtttattgacagtttttaacatgaagcagtgtttaattttattaaaagtcttttctgcctctgttgagatagtcatgtggcttttgtctttagttctgtttatgtgatgaatcacatttgttgatttccttatgttggaccaaccttgcatcccagggatgaagcctacttgattgtggtggtttagctttttgatatactactggattcagtttgcaagtattttgttgaggatttttgcattgatgttcatcacggatatcggcctgaagtttctttttttgttgtgtctctgtcaggttttggtatcagaatgatgctggcctcctagaatgagttggggaggagttcctcctcctcaatttttttggaataggttctgtaggaatggtaccagctcttctttatacatctggtagagtttggctgtgaagccatcaggtcctgggattttttagttggtagggtatttattactgattccctaaatagaccgataatgattttagaagtggagtcggttttttcctggtccagtcttgggaaggtgtatgtatccaggaatttatttagctcttctaggttttctagtttgtgtgcatatgggtgttcatagtagtttctgatggttgtttttatttccgtgggatcagtggtaacattctcttcatcatttcttttttttttttttttttttttttttgagacggagtctcgctctgtcgcccaggctggagtgcagtggcgcgatctcggctcactgcaagctccgcctcccgggttcacgccattctcctgcctcagcctcccgagtagctgggactacaggcgcccgctaccacgtccggctaattttttgtatttttagtagagacggggtttcaccgtgttagccaggatggtctcgatctcctgacctcgtgatccgcccgcctcggcctcccaaagtgctgggattacaggcgtgagccaccgcgcccggcccatttctaattgtgtttatttgaatcctctctcttctcttctttattaggctagctagtggcctatctatcttattaattttttcaaaaaaccagctcctggatttcttgatcttttgaatggtttttcatgtatcaatccttcagttcagctctgattttggttatttcttgtcttgtgctagctttggggttgacttgttcttgcttctctaattctttcagttctgatgttagtttgttagtttgagatctaactttttgatgtggacatttagtgctataaatttaactcttaacactgccttagctgtgtcccagagagtctggtatgttgtatctttgttctcattagtttgaaaaaacttcttgatttctgtcttaatttaattatttatccaaagtcattcaggaacatgttgtttaatttccatgtaattgcatggttttgagcgattttcttagtcttgacttctatttttattgtaccgtggtctgagggtgtttgatatgactttggttcttttgcatttgctgaggattgttttatgtccaattatgtggttgattttagagtatgtgccatgtggtgatgagaagaatgtatattctgttggttttgggtacagagttctgtagaggtctattagatccatttggtccaatgttgagttcagatcctgaatatctttgctaatttcctgcctccatgatctaatactgtcagtaaagcactgaagtctcctactactattgtgtgggagtctatgtctctttataggtctctaagaacttgctttatgaatctgggtgcttctgtgttggatgcatatatatttaggatagttagatcttcttgttgaattgaaccctttaccattatgtaacgcccttctttgtctttttttttctttgttggtttgaagtcttctttgtctgaaattaggattgcaacccctgcttttttctgttttctgtttgcttggtagattttcctccatccctttattttggacctatgggtgtccttacatattttatctttatctatccatccagccatccagccatccattcatccgtatcatttttaaccaataaggacttttaaaagcgcaaccacaacaccattaacataaccaataaaatctataacaatgataaaatatcatctaatactcagtccatgtccaattttccctcgctatctcaaaatcgtcttcttagaaatggtctgttcaaatgagatcacatggacagaggaaggcgaacctcacactgtggggactgttgtggggtggggggaggggggagggatagcattgggagatatacctaatgctagatgacaagttagtgggtgcagcgcaccagcatggcacatgtatatgtatgtaactaacctgcacaatgtgcacatgtaccctaaaacttaaagtataataataaaaaataaaaataaaaaaataaaaagtgaaaaaagaaaaaaaaaaaaaaaagaaatggtctgttcaaatcacaaaccagattcagaaacaatagccatacattacattttattaatatgtctcttaaatttcttttaatctattacagtctttggaatttttatgtcttcgtttatccttccaattattaaaaaaaaagtatttttgtattcattgaatagacaatgcttgcagaaaagtaaaaaaaaaaaaatttagtacaaaaaggtacatagtgagctgttccttagtctcccttcccagaagcaatgttaccacttttgtacaaatagtctctgcctagacacacatgccagtccctaaggtggctgtaacaaggtggttaagagtgagaacatgaattcaaattcctattatgccactcactaagtataaatcttggtcatggtacatgcctctgtgcctcagtttttaataatggtacctacctcatagggctgttgagagaattaaatcagataagtgcttaaataactattaatatttattattattcacattcccttttggcttttttcccaaatagcagagtggtgcacatatgtcttcatttatttggcttgttttttcacctcacatcacattttgatgaataattccatacatgttgttatagatttgcttcattctttgtaatcattgactaatattccattgtatgaatatgctactgctaaacatgtacgttatttccaacctcttattatcaagaaatgctgcaatgaatatccttgtaatactttagtggattcatgtgcaaaaatattcataggataaaatcctgaaagttaaattgctgagctaaagggtatgtgcattttaatgctttatagattgcccagctgcctcaaaggaggttataacaatttacactcccaagaaaaatgcacaagggtccccatttccccataccctagctaacacaggatattgctaaatgctttcatctttgtaaacatgatgtattgaaaatggtatctcaaagttttaatgtgcatttttctgattgtgagaagataaaggaaatgtagtacaactaaacatcagcagtcaaatgacctggccatgactcctgagtgaggacactggtaaacaccatcaggatccaaacacctctgtatttacgaagaggatgctccctattggatagcactaagcttatttcatgtatgtacatatgtagttagttaattacatccagcggtggcaaagggcttgttctgacccaatgaaactttctctcctggcccccttccagcatgtggtcaggagtagagtgttgtggccatgaggcatgcatttgtacagatgactacttactcctccttgaaacatttttttccatttgcttccctgctgtctcactcatgggtctgctcctaattcacaaatcactcttttcccagtcttcttggttgggttttctcctcttctgtgcttgtagacatgggggagccccagggcttctctcttgaactacagcttctccctgggttcatctccttgggatgtctgttccaatgggtttaaatactagggctaggactagggagaggtcattgaggcacttagcacataaagtttaaggaaacattctttatcaggcccatgcaagtgcaggactggccctggagggtgactaccaccttaccttttccaccctaggcaccttgtttgccttaccctagccccagtcctctttaacaccccagtcttctccctggacctccagaaacataaatcctatttgacatttctacttggaggttttaaggtaactcaaacgtaaaatatctaagacagaactcttgcatcacttcccatcctggggcccaagcctgtctcttctactagtctatctcagttaacagcatcaccatttattcagttgctcaggacaaaaaatttgaagtaatccttgactcttctttttttttttttgagacggattctcactctgttgcccaggctggagtgcagtggtgggaccttggctcactgcaacctctgcctcctgggttcaagcaattctcctgcctcagtcttctgacctcgtgatccactcgcctcggcctcccaaagtgctgggattacaggcgtgagccaccgcacccggcctggattcttttttttttttttaaacaagtcctatcttccatctccaaaatgtatcccaaatctgacaacctctcccaccgtaggccagcccccatctctcccctctgaaaatagcctcccttagatctctggacatttgttcttccccacccccttgtgatcactattcagcattcagaatgatcttttaatattatgaaggagactgtgttcctctcctacttaaaattctctagtggcttcctaataaatttagaataaaaagccaactcctcgccatggtcaccaggccaggatccagtgggtacaacaatctgttcccagcacaataatcccacttctgcctccccaccattcaccaggctccactgcactggctcattcctgcctcagttgtgcttcttttccctggaaagttctttctgtagatctttaaagggtgatttccttctcaacattcaggtgtcagctcgtttcactttcctgaccatgcccatccactcagagatcactcaaaatcccattaccctattttatttctccatcatatgtatcactatctgaaactatcttgttgttgatgcaggctattttcttgaccccttcatgggactcccaacaggggtaccccatttactcagcctgccctgctcaacctcttgcaggagggagcacacgagtgaacgagtgcaggaaccagctggctgctttagtgctgtgaggagtaaactccatgcaggccctgcagcagcaaccaggtaggggtgcctgcaaccccagggccccagagggtgtgttacaatgctctcgtagctctgccatctgtggacagcagtgtgttgtcagctcagtgggccctttgcttcatcatgtagggtggctgccctctgcctgtgagggcaaagggccagggtgacagtctttttgggtacccacaatttgtgcatcctgaattcttgtttggtgcccaagaagaatggggtcacacagatgaactgaaggatggtgaatgcagagaattagcaatgaaagtggctctcagcagagagagaagctgaaaagggaatgggaagggcaggtcactctcccctgaagtcaagtcacatctctccaatgtccagccaccatctctgaagtcaagtttcctctctctgatgtccagccacttctcctctctactggctgagtctggggtatttataggcagaggataggtggtggggcaggccatacataattttggaaaaggcaacattctattggtaaaaagacattattcataaagaaccaattgggaaagagcgggcacacagggatggaagttctcactttgggctgcaggtttcaggcttttcagctcaaaagtgaggtttttccagggacctgccccgtctgcctaaaatttctacacttctgtcattgataccgctggataacagctctcctataaagttcaggggcttaaaacaaaaatttactaattcacccggacagttcttgatagggtctctcctaactgttgcagttacatagcaactgggttggagtcatcttttctgggcttgacatccaggacagcttcttcccttgtgtgtctggtgcctcagtgctcctccaggcagcctttctctccagaagagtagcctggacttcttggcaactcaagcttccaaaagaaaaaaaagcagagactgctggttctcttatcaaacaggcctggaactggcacaatgtacttctgctgccatattcaggaggtcaaagcaatcgaaggccaatccaagttcaaaggcattggagaaaaatgagaagtgtcacttaaatgagaagtgacacatgcgtaaaagggggaaaagcattgattgtggccatttttggagataagctatcacgtttatttgtttgtttgctttctaatggtctgtcttctcccattaggttataagctctgtgagacaacaggaatcttgtccatcttgttttatggctctacttccaacacctagaataatgcctggcacatagtaggtgctcagtgaataacttaagcacttgatacatgtttggggaactaaaatgaacagaattaaacttccccagattggtcctgccagatttgctgatgccaagcatgctgatgcctcaccagatgaaagaagccctaaaatgtagggtttcgctttctctgcaaacaagaaaaacttgccctgaacacaaaatctagaaatagatttggcgtgttttctacattgaaatatttcccgtagtaccagaaattattttcccacagctttgtgctacattaaaatattgaagttgactgaaaatatctccattctttaatcttggtgtagactagaaacaatttttttgtaacaaagtaaatatgaaaacttcctaatatttgaactccccagatatccccagatatctccaaacttaaaatatcattgcaagttaagataaatttttttaaatgactaccgagaaaggtcattaaaggcttggttattaaaatgtacagatttgggttataaagccagaacttatttgtttaaatcattacatatgaccaagcacagaaaataaattacctcaaatctcctctttgctaatttttactggtaaactctataaaatgatcctatctttaaacctttttgtaaaccccttataagttagtaagtgagaatgtattcatcagaaggattttagtgatgtttgaaattaaaaaagagagatttgatttttaaaattatacttgcagactactgctaatgaaacttcttctaaccctagttttgtcttatcttcagtttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcaggtaagtacaaaattctacctctgaagacaaatgtgcttttcaatatgtcaaaaagaccgtctacctaaatataaagttataatcttaacatatatacatggatgcacactgtagtattatacataataacaaaaagtgtggaaataccacacttgctcaacagtagggaattcaataaatactttatggacatctatatgaataactgtgatgctgacattaaattatatttttgaagatgtaatcaagaggataaacgcttgtctcaaaaagttacatgggaaaagcagtatgtaaacttatataaacattgtgaacctaatttgattatatatataaaatatagggaatatacatataaaatacatatatgtatatatgggggctatatatatatatatatgaaagagatagatagatagatagatagatagatagatagacagacagacagacaatacagactccaatctgttggtcgtggttgtctctgacccatgacactatgggggacttttatttttgctcatacttttcaatatttcttagtgttcaataatgtgctattatttatacataataataaaaataaataaatggcatcaaaaaagagtaaagggccagtgttccgcccacatatgagcagccatattcaagcctgtagacactttgtgtagcctaatgctaggtgtatctgggcaaggataaactctaaagccagaaattagttcatcaataaacatgtgctactcaatagctagggctgatggaaaagaatataaaacccagtctgtgccaaatggtgcttactatctgcaagtgggagaaggagaaagacgagaaaatgaaaaatgtgtgtataatttatatgtagctgttctgtaggagatctctgacttcaccccattctaactttgcaaaaagatccaacactttgtcagattcctgggaggcaagtaattttattgatggtttcatggagggatacagaacgataacaactcacacaaagcaaacaatgtaatgaaaatctctattcgactgtttctttttctcctgaagttgccctttggctgccagctaccaggcaccaggctcaaggtactttcttgctcttgacactactcccttctctcatacaattcaaccccaaccacaaacgtgtatagatctctctctctataaaacaaaggcctgtagttaacaggaggtcacttgcagtgtagcctctgttcattgttacttgtgcacactgcttagggtctcaccccatccacattctgctaatcacattattcacccatccaatgtagatctctccagtggagattctgctaatattttctttagatttgtcacaagtatataatacagttttaaattgtacagatgattatcctataacagaagagtctaagccttttacatctttgtatctctaacgaaagcattcagcatgaagctctgtacacagcagacaattcaatatgaatttgctgacttgaaacagcaagcctagaaaggagatgttaacttggtcacttagacagaacaggtttcagcaatcagaattcagatgacatggaactggtagaacaggcgctttgaagcaataggacatgagccagtgaggagagggatggaatatcataaacaaaggccaagggctttgcaatcagagctgaagagccaagagcacaggctcagggtgtgggcagactgaatgagaaagtgattcaatcacatgtgaaagtccagatgagaagagagagttgggattacttctgctcaccaaacatccaaaaccaaacaggtggatccgggtggtgtgctgttttactgatgaccattacacagaattttaacagaaggaatgtaaagcagtggttctcaaactggagttcccagatgagcagcgtctgcatcatctggaaacctgataaagcagcaaattctcaggccctaccccagacccactgaatcagaaacttgggggtctgggggaagatggccatctgtattttaacaatctcccttcaggagattctgaggctggctcaagtttgaactacaggtagttggttcaacaggtgttggtggactgacaaacaaaaagagactccgaggtaactccaagatggtaatgtcagaaagcagctaccacccctagggcttgggggaaccaacaaaagagtttggcattgccagaacctagaatcttgaggagaggccccagagcattgtgtctcagaccttgaggacttggcactgggacaccatgaggggtttctgggtgtgggaaagggctggaaactccccagttgctgccaccagggagaactacaagtgaagtggaaggtgtgggcctttctcccttttcttttctcgtcttctctctccccctggtgctcatgtttgacagaaaacagctgaaaaggcagaactagtttggggagtcttgacctggcatcataaagcagagaaaagcaaagctggagtgaaggtgagacacaacagctcattagcagcaacagccgtctagcgctccagcttctgaatgaaattctgaagaacagcgcacttggaagacaaattatttgacagttctgacagacgaccaaactaacagcatttgaaaagcaagatgactcagagaatacagaatttaatccaaatcccagatcctatctctgcctttggccaggcctaatgcaaggagaacctgagcacacaaatatatgcaggaatgatcaggacctgtgcctgcattctattctgtctcacccaccttcaaatttgttgtaaaaacatgggctcaataaaggtttgtgaatcagggaaggaagagaaggggagaaaggaagggaaggagccagctccagatctgtgtcttgcagaggataaaggccagtgtttttagatcacccagtgtttttctaagcccccaatacttattttgaaatatcaaaatgttcaataactaaaaaaaaaaccgttacaacaataaaacatgtttgagggtcagatggactggcagtttgtgacctctggggataaacaggtcactttggaatcacagacttcctcattccccttaaatctcatatggtacccagaagcccttggaactttggaaggtgtttattcacagttgtaatgtccatgcagaccctggctctaagacccaattgtgtaagggtaggtttgtagcccttatcccaaacattctaagtgtgagccaatgcgtcacacactcagaggccagagactgtattggggtcctttatttcacgtacgagtcacattccattaagagaccccagaagtcagctctcttccactgactggttctcttcccttgtttctcttgccaatgtgtgctgcccaggtggcagtgctcactgtcagcagagaagaaaaatgctttcctccttggacctcttttctctttttctcctccctactcacattcaggttccctaagcttccccgctccttgtgctgaagtcattctatggtcatttcttcaactgtctacttccctgctggatgggcacccaagacttggcatcctggggcatgtagaaaggggaaagggaagggaagggaaaagaagtcctcccaattgtctatctggacctttccacactgcccagagtactgctatgggcatctccttatgtctcccgatgtggtgcatgccagaccctgcaggtagaaaaggaaagaaagcaacccattggaccaggccagcaaaggcttcagtcacacagctggctcatacttatggcttcatattctgttgcctcttgaaccagacatttcttccactctcataacctccagtttagctcgtattcctcagcattctccatgtaatattgttgcatgaaacccatgcaagtcagccaatttgctctttctcatcttgtcatttataaattgatgctgagaagtctttttcccaaggtttttagtaataccttcatcatcccccatagttcattttgggcagtgattcgcttctttgactgtacattagaatcatctgaagaactttctaaaactactgatctcaggtctcacacaacactaattaaattatagtctctggtggggtagggcttgggcactgctattttaccttaagctctctggagtcattctaatttgtagccagtgctaagggttgcaatataagtgaatatatttcacgtatttgtcaaacattgactgagtgcccattatgtgccagccattataataggcactggtgatcccacagtgaatcaggcacacaatgctgtcttcacggagcttgttgtctagtgggagagtcaaacaaaagtgtatatcaataattaagtgattacagattgcaataattacaataagggtgataaacaggttgctataatatacaatagtattgcctttcaccagacatttcttaaagaggtaaatcatctatcagacaccttttaaaaatctcatctaatttcaaaagtgtacataaataattaagtgattacagtttgcaataattacaatgagggtgataaacaagttgctatgaaagagaaagatagcagagatctggctttgagatggtggtcagggaagactgctccaatagctgagttctaaagctaagaaggaactgagaaccttcacagaacgtcccaagtagaagagaaagcacactgaagactctagggaaagaggtctgcttgttgtaggaaccgaaagaaggccaatgtggctaggtgctgggtagtgaggggaaatggcacaaggaaaaaatgaggttagagagatcagttgataccagttaatgttggaccctaaacattaaccatggtaagtcttttagaattgattctaattgcaatgaaaaccttttgaaagattttaaaaagatatctgatagctgatttacctctctaagaaatgtctggtgaaagacaataccatcacattggagatggaaaaagatgaatggattctaaaaacattctgaaagtacattcaaaatgtttttcaggtagcttatgcaactaataaatagtggtggcattctgggtaagacaagggaggagcaggcttgcagtttagggcaagaaaggggtggggagaagagctcagcactaaaatcatgtgttccatttggggcacatcgagtctgagttgctatgagaccaccaagtggagatgccaagtaaatagtcagttacatgaatctggagttcagtgaagaggtctagaagaaagatgtatatttgggcattattcggatatagatattatgtaaagcaataaaattggatgagatcacctagggagagaatgcacatagataaaaactgacctaggaccacttcatgtctaaaacaccaaaagcaatgtcaacaaaagccaaaattgacaaatgggatctaattaaactaaagagcttctgcacagcaaaagaatcagagtgaacaggcaacccacaaaatggaagaaaattttcacaacctactcatctgacaaagggctaatatccagaatctacagtgaactcaaacaaatttacaagaaaaaaacaaacaaccccatcaaaaagtgggtgaaggacatgaacagacacttctcaaaagaagacatttatgcagccaaaaaacacatgaaaaaatgctcaccatcactggccatcagagaaatgcaaatcaaaaccacaatgagataccatctcacaccagttcaaatggcaatcattaaaaagtcaggaaacaacaggtgctggagaggatctggagaaataggaacacttttacactgttggtgggactataaactagttcaaccattggggaagtcattgtggcgattcctcagggatctagaactagaaataccatttgacccagccatcccattactgggtatatacccaaaggactataaatcatgctgctataaagacacatgcacatgtatgtttattgcggcactattcacaatagcaaagacttggaaccaacctaaatgtccaacaatgatagactggattaagaaaatgtggcacatatacaccatggaatactatgcagccataaaaaatgatgagttcatgtcctttgtagggacatggatgaagctggaaaccatcatcctcagcaaactatcgcaaggacaaaaaaccaaacaccgcatgttctcacccataggtgggaattgaacgatgagaacacatggacacgggaaggggaacatcacacactggggactgttgtggggtgggaagaggggggagggatagcattaggagatatacctaatgctaaatgacgagttattgggtgcagcacaccagcatggcacatgtatacatatgtacctaacctgcacattgagcacacgtaccctaaaacttaaagtataataataataaaataaaataaaataaaaaaacaaaaattgatgtaggaccaattcctgaagaacactgacagttaattttttggtttaggaggaggagaagccagcaaacgacactgagtagcaatatccaaagaaaaagaggaaaaaggaaaactgggagattatgagtgtcccagagggaatgtttcaagattaccatcagcagtgagctttgtgtaaaggtggcctcctgtaatagaggtgcgggcaggagaaggcagaatagggaaaagggggtgaaaaagcttccctcaagatttataatacagtggaagagagagacagagagagagaaagagaaagagagagagagaacttaaggaggtagaggaagagagagaaccaaaaaagagggagctgagtatagaagcaattagattcatagtttttagttgcggcagtgatatttgagtgggggccttttatatattccattctaggtgtttcccagttgatgggagagggtcttacctagatctgcatgtaaaagggagtaggccagctggcagacttgacatggatcagtggtagaacatcctagcagttctgtgaatactctctgagaatgacatgaaaggtattggttcaggccttttggaggtgataaaaaccaccaaaatgtggacttattgcaaattgtatttgttaccatttgcaatgattataattattctcttatatataggctttcttacatatagcttctcttacacatagcgtcatgagttatgccttctcttacatatagtgtctgctatgagttatgccaagcagggcaaacaaaattgctgcctttctttaaaaagaggacgctcctagtatgggcctaattaattatgataattacagctatggcatggaacataagcacattcatatacacaaagacaataaaataaagaacagttcaaaaacagaacagttacattatatatcagtttcagtatgaataataccctggactctgaaatatgtctggggcacattattctgtaattggtggtgaaaaaaaatctgcatcttatctctacgccaatccttatgaaggagctgtttttcaggagttcgagaaagagacacagggatgtccagtcatcaaagccgcagagcctgaggaagaataaaggatttgtggcaggaaaacccagtaatgaatgtattctgctagtttctcagcatagaacttagaaaagaggccacagaaggaaaagagaagtaatgttagacagctgatgttggcaatgggcaaagaatttcattctcatatgcagggcagtggctaaaggggcagtgttgtgaggaatcacattccaggtcattcatgtccagggtgtggtaggaggactgtgtttcatttattttgtacatggcccagctatgaccttgtgcaaggaaatgcttaatcattttctatcacagttgcaaagagattcttatcctactcagaatgtacgtcttctcttctgtttcatttacagtcacaacccaagtccttgctttgacctccaaagcaccacttgatgtatccacttcagaaacacacacagacagctcacctctttctatcccttaccttcctcccctcttcactcaaaccacctacttcctttgcattcactcttccttcagcctgaaataatcttcctccaaatatctaccttctcactccctcacttctctcaagatacacttaaatgttatattccctatgaggcctcccctggccatccctccccagccttcctatcccccctctctgctttattttattctccttaatatatatcacactctgataaaccattgaatgtacttatcaagttattgcctctctctccctttccattgtctccatcactgagagctctgtgaagaaaaggatttgtacctgtttcatttagtgctgtacccccagttcccacaacagcgcaacaggcactcaataaatagttgttgaataagtgaataaaacagaagtagctgcatattttctggtaacaaatgatattcttctgaaaatgtcatattttcagacatatttccgaaaataaattcaaattagataaacattgtatttttagaccatttcttctttgcattaatcatccttctcaataatataacatttgtaaaacttaggttagatatgggctcttcaactttccattacagaagataaagtgaaaaggctagacccaatggtgttattccttcatctacatctatcctttggaaacacatgaccaaattgcttgccatcacaatctcaaaatctaccctttggtattaactcacttcacttgtccctctgtccctttttagatggtagccatcggtctctggagcagtgtagagtcagaacaacttctatttggggaagaaatcattgctggtgaccttactttcaattactaactttctagtgacatttacataattttagagaaaattaacacctacacttgtaaagttgtggctttcccacacctatttatcatctctcaatattccttgaaaaggaaattatcaatttatcattctatattggcaatgaaatgcccctaatatctgtcacctataagacaattgaagatgatgtgttgaaagctttctgaaaatgctgatcattactttaaatggaattgaaattccagtttattatttccaaaaatatgatcttactgatcataggataacatttcataacatttcagagatttcttccccttcgaggagccaaacccataggacctctggactcccacagatcctggcagggagttcccactcataaaagcacaggtgccctcagagtcattcagggatgaagaagcaaccctcattggccatgtcctacgttccccatatagtaaggactggaggagaccagtgctcaattctgcagtctcagacagctgtcagaggagagtcatgaatgtgcagtgtctagcacattacaaacgtttgttaattgactgatcattcatggtgtgacagccctataactcagctatcctattcagtcagaaattaactcagtaatcaaagtcattaaaaggaagaaaaaaaaaacctacagtaccagacagatggtggggaaatcagacagatgaaaggaaaaatggctgtaggtcattgagtaagacactgggcagcaaaacctgggccttgtgcctggttatactccacattatagctccagcaaggtttggcaggatttccacagtcctggcttattctaacctttcttgggagcaggagcagtgttggtcagatagacagacacataaggaatctgtccaactggcaccgtgtgaatttgggctcttggtgtacatggataactgggaaaaagaggagagagacatgtaggactgatcctaccgtttgtgaagtcttgggcaagagtatgaatgaaaacccacttctcttcccctgcctggctccactgcacacagtaaagagcctcaagcataggtgtgtggacattgcaccatgtatccaagctctgaccatgcctcttgaaacagctattcctcagccaccctctgaccatgggaggaatgacccaggagaaatgaccacataggtcttgaaaatgggctcagggctatttacgaagtcaattccggggtcccaggagtatggactaaaatgtgagtcaggcatgccagatgggtatgttctattgacttcaaggattcctcatgctgtgggaaggaacctctccagaagagagacagagcagaaccctctaaatgtggggcacaaagcaggagcccctcttgctggattcaaagggtcatactggaagagtgtaggttgagtcttattctcacatcactcatatcacttacacacttcttttatagccttagcagccatgccacaaagagaaactcttcatgcatatcttttggtccataagtcataaatagttatccttgatcccatgtcttttttagagccatggacagagagaagcaaaaatataccaagttcacactgagttgtctcccttcatatctcttagcagtcactgaaaggttatgagactcaggctgggtttctatcctctgtccctgaaacgacaacgttgacctcgtgatcaaccctagaatccagagcaagatctcagactgtcctctctactaccagacagcacactttgttttgggggctgtgtgcttgaaatattagctatggcaaaaggctttgagtcttatgacaccccaagtaacttttactttaggaatttgaaatacagccttgctgtaatgctgtctccttaacaaagcagtacctttgaaatatttaacaacttgaaaaggaaaccgagcttgaattttcctttcaggtgctcaggaaataatgtttcacttctgtctgaaattcaccatctcctcagacaaagaaggctcttatggtaaaaggaatggcattttctccacaattttcgaataaaagataaagagaaaacagcactgcagcctttttgttaggatctaacaataaagaaataatacggttttgccaggggagagctctggttttaagctcagaatacaaaaataggctgacaaaattttacaaaggaatattctcagctaccactctgaggatggtagaaagtgaaatttcaagaaaattatattatttgattatttgatgatgattaagctgattggccagtcctatgtgaaattctaaagtagaagaaatgtgatgttgtcttttctgctcacctctcccctcattcctacccccaaatctctgcctctaccccaaacccagcctgacctttgggaaaggaatgggggctgtcacttgcaccgtagctcctcctgcctgcagtactctccccccacagtccagcctccctctgctcagctaacttttcctatagctccttctggctacttctaaggaaccttccataatgctgcccaccctctcagtaacagcccctctactatcccttgattacatgcactgtaattggttagtaattgattttatgtcctctgccaaattatactccatgagagcaaaaatcatgagtattatctttaatgtttaaatctccacaactatcccccatataagtctagagaataaatgaatgagtgaattaatgaatagaaactcaagccattatgttgccactcctaggatatttggattaactttaactgaagagtaaaaagcatttacctgtcctaaaggagacataaaattagtgggagagtatttggagaaaaaaaagacactgtaatacattcttttgtgttgcctaccctgatgtagtcaggtgtccctgatatggggtgggctgaggatttgaaataaaatacctaatttgacattgtaagtggaggaatcaggatttgaaaccaaattggtttgacctaaatcaagctattatgataaggacttgcaagaaaaaaggaatccataaaaaccatatgaatagctaccaattagtaagcatttatatgtgtcaattacaaagccaaatgcaaatcatgctttatttatattagccaccttttatagatgaagaaattgagacctgaatattaaaattgcctacttttacatagtaagtaaaggaatcagggtttgaacccaaattggtttcacctaaggtagaaaaccatcccagcaagtctcctattaactggaaccctattgtggtggcctgagatataacagtagctgtggaagcgctgtagagtcctggccatcctatgtgctcctgatctggtccctcctgccacctgcttctgctccctgtgccatccacccatctggaagtctcccagtgtccatcttcgggggagacactcaccagagtttccagcttccagccagtatggagtgcccctgtcccacagcaatctcaccgaaatcacagctacatctgttaaaattaggctaccaatgagtgatagatgagggggaaaaataataatagtgtactaaacaaaacaaatgtttatttttctcacacataaaaatctagaggttgaagtccagggctggtccagaggctccaaggatctgggatttagactccctctttcttgtttttccacagcatatggcttccatttctggggccacattggtccaaaatgtatgctggggctccagccattgcatccatatttcagccacaggaaggaggaagtggggaagaaaggacaggcccctaatacctgtatagttcaagaagactatcccgcccatacttcccaaccacccttagttgaacaatgctgtcttaattcaagacactcacatgtctagccaaaaatctgaattctgttacaaacaaggagaatagagatgtgcgccacctcaatacctcatccatagctaccttttcctttgtgcagctgtggccaagtgaaagctgaaggagctgtggtaacccttctgaagggaggctggggcctttcacaagaggctgcatgattgacatttatcctgcatggcctgtgaagtacagagaaatattttctcttgaagccacatcatagcagtggctgctttgtagcctgattccaccattatgcctttaaagtgcctagcaattcagccttcacatcatgcaaagaggaatatctcccagtctttgtaagatcagcttaattctaaccacctccttacctcccactgcactcctacacgcacacacaaatcttcttcactcagagcagaaccataacccaagccctaccacctagagactgaagaatcaggctcatgattacaaatatgcaataattttttgtgtggataatgtcaatggggatgatggtaagagaattccttggtttacacattgaccctcttccctgtcccttacaatcaggaaatatttgtcccaacaccttgtttcttctgttgcaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctgggtcattttcttttgcttttttgttcattttctgaatgggctcctaatacgacttgctggatttcagttcccttttgcaaaatctaaagacgcagaaacaactaatgtagctgcagatctaatgtactgaaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaagcttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctggtttttccccttaaatcaggaaatacacctggacaatttctagaagactacaattcagtctagccacaaaggggattttttttttttggtaacaggctagagcccggttctgtaagtctttagctgaaatggtccagtacaaaagcactggaaatgagtgggctaggaggacaaggaccgtctcctgcgtgaggagttggttggaggtccccaaggccaggtaccccctgcactcttattggattcctctctgtcttcttggagttttgaaaaactccttcgaacaccaggcttttttctttagaaaacaagtctccaatcgttctctgttccgtagaaagagaaagaaaacctggagcagctgctgaaaaatctaatgaggaactaagaggcaaacccacca

SEQ ID NO: 10 - protein, 250 aa, Mus musculus
MPSQQKKIIFCMAGVLSFLCALGVVTAVGTPLWVKATILCKTGALLVNASGKELDKFMGEMQYGLFHGEGVRQCGLGARPFRFSSRSMKERYSLYEDKGETAVFPDLVQAIPVSIHINIILFSMILVVLTMVGTAFFMYNAFGKPFETLHGPLGLYLVSFISGSCGCLVMILFASEVKVHRLSEKIANFKEGTYAYRTQNENYTTSFWVVFICFFVHFLNGLLIRLAGFQFPFTKSKETETTNVASDLMY

SEQ ID NO: 11 - protein, 232 aa, Canis lupus
MPNQQKKVVFCTAGVLSFVCALGVVTALGTPLWIKATFLCKTGALLVNASGQELDKFMGEMQYGLFHGEGIRQCGLGARPFRFSLFPDLLKVIPVSIHVNVILFSTILVVLTMVGTAFFMYNAFGKPFETLHGPLGLYLLSFISGSCGCLVMILFASEVKIHHLSEKIANYKEGTYAYKTQSEKYTTSFWVVFICFLVHLLNGLLIRLAGFQFPFAKSKDTETTNVAADLMY

SEQ ID NO: 12 - DNA, 291 bp, Artificial
  5′ untranslated region
aggagatacttgaaggcagtttgaaagacttgttttacagattcttagtccaaagatttccaattagggagaagaagcagcagaaaaggagaaaagccaagtatgagtgatgatgaggccttcatctactgacatttaacctggcgagaaccgtcgatggtgaagttgccttttcagctgggagctgtccgttcagcttccgtaataaatgcagtcaaagaggcagtcccttcccattgctcacaaaggtcttgtttttgaacctcgccctcacagaagccgtttctcatc

SEQ ID NO: 13 - DNA, 130 bp, Artificial
  5′ inverted terminal repeat
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 14 - DNA, 141 bp, Artificial
  3′ inverted terminal repeat
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 15 - DNA, 1773 bp, Artificial
  5′ untranslated region
aaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaagcttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctggtttttccccttaaatcaggaaatacacctggacaatttctagaagactacaattcagtctagccacaaaggggattttttttttttggtaacaggctagagcccggttctgtaagtctttagctgaaatggtccagtacaaaagcactggaaatgagtgggctaggaggacaaggaccgtctcctgcgtgaggagttggttggaggtccccaaggccaggtaccccctgcactcttattggattcctctctgtcttcttggagttttgaaaaactccttcgaacaccaggcttttttctttagaaaacaagtctccaatcgttctctgttccgtagaaagagaaagaaaacctggagcagctgctgaaaaatctaatgaggaactaagaggcaaacccacca

SEQ ID NO: 16 - DNA, 1013 bp, Artificial
  Chimeric intron
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 17 - DNA, 380 bp, Artificial
  CMV enhancer
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 18 - DNA, 276 bp, Artificial
  CBA promoter
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 19 - protein, 232 aa, Artificial
  tGFP
MESDESGLPAMEIECRITGTLNGVEFELVGGGEGTPEQGRMTNKMKSTKGALTFSPYLLSHVMGYGFYHFGTYPSGYENPFLHAINNGGYTNTRIEKYEDGGVLHVSFSYRYEAGRVIGDFKVMGTGFPEDSVIFTDKIIRSNATVEHLHPMGDNDLDGSFTRTFSLRDGGYYSSVVDSHMHFKSAIHPSILQNGGPMFAFRRVEEDHSNTELGIVEYQHAFKTPDADAGEE

SEQ ID NO: 20 - DNA, 225 bp, Bos taurus
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 21 - DNA, 17 bp, Homo sapiens
aaataaaatacgaaatg

SEQ ID NO: 22 - DNA, 82 bp, Artificial
  Splice donor
gtaagtatcaaggttacaagacaggtttaaggagaccaatagaaactgggcttgtcgagacagagaagactcttgcgtttct

SEQ ID NO: 23 - DNA, 51 bp, Artificial
  Splice acceptor
gataggcacctattggtcttactgacatccactttgcctttctctccacag

SEQ ID NO: 24 - DNA, 141 bp, Artificial
  3′ inverted terminal repeat
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 25 - DNA, 145 bp, Artificial
  ITR
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 26 - DNA, 245 bp, Artificial
  sh-chimeric intron
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacag

SEQ ID NO: 27 - DNA, 600 bp, Artificial
  3′UTR 600A
aaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacag

SEQ ID NO: 28 - DNA, 600 bp, Artificial
  3′UTR 600B
gaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaacgttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaaca

SEQ ID NO: 29 - DNA, 600 bp, Artificial
  3′UTR 600C
aggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctggtttttccccttaaatcaggaaatacacctggacaatttctagaagactacaattcagtctagccacaaaggggattttttttttttggtaacaggctagagcccggttctgtaagtctttagctgaaatggtccagtacaaaagcactggaaatgagtgggctaggaggacaaggaccgtctcctgcgtgaggagttggttggaggtccccaaggccaggtaccccctgcactcttattggattcctctctgtcttcttggagttttgaaaaactccttcgaacaccaggcttttttctttagaaaacaagtctccaatcgttctctgttccgtagaaagagaaagaaaacctggagcagctgctgaaaaatctaatgaggaactaagaggcaaacccacca

SEQ ID NO: 30 - protein, 6 aa, Artificial
  FP
KRKRRG

SEQ ID NO: 31 - protein, 18 aa, Artificial
  T2A
EGRGSLLTCGDVEENPGP

SEQ ID NO: 32 - protein, 239 aa, Artificial
  Enhanced GFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 33 - protein, 30 aa, Artificial
  Myc tag sequence
EQKLISEEDLEQKLISEEDLEQKLISEEDL

SEQ ID NO: 34 - protein, 9 aa, Artificial
  HA tag sequence
YPYDVPDYA

SEQ ID NO: 35 - protein, 22 aa, Artificial
  FLAG tag sequence
DYKDHDGDYKDHDIDYKDDDDK

SEQ ID NO: 36 - DNA, 1357 bp, Artificial
  3′UTR 1357
aaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagtctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaacgttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgct

SEQ ID NO: 37 - DNA, 1406 bp, Artificial
  3′UTR 1406
aaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatatgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaacgttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctggtttttccccttaaatcaggaaatacacctggacaatttctagaagac

SEQ ID NO: 38 - protein, 20 aa, Artificial
  CLRN1 antibody epitope
EKIANYKEGTYVYKTQSEKY

SEQ ID NO: 39 - DNA, 4732 bp, Artificial
  CLRN-0 vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtactaagagctcaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaacgttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctggtttttccccttaaatcaggaaatacacctggacaatttctagaagactacaattcagtctagccacaaaggggattttttttttttggtaacaggctagagcccggttctgtaagtctttagctgaaatggtccagtacaaaagcactggaaatgagtgggctaggaggacaaggaccgtctcctgcgtgaggagttggttggaggtccccaaggccaggtaccccctgcactcttattggattcctctctgtcttcttggagttttgaaaaactccttcgaacaccaggcttttttctttagaaaacaagtctccaatcgttctctgttccgtagaaagagaaagaaaacctggagcagctgctgaaaaatctaatgaggaactaagaggcaaacccaccactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 40 - DNA, 3397 bp, Artificial
  CLRN-1 vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtacggcagcggagaaggcagaggcagcctgcttacatgtggcgacgtggaagagaaccccggacctatgcaggctctgcagcagcagccagtgttccccgatctgctgaaagccattcctgtcagcatccatgtcaacgtcatcctcttctctgccatcctcattgtcctcactatggttggaacggccttttttatgtataatgcctttgggaagccgtttgaaaccctgcacggacccctgggactctatctcctgagcttcatctccggctcttgcggctgcctcgtgatgatcctctttgcctctgaagtcaaaattcaccacctgtctgagaaaattgctaattacaaagaagggacatacgtttacaaaacgcagagcgaaaagtatacgaccagcttctggctgaccaagggccactcttaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 41 - DNA, 4177 bp, Artificial
  CLRN-2eGFP vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtacggcagcggagaaggcagaggcagcctgcttacatgtggcgacgtggaagagaaccccggacctatgcaggctctgcagcagcagccagtgttccccgatctgctgaaagccattcctgtcagcatccatgtcaacgtcatcctcttctctgccatcctcattgtcctcactatggttggaacggccttttttatgtataatgcctttgggaagccgtttgaaaccctgcacggacccctgggactctatctcctgagcttcatctccggctcttgcggctgcctcgtgatgatcctctttgcctctgaagtcaaaattcaccacctgtctgagaaaattgctaattacaaagaagggacatacgtttacaaaacgcagagcgaaaagtatacgaccagcttctggctgaccaagggccactctggatcaggcgaaggcaggggatctctgctgacctgcggagatgtcgaagagaatcctggaccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 42 - DNA, 4607 bp, Artificial
  CLRN-3
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtaggagatacttgaaggcagtttgaaagacttgttttacagattcttagtccaaagatttccaattagggagaagaagcagcagaaaaggagaaaagccaagtatgagtgatgatgaggccttcatctactgacatttaacctggcgagaaccgtcgatggtgaagttgccttttcagctgggagctgtccgttcagcttccgtaataaatgcagtcaaagaggcagtcccttcccattgctcacaaaggtcttgtttttgaacctcgccctcacagaagccgtttctcatcgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtactaagagctcaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagtctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaacgttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 43 - DNA, 4794 bp, Artificial
  CLRN-4 vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtacggcagcggagaaggcagaggcagcctgcttacatgtggcgacgtggaagagaaccccggacctatgcaggctctgcagcagcagccagtgttccccgatctgctgaaagccattcctgtcagcatccatgtcaacgtcatcctcttctctgccatcctcattgtcctcactatggttggaacggccttttttatgtataatgcctttgggaagccgtttgaaaccctgcacggacccctgggactctatctcctgagcttcatctccggctcttgcggctgcctcgtgatgatcctctttgcctctgaagtcaaaattcaccacctgtctgagaaaattgctaattacaaagaagggacatacgtttacaaaacgcagagcgaaaagtatacgaccagcttctggctgaccaagggccactcttaagagctcaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatatgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaacgttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctggtttttccccttaaatcaggaaatacacctggacaatttctagaagacaggcctctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 44 - DNA, 4756 bp, Artificial
  CLRN-6eGFP vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagagctcaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaacgttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctggtttttccccttaaatcaggaaatacacctggacaatttctagaagactacaattcagtctagccacaaaggggattttttttttttggtaacaggctagagcccggttctgtaagtctttagctgaaatggtccagtacaaaagcactggaaatgagtgggctaggaggacaaggaccgtctcctgcgtgaggagttggttggaggtccccaaggccaggtaccccctgcactcttattggattcctctctgtcttcttggagttttgaaaaactccttcgaacaccaggcttttttctttagaaaacaagtctccaatcgttctctgttccgtagaaagagaaagaaaacctggagcagctgctgaaaaatctaatgaggaactaagaggcaaacccaccactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 45 - DNA, 3580 bp, Artificial
  CLRN-7eGFP vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagagctcaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 46 - DNA, 3982 bp, Artificial
  CLRN-8 vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtacggcagcggagaaggcagaggcagcctgcttacatgtggcgacgtggaagagaaccccggacctatgcaggctctgcagcagcagccagtgttccccgatctgctgaaagccattcctgtcagcatccatgtcaacgtcatcctcttctctgccatcctcattgtcctcactatggttggaacggccttttttatgtataatgcctttgggaagccgtttgaaaccctgcacggacccctgggactctatctcctgagcttcatctccggctcttgcggctgcctcgtgatgatcctctttgcctctgaagtcaaaattcaccacctgtctgagaaaattgctaattacaaagaagggacatacgtttacaaaacgcagagcgaaaagtatacgaccagcttctggctgaccaagggccactcttaagagctcgaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaacgttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 47 - DNA, 3982 bp, Artificial
  CLRN-9 vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtacggcagcggagaaggcagaggcagcctgcttacatgtggcgacgtggaagagaaccccggacctatgcaggctctgcagcagcagccagtgttccccgatctgctgaaagccattcctgtcagcatccatgtcaacgtcatcctcttctctgccatcctcattgtcctcactatggttggaacggccttttttatgtataatgcctttgggaagccgtttgaaaccctgcacggacccctgggactctatctcctgagcttcatctccggctcttgcggctgcctcgtgatgatcctctttgcctctgaagtcaaaattcaccacctgtctgagaaaattgctaattacaaagaagggacatacgtttacaaaacgcagagcgaaaagtatacgaccagcttctggctgaccaagggccactcttaagagctcaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctggtttttccccttaaatcaggaaatacacctggacaatttctagaagactacaattcagtctagccacaaaggggattttttttttttggtaacaggctagagcccggttctgtaagtctttagctgaaatggtccagtacaaaagcactggaaatgagtgggctaggaggacaaggaccgtctcctgcgtgaggagttggttggaggtccccaaggccaggtaccccctgcactcttattggattcctctctgtcttcttggagttttgaaaaactccttcgaacaccaggcttttttctttagaaaacaagtctccaatcgttctctgttccgtagaaagagaaagaaaacctggagcagctgctgaaaaatctaatgaggaactaagaggcaaacccaccactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 48 - DNA, 3511 bp, Artificial
  CLRN-10 vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtacggatcctatccctatgatgtgccagactatgctaagcggaagagaagaggcgaaggcagaggcagcctgcttacatgtggcgacgtggaagagaaccccggacctatgcaggctctgcagcagcagccagtgttccccgatctgctgaaagccattcctgtcagcatccatgtcaacgtcatcctcttctctgccatcctcattgtcctcactatggttggaacggccttttttatgtataatgcctttgggaagccgtttgaaaccctgcacggacccctgggactctatctcctgagcttcatctccggctcttgcggctgcctcgtgatgatcctctttgcctctgaagtcaaaattcaccacctgtctgagaaaattgctaattacaaagaagggacatacgtttacaaaacgcagagcgaaaagtatacgaccagcttctggctgaccaagggccactctggatccgactacaaagaccatgacggtgattataaagatcatgacatcgattacaaggatgacgatgacaagtaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 49 - DNA, 3574 bp, Artificial
  CLRN-10myc
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtacggatccgagcagaaactcatctctgaagaagatctggaacaaaagttgatttcagaagaagatctggaacagaagctcatctctgaggaagatctgaagcggaagagaagaggcgaaggcagaggcagcctgcttacatgtggcgacgtggaagagaaccccggacctatgcaggctctgcagcagcagccagtgttccccgatctgctgaaagccattcctgtcagcatccatgtcaacgtcatcctcttctctgccatcctcattgtcctcactatggttggaacggccttttttatgtataatgcctttgggaagccgtttgaaaccctgcacggacccctgggactctatctcctgagcttcatctccggctcttgcggctgcctcgtgatgatcctctttgcctctgaagtcaaaattcaccacctgtctgagaaaattgctaattacaaagaagggacatacgtttacaaaacgcagagcgaaaagtatacgaccagcttctggctgaccaagggccactctggatccgactacaaagaccatgacggtgattataaagatcatgacatcgattacaaggatgacgatgacaagtaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 50 - DNA, 3499 bp, Artificial
  CLRN-10NF vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtacggatcctatccctatgatgtgccagactatgctaagggcgaaggcagaggcagcctgcttacatgtggcgacgtggaagagaaccccggacctatgcaggctctgcagcagcagccagtgttccccgatctgctgaaagccattcctgtcagcatccatgtcaacgtcatcctcttctctgccatcctcattgtcctcactatggttggaacggccttttttatgtataatgcctttgggaagccgtttgaaaccctgcacggacccctgggactctatctcctgagcttcatctccggctcttgcggctgcctcgtgatgatcctctttgcctctgaagtcaaaattcaccacctgtctgagaaaattgctaattacaaagaagggacatacgtttacaaaacgcagagcgaaaagtatacgaccagcttctggctgaccaagggccactctggatccgactacaaagaccatgacggtgattataaagatcatgacatcgattacaaggatgacgatgacaagtaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 51 - DNA, 4908 bp, Artificial
  CLRN-11 vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtacggatcctatccctatgatgtgccagactatgctaagcggaagagaagaggcgaaggcagaggcagcctgcttacatgtggcgacgtggaagagaaccccggacctatgcaggctctgcagcagcagccagtgttccccgatctgctgaaagccattcctgtcagcatccatgtcaacgtcatcctcttctctgccatcctcattgtcctcactatggttggaacggccttttttatgtataatgcctttgggaagccgtttgaaaccctgcacggacccctgggactctatctcctgagcttcatctccggctcttgcggctgcctcgtgatgatcctctttgcctctgaagtcaaaattcaccacctgtctgagaaaattgctaattacaaagaagggacatacgtttacaaaacgcagagcgaaaagtatacgaccagcttctggctgaccaagggccactctggatccgactacaaagaccatgacggtgattataaagatcatgacatcgattacaaggatgacgatgacaagtaagagctcaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatatgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaacgttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctggtttttccccttaaatcaggaaatacacctggacaatttctagaagacaggcctctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 52 - DNA, 4971 bp, Artificial
  CLRN-11myc vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtacggatccgagcagaaactcatctctgaagaagatctggaacaaaagttgatttcagaagaagatctggaacagaagctcatctctgaggaagatctgaagcggaagagaagaggcgaaggcagaggcagcctgcttacatgtggcgacgtggaagagaaccccggacctatgcaggctctgcagcagcagccagtgttccccgatctgctgaaagccattcctgtcagcatccatgtcaacgtcatcctcttctctgccatcctcattgtcctcactatggttggaacggccttttttatgtataatgcctttgggaagccgtttgaaaccctgcacggacccctgggactctatctcctgagcttcatctccggctcttgcggctgcctcgtgatgatcctctttgcctctgaagtcaaaattcaccacctgtctgagaaaattgctaattacaaagaagggacatacgtttacaaaacgcagagcgaaaagtatacgaccagcttctggctgaccaagggccactctggatccgactacaaagaccatgacggtgattataaagatcatgacatcgattacaaggatgacgatgacaagtaagagctcaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatatgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaacgttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctggtttttccccttaaatcaggaaatacacctggacaatttctagaagacaggcctctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 53 - DNA, 4896 bp, Artificial
  CLRN-11NF vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtacggatcctatccctatgatgtgccagactatgctaagggcgaaggcagaggcagcctgcttacatgtggcgacgtggaagagaaccccggacctatgcaggctctgcagcagcagccagtgttccccgatctgctgaaagccattcctgtcagcatccatgtcaacgtcatcctcttctctgccatcctcattgtcctcactatggttggaacggccttttttatgtataatgcctttgggaagccgtttgaaaccctgcacggacccctgggactctatctcctgagcttcatctccggctcttgcggctgcctcgtgatgatcctctttgcctctgaagtcaaaattcaccacctgtctgagaaaattgctaattacaaagaagggacatacgtttacaaaacgcagagcgaaaagtatacgaccagcttctggctgaccaagggccactctggatccgactacaaagaccatgacggtgattataaagatcatgacatcgattacaaggatgacgatgacaagtaagagctcaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatatgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaacgttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctggtttttccccttaaatcaggaaatacacctggacaatttctagaagacaggcctctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 54 - DNA, 4640 bp, Artificial
  CLRN-12 vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtaggagatacttgaaggcagtttgaaagacttgttttacagattcttagtccaaagatttccaattagggagaagaagcagcagaaaaggagaaaagccaagtatgagtgatgatgaggccttcatctactgacatttaacctggcgagaaccgtcgatggtgaagttgccttttcagctgggagctgtccgttcagcttccgtaataaatgcagtcaaagaggcagtcccttcccattgctcacaaaggtcttgtttttgaacctcgccctcacagaagccgtttctcatcgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtacggatcctatccctatgatgtgccagactatgcttaagagctcaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagtctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaacgttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 55 - DNA, 4291 bp, Artificial
  CLRN-13 vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttccggttcagctgctactttctggaccccttcatgggcctgcctaccggcgttccacatctgctgtctctgccttgcagcaccagctgcagaagagagcacaccagcgagagagtgcaagagcctgccggctgtttttctgccgtgcggtctaaactgcacgccggacctgctgccgccaccagcttttctagattcgcccagagcaaccccagcgagcaccctagacagtgtcacagcctgctgtgtcacccctactgtgtgaatcacggcggcgattctctgctgcatgtgcagtgcttttggaaaaccttcggcagcgactacaaggaccacgacggcgattataaggatcacgatatcgattacaaggacgatgacgacaagggcgaaggcagaggctccctgctgacatgcggagatgtcgaagagaaccccggacctatgccttctcagcagaaaaagattattttctgtatggctggggtgttctccttcgcttgcgccctgggtgttgttaccgctctcggaacaccactgtggattaaggctaccgtcctgtgtaaaaccggcgctctgctcgtgaatgccagcggacaagaactggataagtttatgggagaaatgcaatatgggctctttcacggcgagggtgttagacagtgcggactcggcgctagacccttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccacaagcttttgggttatcttcttctgtttcttcgtgcacttcctgaacggcctgctgatcagactggccggcttccagtttccattcgccaagtccaaggacgccgaaaccacaaacgtggccgccgacctgatgtacagcggctacccctacgacgtgccagattatgcataagagctcaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacaggctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 56 - DNA, 4192 bp, Artificial
  CLRN-14 vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttccggttcagctgctactttctggaccccttcatgggcctgcctaccggcgttccacatctgctgtctctgccttgcagcaccagctgcagaagagagcacaccagcgagagagtgcaagagcctgccggctgtttttctgccgtgcggtctaaactgcacgccggacctgctgccgccaccagcttttctagattcgcccagagcaaccccagcgagcaccctagacagtgtcacagcctgctgtgtcacccctactgtgtgaatcacggcggcgattctctgctgcatgtgcagtgcttttggaaaaccttcggcagcggcgaaggcagaggctccctgctgacatgcggagatgtcgaagagaaccccggacctatgccttctcagcagaaaaagattattttctgtatggctggggtgttctccttcgcttgcgccctgggtgttgttaccgctctcggaacaccactgtggattaaggctaccgtcctgtgtaaaaccggcgctctgctcgtgaatgccagcggacaagaactggataagtttatgggagaaatgcaatatgggctctttcacggcgagggtgttagacagtgcggactcggcgctagacccttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccacaagcttttgggttatcttcttctgtttcttcgtgcacttcctgaacggcctgctgatcagactggccggcttccagtttccattcgccaagtccaaggacgccgaaaccacaaacgtggccgccgacctgatgtactaagagctcaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacaggctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 57 - DNA, 3505 bp, Artificial
  CLRN-15 vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttccggttcagctgctactttctggaccccttcatgggcctgcctaccggcgttccacatctgctgtctctgccttgcagcaccagctgcagaagagagcacaccagcgagagagtgcaagagcctgccggctgtttttctgccgtgcggtctaaactgcacgccggacctgctgccgccaccagcttttctagattcgcccagagcaaccccagcgagcaccctagacagtgtcacagcctgctgtgtcacccctactgtgtgaatcacggcggcgattctctgctgcatgtgcagtgcttttggaaaaccttcggcagcgactacaaggaccacgacggcgattataaggatcacgatatcgattacaaggacgatgacgacaagtaagagctcaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacaggctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 58 - DNA, 3439 bp, Artificial
  CLRN-16 vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttccggttcagctgctactttctggaccccttcatgggcctgcctaccggcgttccacatctgctgtctctgccttgcagcaccagctgcagaagagagcacaccagcgagagagtgcaagagcctgccggctgtttttctgccgtgcggtctaaactgcacgccggacctgctgccgccaccagcttttctagattcgcccagagcaaccccagcgagcaccctagacagtgtcacagcctgctgtgtcacccctactgtgtgaatcacggcggcgattctctgctgcatgtgcagtgcttttggaaaaccttcggcagctaagagctcaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacaggctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 59 - DNA, 130 bp, Artificial; identical to SEQ ID NO: 13
  CLRN-17 vector

SEQ ID NO: 60 - DNA, 4277 bp, Artificial
  CLRN-18 vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaggagatacttgaaggcagtttgaaagacttgttttacagattcttagtccaaagatttccaattagggagaagaagcagcagaaaaggagaaaagccaagtatgagtgatgatgaggccttcatctactgacatttaacctggcgagaaccgtcgatggtgaagttgccttttcagctgggagctgtccgttcagcttccgtaataaatgcagtcaaagaggcagtcccttcccattgctcacaaaggtcttgtttttgaacctcgccctcacagaagccgtttctcatcaccggtgccaccatgcctagccagcagaagaaaatcatcttctgcatggccggcgtgttcagcttcgcctgtgctctgggagttgtgacagccctgggaacccctctgtggatcaaagccacagtgctgtgcaagacaggcgccctgctggttaatgcctctggccaagagctggacaagttcatgggcgagatgcagtacggcctgttccatggcgaaggcgtcagacagtgtggcctgggagccagacctttcagattcagcttcttcccagacctgctgaaggctatccccgtgtccatccacgtgaacgtgatcctgttcagcgccatcctgatcgtgctgacaatggtcggaaccgccttcttcatgtacaacgccttcggcaagcccttcgagacactgcatggacctctgggcctgtacctgctgagctttatcagcggcagctgtggctgcctggtcatgattctgttcgccagcgaagtgaagatccaccacctgagcgagaagatcgccaactacaaagagggcacctacgtctacaagacccagtccgagaagtacaccaccagcttttgggttatcttcttctgtttcttcgtgcacttcctgaacggcctgctgatcagactggccggcttccagtttccattcgccaagagcaaggacgccgaaaccacaaacgtggccgccgatctgatgtactaagagctcaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaacgttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgctggtttttccccttaaatcaggaaatacacctggacaatttctagaagactacaattcagtctagccacaaaggggattttttttttttggtaacaggctagagcccggttctgtaagtctttagctgaaatggtccagtacaaaagcactggaaatgagtgggctaggaggacaaggaccgtctcctgcgtgaggagttggttggaggtccccaaggccaggtaccccctgcactcttattggattcctctctgtcttcttggagttttgaaaaactccttcgaacaccaggcttttttctttagaaaacaagtctccaatcgttctctgttccgtagaaagagaaagaaaacctggagcagctgctgaaaaatctaatgaggaactaagaggcaaacccaccactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

(19) United States
(12) Patent Application Publication
(10) Pub. No.: US 2021/0277417 A1
(43) Pub. Date: Sep. 9, 2021

(54) METHODS OF TREATING CLRN1-ASSOCIATED HEARING LOSS AND/OR VISION LOSS

(71) Applicant: Akouos, Inc., Boston, MA (US)

(72) Inventors: Emmanuel John Simons, Brookline, MA (US); Robert Ng, Newton, MA (US)

(21) Appl. No.: 17/253,658

(22) PCT Filed: Jun. 25, 2019
(86) PCT No.: PCT/US19/39030

§ 371 Date: Dec. 18, 2020

Related U.S. Application Data
(60) Provisional application No. 62/689,660, filed on Jun. 25, 2018.

(51) Int. Cl. C12N 15/86; C12N 15/67; A61P 27/16; A61K 31/713

(52) U.S. Cl. CPC C12N 15/86; C12N 15/67; C12N 2750/14143; A61K 31/713; C12N 2830/50; A61P 27/16

(57) ABSTRACT

Provided herein are compositions that include a single nucleic acid vector or two different nucleic acid vectors, and the use of these compositions to treat hearing loss and/or vision loss in a subject.

### CROSS-REFERENCE TO RELATED APPLICATIONS

This application claims priority to U.S. Provisional Patent Application Ser. No. 62/689,660, filed Jun. 25, 2018; the entire contents of which are herein incorporated by reference.

### TECHNICAL FIELD

The present disclosure relates generally to the use of nucleic acids to treat hearing loss, vision loss, or both, in a human subject.

### BACKGROUND OF THE INVENTION

Current treatments for hearing loss consist mainly of hearing amplification for mild to severe hearing loss and cochlear implants for severe to profound hearing loss; however, a long-felt need remains for agents and methods for preventing or reversing syndromic deafness.

Hearing loss can be conductive (arising from the ear canal or middle ear), sensorineural (arising from the inner ear or auditory nerve), or mixed. Most forms of syndromic deafness are associated with permanent hearing loss caused by damage to structures in the inner ear (sensorineural deafness), although some forms may involve changes in the middle ear (conductive hearing loss). The great majority of human sensorineural hearing loss is caused by abnormalities in the hair cells of the organ of Corti in the cochlea (poor hair cell function). The hair cells may be abnormal at birth, or may be damaged during the lifetime of an individual (e.g., as a result of noise trauma or infection).

### SUMMARY

The present invention relates to a composition including at least two different nucleic acid vectors, where each of the at least two different vectors includes a coding sequence that encodes a different portion of a CLRN1 protein, can be used to generate a sequence encoding an active CLRN1 protein (e.g., a full-length CLRN1 protein) in a mammalian cell, and thereby treat CLRN1-associated hearing loss and/or vision loss in a subject in need thereof. The invention also related to compositions including a single nucleic acid vector that includes a coding sequence for a first and/or second isoform of CLRN1 protein.

Provided herein are compositions including at least two different nucleic acid vectors, where: each of the at least two different vectors includes a coding sequence that encodes a different portion of a CLRN1 protein, each of the encoded portions being at least 30 amino acid residues in length, wherein the amino acid sequence of each of the encoded portions may optionally partially overlap with the amino acid sequence of a different one of the encoded portions; no single vector of the at least two different vectors encodes a full-length CLRN1 protein; at least one of the coding sequences includes a nucleotide sequence spanning two consecutive exons of CLRN1 genomic DNA, and lacking an intronic sequence between the two consecutive exons; and when introduced into a mammalian cell, the at least two different vectors undergo homologous recombination with each other, thereby forming a recombined nucleic acid that encodes a full-length CLRN1 protein. In some embodiments of any of the compositions provided herein, each of the at least two different vectors includes a coding sequence that encodes a different portion of a first isoform of the CLRN1 protein. In some embodiments of any of the compositions provided herein, the first isoform of the CLRN1 protein includes a sequence that is at least 95% identical to SEQ ID NO: 3. In some embodiments of any of the compositions provided herein, the first isoform of the CLRN1 protein includes SEQ ID NO: 3. In some embodiments of any of the compositions provided herein, the first isoform of the CLRN1 protein consists of SEQ ID NO: 3. In some embodiments of any of the compositions provided herein, one of the at least two different nucleic acid vectors further includes a sequence that encodes a second isoform of the CLRN1 protein. In some embodiments of any of the compositions provided herein, the second isoform of the CLRN1 protein includes a sequence that is at least 95% identical to SEQ ID NO: 5. In some embodiments of any of the compositions provided herein, the second isoform of the CLRN1 protein includes SEQ ID NO: 5. In some embodiments of any of the compositions provided herein, the second isoform of the CLRN1 protein consists of SEQ ID NO: 5. In some embodiments of any of the compositions provided herein, each of the at least two different vectors includes a coding sequence that encodes a different portion of a second isoform of the CLRN1 protein. In some embodiments of any of the compositions provided herein, the second isoform of the CLRN1 protein includes a sequence that is at least 95% identical to SEQ ID NO: 5. In some embodiments of any of the compositions provided herein, the second isoform of the CLRN1 protein includes SEQ ID NO: 5. In some embodiments of any of the compositions provided herein, the second isoform of the CLRN1 protein consists of SEQ ID NO: 5. In some embodiments of any of the compositions provided herein, one of the at least two different nucleic acid vectors further includes a sequence that encodes a first isoform of the CLRN1 protein. In some embodiments of any of the compositions provided herein, the first isoform of the CLRN1 protein includes a sequence that is at least 95% identical to SEQ ID NO: 3. In some embodiments of any of the compositions provided herein, the first isoform of the CLRN1 protein includes SEQ ID NO: 3. In some embodiments of any of the compositions provided herein, the first isoform of the CLRN1 protein consists of SEQ ID NO: 3.

In some embodiments of any of the compositions provided herein, at least one of the at least two different vectors includes a 5′ untranslated region (UTR), a 3′ UTR, or both. In some embodiments of any of the compositions provided herein, the 5′ UTR comprises at least 10 contiguous nucleotides from anywhere within SEQ ID NO: 12. In some embodiments of any of the compositions provided herein, the 5′ UTR includes at least 20 contiguous nucleotides from anywhere within SEQ ID NO: 12. In some embodiments of any of the compositions provided herein, the 5′ UTR includes at least 50 contiguous nucleotides from anywhere within SEQ ID NO: 12. In some embodiments of any of the compositions provided herein, the 5′ UTR includes at least 80 contiguous nucleotides from anywhere within SEQ ID NO: 12. In some embodiments of any of the compositions provided herein, the 5′UTR includes a sequence that is at least 80% identical to SEQ ID NO: 12. In some embodiments of any of the compositions described herein, the 3′ UTR includes at least 10 contiguous nucleotides from anywhere within SEQ ID NO: 15. In some embodiments of any of the compositions described herein, the 3′ UTR includes at least 20 contiguous nucleotides from anywhere within SEQ ID NO: 15. In some embodiments of any of the compositions provided herein, the 3′ UTR includes at least 50 contiguous nucleotides from anywhere within SEQ ID NO: 15. In some embodiments of any of the compositions provided herein, the 3′ UTR includes at least 80 contiguous nucleotides from anywhere within SEQ ID NO: 15. In some embodiments of any of the compositions provided herein, the 3′ UTR includes a sequence that is at least 80% identical to SEQ ID NO: 15.

In some embodiments of any of the compositions provided herein, each of the at least two different vectors is a plasmid, a transposon, a cosmid, an artificial chromosome, or a viral vector. In some embodiments of any of the compositions provided herein, each of the at least two different vectors is a human artificial chromosome (HAC), yeast artificial chromosome (YAC), bacterial artificial chromosome (BAC), or a P1-derived artificial chromosome (PAC). In some embodiments of any of the compositions provided herein, each of the at least two different vectors is a viral vector selected from an adeno-associated virus (AAV) vector, an adenovirus vector, a lentivirus vector, or a retrovirus vector. In some embodiments of any of the compositions provided herein, each of the at least two different vectors is an AAV vector.

In some embodiments of any of the compositions provided herein, the amino acid sequence of none of the encoded portions overlaps with the amino acid sequence of a different one of the encoded portions. In some embodiments of any of the compositions provided herein, the amino acid sequence of each of the encoded portions partially overlaps with the amino acid sequence of a different one of the encoded portions. In some embodiments of any of the compositions provided herein, the overlapping amino acid sequence is between about 30 amino acid residues to about 202 amino acid residues in length. In some embodiments of any of the compositions provided herein, the vectors include two different vectors, each of which includes a different segment of an intron, wherein the intron includes the nucleotide sequence of an intron that is present in CLRN1 genomic DNA, and wherein the two different intron segments overlap in sequence by at least 100 nucleotides. In some embodiments of any of the compositions provided herein, the two different intron segments overlap in sequence by 100 nucleotides to about 800 nucleotides.

In some embodiments of any of the compositions provided herein, the entire nucleotide sequence of each of the at least two different vectors is between about 500 nucleotides to about 10,000 nucleotides in length. In some embodiments of any of the compositions provided herein, the entire nucleotide sequence of each of the at least two different vectors is between 500 nucleotides to 5,000 nucleotides in length. In some embodiments of any of the compositions provided herein, the number of different vectors in the composition is two. In some embodiments of any of the compositions provided herein, a first of the two different vectors includes a coding sequence that encodes an N-terminal portion of the CLRN1 protein. In some embodiments of any of the compositions provided herein, the N-terminal portion of the CLRN1 protein is between 30 amino acids to 202 amino acids in length. In some embodiments of any of the compositions provided herein, the N-terminal portion of the CLRN1 protein is between 60 amino acids to 170 amino acids in length. In some embodiments of any of the compositions provided herein, the first vector further includes a 5′ UTR sequence. In some embodiments of any of the compositions provided herein, the 5′ UTR comprises at least 10 contiguous nucleotides from anywhere within SEQ ID NO: 12. In some embodiments of any of the compositions provided herein, the 5′ UTR includes at least 20 contiguous nucleotides from anywhere within SEQ ID NO: 12. In some embodiments of any of the compositions provided herein, the 5′ UTR includes at least 50 contiguous nucleotides from anywhere within SEQ ID NO: 12. In some embodiments of any of the compositions provided herein, the 5′ UTR includes at least 80 contiguous nucleotides from anywhere within SEQ ID NO: 12. In some embodiments of any of the compositions provided herein, the 5′UTR includes a sequence that is at least 80% identical to SEQ ID NO: 12. In some embodiments of any of the compositions provided herein, the first vector further includes one or both of a promoter and a Kozak sequence. In some embodiments of any of the compositions provided herein, the first vector includes a promoter that is an inducible promoter, a constitutive promoter, or a tissue-specific promoter. In some embodiments of any of the compositions provided herein, the second of the two different vectors includes a coding sequence that encodes a C-terminal portion of the CLRN1 protein. In some embodiments of any of the compositions provided herein, the C-terminal portion of the CLRN1 protein is between 30 amino acids to 202 amino acids in length. In some embodiments of any of the compositions provided herein, the C-terminal portion of the CLRN1 protein is between 60 amino acids to 170 amino acids in length. In some embodiments of any of the compositions provided herein, the second vector further includes a polyadenylation signal sequence. In some embodiments of any of the compositions provided herein, the second vector further includes a 3′UTR sequence. In some embodiments of any of the compositions provided herein, the 3′ UTR includes at least 10 contiguous nucleotides from anywhere within SEQ ID NO: 15. In some embodiments of any of the compositions provided herein, the 3′ UTR includes at least 20 contiguous nucleotides from anywhere within SEQ ID NO: 15. In some embodiments of any of the compositions provided herein, the 3′ UTR includes at least 50 contiguous nucleotides from anywhere within SEQ ID NO: 15. In some embodiments of any of the compositions provided herein, the 3′ UTR includes at least 80 contiguous nucleotides from anywhere within SEQ ID NO: 15. In some embodiments of any of the compositions provided herein, the 3′ UTR includes a sequence that is at least 80% identical to SEQ ID NO: 15.

Also provided herein are compositions that include a single nucleic acid vector, where the vector includes one or both of (i) a first coding sequence encoding a first isoform of CLRN1 protein, and (ii) a second coding sequence encoding a second isoform of CLRN1 protein, where one or both of the first and second coding sequences includes a nucleotide sequence spanning two consecutive exons of a CLRN1 genomic DNA, and lacking an intronic sequence between the two consecutive introns. In some embodiments of any of the compositions provided herein, the single nucleic acid vector contains the first coding sequence and not the second coding sequence. In some embodiments of any of the compositions provided herein, the single nucleic acid vector contains the second coding sequence and not the first coding sequence. In some embodiments of any of the compositions provided herein, the single nucleic acid vector contains both the first coding sequence and the second coding sequence. In some embodiments of any of the compositions provided herein, the first isoform of the CLRN1 protein includes a sequence that is at least 95% identical to SEQ ID NO: 3. In some embodiments of any of the compositions provided herein, the first isoform of the CLRN1 protein includes SEQ ID NO: 3. In some embodiments of any of the compositions provided herein, the first isoform of the CLRN1 protein consists of SEQ ID NO: 3. In some embodiments of any of the compositions provided herein, the second isoform of the CLRN1 protein includes a sequence that is at least 95% identical to SEQ ID NO: 5. In some embodiments of any of the compositions provided herein, the second isoform of the CLRN1 protein includes SEQ ID NO: 5. In some embodiments of any of the compositions provided herein, the second isoform of the CLRN1 protein consists of SEQ ID NO: 5.

In some embodiments of any of the compositions provided herein, the single nucleic acid vector further includes a 5′ untranslated region (UTR), a 3′ UTR, or both. In some embodiments of any of the compositions provided herein, the 5′ UTR includes at least 10 contiguous nucleotides from anywhere within SEQ ID NO: 12. In some embodiments of any of the compositions provided herein, the 5′ UTR includes at least 20 contiguous nucleotides from anywhere within SEQ ID NO: 12. In some embodiments of any of the compositions provided herein, the 5′ UTR includes at least 50 contiguous nucleotides from anywhere within SEQ ID NO: 12. In some embodiments of any of the compositions provided herein, the 5′ UTR includes at least 80 contiguous nucleotides from anywhere within SEQ ID NO: 12. In some embodiments of any of the compositions provided herein, the 5′ UTR includes a sequence that is at least 80% identical to SEQ ID NO: 12. In some embodiments of any of the compositions provided herein, the 3′ UTR includes at least 10 contiguous nucleotides from anywhere within SEQ ID NO: 15. In some embodiments of any of the compositions provided herein, the 3′ UTR includes at least 20 contiguous nucleotides from anywhere within SEQ ID NO: 15. In some embodiments of any of the compositions provided herein, the 3′ UTR includes at least 50 contiguous nucleotides from anywhere within SEQ ID NO: 15. In some embodiments of any of the compositions provided herein, the 3′ UTR includes at least 80 contiguous nucleotides from anywhere within SEQ ID NO: 15. In some embodiments of any of the compositions provided herein, the 3′ UTR includes a sequence that is at least 80% identical to SEQ ID NO: 15.

In some embodiments of any of the compositions provided herein, the single nucleic acid vector is a plasmid, a transposon, a cosmid, an artificial chromosome, or a viral vector. In some embodiments of any of the compositions provided herein, the single nucleic acid vector is a human artificial chromosome (HAC), yeast artificial chromosome (YAC), bacterial artificial chromosome (BAC), or a P1-derived artificial chromosome (PAC). In some embodiments of any of the compositions provided herein, the single nucleic acid vector is a viral vector selected from an adeno-associated virus (AAV) vector, an adenovirus vector, a lentivirus vector, or a retrovirus vector. In some embodiments of any of the compositions provided herein, the single nucleic acid vector is an AAV vector. In some embodiments of any of the compositions provided herein, the single nucleic acid vector further includes one or both of a promoter and a Kozak sequence. In some embodiments of any of the compositions provided herein, the first vector includes a promoter that is an inducible promoter, a constitutive promoter, or a tissue-specific promoter. In some embodiments of any of the compositions provided herein, the single nucleic acid vector further includes a polyadenylation signal sequence. Some embodiments of any of the compositions provided herein further include a pharmaceutically acceptable excipient.

Also provided herein are kits that include any of the compositions provided herein. Some embodiments of any of the kits provided herein further include a pre-loaded syringe including or containing any of the compositions described herein.

Also provided herein are methods that include introducing into a cochlea of a mammal a therapeutically effective amount of any of the compositions provided herein. In some embodiments of any of the methods provided herein, the mammal is a human. In some embodiments of any of the methods provided herein, the mammal has been previously identified as having a defective CLRN1 gene.

Also provided herein are methods of increasing expression of a full-length CLRN1 protein in a mammalian cell that include introducing any of the compositions provided herein into the mammalian cell. In some embodiments of any of the methods provided herein, the mammalian cell is a cochlear inner hair cell or a cochlear outer hair cell. In some embodiments of any of the methods provided herein, the mammalian cell is a retinal cell. In some embodiments of any of the methods provided herein, the mammalian cell is a human cell. In some embodiments of any of the methods provided herein, the mammalian cell has previously been determined to have a defective CLRN1 gene.

Also provided herein are methods of increasing expression of a full-length CLRN1 protein in an inner hair cell, an outer hair cell, or both, in a cochlea of a mammal, that include: introducing into the cochlea of the mammal a therapeutically effective amount of any of the compositions provided herein. Also provided herein are methods of increasing expression of a full-length CLRN1 protein in an eye of a mammal that include: intraocularly administering to the eye of the mammal a therapeutically effective amount of any of the compositions provided herein. In some embodiments of any of the methods provided herein, the mammal has been previously identified as having a defective CLRN1 gene. In some embodiments of any of the methods provided herein, the mammal is a human.

Also provided herein are methods of treating hearing loss in a subject identified as having a defective CLRN1 gene that include: administering a therapeutically effective amount of any of the compositions provided herein into the cochlea of the subject. Also provided herein are methods of treating vision loss in a subject identified as having a defective CLRN1 gene that include: administering a therapeutically effective amount of any of the compositions provided herein into the eye of the subject. In some embodiments of any of the methods provided herein, the subject has Usher syndrome type III. In some embodiments of any of the methods provided herein, the subject is a human. Some embodiments of any of the methods provided herein further include, prior to the administering step, determining that the subject has a defective CLRN1 gene.

Also provided herein are compositions that include two different nucleic acid vectors, where: a first nucleic acid vector of the two different nucleic acid vectors includes a promoter, a first coding sequence that encodes an N-terminal portion of a CLRN1 protein positioned 3′ of the promoter, and a splice donor sequence positioned at the 3′ end of the first coding sequence; and a second nucleic acid vector of the two different nucleic acid vectors includes a splice acceptor sequence, a second coding sequence that encodes a C-terminal portion of a CLRN1 protein positioned at the 3′ end of the splice acceptor sequence, and a polyadenylation signal sequence at the 3′ end of the second coding sequence; where each of the encoded portions is at least 30 amino acid residues in length, where the amino acid sequences of the two encoded portions do not overlap with each other; where no single vector of the two different vectors encodes a full-length CLRN1 protein; and when introduced into a mammalian cell, splicing occurs between the splice donor sequence and the splice acceptor sequence, thereby forming a recombined nucleic acid that encodes a full-length CLRN1 protein. In some embodiments of any of the compositions provided herein, the first coding sequence encodes an N-terminal portion of a first isoform of CLRN1 protein, and the second coding sequence encodes a C-terminal portion of the first isoform of CLRN1 protein. In some embodiments of any of the compositions provided herein, the first isoform of the CLRN1 protein includes a sequence that is at least 95% identical to SEQ ID NO: 3. In some embodiments of any of the compositions provided herein, the first isoform of the CLRN1 protein includes SEQ ID NO: 3. In some embodiments of any of the compositions provided herein, the first isoform of the CLRN1 protein consists of SEQ ID NO: 3. In some embodiments of any of the compositions provided herein, the first nucleic acid vector of the second nucleic acid vector further includes a sequence that encodes a second isoform of the CLRN1 protein. In some embodiments of any of the compositions provided herein, the second isoform of the CLRN1 protein includes a sequence that is at least 95% identical to SEQ ID NO: 5. In some embodiments of any of the compositions provided herein, the second isoform of the CLRN1 protein includes SEQ ID NO: 5. In some embodiments of any of the compositions provided herein, the second isoform of the CLRN1 protein consists of SEQ ID NO: 5.

In some embodiments of any of the compositions provided herein, the first coding sequence encodes an N-terminal portion of a second isoform of CLRN1 protein, and the second coding sequence encodes a C-terminal portion of the second isoform of CLRN1 protein. In some embodiments of any of the compositions provided herein, the second isoform of the CLRN1 protein includes a sequence that is at least 95% identical to SEQ ID NO: 5. In some embodiments of any of the compositions provided herein, the second isoform of the CLRN1 protein includes SEQ ID NO: 5. In some embodiments of any of the compositions provided herein, the second isoform of the CLRN1 protein consists of SEQ ID NO: 5. In some embodiments of any of the compositions provided herein, the first nucleic acid vector or the second nucleic acid vector further includes a sequence that encodes a first isoform of the CLRN1 protein. In some embodiments of any of the compositions provided herein, the first isoform of the CLRN1 protein includes a sequence that is at least 95% identical to SEQ ID NO: 3. In some embodiments of any of the compositions provided herein, the first isoform of the CLRN1 protein includes SEQ ID NO: 3. In some embodiments of any of the compositions provided herein, the first isoform of the CLRN1 protein consists of SEQ ID NO: 3.

In some embodiments of any of the compositions provided herein, one or both of the first nucleic acid vector and the second nucleic acid vector includes a 5′ untranslated region (UTR), a 3′ UTR, or both. In some embodiments of any of the compositions provided herein, the 5′ UTR includes at least 10 contiguous nucleotides from anywhere within SEQ ID NO: 12. In some embodiments of any of the compositions provided herein, the 5′ UTR includes at least 20 contiguous nucleotides from anywhere within SEQ ID NO: 12. In some embodiments of any of the compositions provided herein, the 5′ UTR includes at least 50 contiguous nucleotides from anywhere within SEQ ID NO: 12. In some embodiments of any of the compositions provided herein, the 5′ UTR includes at least 80 contiguous nucleotides from anywhere within SEQ ID NO: 12. In some embodiments of any of the compositions provided herein, the 5′UTR includes a sequence that is at least 80% identical to SEQ ID NO: 12. In some embodiments of any of the compositions provided herein, the 3′ UTR includes at least 10 contiguous nucleotides from anywhere within SEQ ID NO: 15. In some embodiments of any of the compositions provided herein, the 3′ UTR includes at least 20 contiguous nucleotides from anywhere within SEQ ID NO: 15. In some embodiments of any of the compositions provided herein, the 3′ UTR includes at least 50 contiguous nucleotides from anywhere within SEQ ID NO: 15. In some embodiments of any of the compositions provided herein, the 3′ UTR includes at least 80 contiguous nucleotides from anywhere within SEQ ID NO: 15. In some embodiments of any of the compositions provided herein, the 3′ UTR includes a sequence that is at least 80% identical to SEQ ID NO: 15.

In some embodiments of any of the compositions provided herein, each of the first nucleic acid vector and the second nucleic acid vector is a plasmid, a transposon, a cosmid, an artificial chromosome, or a viral vector. In some embodiments of any of the compositions provided herein, each of the first nucleic acid vector and the second nucleic acid vector is a human artificial chromosome (HAC), yeast artificial chromosome (YAC), bacterial artificial chromosome (BAC), or a P1-derived artificial chromosome (PAC). In some embodiments of any of the compositions provided herein, each of the first nucleic acid vector and the second nucleic acid vector is a viral vector selected from an adeno-associated virus (AAV) vector, an adenovirus vector, a lentivirus vector, or a retrovirus vector. In some embodiments of any of the compositions provided herein, each of the first nucleic acid vector and the second nucleic acid vector is an AAV vector. In some embodiments of any of the compositions provided herein, at least one of the coding sequences includes a nucleotide sequence spanning two consecutive exons of CLRN1 genomic DNA, and lacking an intronic sequence between the two consecutive exons.

Also provided herein are compositions that include two different nucleic acid vectors, where: a first nucleic acid vector of the two different nucleic acid vectors includes a promoter, a first coding sequence that encodes an N-terminal portion of a CLRN1 protein positioned 3′ of the promoter, a splice donor sequence positioned at the 3′ end of the first coding sequence, and a first detectable marker gene positioned 3′ of the splice donor sequence; and a second nucleic acid vector of the two different nucleic acid vectors includes a second detectable marker gene, a splice acceptor sequence positioned 3′ of the second detectable marker gene, a second coding sequence that encodes a C-terminal portion of a CLRN1 protein positioned at the 3′ end of the splice acceptor sequence, and a polyadenylation signal sequence positioned at the 3′ end of the second coding sequence; where each of the encoded portions is at least 30 amino acid residues in length, where the amino acid sequences of the encoded portions do not overlap with each other; where no single vector of the two different vectors encodes a full-length CLRN1 protein; and when introduced into a mammalian cell, splicing occurs between the splice donor sequence and the splice acceptor sequence, thereby forming a recombined nucleic acid that encodes a full-length CLRN1 protein. In some embodiments of any of the compositions provided herein, the first coding sequence encodes an N-terminal portion of a first isoform of CLRN1 protein, and the second coding sequence encodes a C-terminal portion of the first isoform of CLRN1 protein. In some embodiments of any of the compositions provided herein, the first isoform of the CLRN1 protein includes a sequence that is at least 95% identical to SEQ ID NO: 3. In some embodiments of any of the compositions provided herein, the first isoform of the CLRN1 protein includes SEQ ID NO: 3. In some embodiments of any of the compositions provided herein, the first isoform of the CLRN1 protein consists of SEQ ID NO: 3. In some embodiments of any of the compositions provided herein, the first nucleic acid vector of the second nucleic acid vector further includes a sequence that encodes a second isoform of the CLRN1 protein. In some embodiments of any of the compositions provided herein, the second isoform of the CLRN1 protein includes a sequence that is at least 95% identical to SEQ ID NO: 5. In some embodiments of any of the compositions provided herein, the second isoform of the CLRN1 protein includes SEQ ID NO: 5. In some embodiments of any of the compositions provided herein, the second isoform of the CLRN1 protein consists of SEQ ID NO: 5. In some embodiments of any of the compositions provided herein, the first coding sequence encodes an N-terminal portion of a second isoform of CLRN1 protein, and the second coding sequence encodes a C-terminal portion of the second isoform of CLRN1 protein. In some embodiments of any of the compositions provided herein, the second isoform of the CLRN1 protein includes a sequence that is at least 95% identical to SEQ ID NO: 5. In some embodiments of any of the compositions provided herein, the second isoform of the CLRN1 protein includes SEQ ID NO: 5. In some embodiments of any of the compositions provided herein, the second isoform of the CLRN1 protein consists of SEQ ID NO: 5. In some embodiments of any of the compositions provided herein, the first nucleic acid vector or the second nucleic acid vector further includes a sequence that encodes a first isoform of the CLRN1 protein. In some embodiments of any of the compositions provided herein, the first isoform of the CLRN1 protein includes a sequence that is at least 95% identical to SEQ ID NO: 3. In some embodiments of any of the compositions provided herein, the first isoform of the CLRN1 protein includes SEQ ID NO: 3. In some embodiments of any of the compositions provided herein, the first isoform of the CLRN1 protein consists of SEQ ID NO: 3.

In some embodiments of any of the compositions provided herein, one or both of the first nucleic acid vector and the second nucleic acid vector includes a 5′ untranslated region (UTR), a 3′ UTR, or both. In some embodiments of any of the compositions provided herein, the 5′ UTR includes at least 10 contiguous nucleotides from anywhere within SEQ ID NO: 12. In some embodiments of any of the compositions provided herein, the 5′ UTR includes at least 20 contiguous nucleotides from anywhere within SEQ ID NO: 12. In some embodiments of any of the compositions provided herein, the 5′ UTR includes at least 50 contiguous nucleotides from anywhere within SEQ ID NO: 12.

In some embodiments of any of the compositions provided herein, the 5′ UTR comprises at least 80 contiguous nucleotides from anywhere within SEQ ID NO: 12. In some embodiments of any of the compositions provided herein, the 5′ UTR includes a sequence that is at least 80% identical to SEQ ID NO: 12. In some embodiments of any of the compositions provided herein, the 3′ UTR includes at least 10 contiguous nucleotides from anywhere within SEQ ID NO: 15. In some embodiments of any of the compositions provided herein, the 3′ UTR includes at least 20 contiguous nucleotides from anywhere within SEQ ID NO: 15. In some embodiments of any of the compositions provided herein, the 3′ UTR includes at least 50 contiguous nucleotides from anywhere within SEQ ID NO: 15. In some embodiments of any of the compositions provided herein, the 3′ UTR includes at least 80 contiguous nucleotides from anywhere within SEQ ID NO: 15. In some embodiments of any of the compositions provided herein, the 3′ UTR includes a sequence that is at least 80% identical to SEQ ID NO: 15.

In some embodiments of any of the compositions provided herein, each of the first nucleic acid vector and the second nucleic acid vector is a plasmid, a transposon, a cosmid, an artificial chromosome, or a viral vector. In some embodiments of any of the compositions provided herein, each of the first nucleic acid vector and the second nucleic acid vector is a human artificial chromosome (HAC), yeast artificial chromosome (YAC), bacterial artificial chromosome (BAC), or a P1-derived artificial chromosome (PAC). In some embodiments of any of the compositions provided herein, each of the first nucleic acid vector and the second nucleic acid vector is a viral vector selected from an adeno-associated virus (AAV) vector, an adenovirus vector, a lentivirus vector, or a retrovirus vector. In some embodiments of any of the compositions provided herein, each of the first nucleic acid vector and the second nucleic acid vector is an AAV vector. In some embodiments of any of the compositions provided herein, at least one of the coding sequences comprises a nucleotide sequence spanning two consecutive exons of CLRN1 genomic DNA, and lacking an intronic sequence between the two consecutive exons. In some embodiments of any of the compositions provided herein, the first or second detectable marker gene is alkaline phosphatase.

Also provided herein are compositions that include two different nucleic acid vectors, where: a first nucleic acid vector of the two different nucleic acid vectors includes a promoter, a first coding sequence that encodes an N-terminal portion of a CLRN1 protein positioned 3′ to the promoter, a splice donor sequence positioned at the 3′ end of the first coding sequence, and a F1 phage recombinogenic region positioned 3′ to the splice donor sequence; and a second nucleic acid vector of the two different nucleic acid vectors includes a F1 phage recombinogenic region, a splice acceptor sequence positioned 3′ of the F1 phage recombinogenic region, a second coding sequence that encodes a C-terminal portion of a CLRN1 protein positioned at the 3′ end of the splice acceptor sequence, and a polyadenylation signal sequence positioned at the 3′ end of the second coding sequence; where each of the two encoded portions is at least 30 amino acid residues in length, where the amino acid sequences of the two encoded portions do not overlap with each other; where no single vector of the two different vectors encodes a full-length CLRN1 protein; and when introduced into a mammalian cell, splicing occurs between the splice donor sequence and the splice acceptor sequence, thereby forming a recombined nucleic acid that encodes a full-length CLRN1 protein. In some embodiments of any of the compositions provided herein, the first coding sequence encodes an N-terminal portion of a first isoform of CLRN1 protein, and the second coding sequence encodes a C-terminal portion of the first isoform of CLRN1 protein. In some embodiments of any of the compositions provided herein, the first isoform of the CLRN1 protein includes a sequence that is at least 95% identical to SEQ ID NO: 3. In some embodiments of any of the compositions provided herein, the first isoform of the CLRN1 protein includes SEQ ID NO: 3. In some embodiments of any of the compositions provided herein, the first isoform of the CLRN1 protein consists of SEQ ID NO: 3. In some embodiments of any of the compositions provided herein, the first nucleic acid vector of the second nucleic acid vector further includes a sequence that encodes a second isoform of the CLRN1 protein. In some embodiments of any of the compositions provided herein, the second isoform of the CLRN1 protein includes a sequence that is at least 95% identical to SEQ ID NO: 5. In some embodiments of any of the compositions provided herein, the second isoform of the CLRN1 protein includes SEQ ID NO: 5. In some embodiments of any of the compositions provided herein, the second isoform of the CLRN1 protein consists of SEQ ID NO: 5.

In some embodiments of any of the compositions provided herein, the first coding sequence encodes an N-terminal portion of a second isoform of CLRN1 protein, and the second coding sequence encodes a C-terminal portion of the second isoform of CLRN1 protein. In some embodiments of any of the compositions provided herein, the second isoform of the CLRN1 protein includes a sequence that is at least 95% identical to SEQ ID NO: 5. In some embodiments of any of the compositions provided herein, the second isoform of the CLRN1 protein includes SEQ ID NO: 5. In some embodiments of any of the compositions provided herein, the second isoform of the CLRN1 protein consists of SEQ ID NO: 5. In some embodiments of any of the compositions provided herein, the first nucleic acid vector or the second nucleic acid vector further includes a sequence that encodes a first isoform of the CLRN1 protein. In some embodiments of any of the compositions provided herein, the first isoform of the CLRN1 protein includes a sequence that is at least 95% identical to SEQ ID NO: 3. In some embodiments of any of the compositions provided herein, the first isoform of the CLRN1 protein includes SEQ ID NO: 3. In some embodiments of any of the compositions provided herein, the first isoform of the CLRN1 protein consists of SEQ ID NO: 3.

In some embodiments of any of the compositions provided herein, one or both of the first nucleic acid vector and the second nucleic acid vector comprises a 5′ untranslated region (UTR), a 3′ UTR, or both. In some embodiments of any of the compositions provided herein, the 5′ UTR includes at least 10 contiguous nucleotides from anywhere within SEQ ID NO: 12. In some embodiments of any of the compositions provided herein, the 5′ UTR includes at least 20 contiguous nucleotides from anywhere within SEQ ID NO: 12. In some embodiments of any of the compositions provided herein, the 5′ UTR includes at least 50 contiguous nucleotides from anywhere within SEQ ID NO: 12. In some embodiments of any of the compositions provided herein, the 5′ UTR includes at least 80 contiguous nucleotides from anywhere within SEQ ID NO: 12. In some embodiments of any of the compositions provided herein, the 5′ UTR includes a sequence that is at least 80% identical to SEQ ID NO: 12. In some embodiments of any of the compositions provided herein, the 3′ UTR includes at least 10 contiguous nucleotides from anywhere within SEQ ID NO: 15. In some embodiments of any of the compositions provided herein, the 3′ UTR includes at least 20 contiguous nucleotides from anywhere within SEQ ID NO: 15. In some embodiments of any of the compositions provided herein, the 3′ UTR includes at least 50 contiguous nucleotides from anywhere within SEQ ID NO: 15. In some embodiments of any of the compositions provided herein, the 3′ UTR includes at least 80 contiguous nucleotides from anywhere within SEQ ID NO: 15. In some embodiments of any of the compositions provided herein, the 3′ UTR includes a sequence that is at least 80% identical to SEQ ID NO: 15.

In some embodiments of any of the compositions provided herein, each of the first nucleic acid vector and the second nucleic acid vector is a plasmid, a transposon, a cosmid, an artificial chromosome, or a viral vector. In some embodiments of any of the compositions provided herein, each of the first nucleic acid vector and the second nucleic acid vector is a human artificial chromosome (HAC), yeast artificial chromosome (YAC), bacterial artificial chromosome (BAC), or a P1-derived artificial chromosome (PAC). In some embodiments of any of the compositions provided herein, each of the first nucleic acid vector and the second nucleic acid vector is a viral vector selected from an adeno-associated virus (AAV) vector, an adenovirus vector, a lentivirus vector, or a retrovirus vector. In some embodiments of any of the compositions provided herein, each of the first nucleic acid vector and the second nucleic acid vector is an AAV vector. In some embodiments of any of the compositions provided herein, at least one of the coding sequences includes a nucleotide sequence spanning two consecutive exons of CLRN1 genomic DNA, and lacking an intronic sequence between the two consecutive exons.

Also provided herein are kits that include any of the compositions provided herein. Some embodiments of any of the kits provided herein further include a pre-loaded syringe including the composition.

Also provided herein are methods that include introducing into a cochlea of a mammal a therapeutically effective amount of any of the compositions provided herein. In some embodiments of any of the methods provided herein, the mammal is a human. In some embodiments of any of the methods provided herein, the mammal has been previously identified as having a defective CLRN1 gene.

Also provided herein are methods of increasing expression of a full-length CLRN1 protein in a mammalian cell that include introducing any of the compositions provided herein into the mammalian cell. In some embodiments of any of the methods provided herein, the mammalian cell is a cochlear inner hair cell or a cochlear outer hair cell. In some embodiments of any of the methods provided herein, the mammalian cell is a retinal cell. In some embodiments of any of the methods provided herein, the mammalian cell is a human cell. In some embodiments of any of the methods provided herein, the mammalian cell has previously been determined to have a defective CLRN1 gene.

Also provided herein are methods of increasing expression of a full-length CLRN1 protein in an inner hair cell, an outer hair cell, or both, in a cochlea of a mammal, that include: introducing into the cochlea of the mammal a therapeutically effective amount of any of the compositions described herein. Also provided herein are methods of increasing expression of a full-length CLRN1 protein in an eye of a mammal that include: intraocularly administering to the eye of the mammal a therapeutically effective amount of any of the compositions provided herein. In some embodiments of any of the methods provided herein, the mammal has been previously identified as having a defective CLRN1 gene. In some embodiments of any of the methods provided herein, the mammal is a human.

Also provided herein are methods of treating hearing loss in a subject identified as having a defective CLRN1 gene that include: administering a therapeutically effective amount of any of the compositions provided herein into the cochlea of the subject. Also provided herein are methods of treating vision loss in a subject identified as having a defective CLRN1 gene that include: administering a therapeutically effective amount of any of the compositions provided herein into the eye of the subject. In some embodiments of any of the methods provided herein, the subject has Usher syndrome type III. In some embodiments of any of the methods provided herein, the subject is a human. Some embodiments of any of the methods provided herein further include, prior to the administering step, determining that the subject has a defective CLRN1 gene.

The term “a” and “an” refers to one or to more than one (i.e., at least one) of the grammatical object of the article. By way of example, “an element” encompasses one element and more than one element.

The term “mutation in a CLRN1 gene” refers to a modification in a wildtype CLRN1 gene that results in the production of a CLRN1 protein having one or more of: a deletion in one or more amino acids, one or more amino acid substitutions, and one or more amino acid insertions as compared to the wildtype CLRN1 protein, and/or results in a decrease in the expressed level of the encoded CLRN1 protein in a mammalian cell as compared to the expressed level of the encoded CLRN1 protein in a mammalian cell not having a mutation. In some embodiments, a mutation can result in the production of a CLRN1 protein having a deletion in one or more amino acids (e.g., 2, 3, 4, 5, 6, 7, 8, 9, 10, 11, 12, 13, 14, 15 16, 17, 18, 19, or 20 amino acids). In some embodiments, the mutation can result in a frameshift in the CLRN1 gene. The term “frameshift” is known in the art to encompass any mutation in a coding sequence that results in a shift in the reading frame of the coding sequence. In some embodiments, a frameshift can result in a nonfunctional protein. In some embodiments, a point mutation can be a nonsense mutation (i.e., result in a premature stop codon in an exon of the gene). A nonsense mutation can result in the production of a truncated protein (as compared to a corresponding wildtype protein) that may or may not be functional. In some embodiments, the mutation can result in the loss (or a decrease in the level) of expression of CLRN1 mRNA or CLRN1 protein or both the mRNA and protein. In some embodiments, the mutation can result in the production of an altered CLRN1 protein having a loss or decrease in one or more biological activities (functions) as compared to a wildtype CLRN1 protein.

In some embodiments, the mutation is an insertion of one or more nucleotides into a CLRN1 gene. In some embodiments, the mutation is in a regulatory sequence of the CLRN1 gene, i.e., a portion of the gene that is not coding sequence. In some embodiments, a mutation in a regulatory sequence may be in a promoter or enhancer region and prevent or reduce the proper transcription of the CLRN1 gene. The term “conservative mutation” refers to a mutation that does not change the amino acid encoded at the site of the mutation (due to codon degeneracy).

Modifications can be introduced into a nucleotide sequence by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, cysteine, and tryptophan), nonpolar side chains (e.g., alanine, valine, leucine, isoleucine, proline, phenylalanine, and methionine), beta-branched side chains (e.g., threonine, valine, and isoleucine), and aromatic side chains (e.g., tyrosine, phenylalanine, tryptophan, and histidine).

Unless otherwise specified, a “nucleotide sequence encoding an amino acid sequence” includes all nucleotide sequences that are degenerate versions of each other and thus encode the same amino acid sequence.

The term “endogenous” refers to any material originating from within an organism, cell, or tissue.

The term “exogenous” refers to any material introduced from or originating from outside an organism, cell, or tissue that is not produced or does not originate from the same organism, cell, or tissue in which it is being introduced.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “transfected,” “transformed,” or “transduced” refers to a process by which exogenous nucleic acid is transferred or introduced into a cell. A “transfected,” “transformed,” or “transduced” mammalian cell is one that has been transfected, transformed or transduced with exogenous nucleic acid.

The term “expression” refers to the transcription and/or translation of a particular nucleotide sequence encoding a protein.

The term “transient expression” refers to the expression of a non-integrated coding sequence for a short period of time (e.g., hours or days). The coding sequence that is transiently expressed in a cell (e.g., a mammalian cell) is lost upon multiple rounds of cell division.

The term “subject” is intended to include any mammal. In some embodiments, the subject is a rodent (e.g., a rat or mouse), a rabbit, a sheep, a goat, a pig, a dog, a cat, a non-human primate, or a human. In some embodiments, the subject has or is at risk of hearing loss and/or vision loss. In some embodiments, the subject has been previously identified as having a mutation in a CLRN1 gene. In some embodiments, the subject has been identified as having a mutation in a CLRN1 gene and has been diagnosed with hearing loss and/or vision loss. In some embodiments, the subject has been identified as having hearing loss and/or vision loss.

A treatment is “therapeutically effective” when it results in a reduction in one or more of the number, severity, and frequency of one or more symptoms of a disease state (e.g., hearing loss or vision loss) in a subject (e.g., a human). In some embodiments, a therapeutically effective amount of a composition can result in an increase in the expression level of an active CLRN1 protein (e.g., a wildtype, full-length CLRN1 protein or a variant of a CLRN1 protein that has the desired activity) (e.g., as compared to the expression level prior to treatment with the composition). In some embodiments, a therapeutically effective amount of a composition can result in an increase in the expression level of an active CLRN1 protein (e.g., a wildtype, full-length CLRN1 protein or an active variant) in a target cell (e.g., a cochlear inner hair cell). In some embodiments, a therapeutically effective amount of a composition can result in an increase in the expression level of an active CLRN1 protein (e.g., a wildtype, full-length CLRN1 protein or active variant), and/or an increase in one or more activities of a CLRN1 protein in a target cell (e.g., as compared to a reference level, such as the level(s) in a subject prior to treatment, the level(s) in a subject having a mutation in a CLRN1 gene, or the level(s) in a subject or a population of subjects having hearing loss and/or vision loss).

The term “nucleic acid” or “polynucleotide” refers to deoxyribonucleic acid (DNA) or ribonucleic acid (RNA), or a combination thereof, in either single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

The term “active CLRN1 protein” means a protein encoded by DNA that, if substituted for both wildtype alleles encoding full-length CLRN1 protein in auditory hair cells, or ocular cells, of what is otherwise a wildtype mammal, and if expressed in the auditory hair cells, or ocular cells, of that mammal, results in that mammal's having a level of hearing, or vision, approximating the normal level of hearing, or vision, of a similar mammal that is entirely wildtype. Non-limiting examples of active CLRN1 proteins are full-length CLRN1 proteins (e.g., any of the full-length CLRN1 proteins described herein).

For example, an active CLRN1 protein can include a sequence of a wildtype, full-length CLRN1 protein (e.g., a wildtype, human, full-length CLRN1 protein) including 1 amino acid substitution to about 100 amino acid substitutions, 1 amino acid substitution to about 95 amino acid substitutions, 1 amino acid substitution to about 90 amino acid substitutions, 1 amino acid substitution to about 85 amino acid substitutions, 1 amino acid substitution to about 80 amino acid substitutions, 1 amino acid substitution to about 75 amino acid substitutions, 1 amino acid substitution to about 70 amino acid substitutions, 1 amino acid substitution to about 65 amino acid substitutions, 1 amino acid substitution to about 60 amino acid substitutions, 1 amino acid substitution to about 55 amino acid substitutions, 1 amino acid substitution to about 50 amino acid substitutions, 1 amino acid substitution to about 45 amino acid substitutions, 1 amino acid substitution to about 40 amino acid substitutions, 1 amino acid substitution to about 35 amino acid substitutions, 1 amino acid substitution to about 30 amino acid substitutions, 1 amino acid substitution to about 25 amino acid substitutions, 1 amino acid substitution to about 20 amino acid substitutions, 1 amino acid substitution to about 15 amino acid substitutions, 1 amino acid substitution to about 10 amino acid substitutions, 1 amino acid substitution to about 9 amino acid substitutions, 1 amino acid substitution to about 8 amino acid substitutions, 1 amino acid substitution to about 7 amino acid substitutions, 1 amino acid substitution to about 6 amino acid substitutions, 1 amino acid substitution to about 5 amino acid substitutions, 1 amino acid substitution to about 4 amino acid substitutions, 1 amino acid substitution to about 3 amino acid substitutions, about 2 amino acid substitutions to about 100 amino acid substitutions, about 2 amino acid substitutions to about 95 amino acid substitutions, about 2 amino acid substitutions to about 90 amino acid substitutions, about 2 amino acid substitutions to about 85 amino acid substitutions, about 2 amino acid substitutions to about 80 amino acid substitutions, about 2 amino acid substitutions to about 75 amino acid substitutions, about 2 amino acid substitutions to about 70 amino acid substitutions, about 2 amino acid substitutions to about 65 amino acid substitutions, about 2 amino acid substitutions to about 60 amino acid substitutions, about 2 amino acid substitutions to about 55 amino acid substitutions, about 2 amino acid substitutions to about 50 amino acid substitutions, about 2 amino acid substitutions to about 45 amino acid substitutions, about 2 amino acid substitutions to about 40 amino acid substitutions, about 2 amino acid substitutions to about 35 amino acid substitutions, about 2 amino acid substitutions to about 30 amino acid substitutions, about 2 amino acid substitutions to about 25 amino acid substitutions, about 2 amino acid substitutions to about 20 amino acid substitutions, about 2 amino acid substitutions to about 15 amino acid substitutions, about 2 amino acid substitutions to about 10 amino acid substitutions, about 2 amino acid substitutions to about 9 amino acid substitutions, about 2 amino acid substitutions to about 8 amino acid substitutions, about 2 amino acid substitutions to about 7 amino acid substitutions, about 2 amino acid substitutions to about 6 amino acid substitutions, about 2 amino acid substitutions to about 5 amino acid substitutions, about 2 amino acid substitutions to about 4 amino acid substitutions, about 3 amino acid substitutions to about 100 amino acid substitutions, about 3 amino acid substitutions to about 95 amino acid substitutions, about 3 amino acid substitutions to about 90 amino acid substitutions, about 3 amino acid substitutions to about 85 amino acid substitutions, about 3 amino acid substitutions to about 80 amino acid substitutions, about 3 amino acid substitutions to about 75 amino acid substitutions, about 3 amino acid substitutions to about 70 amino acid substitutions, about 3 amino acid substitutions to about 65 amino acid substitutions, about 3 amino acid substitutions to about 60 amino acid substitutions, about 3 amino acid substitutions to about 55 amino acid substitutions, about 3 amino acid substitutions to about 50 amino acid substitutions, about 3 amino acid substitutions to about 45 amino acid substitutions, about 3 amino acid substitutions to about 40 amino acid substitutions, about 3 amino acid substitutions to about 35 amino acid substitutions, about 3 amino acid substitutions to about 30 amino acid substitutions, about 3 amino acid substitutions to about 25 amino acid substitutions, about 3 amino acid substitutions to about 20 amino acid substitutions, about 3 amino acid substitutions to about 15 amino acid substitutions, about 3 amino acid substitutions to about 10 amino acid substitutions, about 3 amino acid substitutions to about 9 amino acid substitutions, about 3 amino acid substitutions to about 8 amino acid substitutions, about 3 amino acid substitutions to about 7 amino acid substitutions, about 3 amino acid substitutions to about 6 amino acid substitutions, about 3 amino acid substitutions to about 5 amino acid substitutions, about 4 amino acid substitutions to about 100 amino acid substitutions, about 4 amino acid substitutions to about 95 amino acid substitutions, about 4 amino acid substitutions to about 90 amino acid substitutions, about 4 amino acid substitutions to about 85 amino acid substitutions, about 4 amino acid substitutions to about 80 amino acid substitutions, about 4 amino acid substitutions to about 75 amino acid substitutions, about 4 amino acid substitutions to about 70 amino acid substitutions, about 4 amino acid substitutions to about 65 amino acid substitutions, about 4 amino acid substitutions to about 60 amino acid substitutions, about 4 amino acid substitutions to about 55 amino acid substitutions, about 4 amino acid substitutions to about 50 amino acid substitutions, about 4 amino acid substitutions to about 45 amino acid substitutions, about 4 amino acid substitutions to about 40 amino acid substitutions, about 4 amino acid substitutions to about 35 amino acid substitutions, about 4 amino acid substitutions to about 30 amino acid substitutions, about 4 amino acid substitutions to about 25 amino acid substitutions, about 4 amino acid substitutions to about 20 amino acid substitutions, about 4 amino acid substitutions to about 15 amino acid substitutions, about 4 amino acid substitutions to about 10 amino acid substitutions, about 4 amino acid substitutions to about 9 amino acid substitutions, about 4 amino acid substitutions to about 8 amino acid substitutions, about 4 amino acid substitutions to about 7 amino acid substitutions, about 4 amino acid substitutions to about 6 amino acid substitutions, about 5 amino acid substitutions to about 100 amino acid substitutions, about 5 amino acid substitutions to about 95 amino acid substitutions, about 5 amino acid substitutions to about 90 amino acid substitutions, about 5 amino acid substitutions to about 85 amino acid substitutions, about 5 amino acid substitutions to about 80 amino acid substitutions, about 5 amino acid substitutions to about 75 amino acid substitutions, about 5 amino acid substitutions to about 70 amino acid substitutions, about 5 amino acid substitutions to about 65 amino acid substitutions, about 5 amino acid substitutions to about 60 amino acid substitutions, about 5 amino acid substitutions to about 55 amino acid substitutions, about 5 amino acid substitutions to about 50 amino acid substitutions, about 5 amino acid substitutions to about 45 amino acid substitutions, about 5 amino acid substitutions to about 40 amino acid substitutions, about 5 amino acid substitutions to about 35 amino acid substitutions, about 5 amino acid substitutions to about 30 amino acid substitutions, about 5 amino acid substitutions to about 25 amino acid substitutions, about 5 amino acid substitutions to about 20 amino acid substitutions, about 5 amino acid substitutions to about 15 amino acid substitutions, about 5 amino acid substitutions to about 10 amino acid substitutions, about 5 amino acid substitutions to about 9 amino acid substitutions, about 5 amino acid substitutions to about 8 amino acid substitutions, about 5 amino acid substitutions to about 7 amino acid substitutions, about 6 amino acid substitutions to about 100 amino acid substitutions, about 6 amino acid substitutions to about 95 amino acid substitutions, about 6 amino acid substitutions to about 90 amino acid substitutions, about 6 amino acid substitutions to about 85 amino acid substitutions, about 6 amino acid substitutions to about 80 amino acid substitutions, about 6 amino acid substitutions to about 75 amino acid substitutions, about 6 amino acid substitutions to about 70 amino acid substitutions, about 6 amino acid substitutions to about 65 amino acid substitutions, about 6 amino acid substitutions to about 60 amino acid substitutions, about 6 amino acid substitutions to about 55 amino acid substitutions, about 6 amino acid substitutions to about 50 amino acid substitutions, about 6 amino acid substitutions to about 45 amino acid substitutions, about 6 amino acid substitutions to about 40 amino acid substitutions, about 6 amino acid substitutions to about 35 amino acid substitutions, about 6 amino acid substitutions to about 30 amino acid substitutions, about 6 amino acid substitutions to about 25 amino acid substitutions, about 6 amino acid substitutions to about 20 amino acid substitutions, about 6 amino acid substitutions to about 15 amino acid substitutions, about 6 amino acid substitutions to about 10 amino acid substitutions, about 6 amino acid substitutions to about 9 amino acid substitutions, about 6 amino acid substitutions to about 8 amino acid substitutions, about 7 amino acid substitutions to about 100 amino acid substitutions, about 7 amino acid substitutions to about 95 amino acid substitutions, about 7 amino acid substitutions to about 90 amino acid substitutions, about 7 amino acid substitutions to about 85 amino acid substitutions, about 7 amino acid substitutions to about 80 amino acid substitutions, about 7 amino acid substitutions to about 75 amino acid substitutions, about 7 amino acid substitutions to about 70 amino acid substitutions, about 7 amino acid substitutions to about 65 amino acid substitutions, about 7 amino acid substitutions to about 60 amino acid substitutions, about 7 amino acid substitutions to about 55 amino acid substitutions, about 7 amino acid substitutions to about 50 amino acid substitutions, about 7 amino acid substitutions to about 45 amino acid substitutions, about 7 amino acid substitutions to about 40 amino acid substitutions, about 7 amino acid substitutions to about 35 amino acid substitutions, about 7 amino acid substitutions to about 30 amino acid substitutions, about 7 amino acid substitutions to about 25 amino acid substitutions, about 7 amino acid substitutions to about 20 amino acid substitutions, about 7 amino acid substitutions to about 15 amino acid substitutions, about 7 amino acid substitutions to about 10 amino acid substitutions, about 7 amino acid substitutions to about 9 amino acid substitutions, about 8 amino acid substitutions to about 100 amino acid substitutions, about 8 amino acid substitutions to about 95 amino acid substitutions, about 8 amino acid substitutions to about 90 amino acid substitutions, about 8 amino acid substitutions to about 85 amino acid substitutions, about 8 amino acid substitutions to about 80 amino acid substitutions, about 8 amino acid substitutions to about 75 amino acid substitutions, about 8 amino acid substitutions to about 70 amino acid substitutions, about 8 amino acid substitutions to about 65 amino acid substitutions, about 8 amino acid substitutions to about 60 amino acid substitutions, about 8 amino acid substitutions to about 55 amino acid substitutions, about 8 amino acid substitutions to about 50 amino acid substitutions, about 8 amino acid substitutions to about 45 amino acid substitutions, about 8 amino acid substitutions to about 40 amino acid substitutions, about 8 amino acid substitutions to about 35 amino acid substitutions, about 8 amino acid substitutions to about 30 amino acid substitutions, about 8 amino acid substitutions to about 25 amino acid substitutions, about 8 amino acid substitutions to about 20 amino acid substitutions, about 8 amino acid substitutions to about 15 amino acid substitutions, about 8 amino acid substitutions to about 10 amino acid substitutions, about 10 amino acid substitutions to about 100 amino acid substitutions, about 10 amino acid substitutions to about 95 amino acid substitutions, about 10 amino acid substitutions to about 90 amino acid substitutions, about 10 amino acid substitutions to about 85 amino acid substitutions, about 10 amino acid substitutions to about 80 amino acid substitutions, about 10 amino acid substitutions to about 75 amino acid substitutions, about 10 amino acid substitutions to about 70 amino acid substitutions, about 10 amino acid substitutions to about 65 amino acid substitutions, about 10 amino acid substitutions to about 60 amino acid substitutions, about 10 amino acid substitutions to about 55 amino acid substitutions, about 10 amino acid substitutions to about 50 amino acid substitutions, about 10 amino acid substitutions to about 45 amino acid substitutions, about 10 amino acid substitutions to about 40 amino acid substitutions, about 10 amino acid substitutions to about 35 amino acid substitutions, about 10 amino acid substitutions to about 30 amino acid substitutions, about 10 amino acid substitutions to about 25 amino acid substitutions, about 10 amino acid substitutions to about 20 amino acid substitutions, about 10 amino acid substitutions to about 15 amino acid substitutions, about 15 amino acid substitutions to about 100 amino acid substitutions, about 15 amino acid substitutions to about 95 amino acid substitutions, about 15 amino acid substitutions to about 90 amino acid substitutions, about 15 amino acid substitutions to about 85 amino acid substitutions, about 15 amino acid substitutions to about 80 amino acid substitutions, about 15 amino acid substitutions to about 75 amino acid substitutions, about 15 amino acid substitutions to about 70 amino acid substitutions, about 15 amino acid substitutions to about 65 amino acid substitutions, about 15 amino acid substitutions to about 60 amino acid substitutions, about 15 amino acid substitutions to about 55 amino acid substitutions, about 15 amino acid substitutions to about 50 amino acid substitutions, about 15 amino acid substitutions to about 45 amino acid substitutions, about 15 amino acid substitutions to about 40 amino acid substitutions, about 15 amino acid substitutions to about 35 amino acid substitutions, about 15 amino acid substitutions to about 30 amino acid substitutions, about 15 amino acid substitutions to about 25 amino acid substitutions, about 15 amino acid substitutions to about 20 amino acid substitutions, about 20 amino acid substitutions to about 100 amino acid substitutions, about 20 amino acid substitutions to about 95 amino acid substitutions, about 20 amino acid substitutions to about 90 amino acid substitutions, about 20 amino acid substitutions to about 85 amino acid substitutions, about 20 amino acid substitutions to about 80 amino acid substitutions, about 20 amino acid substitutions to about 75 amino acid substitutions, about 20 amino acid substitutions to about 70 amino acid substitutions, about 20 amino acid substitutions to about 65 amino acid substitutions, about 20 amino acid substitutions to about 60 amino acid substitutions, about 20 amino acid substitutions to about 55 amino acid substitutions, about 20 amino acid substitutions to about 50 amino acid substitutions, about 20 amino acid substitutions to about 45 amino acid substitutions, about 20 amino acid substitutions to about 40 amino acid substitutions, about 20 amino acid substitutions to about 35 amino acid substitutions, about 20 amino acid substitutions to about 30 amino acid substitutions, about 20 amino acid substitutions to about 25 amino acid substitutions, about 25 amino acid substitutions to about 100 amino acid substitutions, about 25 amino acid substitutions to about 95 amino acid substitutions, about 25 amino acid substitutions to about 90 amino acid substitutions, about 25 amino acid substitutions to about 85 amino acid substitutions, about 25 amino acid substitutions to about 80 amino acid substitutions, about 25 amino acid substitutions to about 75 amino acid substitutions, about 25 amino acid substitutions to about 70 amino acid substitutions, about 25 amino acid substitutions to about 65 amino acid substitutions, about 25 amino acid substitutions to about 60 amino acid substitutions, about 25 amino acid substitutions to about 55 amino acid substitutions, about 25 amino acid substitutions to about 50 amino acid substitutions, about 25 amino acid substitutions to about 45 amino acid substitutions, about 25 amino acid substitutions to about 40 amino acid substitutions, about 25 amino acid substitutions to about 35 amino acid substitutions, about 25 amino acid substitutions to about 30 amino acid substitutions, about 30 amino acid substitutions to about 100 amino acid substitutions, about 30 amino acid substitutions to about 95 amino acid substitutions, about 30 amino acid substitutions to about 90 amino acid substitutions, about 30 amino acid substitutions to about 85 amino acid substitutions, about 30 amino acid substitutions to about 80 amino acid substitutions, about 30 amino acid substitutions to about 75 amino acid substitutions, about 30 amino acid substitutions to about 70 amino acid substitutions, about 30 amino acid substitutions to about 65 amino acid substitutions, about 30 amino acid substitutions to about 60 amino acid substitutions, about 30 amino acid substitutions to about 55 amino acid substitutions, about 30 amino acid substitutions to about 50 amino acid substitutions, about 30 amino acid substitutions to about 45 amino acid substitutions, about 30 amino acid substitutions to about 40 amino acid substitutions, about 30 amino acid substitutions to about 35 amino acid substitutions, about 35 amino acid substitutions to about 100 amino acid substitutions, about 35 amino acid substitutions to about 95 amino acid substitutions, about 35 amino acid substitutions to about 90 amino acid substitutions, about 35 amino acid substitutions to about 85 amino acid substitutions, about 35 amino acid substitutions to about 80 amino acid substitutions, about 35 amino acid substitutions to about 75 amino acid substitutions, about 35 amino acid substitutions to about 70 amino acid substitutions, about 35 amino acid substitutions to about 65 amino acid substitutions, about 35 amino acid substitutions to about 60 amino acid substitutions, about 35 amino acid substitutions to about 55 amino acid substitutions, about 35 amino acid substitutions to about 50 amino acid substitutions, about 35 amino acid substitutions to about 45 amino acid substitutions, about 35 amino acid substitutions to about 40 amino acid substitutions, about 40 amino acid substitutions to about 100 amino acid substitutions, about 40 amino acid substitutions to about 95 amino acid substitutions, about 40 amino acid substitutions to about 90 amino acid substitutions, about 40 amino acid substitutions to about 85 amino acid substitutions, about 40 amino acid substitutions to about 80 amino acid substitutions, about 40 amino acid substitutions to about 75 amino acid substitutions, about 40 amino acid substitutions to about 70 amino acid substitutions, about 40 amino acid substitutions to about 65 amino acid substitutions, about 40 amino acid substitutions to about 60 amino acid substitutions, about 40 amino acid substitutions to about 55 amino acid substitutions, about 40 amino acid substitutions to about 50 amino acid substitutions, about 40 amino acid substitutions to about 45 amino acid substitutions, about 45 amino acid substitutions to about 100 amino acid substitutions, about 45 amino acid substitutions to about 95 amino acid substitutions, about 45 amino acid substitutions to about 90 amino acid substitutions, about 45 amino acid substitutions to about 85 amino acid substitutions, about 45 amino acid substitutions to about 80 amino acid substitutions, about 45 amino acid substitutions to about 75 amino acid substitutions, about 45 amino acid substitutions to about 70 amino acid substitutions, about 45 amino acid substitutions to about 65 amino acid substitutions, about 45 amino acid substitutions to about 60 amino acid substitutions, about 45 amino acid substitutions to about 55 amino acid substitutions, about 45 amino acid substitutions to about 50 amino acid substitutions, about 50 amino acid substitutions to about 100 amino acid substitutions, about 50 amino acid substitutions to about 95 amino acid substitutions, about 50 amino acid substitutions to about 90 amino acid substitutions, about 50 amino acid substitutions to about 85 amino acid substitutions, about 50 amino acid substitutions to about 80 amino acid substitutions, about 50 amino acid substitutions to about 75 amino acid substitutions, about 50 amino acid substitutions to about 70 amino acid substitutions, about 50 amino acid substitutions to about 65 amino acid substitutions, about 50 amino acid substitutions to about 60 amino acid substitutions, about 50 amino acid substitutions to about 55 amino acid substitutions, about 60 amino acid substitutions to about 100 amino acid substitutions, about 60 amino acid substitutions to about 95 amino acid substitutions, about 60 amino acid substitutions to about 90 amino acid substitutions, about 60 amino acid substitutions to about 85 amino acid substitutions, about 60 amino acid substitutions to about 80 amino acid substitutions, about 60 amino acid substitutions to about 75 amino acid substitutions, about 60 amino acid substitutions to about 70 amino acid substitutions, about 60 amino acid substitutions to about 65 amino acid substitutions, about 70 amino acid substitutions to about 100 amino acid substitutions, about 70 amino acid substitutions to about 95 amino acid substitutions, about 70 amino acid substitutions to about 90 amino acid substitutions, about 70 amino acid substitutions to about 85 amino acid substitutions, about 70 amino acid substitutions to about 80 amino acid substitutions, about 70 amino acid substitutions to about 75 amino acid substitutions, about 80 amino acid substitutions to about 100 amino acid substitutions, about 80 amino acid substitutions to about 95 amino acid substitutions, about 80 amino acid substitutions to about 90 amino acid substitutions, about 80 amino acid substitutions to about 85 amino acid substitutions, about 90 amino acid substitutions to about 100 amino acid substitutions, about 90 amino acid substitutions to about 95 amino acid substitutions, or about 95 amino acids to about 100 amino acids.

One skilled in the art would appreciate that amino acids that are not conserved between wildtype CLRN1 proteins from different species can be mutated without losing activity, while those amino acids that are conserved between wildtype CLRN1 proteins from different species should not be mutated as they are more likely (than amino acids that are not conserved between different species) to be involved in activity.

An active CLRN1 protein can include, e.g., a sequence of a wildtype, full-length CLRN1 protein (e.g., a wildtype, human, full-length CLRN1 protein) that has about 1 amino acid to about 100 amino acids, about 1 amino acid to about 95 amino acids, about 1 amino acid to about 90 amino acids, about 1 amino acid to about 85 amino acids, about 1 amino acid to about 80 amino acids, about 1 amino acid to about 75 amino acids, about 1 amino acid to about 70 amino acids, about 1 amino acid to about 65 amino acids, about 1 amino acid to about 60 amino acids, about 1 amino acid to about 55 amino acids, about 1 amino acid to about 50 amino acids, about 1 amino acid to about 45 amino acids, about 1 amino acid to about 40 amino acids, about 1 amino acid to about 35 amino acids, about 1 amino acid to about 30 amino acids, about 1 amino acid to about 25 amino acids, about 1 amino acid to about 20 amino acids, about 1 amino acid to about 15 amino acids, about 1 amino acid to about 10 amino acids, about 1 amino acid to about 9 amino acids, about 1 amino acid to about 8 amino acids, about 1 amino acid to about 7 amino acids, about 1 amino acid to about 6 amino acids, about 1 amino acid to about 5 amino acids, about 1 amino acid to about 4 amino acids, about 1 amino acid to about 3 amino acids, about 2 amino acids to about 100 amino acids, about 2 amino acids to about 95 amino acids, about 2 amino acids to about 90 amino acids, about 2 amino acids to about 85 amino acids, about 2 amino acids to about 80 amino acids, about 2 amino acids to about 75 amino acids, about 2 amino acids to about 70 amino acids, about 2 amino acids to about 65 amino acids, about 2 amino acids to about 60 amino acids, about 2 amino acids to about 55 amino acids, about 2 amino acids to about 50 amino acids, about 2 amino acids to about 45 amino acids, about 2 amino acids to about 40 amino acids, about 2 amino acids to about 35 amino acids, about 2 amino acids to about 30 amino acids, about 2 amino acids to about 25 amino acids, about 2 amino acids to about 20 amino acids, about 2 amino acids to about 15 amino acids, about 2 amino acids to about 10 amino acids, about 2 amino acids to about 9 amino acids, about 2 amino acids to about 8 amino acids, about 2 amino acids to about 7 amino acids, about 2 amino acids to about 6 amino acids, about 2 amino acids to about 5 amino acids, about 2 amino acids to about 4 amino acids, about 3 amino acids to about 100 amino acids, about 3 amino acids to about 95 amino acids, about 3 amino acids to about 90 amino acids, about 3 amino acids to about 85 amino acids, about 3 amino acids to about 80 amino acids, about 3 amino acids to about 75 amino acids, about 3 amino acids to about 70 amino acids, about 3 amino acids to about 65 amino acids, about 3 amino acids to about 60 amino acids, about 3 amino acids to about 55 amino acids, about 3 amino acids to about 50 amino acids, about 3 amino acids to about 45 amino acids, about 3 amino acids to about 40 amino acids, about 3 amino acids to about 35 amino acids, about 3 amino acids to about 30 amino acids, about 3 amino acids to about 25 amino acids, about 3 amino acids to about 20 amino acids, about 3 amino acids to about 15 amino acids, about 3 amino acids to about 10 amino acids, about 3 amino acids to about 9 amino acids, about 3 amino acids to about 8 amino acids, about 3 amino acids to about 7 amino acids, about 3 amino acids to about 6 amino acids, about 3 amino acids to about 5 amino acids, about 4 amino acids to about 100 amino acids, about 4 amino acids to about 95 amino acids, about 4 amino acids to about 90 amino acids, about 4 amino acids to about 85 amino acids, about 4 amino acids to about 80 amino acids, about 4 amino acids to about 75 amino acids, about 4 amino acids to about 70 amino acids, about 4 amino acids to about 65 amino acids, about 4 amino acids to about 60 amino acids, about 4 amino acids to about 55 amino acids, about 4 amino acids to about 50 amino acids, about 4 amino acids to about 45 amino acids, about 4 amino acids to about 40 amino acids, about 4 amino acids to about 35 amino acids, about 4 amino acids to about 30 amino acids, about 4 amino acids to about 25 amino acids, about 4 amino acids to about 20 amino acids, about 4 amino acids to about 15 amino acids, about 4 amino acids to about 10 amino acids, about 4 amino acids to about 9 amino acids, about 4 amino acids to about 8 amino acids, about 4 amino acids to about 7 amino acids, about 4 amino acids to about 6 amino acids, about 5 amino acids to about 100 amino acids, about 5 amino acids to about 95 amino acids, about 5 amino acids to about 90 amino acids, about 5 amino acids to about 85 amino acids, about 5 amino acids to about 80 amino acids, about 5 amino acids to about 75 amino acids, about 5 amino acids to about 70 amino acids, about 5 amino acids to about 65 amino acids, about 5 amino acids to about 60 amino acids, about 5 amino acids to about 55 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 5 amino acids to about 9 amino acids, about 5 amino acids to about 8 amino acids, about 5 amino acids to about 7 amino acids, about 6 amino acids to about 100 amino acids, about 6 amino acids to about 95 amino acids, about 6 amino acids to about 90 amino acids, about 6 amino acids to about 85 amino acids, about 6 amino acids to about 80 amino acids, about 6 amino acids to about 75 amino acids, about 6 amino acids to about 70 amino acids, about 6 amino acids to about 65 amino acids, about 6 amino acids to about 60 amino acids, about 6 amino acids to about 55 amino acids, about 6 amino acids to about 50 amino acids, about 6 amino acids to about 45 amino acids, about 6 amino acids to about 40 amino acids, about 6 amino acids to about 35 amino acids, about 6 amino acids to about 30 amino acids, about 6 amino acids to about 25 amino acids, about 6 amino acids to about 20 amino acids, about 6 amino acids to about 15 amino acids, about 6 amino acids to about 10 amino acids, about 6 amino acids to about 9 amino acids, about 6 amino acids to about 8 amino acids, about 7 amino acids to about 100 amino acids, about 7 amino acids to about 95 amino acids, about 7 amino acids to about 90 amino acids, about 7 amino acids to about 85 amino acids, about 7 amino acids to about 80 amino acids, about 7 amino acids to about 75 amino acids, about 7 amino acids to about 70 amino acids, about 7 amino acids to about 65 amino acids, about 7 amino acids to about 60 amino acids, about 7 amino acids to about 55 amino acids, about 7 amino acids to about 50 amino acids, about 7 amino acids to about 45 amino acids, about 7 amino acids to about 40 amino acids, about 7 amino acids to about 35 amino acids, about 7 amino acids to about 30 amino acids, about 7 amino acids to about 25 amino acids, about 7 amino acids to about 20 amino acids, about 7 amino acids to about 15 amino acids, about 7 amino acids to about 10 amino acids, about 7 amino acids to about 9 amino acids, about 8 amino acids to about 100 amino acids, about 8 amino acids to about 95 amino acids, about 8 amino acids to about 90 amino acids, about 8 amino acids to about 85 amino acids, about 8 amino acids to about 80 amino acids, about 8 amino acids to about 75 amino acids, about 8 amino acids to about 70 amino acids, about 8 amino acids to about 65 amino acids, about 8 amino acids to about 60 amino acids, about 8 amino acids to about 55 amino acids, about 8 amino acids to about 50 amino acids, about 8 amino acids to about 45 amino acids, about 8 amino acids to about 40 amino acids, about 8 amino acids to about 35 amino acids, about 8 amino acids to about 30 amino acids, about 8 amino acids to about 25 amino acids, about 8 amino acids to about 20 amino acids, about 8 amino acids to about 15 amino acids, about 8 amino acids to about 10 amino acids, about 10 amino acids to about 100 amino acids, about 10 amino acids to about 95 amino acids, about 10 amino acids to about 90 amino acids, about 10 amino acids to about 85 amino acids, about 10 amino acids to about 80 amino acids, about 10 amino acids to about 75 amino acids, about 10 amino acids to about 70 amino acids, about 10 amino acids to about 65 amino acids, about 10 amino acids to about 60 amino acids, about 10 amino acids to about 55 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 100 amino acids, about 15 amino acids to about 95 amino acids, about 15 amino acids to about 90 amino acids, about 15 amino acids to about 85 amino acids, about 15 amino acids to about 80 amino acids, about 15 amino acids to about 75 amino acids, about 15 amino acids to about 70 amino acids, about 15 amino acids to about 65 amino acids, about 15 amino acids to about 60 amino acids, about 15 amino acids to about 55 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 100 amino acids, about 20 amino acids to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 65 amino acids, about 20 amino acids to about 60 amino acids, about 20 amino acids to about 55 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 100 amino acids, about 25 amino acids to about 95 amino acids, about 25 amino acids to about 90 amino acids, about 25 amino acids to about 85 amino acids, about 25 amino acids to about 80 amino acids, about 25 amino acids to about 75 amino acids, about 25 amino acids to about 70 amino acids, about 25 amino acids to about 65 amino acids, about 25 amino acids to about 60 amino acids, about 25 amino acids to about 55 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 100 amino acids, about 30 amino acids to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 65 amino acids, about 30 amino acids to about 60 amino acids, about 30 amino acids to about 55 amino acids, about 30 amino acids to about 50 amino acids, about 30 amino acids to about 45 amino acids, about 30 amino acids to about 40 amino acids, about 30 amino acids to about 35 amino acids, about 35 amino acids to about 50 amino acids, about 35 amino acids to about 45 amino acids, about 35 amino acids to about 40 amino acids, about 40 amino acids to about 100 amino acids, about 40 amino acids to about 95 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 65 amino acids, about 40 amino acids to about 60 amino acids, about 40 amino acids to about 55 amino acids, about 40 amino acids to about 50 amino acids, about 40 amino acids to about 45 amino acids, about 45 amino acids to about 50 amino acids, about 50 amino acids to about 100 amino acids, about 50 amino acids to about 95 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 65 amino acids, about 50 amino acids to about 60 amino acids, or about 50 amino acids to about 55 amino acids, deleted. In some embodiments where two or more amino acids are deleted from the sequence of a wildtype, full-length CLRN1 protein, at least two of the two or more deleted amino acids can be contiguous in the sequence of the wildtype, full-length protein. In other examples where two or more amino acids are deleted from the sequence of a wildtype, full-length CLRN1 protein, some or all of the two or more deleted amino acids are not contiguous in the sequence of the wildtype, full-length protein. One skilled in the art would appreciate that amino acids that are not conserved between wildtype, full-length CLRN1 proteins from different species can be deleted without losing activity, while those amino acids that are conserved between wildtype, full-length CLNRN1 proteins from different species should not be deleted as they are more likely (than amino acids that are not conserved between different species) to be involved in activity.

In some examples, an active CLRN1 protein can, e.g., include a sequence of a wildtype, full-length CLRN1 protein that has between 1 amino acid to about 100 amino acids, 1 amino acid to about 95 amino acids, 1 amino acid to about 90 amino acids, 1 amino acid to about 85 amino acids, 1 amino acid to about 80 amino acids, 1 amino acid to about 75 amino acids, 1 amino acid to about 70 amino acids, 1 amino acid to about 65 amino acids, 1 amino acid to about 60 amino acids, 1 amino acid to about 55 amino acids, 1 amino acid to about 50 amino acids, 1 amino acid to about 45 amino acids, 1 amino acid to about 40 amino acids, 1 amino acid to about 35 amino acids, 1 amino acid to about 30 amino acids, 1 amino acid to about 25 amino acids, 1 amino acid to about 20 amino acids, 1 amino acid to about 15 amino acids, 1 amino acid to about 10 amino acids, 1 amino acid to about 9 amino acids, 1 amino acid to about 8 amino acids, 1 amino acid to about 7 amino acids, 1 amino acid to about 6 amino acids, 1 amino acid to about 5 amino acids, 1 amino acid to about 4 amino acids, 1 amino acid to about 3 amino acids, about 2 amino acids to about 100 amino acids, about 2 amino acid to about 95 amino acids, about 2 amino acids to about 90 amino acids, about 2 amino acids to about 85 amino acids, about 2 amino acids to about 80 amino acids, about 2 amino acids to about 75 amino acids, about 2 amino acids to about 70 amino acids, about 2 amino acids to about 65 amino acids, about 2 amino acids to about 60 amino acids, about 2 amino acids to about 55 amino acids, about 2 amino acids to about 50 amino acids, about 2 amino acids to about 45 amino acids, about 2 amino acids to about 40 amino acids, about 2 amino acids to about 35 amino acids, about 2 amino acids to about 30 amino acids, about 2 amino acids to about 25 amino acids, about 2 amino acids to about 20 amino acids, about 2 amino acids to about 15 amino acids, about 2 amino acids to about 10 amino acids, about 2 amino acids to about 9 amino acids, about 2 amino acids to about 8 amino acids, about 2 amino acids to about 7 amino acids, about 2 amino acids to about 6 amino acids, about 2 amino acids to about 5 amino acids, about 2 amino acids to about 4 amino acids, about 3 amino acids to about 100 amino acids, about 3 amino acid to about 95 amino acids, about 3 amino acids to about 90 amino acids, about 3 amino acids to about 85 amino acids, about 3 amino acids to about 80 amino acids, about 3 amino acids to about 75 amino acids, about 3 amino acids to about 70 amino acids, about 3 amino acids to about 65 amino acids, about 3 amino acids to about 60 amino acids, about 3 amino acids to about 55 amino acids, about 3 amino acids to about 50 amino acids, about 3 amino acids to about 45 amino acids, about 3 amino acids to about 40 amino acids, about 3 amino acids to about 35 amino acids, about 3 amino acids to about 30 amino acids, about 3 amino acids to about 25 amino acids, about 3 amino acids to about 20 amino acids, about 3 amino acids to about 15 amino acids, about 3 amino acids to about 10 amino acids, about 3 amino acids to about 9 amino acids, about 3 amino acids to about 8 amino acids, about 3 amino acids to about 7 amino acids, about 3 amino acids to about 6 amino acids, about 3 amino acids to about 5 amino acids, about 4 amino acids to about 100 amino acids, about 4 amino acid to about 95 amino acids, about 4 amino acids to about 90 amino acids, about 4 amino acids to about 85 amino acids, about 4 amino acids to about 80 amino acids, about 4 amino acids to about 75 amino acids, about 4 amino acids to about 70 amino acids, about 4 amino acids to about 65 amino acids, about 4 amino acids to about 60 amino acids, about 4 amino acids to about 55 amino acids, about 4 amino acids to about 50 amino acids, about 4 amino acids to about 45 amino acids, about 4 amino acids to about 40 amino acids, about 4 amino acids to about 35 amino acids, about 4 amino acids to about 30 amino acids, about 4 amino acids to about 25 amino acids, about 4 amino acids to about 20 amino acids, about 4 amino acids to about 15 amino acids, about 4 amino acids to about 10 amino acids, about 4 amino acids to about 9 amino acids, about 4 amino acids to about 8 amino acids, about 4 amino acids to about 7 amino acids, about 4 amino acids to about 6 amino acids, about 5 amino acids to about 100 amino acids, about 5 amino acid to about 95 amino acids, about 5 amino acids to about 90 amino acids, about 5 amino acids to about 85 amino acids, about 5 amino acids to about 80 amino acids, about 5 amino acids to about 75 amino acids, about 5 amino acids to about 70 amino acids, about 5 amino acids to about 65 amino acids, about 5 amino acids to about 60 amino acids, about 5 amino acids to about 55 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 5 amino acids to about 9 amino acids, about 5 amino acids to about 8 amino acids, about 5 amino acids to about 7 amino acids, about 6 amino acids to about 100 amino acids, about 6 amino acid to about 95 amino acids, about 6 amino acids to about 90 amino acids, about 6 amino acids to about 85 amino acids, about 6 amino acids to about 80 amino acids, about 6 amino acids to about 75 amino acids, about 6 amino acids to about 70 amino acids, about 6 amino acids to about 65 amino acids, about 6 amino acids to about 60 amino acids, about 6 amino acids to about 55 amino acids, about 6 amino acids to about 50 amino acids, about 6 amino acids to about 45 amino acids, about 6 amino acids to about 40 amino acids, about 6 amino acids to about 35 amino acids, about 6 amino acids to about 30 amino acids, about 6 amino acids to about 25 amino acids, about 6 amino acids to about 20 amino acids, about 6 amino acids to about 15 amino acids, about 6 amino acids to about 10 amino acids, about 6 amino acids to about 9 amino acids, about 6 amino acids to about 8 amino acids, about 7 amino acids to about 100 amino acids, about 7 amino acid to about 95 amino acids, about 7 amino acids to about 90 amino acids, about 7 amino acids to about 85 amino acids, about 7 amino acids to about 80 amino acids, about 7 amino acids to about 75 amino acids, about 7 amino acids to about 70 amino acids, about 7 amino acids to about 65 amino acids, about 7 amino acids to about 60 amino acids, about 7 amino acids to about 55 amino acids, about 7 amino acids to about 50 amino acids, about 7 amino acids to about 45 amino acids, about 7 amino acids to about 40 amino acids, about 7 amino acids to about 35 amino acids, about 7 amino acids to about 30 amino acids, about 7 amino acids to about 25 amino acids, about 7 amino acids to about 20 amino acids, about 7 amino acids to about 15 amino acids, about 7 amino acids to about 10 amino acids, about 7 amino acids to about 9 amino acids, about 8 amino acids to about 100 amino acids, about 8 amino acid to about 95 amino acids, about 8 amino acids to about 90 amino acids, about 8 amino acids to about 85 amino acids, about 8 amino acids to about 80 amino acids, about 8 amino acids to about 75 amino acids, about 8 amino acids to about 70 amino acids, about 8 amino acids to about 65 amino acids, about 8 amino acids to about 60 amino acids, about 8 amino acids to about 55 amino acids, about 8 amino acids to about 50 amino acids, about 8 amino acids to about 45 amino acids, about 8 amino acids to about 40 amino acids, about 8 amino acids to about 35 amino acids, about 8 amino acids to about 30 amino acids, about 8 amino acids to about 25 amino acids, about 8 amino acids to about 20 amino acids, about 8 amino acids to about 15 amino acids, about 8 amino acids to about 10 amino acids, about 10 amino acids to about 100 amino acids, about 10 amino acid to about 95 amino acids, about 10 amino acids to about 90 amino acids, about 10 amino acids to about 85 amino acids, about 10 amino acids to about 80 amino acids, about 10 amino acids to about 75 amino acids, about 10 amino acids to about 70 amino acids, about 10 amino acids to about 65 amino acids, about 10 amino acids to about 60 amino acids, about 10 amino acids to about 55 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 20 amino acids to about 100 amino acids, about 20 amino acid to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 65 amino acids, about 20 amino acids to about 60 amino acids, about 20 amino acids to about 55 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 30 amino acids to about 100 amino acids, about 30 amino acid to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 65 amino acids, about 30 amino acids to about 60 amino acids, about 30 amino acids to about 55 amino acids, about 30 amino acids to about 50 amino acids, about 30 amino acids to about 45 amino acids, about 30 amino acids to about 40 amino acids, about 30 amino acids to about 35 amino acids, about 40 amino acids to about 100 amino acids, about 40 amino acid to about 95 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 65 amino acids, about 40 amino acids to about 60 amino acids, about 40 amino acids to about 55 amino acids, about 40 amino acids to about 50 amino acids, about 40 amino acids to about 45 amino acids, about 50 amino acids to about 100 amino acids, about 50 amino acid to about 95 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 65 amino acids, about 50 amino acids to about 60 amino acids, about 50 amino acids to about 55 amino acids, about 60 amino acids to about 100 amino acids, about 60 amino acid to about 95 amino acids, about 60 amino acids to about 90 amino acids, about 60 amino acids to about 85 amino acids, about 60 amino acids to about 80 amino acids, about 60 amino acids to about 75 amino acids, about 60 amino acids to about 70 amino acids, about 60 amino acids to about 65 amino acids, about 70 amino acids to about 100 amino acids, about 70 amino acid to about 95 amino acids, about 70 amino acids to about 90 amino acids, about 70 amino acids to about 85 amino acids, about 70 amino acids to about 80 amino acids, about 70 amino acids to about 75 amino acids, about 80 amino acids to about 100 amino acids, about 80 amino acid to about 95 amino acids, about 80 amino acids to about 90 amino acids, about 80 amino acids to about 85 amino acids, about 90 amino acids to about 100 amino acids, about 90 amino acids to about 95 amino acids, or about 95 amino acids to about 100 amino acids, removed from its N-terminus and/or 1 amino acid to 100 amino acids (or any of the subranges of this range described herein) removed from its C-terminus.

In some embodiments, an active CLRN1 protein can, e.g., include the sequence of a wildtype, full-length CLRN1 protein where 1 amino acid to 50 amino acids, 1 amino acid to 45 amino acids, 1 amino acid to 40 amino acids, 1 amino acid to 35 amino acids, 1 amino acid to 30 amino acids, 1 amino acid to 25 amino acids, 1 amino acid to 20 amino acids, 1 amino acid to 15 amino acids, 1 amino acid to 10 amino acids, 1 amino acid to 9 amino acids, 1 amino acid to 8 amino acids, 1 amino acid to 7 amino acids, 1 amino acid to 6 amino acids, 1 amino acid to 5 amino acids, 1 amino acid to 4 amino acids, 1 amino acid to 3 amino acids, about 2 amino acids to 50 amino acids, about 2 amino acids to 45 amino acids, about 2 amino acids to 40 amino acids, about 2 amino acids to 35 amino acids, about 2 amino acids to 30 amino acids, about 2 amino acids to 25 amino acids, about 2 amino acids to 20 amino acids, about 2 amino acids to 15 amino acids, about 2 amino acids to 10 amino acids, about 2 amino acids to 9 amino acids, about 2 amino acids to 8 amino acids, about 2 amino acids to 7 amino acids, about 2 amino acids to 6 amino acids, about 2 amino acids to 5 amino acids, about 2 amino acids to 4 amino acids, about 3 amino acids to 50 amino acids, about 3 amino acids to 45 amino acids, about 3 amino acids to 40 amino acids, about 3 amino acids to 35 amino acids, about 3 amino acids to 30 amino acids, about 3 amino acids to 25 amino acids, about 3 amino acids to 20 amino acids, about 3 amino acids to 15 amino acids, about 3 amino acids to 10 amino acids, about 3 amino acids to 9 amino acids, about 3 amino acids to 8 amino acids, about 3 amino acids to 7 amino acids, about 3 amino acids to 6 amino acids, about 3 amino acids to 5 amino acids, about 4 amino acids to 50 amino acids, about 4 amino acids to 45 amino acids, about 4 amino acids to 40 amino acids, about 4 amino acids to 35 amino acids, about 4 amino acids to 30 amino acids, about 4 amino acids to 25 amino acids, about 4 amino acids to 20 amino acids, about 4 amino acids to 15 amino acids, about 4 amino acids to 10 amino acids, about 4 amino acids to 9 amino acids, about 4 amino acids to 8 amino acids, about 4 amino acids to 7 amino acids, about 4 amino acids to 6 amino acids, about 5 amino acids to 50 amino acids, about 5 amino acids to 45 amino acids, about 5 amino acids to 40 amino acids, about 5 amino acids to 35 amino acids, about 5 amino acids to 30 amino acids, about 5 amino acids to 25 amino acids, about 5 amino acids to 20 amino acids, about 5 amino acids to 15 amino acids, about 5 amino acids to 10 amino acids, about 5 amino acids to 9 amino acids, about 5 amino acids to 8 amino acids, about 5 amino acids to 7 amino acids, about 6 amino acids to 50 amino acids, about 6 amino acids to 45 amino acids, about 6 amino acids to 40 amino acids, about 6 amino acids to 35 amino acids, about 6 amino acids to 30 amino acids, about 6 amino acids to 25 amino acids, about 6 amino acids to 20 amino acids, about 6 amino acids to 15 amino acids, about 6 amino acids to 10 amino acids, about 6 amino acids to 9 amino acids, about 6 amino acids to 8 amino acids, about 7 amino acids to 50 amino acids, about 7 amino acids to 45 amino acids, about 7 amino acids to 40 amino acids, about 7 amino acids to 35 amino acids, about 7 amino acids to 30 amino acids, about 7 amino acids to 25 amino acids, about 7 amino acids to 20 amino acids, about 7 amino acids to 15 amino acids, about 7 amino acids to 10 amino acids, about 7 amino acids to 9 amino acids, about 8 amino acids to 50 amino acids, about 8 amino acids to 45 amino acids, about 8 amino acids to 40 amino acids, about 8 amino acids to 35 amino acids, about 8 amino acids to 30 amino acids, about 8 amino acids to 25 amino acids, about 8 amino acids to 20 amino acids, about 8 amino acids to 15 amino acids, about 8 amino acids to 10 amino acids, about 10 amino acids to 50 amino acids, about 10 amino acids to 45 amino acids, about 10 amino acids to 40 amino acids, about 10 amino acids to 35 amino acids, about 10 amino acids to 30 amino acids, about 10 amino acids to 25 amino acids, about 10 amino acids to 20 amino acids, about 10 amino acids to 15 amino acids, about 15 amino acids to 50 amino acids, about 15 amino acids to 45 amino acids, about 15 amino acids to 40 amino acids, about 15 amino acids to 35 amino acids, about 15 amino acids to 30 amino acids, about 15 amino acids to 25 amino acids, about 15 amino acids to 20 amino acids, about 20 amino acids to 50 amino acids, about 20 amino acids to 45 amino acids, about 20 amino acids to 40 amino acids, about 20 amino acids to 35 amino acids, about 20 amino acids to 30 amino acids, about 20 amino acids to 25 amino acids, about 25 amino acids to 50 amino acids, about 25 amino acids to 45 amino acids, about 25 amino acids to 40 amino acids, about 25 amino acids to 35 amino acids, about 25 amino acids to 30 amino acids, about 30 amino acids to 50 amino acids, about 30 amino acids to 45 amino acids, about 30 amino acids to 40 amino acids, about 30 amino acids to 35 amino acids, about 35 amino acids to 50 amino acids, about 35 amino acids to 45 amino acids, about 35 amino acids to 40 amino acids, about 40 amino acids to 50 amino acids, about 40 amino acids to 45 amino acids, or about 45 amino acids to about 50 amino acids, are inserted. In some examples, the 1 amino acid to 50 amino acids (or any subrange thereof) can be inserted as a contiguous sequence into the sequence of a wildtype, full-length protein. In some examples, the 1 amino acid to 50 amino acids (or any subrange thereof) are inserted in multiple, non-contiguous places in the sequence of a wildtype, full-length protein. As can be appreciated in the art, the 1 amino acid to 50 amino acids can be inserted into a portion of the sequence of a wildtype, full-length protein that is not well-conserved between species.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims

### BRIEF DESCRIPTION OF DRAWINGS

FIG. 1 is an exemplary schematic representation of a genetic map of a CLRN-1 vector (SEQ ID NO: 40; 3397 basepairs (bp)) that can be used in any of the present methods described herein. The vector includes an AAV2 ITR, CMV enhancer (SEQ ID NO: 17), chicken β-actin promoter, chimeric intron (SEQ ID NO: 16), CLRN1 isoform 1 (SEQ ID NO: 1), T2A sequence (SEQ ID NO: 31), CLRN1 isoform 2 (SEQ ID NO: 6), bGH poly(A) signal (SEQ ID NO: 20) and an AAV2 ITR.

FIG. 2 is an exemplary schematic representation of a genetic map of a CLRN-2GFPvector (SEQ ID NO: 41; 4177 bp) that can be used in any of the present methods described herein. The vector includes an AAV2 ITR, CMV enhancer (SEQ ID NO: 17), chicken β-actin promoter, chimeric intron (SEQ ID NO: 16), CLRN1 isoform 1 (SEQ ID NO: 1), T2A sequence (SEQ ID NO: 31), CLRN1 isoform 2 (SEQ ID NO: 6), T2A sequence (SEQ ID NO: 31), an eGFP (SEQ ID NO: 32), bGH poly(A) signal (SEQ ID NO: 20) and an AAV2 ITR.

FIG. 3 is an exemplary schematic representation of a genetic map of a CLRN-3 vector (SEQ ID NO: 42; 4607 bp) that can be used in any of the present methods described herein. The vector includes an AAV2 ITR, CMV enhancer (SEQ ID NO: 17), chicken β-actin promoter, chimeric intron (SEQ ID NO: 16), 5′UTR-291, CLRN1 isoform 1 (SEQ ID NO: 1), 3′UTR-1357 (SEQ ID NO: 36), bGH poly(A) signal (SEQ ID NO: 20) and an AAV2 ITR.

FIG. 4 is an exemplary schematic representation of a genetic map of a CLRN-4 vector (SEQ ID NO: 43; 4796 bp) that can be used in any of the present methods described herein. The vector includes an AAV2 ITR, CMV enhancer (SEQ ID NO: 17), chicken β-actin promoter, chimeric intron (SEQ ID NO: 16), CLRN1 isoform 1 (SEQ ID NO: 1), T2A sequence (SEQ ID NO: 31), CLRN1 isoform 2 (SEQ ID NO: 6), 3′UTR-1406 (SEQ ID NO: 37), bGH poly(A) signal (SEQ ID NO: 20) and an AAV2 ITR.

FIG. 5 is an exemplary schematic representation of a genetic map of a pITR-CBA-5′UTR-tGFP-3′UTR vector (5026 bp) that can be used in any of the present methods described herein. The vector includes an AAV2 ITR, CMV enhancer (SEQ ID NO: 17), chicken β-actin promoter, chimeric intron (SEQ ID NO: 16), 5′UTR-291, tGFP (SEQ ID NO: 19), 3′UTR-1595, bGH poly(A) signal (SEQ ID NO: 20) and an AAV2 ITR.

FIG. 6 is an exemplary schematic representation of a genetic map of a CLRN-6eGFP vector (SEQ ID NO: 44; 4756 bp) that can be used in any of the present methods described herein. The vector includes an AAV2 ITR, CMV enhancer (SEQ ID NO: 17), chicken β-actin promoter, chimeric intron (SEQ ID NO: 16), eGFP (SEQ ID NO: 32), 3′UTR-1406 (SEQ ID NO: 37), bGH poly(A) signal (SEQ ID NO: 20) and an AAV2 ITR.

FIG. 7 is an exemplary schematic representation of a genetic map of a pITR-CBA-3′UTR-600A vector (3982 bp) that can be used in any of the present methods described herein. The vector includes an AAV2 ITR, CMV enhancer (SEQ ID NO: 17), chicken β-actin promoter, chimeric intron (SEQ ID NO: 16), CLRN1 isoform 1 (SEQ ID NO: 4), T2A sequence (SEQ ID NO: 31), CLRN1 isoform 2 (SEQ ID NO: 6), 3′UTR-600 (SEQ ID NO: 27), bGH poly(A) signal (SEQ ID NO: 20) and an AAV2 ITR.

FIG. 8 is an exemplary schematic representation of a genetic map of a CLRN-8 vector (SEQ ID NO: 46; 3982 bp) that can be used in any of the present methods described herein. The vector includes an AAV2 ITR, CMV enhancer (SEQ ID NO: 17), chicken β-actin promoter, chimeric intron (SEQ ID NO: 16), CLRN1 isoform 1 (SEQ ID NO: 1), T2A sequence (SEQ ID NO: 31), CLRN1 isoform 2 (SEQ ID NO: 6), 3′UTR-600B (SEQ ID NO: 28), bGH poly(A) signal (SEQ ID NO: 20) and an AAV2 ITR.

FIG. 9 is an exemplary schematic representation of a genetic map of a CLRN-9 vector (SEQ ID NO: 47; 3982 bp) that can be used in any of the present methods described herein. The vector includes an AAV2 ITR, CMV enhancer (SEQ ID NO: 17), chicken β-actin promoter, chimeric intron (SEQ ID NO: 16), CLRN1 isoform 1 (SEQ ID NO: 1), T2A sequence (SEQ ID NO: 31), CLRN1 isoform 2 (SEQ ID NO: 6), 3′UTR-600C (SEQ ID NO: 29), bGH poly(A) signal (SEQ ID NO: 20) and an AAV2 ITR.

FIG. 10 is an exemplary schematic representation of a genetic map of a CLRN-0 vector (SEQ ID NO: 39; 4732 bp) that can be used in any of the present methods described herein. The vector includes an AAV2 ITR, CMV enhancer (SEQ ID NO: 17), chicken β-actin promoter, a chimeric intron (SEQ ID NO: 16), CLRN1 isoform 1 (SEQ ID NO: 1), a 3′UTR 1773 (SEQ ID NO: 15), bGH poly(A) signal (SEQ ID NO: 20) and an AAV2 ITR.

FIG. 11 is an exemplary schematic representation of a genetic map of a CLRN-7eGFP vector (SEQ ID NO: 45; 3580 bp) that can be used in any of the present methods described herein. The vector includes an AAV2 ITR, CMV enhancer (SEQ ID NO: 17), chicken β-actin promoter, chimeric intron (SEQ ID NO: 16), an eGFP sequence (SEQ ID NO: 32), bGH poly(A) signal (SEQ ID NO: 20) and an AAV2 ITR.

FIG. 12 is an exemplary schematic representation of a genetic map of a CLRN-10 (SEQ ID NO: 48; 3511 bp) that can be used in any of the present methods described herein. The vector includes an AAV2 ITR, CMV enhancer (SEQ ID NO: 17), chicken β-actin promoter, chimeric intron (SEQ ID NO: 16), CLRN1 isoform 1 (SEQ ID NO: 4), a HA sequence (SEQ ID NO: 34), a FP sequence (SEQ ID NO: 30), T2A sequence (SEQ ID NO: 31), CLRN1 isoform 2 (SEQ ID NO: 5), 3× FLAG tag sequence (SEQ ID NO: 35), bGH poly(A) signal (SEQ ID NO: 20) and an AAV2 ITR.

FIG. 13 is an exemplary schematic representation of a genetic map of a CLRN-10myc vector (SEQ ID NO: 49; 3574 bp) that can be used in any of the present methods described herein. The vector includes an AAV2 ITR, CMV enhancer (SEQ ID NO: 17), chicken β-actin promoter, chimeric intron (SEQ ID NO: 16), CLRN1 isoform 1 (SEQ ID NO: 1), a myc sequence (SEQ ID NO: 33), a FP sequence (SEQ ID NO: 30), T2A sequence (SEQ ID NO: 31), 3× FLAG tag sequence (SEQ ID NO: 35), bGH poly(A) signal (SEQ ID NO: 20) and an AAV2 ITR.

FIG. 14 is an exemplary schematic representation of a genetic map of a CLRN-10NF vector (SEQ ID NO: 50; 3499 bp) that can be used in any of the present methods described herein. The vector includes an AAV2 ITR, CMV enhancer (SEQ ID NO: 17), chicken β-actin promoter, chimeric intron (SEQ ID NO: 16), CLRN1 isoform 1 (SEQ ID NO: 1), T2A sequence (SEQ ID NO: 31), an HA sequence (SEQ ID NO: 34), CLRN-1 isoform 2 (SEQ ID NO: 5), 3× FLAG tag sequence (SEQ ID NO: 35), bGH poly(A) signal (SEQ ID NO: 20) and an AAV2 ITR.

FIG. 15 is an exemplary schematic representation of a genetic map of a CLRN-11 vector (SEQ ID NO: 51; 4908 bp) that can be used in any of the present methods described herein. The vector includes an AAV2 ITR, CMV enhancer (SEQ ID NO: 17), chicken β-actin promoter, chimeric intron (SEQ ID NO: 16), CLRN1 isoform 1 (SEQ ID NO: 1), a HA sequence (SEQ ID NO: 34), a FP sequence (SEQ ID NO: 30), T2A sequence (SEQ ID NO: 31), CLRN-1 isoform 2 (SEQ ID NO: 5), 3× FLAG tag sequence (SEQ ID NO: 35), 3′UTR-1406 (SEQ ID NO: 37), bGH poly(A) signal (SEQ ID NO: 20) and an AAV2 ITR.

FIG. 16 is an exemplary schematic representation of a genetic map of a CLRN-11myc vector (SEQ ID NO: 52; 4971 bp) that can be used in any of the present methods described herein. The vector includes an AAV2 ITR, CMV enhancer (SEQ ID NO: 17), chicken β-actin promoter, chimeric intron (SEQ ID NO: 16), CLRN1 isoform 1 (SEQ ID NO: 1), a myc sequence (SEQ ID NO: 33), a FP sequence (SEQ ID NO: 30), T2A sequence (SEQ ID NO: 31), CLRN1 isoform 2 (SEQ ID NO: 5), 3× FLAG tag sequence (SEQ ID NO: 35), 3′UTR-1406 (SEQ ID NO: 37), bGH poly(A) signal (SEQ ID NO: 20) and an AAV2 ITR.

FIG. 17 is an exemplary schematic representation of a genetic map of a CLRN-11NF vector (SEQ ID NO: 53; 4896 bp) that can be used in any of the present methods described herein. The vector includes an AAV2 ITR, CMV enhancer (SEQ ID NO: 17), chicken β-actin promoter, chimeric intron (SEQ ID NO: 16), CLRN1 isoform 1 (SEQ ID NO: 1), an HA sequence (SEQ ID NO: 34), T2A sequence (SEQ ID NO: 31), CLRN1 isoform 2 (SEQ ID NO: 5), 3× FLAG sequence (SEQ ID NO: 33), 3′UTR-1406 (SEQ ID NO: 37), bGH poly(A) signal (SEQ ID NO: 20) and an AAV2 ITR.

FIG. 18 is an exemplary schematic representation of a genetic map of a CLRN-12 vector (SEQ ID NO: 54; 4640 bp) that can be used in any of the present methods described herein. The vector includes an AAV2 ITR, CMV enhancer (SEQ ID NO: 17), chicken β-actin promoter, chimeric intron (SEQ ID NO: 16), a 5′UTR-291 sequence (SEQ ID NO: 12), CLRN1 isoform 1 (SEQ ID NO: 1), an HA sequence (SEQ ID NO: 34), 3′UTR-1357 (SEQ ID NO: 36), bGH poly(A) signal (SEQ ID NO: 20) and an AAV2 ITR.

FIG. 19 is an exemplary schematic representation of a genetic map of a CLRN-13 vector (SEQ ID NO: 55; 4291 bp) that can be used in any of the present methods described herein. The vector includes an AAV2 ITR, CMV enhancer (SEQ ID NO: 17), chicken β-actin promoter, chimeric intron (SEQ ID NO: 16), CLRN1 isoform 4 (SEQ ID NO: 7), 3× FLAG tag sequence (SEQ ID NO: 35), T2A sequence (SEQ ID NO: 31), CLRN1 isoform 1 (SEQ ID NO: 1), an HA sequence (SEQ ID NO: 34), 3′UTR-600 (SEQ ID NO: 27), bGH poly(A) signal (SEQ ID NO: 20) and an AAV2 ITR.

FIG. 20 is an exemplary schematic representation of a genetic map of a CLRN-14 vector (SEQ ID NO: 56; 4192 bp) that can be used in any of the present methods described herein. The vector includes an AAV2 ITR, CMV enhancer (SEQ ID NO: 17), chicken β-actin promoter, chimeric intron (SEQ ID NO: 16), CLRN1 isoform 4 (SEQ ID NO: 7), T2A sequence (SEQ ID NO: 31), CLRN1 isoform 1 (SEQ ID NO: 1), 3′UTR-600 (SEQ ID NO: 27), bGH poly(A) signal (SEQ ID NO: 20) and an AAV2 ITR.

FIG. 21 is an exemplary schematic representation of a genetic map of a CLRN-15 vector (SEQ ID NO: 57; 3505 bp) that can be used in any of the present methods described herein. The vector includes an AAV2 ITR, CMV enhancer (SEQ ID NO: 17), chicken β-actin promoter, chimeric intron (SEQ ID NO: 16), CLRN1 isoform 4 (SEQ ID NO: 7), 3× FLAG tag sequence (SEQ ID NO: 35), 3′UTR-600 (SEQ ID NO: 27), bGH poly(A) signal (SEQ ID NO: 20) and an AAV2 ITR.

FIG. 22 is an exemplary schematic representation of a genetic map of a CLRN-16 vector (SEQ ID NO: 58; 3439 bp) that can be used in any of the present methods described herein. The vector includes an AAV2 ITR, CMV enhancer (SEQ ID NO: 17), chicken β-actin promoter, chimeric intron (SEQ ID NO: 16), CLRN1 isoform 4 (SEQ ID NO: 7), 3′UTR-600 (SEQ ID NO: 27), bGH poly(A) signal (SEQ ID NO: 20) and an AAV2 ITR.

FIG. 23 is an exemplary schematic representation of a genetic map of a CLRN-17 vector (SEQ ID NO: 59; 130 bp) that can be used in any of the present methods described herein. The vector includes an AAV2 ITR, CMV enhancer (SEQ ID NO: 17), chicken β-actin promoter, a sh-chimeric intron (SEQ ID NO: 26), 5′UTR-291 (SEQ ID NO: 12), CLRN1 isoform 1 (SEQ ID NO: 1), an HA sequence (SEQ ID NO: 34), 3′UTR-1773 (SEQ ID NO: 15), bGH poly(A) signal (SEQ ID NO: 20) and an AAV2 ITR.

FIG. 24 is an exemplary schematic representation of a genetic map of a CLRN-18 vector (SEQ ID NO: 60; 4277 bp) that can be used in any of the present methods described herein. The vector includes an AAV2 ITR, CMV enhancer (SEQ ID NO: 17), chicken β-actin promoter, a sh-chimeric intron (SEQ ID NO: 26), CLRN1 isoform 1 (SEQ ID NO: 1), 3′UTR-1773 (SEQ ID NO: 15), bGH poly(A) signal (SEQ ID NO: 20) and an AAV2 ITR.

FIG. 25 is an image of an immunoblot of CLRN1 protein levels from transfected HEK293FT cells 48-hours post-transfection using anti-HA and anti-FLAG antibodies. Lane 1—PageRuler Plus Prestained; Lane 2—CLRN-10 (37° C. denaturing); Lane 3—CLRN-11 (37° C. denaturing); Lane 4—CLRN-12 (37° C. denaturing); Lane 5—negative control; Lane 6—CLRN-10 (56° C. denaturing); Lane 7—CLRN-11 (56° C. denaturing); Lane 8—CLRN-12 (56° C. denaturing); Lane 9—negative control. CLRN-1 isoform protein is a glycosylated protein and often migrates as smear bands.

FIG. 26 is an image of an immunoblot of CLRN1 protein levels from transfected HEK293FT cells 48-hours post-transfection using an anti-FLAG antibody. Lane 1—PageRuler Plus Prestained; Lane 2—CLRN-10; Lane 3—CLRN-11; Lane 4—CLRN-12; Lane 5—CLRN-13; Lane 6—CLRN-15; Lane 7—negative control

FIG. 27 is an image of an immunoblot of CLRN1 protein levels from transfected HEK293FT cells 48-hours post-transfection using an anti-FLAG antibody. Lane 1—PageRuler Plus Prestained; Lane 2—CLRN-10; Lane 3—CLRN-10NF; Lane 4—CLRN-10myc; Lane 5—CLRN-11NF; Lane 6—CLRN-11myc; Lane 7—negative control. All samples were kept at room temperature.

FIG. 28 is an image of an immunoblot of CLRN1 protein levels from transfected HEK293FT cells 48-hours post-transfection using an anti-myc antibody. Lane 1—PageRuler Plus Prestained; Lane 2—CLRN-10; Lane 3—CLRN-10NF; Lane 4—CLRN-10myc; Lane 5—CLRN-11NF; Lane 6—CLRN-11myc; Lane 7—negative control.

FIG. 29 is an image of an immunoblot of CLRN1 protein levels harvested from transfected HEK293FT cells 48 hours post-transfection using anti-HA and anti-FLAG antibodies. Lane 1—PageRuler Plus Prestained; Lane 2—CLRN-13; Lane 3—CLRN-10; Lane 4—CLRN-10NF; Lane 5—CLRN-10myc; Lane 6—CLRN-11NF; Lane 7—CLRN-11myc; Lane 8—negative control.

FIG. 30 is an image of an immunoblot using anti-CLRN (EKIANYKEGTYVYKTQSEKY; SEQ ID NO: 38) rabbit polyclonal antibody of CLRN1 isoform 1 protein levels harvested from HEK239FT cells transfected with plasmids described herein 48 hours post-transfection. Lane 1—PageRuler Plus Prestained; Lane 2—CLRN-10; Lane 3—CLRN-1; Lane 4—CLRN-2; Lane 5—CLRN-3; Lane 6—CLRN-4; Lane 7—CLRN-8; Lane 8—CLRN-9; Lane 9—CLRN-10; Lane 10—CLRN-11; Lane 11—CLRN-12; Lane 12—CLRN-13, Lane 13—CLRN-14; Lane 14—CLRN-15; Lane 15—CLRN-16; Lane 16—negative control; Lane 17—negative control.

FIG. 31 is an image of an immunoblot CLRN1 protein levels harvested from transfected HEK293FT cells 48 hours post-transfection using an anti-CLRN rabbit polyclonal antibody. Lane 1—PageRuler Plus Prestained; Lane 2—Anc80-CLRN-0; Lane 3—Anc80-CLRN-0+PNGase F; Lane 4-Anc80-CLRN-3; Lane 5—Anc80-CLRN-3+PNGase F; Lane 6—Anc80-CLRN-6eGFP; Lane 7—Anc80-CLRN-6eGFP+PNGase F; Lane 8—Anc80-CLRN-13; Lane 9—Anc80-CLRN-13+PNGase F; Lane 10—no vector; Lane 11—no vector+PNGase F. CLRN-1 isoform protein is a glycosylated protein and often migrates as smear bands (lanes 2, 4 and 8). After treatment with PNGase F, the smeared bands disappeared and shifted to distinct bands (alnes 3, 5 and 8).

FIG. 32 is a set of immunofluorescent images of AAVanc80-CLRN6eGFP transduced HEK293FT cells taken 24 hours and 48 hours post-transfection at MOI 8.41E+04 and MOI 2.53E+05, respetively.

FIG. 33 is a bar graph showing the relative CLRN1 and GFP expression in HEK293FT cells transduced with AAVanc80-CLRN-6eGFP (at MOI 1.05E+05 and MOI 3.15E+05), AAVanc80-CLRN-0 (at MOI 8.23E+04 and MOI 2.47E+05), AAVanc80-CLRN-3 (at MOI 8.41E+04 and MOI 2.53E+04), and AAVanc80-CLRN-13 (at MOI 8.33E+04 and MOI 2.50E+05), respectively.

FIG. 34 is a bar graph showing the relative CLRN1 and GFP expression in P2 cochlear explants from WT mice infected 16-hours with AAVanc80-CLRN-6eGFP (at MOI 2.0E+05), AAVanc80-CLRN-0 (at MOI 2.5E+05 and MOI 7.6E+0.5), AAVanc80-CLRN-3 (at MOI 2.0E+05 and 6.03E+05) and AAVanc80-CLRN-13 (at MOI 2.0E+05 and MOI 6.0E+05), respectively.

FIG. 35 is a set of fluorescent images of P2 cochlear explants from WT mice infected 72-hours with 1.3E10 AAVanc80-CLRN-0 VG/cochlea, 9.9E9 AAVanc80-CLRN-3 VG/cochlea and 1.0E10 AAVanc80-CLRN-13 VG/cochlea showing Myo7a and DAPI staining.

FIG. 36 is a set of fluorescent images of P2 cochlear explants from WT mice infected 72-hours with 1E09 VG/cochlea AAV Anc80.CAG.eGFP.3′UTR showing eGFP, Myo7a and DAPI staining.

### DETAILED DESCRIPTION

Deficiency or mutations in “clarin 1,” the protein encoded by the CLRN1 gene, causes hearing loss and vision loss. For example, mutations in CLRN1 lead to Usher syndrome type III and retinitis pigmentosa.

Provided herein are compositions that include at least two different nucleic acid vectors, wherein: each of the at least two different vectors comprises a coding sequence that encodes a different portion of a CLRN1 protein, each of the encoded portions being at least 30 amino acid residues in length, wherein the amino acid sequence of each of the encoded portions may optionally partially overlap with the amino acid sequence of a different one of the encoded portions; no single vector of the at least two different vectors encodes a full-length CLRN1 protein; at least one of the coding sequences comprises a nucleotide sequence spanning two consecutive exons of CLRN1 genomic DNA, and lacking an intronic sequence between the two consecutive exons; and when introduced into a mammalian cell, the at least two different vectors undergo homologous recombination with each other, thereby forming a recombined nucleic acid that encodes a full-length CLRN1 protein.

Provided herein are compositions that include a single nucleic acid vector, wherein the vector comprises one or both of (i) a first coding sequence encoding a first isoform of CLRN1 protein; and (ii) a second coding sequence encoding a second isoform of CLRN1 protein, where one or both of the first and second coding sequences comprises a nucleotide sequence spanning two consecutive exons of a CLRN1 genomic DNA, and lacking an intronic sequence between the two consecutive introns.

Provided herein are compositions that include two different nucleic acid vectors, wherein: a first nucleic acid vector of the two different nucleic acid vectors comprises a promoter, a first coding sequence that encodes an N-terminal portion of a CLRN1 protein positioned 3′ of the promoter, and a splice donor sequence positioned at the 3′ end of the first coding sequence; and a second nucleic acid vector of the two different nucleic acid vectors comprises a splice acceptor sequence, a second coding sequence that encodes a C-terminal portion of a CLRN1 protein positioned at the 3′ end of the splice acceptor sequence, and a polyadenylation signal sequence at the 3′ end of the second coding sequence; wherein each of the encoded portions is at least 30 amino acid residues in length, wherein the amino acid sequences of the two encoded portions do not overlap with each other; wherein no single vector of the two different vectors encodes a full-length CLRN1 protein; and when introduced into a mammalian cell, splicing occurs between the splice donor sequence and the splice acceptor sequence, thereby forming a recombined nucleic acid that encodes a full-length CLRN1 protein.

Provided herein are compositions that include two different nucleic acid vectors, wherein: a first nucleic acid vector of the two different nucleic acid vectors comprises a promoter, a first coding sequence that encodes an N-terminal portion of a CLRN1 protein positioned 3′ of the promoter, a splice donor sequence positioned at the 3′ end of the first coding sequence, and a first detectable marker gene positioned 3′ of the splice donor sequence; and a second nucleic acid vector of the two different nucleic acid vectors comprises a second detectable marker gene, a splice acceptor sequence positioned 3′ of the second detectable marker gene, a second coding sequence that encodes a C-terminal portion of a CLRN1 protein positioned at the 3′ end of the splice acceptor sequence, and a polyadenylation signal sequence positioned at the 3′ end of the second coding sequence; wherein each of the encoded portions is at least 30 amino acid residues in length, wherein the amino acid sequences of the encoded portions do not overlap with each other; wherein no single vector of the two different vectors encodes a full-length CLRN1 protein; and when introduced into a mammalian cell, splicing occurs between the splice donor sequence and the splice acceptor sequence, thereby forming a recombined nucleic acid that encodes a full-length CLRN1 protein.

Also provided herein are compositions that include two different nucleic acid vectors, wherein: a first nucleic acid vector of the two different nucleic acid vectors comprises a promoter, a first coding sequence that encodes an N-terminal portion of a CLRN1 protein positioned 3′ to the promoter, a splice donor sequence positioned at the 3′ end of the first coding sequence, and a F1 phage recombinogenic region positioned 3′ to the splice donor sequence; and a second nucleic acid vector of the two different nucleic acid vectors comprises a F1 phage recombinogenic region, a splice acceptor sequence positioned 3′ of the F1 phage recombinogenic region, a second coding sequence that encodes a C-terminal portion of a CLRN1 protein positioned at the 3′ end of the splice acceptor sequence, and a polyadenylation signal sequence positioned at the 3′ end of the second coding sequence; wherein each of the two encoded portions is at least 30 amino acid residues in length, wherein the amino acid sequences of the two encoded portions do not overlap with each other; wherein no single vector of the two different vectors encodes a full-length CLRN1 protein; and when introduced into a mammalian cell, splicing occurs between the splice donor sequence and the splice acceptor sequence, thereby forming a recombined nucleic acid that encodes a full-length CLRN1 protein.

Provided herein are methods that include: introducing into a cochlea of a mammal a therapeutically effective amount of any of the compositions described herein.

Provided herein are methods of increasing expression of a full-length CLRN1 protein in a mammalian cell, that include: introducing any of the compositions described herein into the mammalian cell.

Provided herein are methods of increasing expression of a full-length CLRN1 protein in an inner hair cell, an outer hair cell, or both, in a cochlea of a mammal that include: introducing into the cochlea of the mammal a therapeutically effective amount of any of the compositions described herein.

Provided herein are methods of increasing expression of a full-length CLRN1 protein in an eye of a mammal that include: intraocularly administering to the eye of the mammal a therapeutically effective amount of any of the compositions described herein.

Provided herein are methods of treating hearing loss in a subject identified as having a defective CLRN1 gene that include: administering a therapeutically effective amount of any of the compositions described herein into the cochlea of the subject.

Also provided herein are methods of treating vision loss in a subject identified as having a defective CLRN1 gene that include: administering a therapeutically effective amount of any of the compositions described herein into the eye of the subject.

Additional non-limiting aspects of the compositions, kits, and methods are described herein and can be used in any combination without limitation.

### CLRN1

The CLRN1 gene encodes “clarin 1” (CLRN1), a protein that is expressed in hair cells of the inner ear (e.g., inner ear hair cells, outer ear hair cells) and in the retina.

The human CLRN1 gene is located on chromosome 3q25.1. It contains 7 exons encompassing ˜47 kilobases (kb) (Vastinsalo et al. (2011) Eur J Hum Genet 19(1): 30-35; NCBI Accession No. NG 009168.1).

Various mutations in the CLRN1 genes have been associated with Usher syndrome type III (e.g., Usher syndrome type IIIA (MIM #606397) (see, e.g., Fields et al. (2002) Am J Hum Genet 71: 607-617, and Joensuu et al. (2001) Am J Hum Genet 69: 673-684) and retinitis pigmentosa (see, e.g., Khan et al. (2011) Ophthalmology 118: 1444-1448). Usher syndrome type III-causing mutations have been predominantly found in exon 3 of CLRN1. Usher syndrome type III-deafness can be modeled by generating CLRN1-deficient mice (see, e.g., Geng et al. (2017) Sci Rep 7(1): 13480). Exemplary mutations CLRN1-associated with Usher syndrome type III include: T528G, M120K, M44K, N48K, and C40G.

Exemplary mutations CLRN1-associated with retinitis pigmentosa include L154W and P31L (see, e.g., Khan et al. (2011) Ophthalmology 118: 1444-1448).

Additional exemplary mutations in a CLRN1 gene that have been detected in subjects having hearing loss and methods of sequencing a nucleic acid encoding CLRN1 are described in, e.g., Fields et al. (2002) Am J Hum Genet 71: 607-617, Joensuu et al. (2001) Am J Hum Genet 69: 673-684, Adato et al. (2002) Europ J Hum Genet 10: 339-350, Aller et al. (2004), Clin Genet 66: 525-529. Methods of detecting mutations in a gene are well-known in the art. Non-limiting examples of such techniques include: real-time polymerase chain reaction (RT-PCR), PCR, sequencing, Southern blotting, and Northern blotting.

An exemplary human wildtype CLRN1 protein is or includes the sequence of SEQ ID NO: 1, SEQ ID NO: 3, SEQ ID NO: 5 and SEQ ID NO: 7. Non-limiting examples of nucleotide sequences encoding a wildtype CLRN1 protein are or include SEQ ID NO: 2, SEQ ID NO: 4, SEQ ID NO: 6 and SEQ ID NO: 8.

In some embodiments of any of the compositions described herein, the CLRN1 protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 1.

In some embodiments of any of the compositions described herein, the CLRN1 protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 3.

In some embodiments of any of the compositions described herein, the CLRN1 protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 5.

In some embodiments of any of the compositions described herein, the CLRN1 protein comprises a sequence that is at least 75% (e.g., at least 80%, at least 85%, at least 90%, at least 91%, at least 92%, at least 93%, at least 94%, at least 95%, at least 96%, at least 97%, at least 98%, at least 99%) identical to SEQ ID NO: 7.

| Human Full-length Wildtype CLRN1 Protein Isoform D | |
|---|---|
| (SEQ ID NO: 1) | |
| MPSQQKKIIFCMAGVFSFACALGVVTALGTPLWIKATVLCKTGALLVNASGQELDKFMGEMQYGLFHGEGV | |
| RQCGLGARPFRFSFFPDLLKAIPVSIHVNVILFSAILIVLTMVGTAFFMYNAFGKPFETLHGPLGLYLLSF | |
| ISVALWLPATRHQAQGSCGCLVMILFASEVKIHHLSEKIANYKEGTYVYKTQSEKYTTSFWVIFFCFFVHF | |
| LNGLLIRLAGFQFPFAKSKDAETTNVAADLMY | |
| Human Wildtype CLRN1 Isoform D cDNA | |
| (SEQ ID NO: 2) | |
| atgccaagc caacagaaga aaatcatttt ttgcatggcc ggagtgttca gttttgcatg | |
| tgccctcgga gttgtgacag ccttggggac accgttgtgg atcaaagcca ctgtcctctg | |
| caaaacggga gctctgctcg tcaatgcctc agggcaggag ctggacaagt ttatgggtga | |
| aatgcagtac gggcttttcc acggagaggg tgtgaggcag tgtgggttgg gagcaaggcc | |
| ctttcggttc tcattttttc cagatttgct caaagcaatc ccagtgagca tccacgtcaa | |
| tgtcattctc ttctctgcca tccttattgt gttaaccatg gtggggacag ccttcttcat | |
| gtacaatgct tttggaaaac cttttgaaac tctgcatggt cccctagggc tgtacctttt | |
| gagcttcatt tcagttgccc tttggctgcc agctaccagg caccaggctc aaggctcctg | |
| tggctgtctt gtcatgatat tgtttgcctc tgaagtgaaa atccatcacc tctcagaaaa | |
| aattgcaaat tataaagaag ggacttatgt ctacaaaacg caaagtgaaa aatataccac | |
| ctcattctgg gtcattttct tttgcttttt tgttcatttt ctgaatgggc tcctaatacg | |
| acttgctgga tttcagttcc cttttgcaaa atctaaagac gcagaaacaa ctaatgtagc | |
| tgcagatcta atgtactga | |
| Human Full-length Wildtype CLRN1 Protein Isoform A | |
| (SEQ ID NO: 3) | |
| MPSQQKKIIFCMAGVFSFACALGVVTALGTPLWIKATVLCKTGALLVNASGQELDKFMGEMQYGLFHGEGV | |
| RQCGLGARPFRFSFFPDLLKAIPVSIHVNVILFSAILIVLTMVGTAFFMYNAFGKPFETLHGPLGLYLLSF | |
| ISGSCGCLVMILFASEVKIHHLSEKIANYKEGTYVYKTQSEKYTTSFWVIFFCFFVHFLNGLLIRLAGFQF | |
| PFAKS KDAETTNVAADLMY | |
| Human Wildtype CLRN1 Isoform A cDNA | |
| (SEQ ID NO: 4) | |
| atgccaagc caacagaaga aaatcatttt ttgcatggcc ggagtgttca gttttgcatg | |
| tgccctcgga gttgtgacag ccttggggac accgttgtgg atcaaagcca ctgtcctctg | |
| caaaacggga gctctgctcg tcaatgcctc agggcaggag ctggacaagt ttatgggtga | |
| aatgcagtac gggcttttcc acggagaggg tgtgaggcag tgtgggttgg gagcaaggcc | |
| ctttcggttc tcattttttc cagatttgct caaagcaatc ccagtgagca tccacgtcaa | |
| tgtcattctc ttctctgcca tccttattgt gttaaccatg gtggggacag ccttcttcat | |
| gtacaatgct tttggaaaac cttttgaaac tctgcatggt cccctagggc tgtacctttt | |
| gagcttcatt tcaggctcct gtggctgtct tgtcatgata ttgtttgcct ctgaagtgaa | |
| aatccatcac ctctcagaaa aaattgcaaa ttataaagaa gggacttatg tctacaaaac | |
| gcaaagtgaa aaatatacca cctcattctg ggtcattttc ttttgctttt ttgttcattt | |
| tctgaatggg ctcctaatac gacttgctgg atttcagttc ccttttgcaa aatctaaaga | |
| cgcagaaaca actaatgtag ctgcagatct aatgtactga | |
| Human Full-length Wildtype CLRN1 Protein Isoform C | |
| (SEQ ID NO: 5) | |
| MQALQQQPVFPDLLKAIPVSIHVNVILFSAILIVLTMVGTAFFMYNAFGKPFETLHGPLGLYLLSFISGSC | |
| GCLVMILFASEVKIHHLSEKIANYKEGTYVY KTQSEKYTTSFWLTKGHS | |
| Human Wildtype CLRN1 Isoform C cDNA | |
| (SEQ ID NO: 6) | |
| atgcaggc cctgcagcag caaccagttt ttccagattt gctcaaagca atcccagtga | |
| gcatccacgt caatgtcatt ctcttctctg ccatccttat tgtgttaacc atggtgggga | |
| cagccttctt catgtacaat gcttttggaa aaccttttga aactctgcat ggtcccctag | |
| ggctgtacct tttgagcttc atttcaggct cctgtggctg tcttgtcatg atattgtttg | |
| cctctgaagt gaaaatccat cacctctcag aaaaaattgc aaattataaa gaagggactt | |
| atgtctacaa aacgcaaagt gaaaaatata ccacctcatt ctggctgact aaaggccaca gctga | |
| Human Full-length Wildtype CLRN1 Protein Isoform E | |
| (SEQ ID NO: 7) | |
| MPSQQKKIIFCMAGVFSFACALGVVTALGTPLWIKATVLCKTGALLVNASGQELDKFMGEMQYGLFHGEGV | |
| RQCGLGARPFRFSCYFLDPFMGLPTGVPHLLSLPCSTSCRREHTSERVQEPAGCFSAVRSKLHAGPAAATS | |
| FSRFAQSNPSEHPRQCHS LLCHPYCVNHGGDSLLHVQCFWKTF | |
| Human Wildtype CLRN1 Isoform E cDNA | |
| (SEQ ID NO: 8) | |
| atgccaagc caacagaaga aaatcatttt ttgcatggcc ggagtgttca gttttgcatg | |
| tgccctcgga gttgtgacag ccttggggac accgttgtgg atcaaagcca ctgtcctctg | |
| caaaacggga gctctgctcg tcaatgcctc agggcaggag ctggacaagt ttatgggtga | |
| aatgcagtac gggcttttcc acggagaggg tgtgaggcag tgtgggttgg gagcaaggcc | |
| ctttcggttc tcatgctatt ttcttgaccc cttcatggga ctcccaacag gggtacccca | |
| tttactcagc ctgccctgct caacctcttg caggagggag cacacgagtg aacgagtgca | |
| ggaaccagct ggctgcttta gtgctgtgag gagtaaactc catgcaggcc ctgcagcagc | |
| aaccagtttt tccagatttg ctcaaagcaa tcccagtgag catccacgtc aatgtcattc | |
| tcttctctgc catccttatt gtgttaacca tggtggggac agccttcttc atgtacaatg | |
| cttttggaaa accttttga | |
| Human Wildtype CLRN1 Genomic sequence | |
| (SEQ ID NO: 9) | |
| aggagatact tgaaggcagt ttgaaagact tgttttacag attcttagtc caaagatttc | 61 |
| caattaggga gaagaagcag cagaaaagga gaaaagccaa gtatgagtga tgatgaggcc | 121 |
| ttcatctact gacatttaac ctggcgagaa ccgtcgatgg tgaagttgcc ttttcagctg | 181 |
| ggagctgtcc gttcagcttc cgtaataaat gcagtcaaag aggcagtccc ttcccattgc | 241 |
| tcacaaaggt cttgtttttg aacctcgccc tcacagaagc cgtttctcat catgccaagc | 301 |
| caacagaaga aaatcatttt ttgcatggcc ggagtgttca gttttgcatg tgccctcgga | 361 |
| gttgtgacag ccttggggac accgttgtgg atcaaagcca ctgtcctctg caaaacggga | 421 |
| gctctgctcg tcaatgcctc agggcaggag ctggacaagt ttatgggtga aatgcagtac | 481 |
| gggcttttcc acggagaggg tgtgaggcag tgtgggttgg gagcaaggcc ctttcggttc | 541 |
| tcatgtaagt agcaattgca tttgagttat ttaatgcttt aggcagactc ttcccagtgt | 601 |
| tgcgaggaat tatatttgag aattttgccg tgtttactgc aggacttttt aaatcggtgt | 661 |
| gaaccatatg aaaaacctat gactctgagc aatttcttct tcctagtttt tattatttta | 721 |
| tacttgcttt ttattataat atagagttaa ttcattgtta cataattaag gtttttggaa | 781 |
| atattggcaa ttaagatgct taagtattaa tatttatgta aaaaattatg gagtcttttt | 841 |
| aaaaaagtaa acttggggaa ataggaaagc tgtaaagaat gatctttatg ctttttgttc | 901 |
| tttataaaaa gaaccaaggt catgggctcc gtatttaacc aggttgccac ctttctcatg | 961 |
| attttgtttc ctgctcccca ctccctccca ttattcctgc taagaccttt cctgctgcta | 1021 |
| aatattcagt tttcattttt aactaatttg gaatcatttg gctatagaaa tttaaaatga | 1081 |
| tctgctgtgc taactgggaa agaaatggat gcctatttag tatagaacat tttaaactga | 1141 |
| ttgacctgca aatcatgtag agaatatgag agagattttc ttgttgtgat ttttgtgaaa | 1201 |
| tggaagtgta atccacagta tttataacct gtttatctta agaagagaat ttttaaaaat | 1261 |
| taccatgtga ataggcaact cattaaatga aaattaatag gaagtcattt gttatatctc | 1321 |
| ttacaacaca cattcagaag ttattattat ttcagaaggg ctggtttgga acaaccttat | 1381 |
| gaagacacag tcagtaaatt actgcataaa tcactcttca ggaaaggagg ttaccaactg | 1441 |
| aagcatttaa aatgaattat tattttgccc aggttttttt tttctttcta gtataggtag | 1501 |
| aaggctaaat taattgaatt tattattaac atatgcagtg cctaattaaa tttcagtgct | 1561 |
| ggtctattta tatttctgca acattcctta tatcttctta gcagtcattg gacaccaacc | 1621 |
| ttcagctcac ataggttact aagtgatatg aattttcata gggctccaga aaatttccaa | 1681 |
| gaattggttg ttagcttttt aattgatgaa gtggatacca gttcttttca ctgaatggct | 1741 |
| tttattcatt aaggtaatgg ggctgttaga gttgcttagt tttcctgggg aaggggaagg | 1801 |
| aagaaaacaa agcagaatgt catgtgatat gcaactgtat taaaaaaccg aaaaggaaaa | 1861 |
| aagttgagag agatgattta accgtgagtc accggcagcc aaagcgtgag taaagcttct | 1921 |
| cacagatgaa tttagacaaa agcggagaag gtactggtga attttctgga gcctttacat | 1981 |
| tttctacagt gaaatggaga taaactttac tcatgccata ggacatgttt caaaacaata | 2041 |
| ataagatgtt ttctgaacac ttactacata ctaagcactt tatatgcttt gtctcattta | 2101 |
| atccttacac agccacattc ttctggggtt tagcgaatga tttttgtggt tgtgtcctat | 2161 |
| gcttgtcctg tctaaggatg aagttgttct aattgggtgc ccctcctttt gctttctgtg | 2221 |
| aggacttgca gaactggtgg ggtttaaaca gtaccctcac ttatctcaca gaatttcatt | 2281 |
| agctcccaga tacccctgac attctccccc tagcctagtg aagaaaatct tccatttact | 2341 |
| tgttcattct gcagtgacag ctccatcaat atacaataga ctatacatat taagtgtact | 2401 |
| gtatatacta tacatgttaa aaatctcatt cattttggtg aggcccagct aagaatactt | 2461 |
| acagtagagc tttttttttt ttcctaagca taaaagtatc tttttcaatg cagcatgaga | 2521 |
| cagagttggg aaaaccaaaa taaatagatc caatggactc cccaaagagg ataatattca | 2581 |
| tttaaataaa cacccctctc agtgttaaaa ctttctaatc aacatgcctt tgggacacat | 2641 |
| tgcaccctca aagtttacac tcccattgca acgcagcttt gtggttcacg ttttttccat | 2701 |
| tcagaatgtc attaccctgt caatgatgtt tcatcaacgt ttgcttggat gagaatcctc | 2761 |
| tgatattctt cctgatagaa atgtataagc cctgttcata taaatgaata aaagatctaa | 2821 |
| ccttactttc tcagtagtgg cttccttgga gcaaaaagca gggacctcca gagagctcag | 2881 |
| gtggatgact cttttctgtt tcttccagag ctcaacttac aattagtgca caattcattt | 2941 |
| cccagaatgt cttctttctt attgtgcctt tagaaagtta ttaagcaaac atttgaattc | 3001 |
| acagaatctt accagtgtaa gaggaatgga aaaggtaact tatcaaggta acaatcactt | 3061 |
| cgtggccagt tttttcggct cactgcaact acccctcctg ggttcaagcg attctcctgc | 3121 |
| ctcagcctcc caagtagctg ggactacagg cgtgcaccac catgcccagc taattttttt | 3181 |
| tttgtatttt tagtaaagac agggtttcac catgtgggcc aggctggtct catggcaagt | 3241 |
| tttctttgtg ttgtcatgtt attatcaatt aataggaatt tatatttcag ttctgttagg | 3301 |
| tggataaaca ctattttgca tacctaaatg tttcatttat atcagcactg gccaataaaa | 3361 |
| atatactata agcaggccgg gtgcagtggc tcacgcctgt aatcccaact tttgggaggc | 3421 |
| caacactttg ggaggacaca gggtcaggag atcgagacca tcctggctaa cctggtgaaa | 3481 |
| tcccgtctct actaaaaata caaaaaatta gccgggcgtg gcgggggcgc ctgtagtccc | 3541 |
| agctacttgg gaggctgagg caggagaatg gcacgaaccc gggaggtgga gcttgcagtg | 3601 |
| agccgagatc tcgccactgc actccagcct tggtgacaga gcaagactct gtctcaaaaa | 3661 |
| aaaaaaaaaa aaaaaaaaaa aaaaaatata tatatatata tatatattac aagccacaag | 3721 |
| ccacatatgt acttttaaat gtcctagtag ccatattaga aaacaaaagt aaaaaggaat | 3781 |
| agatgaaatt aattttaatg atttttttaa acccaagata tccaaaatat tatcatttta | 3841 |
| acatataatt aaaaatttat tgagatgttt tacattgttt ttttttttct tgacgctgtc | 3901 |
| ttggaaatct agagtgtatt ttacatttac attacatcta attcagtcta gccacatttc | 3961 |
| acatgctcat ttttttgtgt gtggttttat ggagcagaga gtttaatagg caagaaagaa | 4021 |
| aagagaaggc agaagaaaat ggctcccctg tacagagacg cgggggtggg gcgctccaaa | 4081 |
| gccaaaagag gaggtcccta agtatggtag acaccagcca ggaatatatg cagtgtctgg | 4141 |
| aggaggggat gtctgatttg catagggtca catgctcatt tttatggcta ctgtattggt | 4201 |
| cagtacagat ttagatgggg atttgctctg aacaagttgg tgattgatgg tgttcatatt | 4261 |
| ttaattgaat ttttcctggg ttctgctgta cacttgtatg tgtgttagtt tcatgtgcaa | 4321 |
| tgcttgtgtc acttttaaaa cccagatata tggcataaca tgagaatgaa aaatggacca | 4381 |
| gaaaaatagt ttggcaatgt agtcatgttt gttcctatta aatgttccct attgaccact | 4441 |
| ctatctcttt taattataac aagaatctgc cctgccagca tgcccagtta cgctgggaaa | 4501 |
| acttctgcct catttactct ggctgattct catccactta tgggtcagtg gttcattttc | 4561 |
| tagaggtcac cagcattcat acctagcata caatttcatt cattataatg aaggaatgtt | 4621 |
| ttcccttcaa agagacacaa ctagtgggct taatttttct tgatatgtca cctgtaaaat | 4681 |
| tttaatgatg atgtttaaac tctaaatgta gccatcaaga caaaaactgc aaattttgag | 4741 |
| cctcagtgtg tgtgggtggg tttctgtttc ggtaatttga aacattgcag aatatcatca | 4801 |
| aaatatgata cccaagaatc atatggtatc aatcattcct agatatactg atctattcat | 4861 |
| tgccaagata gttcaatgag ctggcaaaaa catatggaat tattttctta aaatgtgaaa | 4921 |
| aataaaattt aaccaatcat gtatcacagc ttgcaacttt agtcatactt tgaaaagcat | 4981 |
| tttaatttgg acctcatgat tgaaaattta taaaaagctg aacagaaatt agttcacttc | 5041 |
| atattttaga aaagcagagt ttctttacta aatgaggcat ttgacccaaa ttggagagaa | 5101 |
| aatgttgaaa ctacttctgt gagcaagcag gtggcttctc aaacacatgt tgggatgaaa | 5161 |
| tggttgggcc tcagggtctc agtgcctgtc actgagagtt ggcactctct atctccatgg | 5221 |
| tctcctccaa gtgtgactct tgtctcttgc tgacctgacc tgccccaagt gactcactgg | 5281 |
| tcatgaccct gcacaccttg cgtctctcct atcaccctgc cgatggcaga gctacaaagg | 5341 |
| tctttgatgt agctctgtct gatatcctgt gtttccccct atggtctgtg tggaagcagg | 5401 |
| tatgggggtg tgtaaagggg aagcctatga agttcatctg caaagactac ctggttaggg | 5461 |
| gaggagagga agaagctata tgcaccattt caccagcaag catgggctct tctgcctttt | 5521 |
| agcttagggg tcctgttgtc tagtctcact cacctattaa aacagtccag caaatagagg | 5581 |
| ttttgtttac ctcccattaa aaaaaaagca attaatggaa tagaagataa taatgtatga | 5641 |
| gaagcactat tgtgaaagaa aaaccttcaa cttctctcag ccaaataatt gcttcctcct | 5701 |
| ctgtccttcc cagaccttga tgtttgctct attatttcaa aacaactata ttataaatat | 5761 |
| ttgagaatgt gtatttccct gcaaggagat cttaatcccc aagaaggcag gagctgtgta | 5821 |
| ttattcatcc cagtgctcct tcaaaccagg gcctcagaca gtgcatggcc caaagaggta | 5881 |
| cttaataaac gtttggtaaa ctgacactat tgaaattaag caacctggat ttgaggtggg | 5941 |
| tctctgccac tcacaagaga ttacgctttg agaaaattcc atcacttcat tgattttcag | 6001 |
| ttcttgcatc tgtatatggg agacgatact aggtgatttc tgacatctct caccgtttaa | 6061 |
| atgctctgtg atctatacaa cgaggggctc gctgttctag acaagttcct tccagcttta | 6121 |
| cagttgcata acccttctaa tcttagtcac atgatgactc cactgacaga tttttggcca | 6181 |
| ccatcattag acatgctgag ttacgtgtgc ctttgctctg atcctcaaaa ctcatgattt | 6241 |
| ttaaagtttt ctgaaatatc taccatttat caggatccag atggatttca tgaccaaagt | 6301 |
| ggatgtttct tttctctccc attacaatct tttacttttt gtgtgggaaa ttgcatgtta | 6361 |
| aagaaaggga aattgaagaa tgggatgctt tggaattctg gcaagatgga ttagtgggtt | 6421 |
| ccagaaagta ggggcagcca caaataccga aataaatgag atcgtattat tgagaaagca | 6481 |
| caaatggaag aaggtcaaaa gcaagaagaa gctgacatcc tgcttcctcc aatttttgct | 6541 |
| ttctctgttt ttccaagaaa ctcctcttcc aagccttgct gaaaaactcc actttcctaa | 6601 |
| atctaacttc ttaaactgat aatggcaaga agttaggaat gaccaatatg tttaacactc | 6661 |
| caagagtatt tgttttgttt tgttttgaga ctgggtctca ctctgtcgtc cagcctggag | 6721 |
| tgcagtggtg tgatcacagc tcactgcagc cttgacctct cctgcccaag gaatcctccc | 6781 |
| acctcagcct cctgagtaga agggaccaca ggcatgtgcc actacacctg gctaactttt | 6841 |
| aaaaaatttt tttgtagaga tggggttgcc caggcaggtc tcaaactgct gggctcaagc | 6901 |
| aatcctcctg cattggcctc ccaaagtgct ggaattatgg gcataagcca ctacgcctga | 6961 |
| cctctccaag ggcattcttt acccagaaga ggaacttggc agaacttatc ctccaattgg | 7021 |
| tgaggaatat ggagaaaatg actttaagca aaggaacttc tggttctgcc tacctaatcc | 7081 |
| agaaaaagaa gttttatttc tcccttcccc tagtaactat cttcccatat tcacataaaa | 7141 |
| aagtacagaa tcaacattgt tcaagaatta taattttact tgtaagcaca tgtgcacacg | 7201 |
| cacacccata taccttcctt ccctttaaat catcccacac cctaatagta gtaaaatcat | 7261 |
| tgacccgagc atacctggga gaggaagagg agtctgacag gggcaggttc taagtggcac | 7321 |
| tcctggaact taaccctggt gtatatgaac tttacctatt gaaggatgac tcctcaactg | 7381 |
| ttctcacaat ttgctgctct gctttctttt ctaatttctg aaggtgactc atcttcccca | 7441 |
| aggactttca gacttctcag aagaaaaaaa tattgggtgg gtctctgcca ctggcaaaag | 7501 |
| attagacttt gagaatcata aaagtatatc agtatatact cattaatatt gaattactat | 7561 |
| aattaatatt atgatattga tataatgata gaatgatatt gataaaagca atattcaata | 7621 |
| atgaatatta tttcagctgc ccacttattg ggtgcctcat aggtgccagg cattttgtat | 7681 |
| gtattatcta caacccttac atgggacata ttatgatgct gtttctcttg aagaaatatg | 7741 |
| gaaactggaa acagagaggt caccacaatt ttccaaagtc acatagctaa taggtagcag | 7801 |
| acttgggatt caaattcata tgcatatggt aaatcatgct cttcctctgc tacattttgc | 7861 |
| ccccttagaa tatgaaaaag ggatacaaag agatgaagaa aatatgtaag attatccttc | 7921 |
| aatttcacta tcttttttaa agtttttttt attatacttt aaattctgaa atacatgtgc | 7981 |
| agaacatgca ggtttgttac ataggtatac acgtgccatg atggtttgtt gcacccatca | 8041 |
| acctgtcatc tacattaggt atttttccta atgctatccc tcccctagtc ccccacccac | 8101 |
| cgacaagccc cggtgtgtga tattcccctc cctgtgtcca tgtgttctca ttgttcaact | 8161 |
| cccacttatg agtgagaaca tgcggtgttt ggttttctgt tcctgcgtca gtttgctgag | 8221 |
| aatgatggtt tccagcttca ttcatgttcc tgcaaaaggc atgaactcat tttatggctg | 8281 |
| cataccattc tatggtatac atgtgccaca ttttcttaat ctagtctatc attgatgggc | 8341 |
| atttggctta gttccaagtc cttgctattg tgaacagtgc tgcaataaac atatgtgggc | 8401 |
| atatgtcttt atagtagaat gatttataat cctttgggta tagacccagt aatgggattg | 8461 |
| ctgggtcaaa tggcatttct ggttctaggt ccttcaggaa ttgccacact gtcttccaca | 8521 |
| atagctgaac tagtttacac tcccaccaac agtgtaaaag cgttcctatt tctccacatc | 8581 |
| ctctccaaca tctgttgctt cctgactttt taatgattgc cattctaatt ggagtgagat | 8641 |
| ggtatctcag tgtggttttg attagcattt ctttaatgac aagtgatgat gagctttttt | 8701 |
| tcatgtttgt tggccgtata aatgtcttct tttgagaagt gtccgttcat atcctttgcc | 8761 |
| cactttttaa cggggttttt tcttgtaaat ttgtttaact tccttgtaga ttctggatat | 8821 |
| tagtcctttg tcagatgggt agattgcaaa aattttcttc cattctgcag gttgcccgtt | 8881 |
| cactctgata atagtttctt ttgctgcgca gaagtttttt tagtttaatt agatcccatt | 8941 |
| tgtcaatttt ggcttttgtt gccattgctt ttggtgttta gtcatgaagt ctttgcccac | 9001 |
| gcctatgtcc tgaatggtaa tgcctaggtt ttcttctagg atttttatgg ttttaggtct | 9061 |
| tatgtttaaa tctttaatcc atcttgagtt aatttttgta taaggtataa ggaaggggtc | 9121 |
| cagtttagtt ttctgcatat ggctagccag ttttcccaac accatttatt aaatagggaa | 9181 |
| tcctttcccc gttgcttgtt tttgtcaggt ttgtcaaaga gcagatggtt gtagatgtgt | 9241 |
| ggcattattt ctgaggcctc tgttctgttt cattggtctc tgtatctgtt ttgatacaag | 9301 |
| taccatgctg ttttggttac tgtagacttg tagtataatt tgaagtcagg tagcgtaata | 9361 |
| cctccagttt tgttcttttt gcttaggatt gtcttgacta ttcaggctct tttttggttc | 9421 |
| catatgaaat ttaaagtagt tttttctaat tctgtgaaga aagtcaatgg tagcttgatg | 9481 |
| ggaaaagcat tgaatctata agttactttg ggcagtatgg ccattttcat gatattaatt | 9541 |
| cttcctatcc gtgagcatgg aatgtttttc catttgtttg tgtcctttct tatttccttg | 9601 |
| agcagtagtt tgtagttctc cttgaagagg tccatcacat cccttgtaag ttgtattcct | 9661 |
| aggtattttg ttctctttgt agcaattgtg aatggaagtt cactcataag tttgctctct | 9721 |
| gtttgtctgt tattggtgta taggaatgct tgtgattttt gcacattgat tttgtatcct | 9781 |
| gagactttgc cgaagttgct tatcagctta aggagatttt gggctgagac gacagggttt | 9841 |
| tctaaatata caatcatctc atctgcaaac agagacaatt tgacttcctc tcttcctatt | 9901 |
| tgaatacgct ttatttcttt ctcttgcctg tttgccctgg ccagaacttc caatactgtg | 9961 |
| ttgaatagga gtgttcacca cctattttaa gaatagtatt gaagcctcac aaaagctggt | 10021 |
| tctcatgtaa ccatctgaga atatttggcc ttatgacttg aattcattca ttgccttttt | 10081 |
| atttcacatt ttagtgatcc tgatgtctaa atcttaatct ttgatccttg caaggtaaaa | 10141 |
| tagccaagtc aagcctgttt aataatattg gttgaggaag tcacatgctt atgatcaatc | 10201 |
| tttgggttat gtaattatat taccttaatg ttggcagttt aggtgtaagg cagagatatc | 10261 |
| tgatcacatg tgtggttagc taatttaaga tcactgccaa ctaaaatctt catggtatga | 10321 |
| tcttcaaagt tagctacttt gaccacagca atgatttcac cacagcaatt aacaaaatgg | 10381 |
| cagactcttt cctgaggtgg catgaacagt tttaaaacaa agtcaaggac caaaagaaaa | 10441 |
| gcaggcacat ggcatttgat tcatttcaaa aactagtatt gtattaagag ccaaggggat | 10501 |
| agaattgtag catcaattaa aatcttgttt gaaaaaaaat aaaaacaaac gtccattttt | 10561 |
| atctctcaaa tatattaggg ttttcataaa gttataggtg tatttttaaa aaaacaaaac | 10621 |
| tcatatacat tagactgaaa aatttgcctg tcatctcatc atgcagctaa atgcaattgt | 10681 |
| ctatggcgag atacactctt attagaggta tgatagcact aactaatagc aaactttgta | 10741 |
| cctggtagtc taatttatgc agggttcata tttcgctccc tctcagcatg ctgtaactgt | 10801 |
| ggcaaagcca ttctcaggag ttattacccc aacataatca tacccctgtg gattaggagc | 10861 |
| agttaaatgg gtcctgttat cagagacaca tatgtgccac agccgctgtc atccctaagc | 10921 |
| caccgtgggt gattaagact cactgatggg actacctctg aataggcttg agtgaggtgg | 10981 |
| atacacttca gctgagagaa attcaggtaa ggggctgaga aaatcagatt ttgaatggtt | 11041 |
| ttatcatacc atcaggtctc cttttaagtg ctggggtcat ggatttcatt caacctgacc | 11101 |
| acatagcctt tggaagcttg gctcaatacc tgagtgtgag attatgggtg atattaaagg | 11161 |
| agatgaatgc attgagctga tgtcagagaa tgtcttttac tggattttca taatgactgg | 11221 |
| ctgcagatgg gctgagggga aagtcagatc aagagcattt ctgtaagaag aaagaaatct | 11281 |
| tccctcttat tctctttcaa gaaaatgaat agctgagcac caagaggcca aatacttttt | 11341 |
| taaaaaacac atccttttat gtagagaagg acaggttgag acgaaaaaca ggacctctga | 11401 |
| aactgtcttt atagctttaa tctaggaaga aattgcggca ccattgctga catcattatc | 11461 |
| agaggctgcc ttagttctga gagcttcaca gatggccttt tctgcatttt acatctggcc | 11521 |
| agatgaaggc aaaaaggttg atgaaaccaa aactattaga tcagtggtcc ataactctgg | 11581 |
| ctgcacttta gaatcatctg gggagtcctt aaaactactg atcctgaggc tccatcccag | 11641 |
| accaattgaa tcaggatctc tgggggtggg acctgagcat tggaatgctt taaaagttcc | 11701 |
| tcagggactc taatgtgcag ccaaggctga gaatgactga ggtggatggt ggccaagaca | 11761 |
| ggtgaggcca agagttagaa gcccttactg ttagggaagg cagaagccac aaggaggagg | 11821 |
| ggagggggaa ggagcagtat ttagcatttc ttccacacag ttggggggtt ttctgatcaa | 11881 |
| aattaggctg ggatctttcg cttccatttt catgaaggtc ttatgctttg tccacagcca | 11941 |
| cctggcctca gggcaatgag caatctgatt gatgaatttt cagtaagaaa ctgagcacac | 12001 |
| ttggctctca gccccagtgg cttcccctgt ttccaaatct gcccaccagt tacaggagcc | 12061 |
| tgctcaccaa ctggttgggt taaagaagtc ggctctgtcc ttggcaggga ggccttcagc | 12121 |
| tgtctggccc tgtctgtgac tgcgtgggtg aagctgccta atttggggaa cttgatggaa | 12181 |
| gatctaagct atgttctcta acagttttat cagaaataaa gttaactttt gacctccatc | 12241 |
| tgcctgtctc ctgtggaagg gctctgctcc ttccaaagag actctcaggg gttctcctta | 12301 |
| gaggtgtgtt atcagtccaa agatcatttt agaccagcca ttacagagga tgtccaagaa | 12361 |
| attgcacaag ggaaatagaa gtaagaatga gagcaatata tcagatagta aggaaagtat | 12421 |
| gtgactggtt tcaaataagt aattaaccat aaatccaaag ttcctgcatt agttatagca | 12481 |
| atagtcatcc acctggtcag gaaaaaaaaa gtaaaagact gagctgcaag atagaaagtt | 12541 |
| tgcctgaatt ccacgtactt caaggactac tatggaggat tccttggtcc caatgcagag | 12601 |
| acatgtactc tgaccaccca tgggccaaat ccctcttacc caccctcagt gattcctcct | 12661 |
| gggacctcac tacattgagt tcttacattc cccactcttt tcgggggaaa tataaccctt | 12721 |
| ctgctcttct catgatgtta aaaatattta gacaattact taaaatttac aacaatcttc | 12781 |
| cacataaatg atctcactta tgtttcttgt gagctctgtg atttatttct attatcatct | 12841 |
| atgctgatag ttaaggaaac tgagatatcg agaccaacat gcatagtaaa tggcaaaacc | 12901 |
| agatgaattt taaactttcc taactccaaa agccacatcc tgcccaaccc gccatgctgc | 12961 |
| cgctcagtta atgcctggct gtttgtctcc ccattggccc ctctacccat tgtgctttga | 13021 |
| tggcacactg tattccaact gcctgagact cctggttaat gccattacgt acagacttag | 13081 |
| gttgaattta ctaggatttt taagatttgt aggataagag atatgactgt tagactggaa | 13141 |
| tcagcaatag ataaaaggtt aacaaagttt cagaataaaa tataatagaa aaccccagca | 13201 |
| gatagaagta aaatgaatgg taagacaact gaaatgaaag ccatacattt agaaatatca | 13261 |
| aataaaacac agattaatgg cagacaataa aggaacatac ttagcagtta gtaaaaacac | 13321 |
| tttttacctt attcttatta ccctccagta accttttttt tttttttttt tgagacaaag | 13381 |
| tctcgctgtg tcgtccaggc tggagggtag tgatgtgatc tcggctcact gcaacttctg | 13441 |
| cctcttgggc tgaagagatt ctcctgcccc agcctcctga gtagctggga ctataggggc | 13501 |
| ccatcactgc acctggctaa tttttgtatt tttagtagag acggggtttc accatgttgg | 13561 |
| ccaggctggt cttgaactcc tggcctcagg tgatccaccc gcctcagcct cccaaagtgc | 13621 |
| tgagattaca agcgtgagcc accaagcccg gctgtaacct attgaaaata acattacttg | 13681 |
| acatgtgaga caaattattt gtaagttaaa gagtttatgt gccttcaatg ctcccaccct | 13741 |
| tccctcccct aaaaagatta ttgagtgccc atgatgtgcc taagccttct ggtagactct | 13801 |
| gagaatgtga agagagttag aggatacttg ttcaacaacc cagaatctag cagaagtgtt | 13861 |
| ttgaaatgac cagccacttg ggagagctat gctagcatat cattcaggat gggctgggtc | 13921 |
| atgttttaat aacaagtaag tttgaacttt ataggtttga aacaaaaaag atgtgtttct | 13981 |
| tgctcacagt tcatattttt tggagattgg ctggagcttc attccaggat actgccacca | 14041 |
| tctggaatgt tgacagttac cgtaacagag aaagagttgt ggaggctgtc acactggcaa | 14101 |
| ttaaattcgc tgacctggaa ctgacacatg ccacctccac ttatatttta ttggccaagg | 14161 |
| cttgttacac agccacatct aacttcagag aggtcaagat gagcaaatcc taccacgagc | 14221 |
| tatagatgga tgagtagcac tcattattat cacaattatt ttatttgtta taaaaactcc | 14281 |
| aagaaggaga ggtactctat gtgaggaata ggcataggaa aatcagaggg aagttcttaa | 14341 |
| agatgagcta gatttcacta gatggcattg aagaaacatt ccaagtaaat gaacagcata | 14401 |
| agcaaatgca tgaagaactt attgtggtcc tagtacagac gatgcgtgag agtgtggagg | 14461 |
| agggaaaagg taagactgga gaggaaggca ggaaccagaa caggacagac tgtgttcact | 14521 |
| gtcaggcagt taagtctatc ttgtaggcaa catgaagcct tttaaggaag gcaagcagct | 14581 |
| atgtgacagg acagaagatg gattggaaga aaatcaaaag agaagtgggg accagtcata | 14641 |
| aggctcctcc aatatttggg gatccaaacc aaagcactgg cagagaaata gaaaggaagg | 14701 |
| gaaatatttc caaaatattc aaaacagaaa ccaagagaac ttgatgacag aagatgaacc | 14761 |
| caggtttcta ctgaatggac agttgttaca ttctctgaga taaggaatac agaaggaaaa | 14821 |
| agttgagagg gaatatgaaa ttatttttaa tcatgtttaa tttgatcact tgtggaacat | 14881 |
| caaccagaga tgtccatcag ttacaccaat ttgtaggttt tgagagaggc ctgaggtaga | 14941 |
| ggcagagaag tgggaatcag cagattagcg gcagtagttg gaaccataac tgaagatgag | 15001 |
| atttcccagg gaggggagtt gaacaggaag gtagacaaag gctgagtgca gtggctcata | 15061 |
| cctgtaatcc cagcactttg ggaggctgca gtgggtagat cacaaacaag atcaggagtt | 15121 |
| caagaccagc ctggccaaca tggtgaaacc ccgtctctac taaaaaaaca caaaaattag | 15181 |
| ccgggcgtgg tggcacacac ttgtaatccc agcctcagga ggctgaggca ggagaattgc | 15241 |
| ttgaacccgg gaggcagagg ttgcagtgag ccaagatcgt gccattgcac tccagcctgg | 15301 |
| gcgacagagc gagactccgt ccccaccgac cccctaaaaa agaaagtaga caaagatgtg | 15361 |
| tcctagaaaa cactaatatg aatgggtaga gaggggagac ttgtcaagga gatcgagggg | 15421 |
| agagtcagtg aggtgaaacc ttagaggata gaccttcacc aaggaagtaa cagtcaacag | 15481 |
| gcctaaatgc cacagagatg tcaaatgaga gacactggaa atggttcttt gaatattaca | 15541 |
| gccagaacat cacaggagac catttccaaa gcagtcttga tgaagtggtg gggaagggag | 15601 |
| gtccctgaag gagctaagga gggactggga gatcatgacc cagagataag tgttgcaggt | 15661 |
| ataaaaggga aagactgaga tcaggaaata gccacaggat ccctaaggcc aggcttcggg | 15721 |
| tggggtggtg tgtgtgtgtg tgtgtgtgtg tgtgtgtgtg tataaaatgg gaagaccttg | 15781 |
| agtataattc tgtactaaga agatagagca gtagagagga aaaggctgaa gacagatggg | 15841 |
| agggtaaata gtgaattaga aaggtctgtg caaagatgag aaaggatgag tctgaaagca | 15901 |
| cagggagaag ggccagcctt ggacaggaga aaacatttct ttcactaaga ataaagggaa | 15961 |
| ggttgggtat acaccacaaa gaaggtgtag atgggtggca atggagttct gtcaagttga | 16021 |
| gggcattcca tgatagcctc acctttctct gtgaaatgag agagtttagt ttacaagata | 16081 |
| tttctgagca cttcataagc caggttcata gactgaaaga agtggaggtg gagtgtgata | 16141 |
| ggtccttaag aataggggaa gtttggaata tctgacaagg gacagagagg aaaaaactag | 16201 |
| aaaaggcttt gcagaatgtg ggcccacaga tcagaggcta aggggtcccc atttgtgcag | 16261 |
| gagagtaagg gcagggaggc aaggctcaca gcccaaaata tagagcccct caccaaatga | 16321 |
| ctgcaggagg gcagctttcc tatgagagca tccctatcac tgttttcact ccgagtcatt | 16381 |
| aacttacgac ttactcagct ctgtttcgta atagcagact cgagtaatga gggtatgaca | 16441 |
| gcctctctct gcatgccaag gtatgcagcg tggatttcct ttttcgcttt ctctctcctg | 16501 |
| tggcttaggt gccttctgtt ctgctaccag gatagagaac ccagtgacta gtttcttcta | 16561 |
| gctctctttt tctgactagg tatcttgtca gaaatttctg cttaccagac ttcatggaga | 16621 |
| gggaatcaag ctttgaatca gggttgaaaa agtagagctt aatatatata ttacaaaatg | 16681 |
| ccactcacgt tcttgaggtt accttgtatc tataccacaa ctagcattct tttagaaagc | 16741 |
| accattaccg aagtaatccc tttcctggga attcacccaa aaaggttatt cccacttatc | 16801 |
| ccccatctcc aaaataaaaa agaaaaatgt gtgtgcttag agatgttcct ggaagcatga | 16861 |
| gctgtaatac tgaaccaata gaagacgaac taacagattg cagggcatcc gtttggcaaa | 16921 |
| aaacttatgc agttatctaa atgatagtta tgaagacaat atgtataaca cattatattc | 16981 |
| tgagtgaaaa gaacagaagg tgatttcaaa actgcattgg gataatagta atataggaaa | 17041 |
| tagtgagatg aacaaagatt tcaaaggaac aaaaataaag aaaaattttg ctttttatat | 17101 |
| tggtaggcgt atgggtgaaa ttccaatttt aattttaatt tcatagttat aaaattgttc | 17161 |
| ataaaaaaag gtcccccata gacagttggg ctttgggaca aactaacaga aacagagtag | 17221 |
| gaagaaaatt catcttcctt caatcccctt tctctgctta aaacaaaaca aaaaagagct | 17281 |
| ttgtcatgtt caggtgtgca acgaattctt tttccaaatc tggaacttta catctgctat | 17341 |
| taaacaggag tcagtttcca tgtaacatgt tgacaatccc ccaagtgtgt tggaataatt | 17401 |
| ttttttaatg aggagatttg aaattccatt tcaattgcca acctgcctct ttcaacttct | 17461 |
| aaaaacaaag taaaacaaaa caaaaacaca ctgggtccta tcaccccctc ttgctactac | 17521 |
| tattttatct ccattgccct gaattctttc caaacttctt tccacccagc tttgatttgt | 17581 |
| tttcagtcgg gtttattgag gcataattta cgtacagtaa aattcatcct tcttagattt | 17641 |
| agaggtctat gcattttgac taatgcatat tggcttataa tgactaccac aacaaagata | 17701 |
| tagaacacac ccatcactcc cgggttctcc ctgtcccttt tttggtccat ctcctctcct | 17761 |
| acccccaacc ccttggcaac cactgatctg ttttctgtcc ttatcatttt gctttttcca | 17821 |
| ggatgttgta tataaggaat catgcagcat gcagcctctc gagtctgact tcttccagtt | 17881 |
| agcacagtta tttaagatct atccgagtta ttgtgagtag cagcatcttt tttattgctg | 17941 |
| actagtattt catcacatgg atgggccaca acttgtttat ctgttcacct gtcaatggat | 18001 |
| actaagttgt ttccagtttt tggcaaatat gaataaagta aacatttgca tacagatttt | 18061 |
| tgtgtggaca catgttttca attctcttag gtaaatactg agcaatggga ttgctgggtt | 18121 |
| atatgttaag tctatgttca gttttctaag ttctgaaaca attggatatc tatatgcaaa | 18181 |
| aatacaaaat taaccttgac tcaaacttgt accatacaca aaaaataacc tgaaagagat | 18241 |
| cacaggtgta aatgtaaacc tagaactaaa aacttcaagg agagaaacat agcagaaaat | 18301 |
| atttgtgacc ttgcattagg caaagacttc ttagatttga catttgaaac atgatacata | 18361 |
| aaaaatcttg ataaattgga gttcataaaa ataagaacta ctcttcaaaa gacactgata | 18421 |
| agagaatgaa aatacaagcc acagacagag aaaatatttg tgaatttctt atctgataaa | 18481 |
| gggtttgtat ccagcatgca taaagacttt tcaaaactca ataataaaca atccaataag | 18541 |
| aaatgcacaa aagatacaaa cacatcatcg aaaatgacct atgaaaggca aataagccca | 18601 |
| caaaaaaatt ctcaacatca ttagacactt gggaaatgca aattaaaaca acactgagat | 18661 |
| aaactacata tctattaaat ggctaccact ttaaaaacct gtcaagtgcc agccagaatg | 18721 |
| tggaacaagt aggactctct tacattgcta gtgggaaggt gaatggtaca gccactttgg | 18781 |
| aaaacactcc gcagcttctt atagttacac ataggacctg ggggtagagg atggattgac | 18841 |
| tgtaaagggg caaaacttcc tggtaggggt ggggaaagtt tttagaaggg tgaaattgtt | 18901 |
| ctatatcttg attattgtgg tggttacaca actgtctgca tttgtcaaaa ctcacagaac | 18961 |
| tgtacactaa aaaggtatat ttttatgctc tgctaatttt actttaatct taaaaatagg | 19021 |
| aaggaaaaaa taaaatcaat gccactgtgc gactttgggc aagttacttc acttctctgt | 19081 |
| gccttggtct tttgaaatct atacattaag gataaaataa taccttcctc atactgttag | 19141 |
| aattaaatgt gctaatttat gttttatata tataaagtac ttggccgggt gtggtgactc | 19201 |
| acacctgtaa tcccagcact gtgggaggcc gaggtgggca gatcacttga ggacaggagt | 19261 |
| tcaagactag cctggtcaac atggtgaaac cctgtctcta ctaaaaatac aaaaattagc | 19321 |
| tgggcttggt ggtgcgtgct tgtaatccca gctacttgag tggctgaggt gggaggatca | 19381 |
| cttgaactcc agaggtggag gctgcagtgg gctccaccca ctgcctgggt gacagagcta | 19441 |
| gactccatct cgaagaaaaa ataagtactt gaaacatagc aaatgtttta taattattgg | 19501 |
| cttttttttc ttattgttat tacttgcatt attgctgttt gaagaagttt ggtgataatg | 19561 |
| gagagaaaag ggcaattagg ggtctgggat ggtttaagta tgaggagacc gagacacatt | 19621 |
| gactcagagt gaagaaatca aagataagag aatgaaagaa agggagggta attctgaacg | 19681 |
| cacagacaaa gttatgttac taacgtggca tcggctgtgt gttgtaataa ataactccct | 19741 |
| ttcacttgtc aatagctaat caaatacttt ctggagacca gaagtgactt gctggatcaa | 19801 |
| tgacaactcc tccacagatc aaaatgttca aatccttttt ctgtgttgta atcctaaatc | 19861 |
| taaaagaaca gagagaccaa gcaaatctac ctcccaacat cattaaagtg acaactctca | 19921 |
| gtatttattt gaatggtctg ctctcagctt caaccaagga aaagtcaaat tagtgttggt | 19981 |
| cagaaaacag aagggtgtta cgagagttct ggctggtcat tacagacttg gggatttttg | 20041 |
| attaaaagaa gaagaagaag aagaaacctg ataaagtgta aatatagcaa gcagggatta | 20101 |
| gtgtcctgct gggtcatgtt ctcacaacag tgagaatttc agagatttca taagaattaa | 20161 |
| actgctccac atgacaattt attttacctt ctggcttttc caggaggcaa atcagtgcaa | 20221 |
| cttctttctg cctttgtttc aatttggtaa caaccctcaa ttttaggaca ggctaaacct | 20281 |
| agccacccta tcagagatga tgaagtagcc atctttttaa caggtgggga gatgaatgga | 20341 |
| atcagggttt gtttgtttgt ttgtttaata actgctagta aaaaccaagt caatagctga | 20401 |
| ctgagtgtaa gggaggctcc agaaggcagg ttattgtagt atagatgtga ctcgacttat | 20461 |
| gatgatgtta cttcccgata aacccgtcat aagttgaaat atcgttaaat tgaaaatgct | 20521 |
| tttaatacac cgaatctacc gaacatcata gcttagctca gcctacgtta aatgtgctca | 20581 |
| ggacgcacat tgcctacagc tgagccaaat cacctggcaa cacaggacac tgtagagtat | 20641 |
| cggttgctgg cccttgtgat gctgtgactg actgggagct gcgcttagtg cctctaccca | 20701 |
| gcattgagag tttcttatcg cttattacta gcctgggaaa agaccaaaat tcaaaactca | 20761 |
| aagtgcggtt tctaccgaat gcttataact ttcagaccat catgatgttg aaaaatcgaa | 20821 |
| ccatcgtagg ttgggatcca tcctataaga cgagctacac tgccggaagt gtaagactgc | 20881 |
| tatgctgccg gaagatgggg catagtggac aactgcaagt cctgacaaca ggaggtcagc | 20941 |
| atctgcgacc tttaacatcc acattgacac taccacagtc ttccaaacag agctgatgat | 21001 |
| atagtttgga tgtcgtccct gcccaaatct catgtcgaat cgtaatcccc agtgttggag | 21061 |
| gtggggcctg gtgggaggtg attgggtcat gggggcagag ttcttatgaa tggtttagca | 21121 |
| cggtcccccc ttggtactgt atagtgagtg agttctcatg cgatctggtt gtttaaaagt | 21181 |
| gtgtggcacc tcccctctct ctctttctcc tactctggcc atgtgaagtg ttggctcccg | 21241 |
| ctttgccttc caccatgatt gttaaattcc cagaggtctc cctagaagct aatgctgcca | 21301 |
| cgtacagcct ggagaactgt gagccaatta aacctcatct ctttttaaat tacccagtct | 21361 |
| caggccgggc gcggtgactg acacctgtaa tctcagcact ttgggaggct caggcaggaa | 21421 |
| gatgatttga ggtcaggagt tcgagaccag cctggccaac atggtgaaac cccatctcta | 21481 |
| ctgaaaatat aaaaattagc caggcatggt ggcgggtgcc tgtaatccca gctacttggg | 21541 |
| aggctgaggc aggagaatcg cttgaacctg ggagtcagag gttgcagaga gccaaaatgg | 21601 |
| agccactgta ctccagcctg ggcaatggag tgagaccctg tctcaaaaaa tatatatata | 21661 |
| ttacccagac tcaggtattt ctttacctga gactatgaga gaatggacta atatagctga | 21721 |
| agaattttat tttattttta aaaaactttt acgtttgggg gtacctgtaa aagtctgtta | 21781 |
| cataggtaaa ctcctgtcat gaggatttgt tgtacagatt ctttcctgct cctccccctc | 21841 |
| ctcccaccct ccatcctcaa gaagatccca gtgtctgttg tttccttctt tgtgttcgta | 21901 |
| agttctcatc atgtagctcc cacgtataag tgagaacatg cagtatttgg ttttctgtcc | 21961 |
| ctgtgttagt ttgctaagga tgatagcctc caactccatc tatcttcctg caaaagacat | 22021 |
| gatctcattc atttttattg ctgcatagta ttccatggtg tatatgtacc acattttctt | 22081 |
| tatccagtct gtcattgatg ggcatttagg ttgattctgt gtcttcagaa ttgtgaatag | 22141 |
| tgctgcaacg aacattcgtg tgcttgtgtc tttatagtag aatgatttct attcttctgg | 22201 |
| tagtaatggg attgctgggt caaatggtcg ttctgctttt agctctttgc agaatcacca | 22261 |
| tactgctttc cacagtggtt gaactaattt acactcccac taacagtgta taagtgttcc | 22321 |
| cttttctctg caaccttgcc agcctctgtt atcttttgac tttttaataa aaaccattct | 22381 |
| aattagtgtg atggtatttc attgttgttt tgatttgcat ttctctaatg atcagtgatg | 22441 |
| ttgagctttt tttcatgttc gttggctgca ggtacatctt cttttgaaaa gtgtctgctc | 22501 |
| atgtcctttg cccacttttt aatggggttg tttttctctt gtaaatttaa gttcctcata | 22561 |
| gatgctgggt attagacctt tgtcagatgt atagcttgca aatattttct cccattctgt | 22621 |
| aggttgtctg cttactcttt tgattgtttc ttttaccatg cagaagctcc taagtttaat | 22681 |
| tagatcccat ttgtcaattt ttgcttttgt tgcaattgct tttggtgtct ttgtcatgaa | 22741 |
| atctttgcca ggtcctatgt ccagaatgat attgcctagg ttgtcttcta gggtttttat | 22801 |
| agttttgggt tttacattta aatctttaat ccatcttgag ttgatttttg tgtttggtgt | 22861 |
| aaggaagggg tccagtttca atattctgca tatggctagc cagttatccc agcattattt | 22921 |
| attgagtaag gagtatctcc tccgttgctt gtttttccca ggtttgttga agatcagatg | 22981 |
| gttgtaggtg tgtggcctta ttttggggct ctctatcctg ttcatttggt ctatgtgcct | 23041 |
| gtttttgtac cagtaccatt ctgttttggt tactgtagcc ctatagcata tttcaaagtt | 23101 |
| gggtaacatg atgcctccag ctttattctt tttgcttaga attaccttgg ccatttgggc | 23161 |
| tctttttggt accatatgaa gtttaaaata gttttttttc tagttatgtg aagaatgtcg | 23221 |
| ttggtaattt gataggaata acatgtaatg atattgattc ttcctatcca tgagcatggg | 23281 |
| atgtttttcc atttgtttgt gtcttctctg atttcttcaa gcagtgtttt gtaactcata | 23341 |
| ttgtagagat tattcacctc cttgcttagc tgtattccta ggtattgtat tctttctgta | 23401 |
| gtaattgtga atgggattgc ttttctgatt tggccctcag cttggtattg ttggtgtata | 23461 |
| ggaatgctag tgattttttg tatcctgaga ctttgctgaa gttatttatc agctgaagga | 23521 |
| gcttttgggc tgagactagg gggtttttta gatatagaat catgttctct gcaaacagat | 23581 |
| ttagtttgac ttcctctctt cctacttgga tgccctttat ttctttctct tgcctgattt | 23641 |
| ccctggccag gacttccagt accatgttga ataggcgtgg tgagagaggg cattcttgtc | 23701 |
| ttgtgccagt tttcagggag aatgcttcca ccttttgccc attcagtacg atgtttgtgg | 23761 |
| tggtttgtca tatatggcta ttattatttt gaggtgtgtt cctttaatac ctagtttatt | 23821 |
| gacagttttt aacatgaagc agtgtttaat tttattaaaa gtcttttctg cctctgttga | 23881 |
| gatagtcatg tggcttttgt ctttagttct gtttatgtga tgaatcacat ttgttgattt | 23941 |
| ccttatgttg gaccaacctt gcatcccagg gatgaagcct acttgattgt ggtggtttag | 24001 |
| ctttttgata tactactgga ttcagtttgc aagtattttg ttgaggattt ttgcattgat | 24061 |
| gttcatcacg gatatcggcc tgaagtttct ttttttgttg tgtctctgtc aggttttggt | 24121 |
| atcagaatga tgctggcctc ctagaatgag ttggggagga gttcctcctc ctcaattttt | 24181 |
| ttggaatagg ttctgtagga atggtaccag ctcttcttta tacatctggt agagtttggc | 24241 |
| tgtgaagcca tcaggtcctg ggatttttta gttggtaggg tatttattac tgattcccta | 24301 |
| aatagaccga taatgatttt agaagtggag tcggtttttt cctggtccag tcttgggaag | 24361 |
| gtgtatgtat ccaggaattt atttagctct tctaggtttt ctagtttgtg tgcatatggg | 24421 |
| tgttcatagt agtttctgat ggttgttttt atttccgtgg gatcagtggt aacattctct | 24481 |
| tcatcatttc tttttttttt tttttttttt ttttttgaga cggagtctcg ctctgtcgcc | 24541 |
| caggctggag tgcagtggcg cgatctcggc tcactgcaag ctccgcctcc cgggttcacg | 24601 |
| ccattctcct gcctcagcct cccgagtagc tgggactaca ggcgcccgct accacgtccg | 24661 |
| gctaattttt tgtattttta gtagagacgg ggtttcaccg tgttagccag gatggtctcg | 24721 |
| atctcctgac ctcgtgatcc gcccgcctcg gcctcccaaa gtgctgggat tacaggcgtg | 24781 |
| agccaccgcg cccggcccat ttctaattgt gtttatttga atcctctctc ttctcttctt | 24841 |
| tattaggcta gctagtggcc tatctatctt attaattttt tcaaaaaacc agctcctgga | 24901 |
| tttcttgatc ttttgaatgg tttttcatgt atcaatcctt cagttcagct ctgattttgg | 24961 |
| ttatttcttg tcttgtgcta gctttggggt tgacttgttc ttgcttctct aattctttca | 25021 |
| gttctgatgt tagtttgtta gtttgagatc taactttttg atgtggacat ttagtgctat | 25081 |
| aaatttaact cttaacactg ccttagctgt gtcccagaga gtctggtatg ttgtatcttt | 25141 |
| gttctcatta gtttgaaaaa acttcttgat ttctgtctta atttaattat ttatccaaag | 25201 |
| tcattcagga acatgttgtt taatttccat gtaattgcat ggttttgagc gattttctta | 25261 |
| gtcttgactt ctatttttat tgtaccgtgg tctgagggtg tttgatatga ctttggttct | 25321 |
| tttgcatttg ctgaggattg ttttatgtcc aattatgtgg ttgattttag agtatgtgcc | 25381 |
| atgtggtgat gagaagaatg tatattctgt tggttttggg tacagagttc tgtagaggtc | 25441 |
| tattagatcc atttggtcca atgttgagtt cagatcctga atatctttgc taatttcctg | 25501 |
| cctccatgat ctaatactgt cagtaaagca ctgaagtctc ctactactat tgtgtgggag | 25561 |
| tctatgtctc tttataggtc tctaagaact tgctttatga atctgggtgc ttctgtgttg | 25621 |
| gatgcatata tatttaggat agttagatct tcttgttgaa ttgaaccctt taccattatg | 25681 |
| taacgccctt ctttgtcttt ttttttcttt gttggtttga agtcttcttt gtctgaaatt | 25741 |
| aggattgcaa cccctgcttt tttctgtttt ctgtttgctt ggtagatttt cctccatccc | 25801 |
| tttattttgg acctatgggt gtccttacat attttatctt tatctatcca tccagccatc | 25861 |
| cagccatcca ttcatccgta tcatttttaa ccaataagga cttttaaaag cgcaaccaca | 25921 |
| acaccattaa cataaccaat aaaatctata acaatgataa aatatcatct aatactcagt | 25981 |
| ccatgtccaa ttttccctcg ctatctcaaa atcgtcttct tagaaatggt ctgttcaaat | 26041 |
| gagatcacat ggacagagga aggcgaacct cacactgtgg ggactgttgt ggggtggggg | 26101 |
| gaggggggag ggatagcatt gggagatata cctaatgcta gatgacaagt tagtgggtgc | 26161 |
| agcgcaccag catggcacat gtatatgtat gtaactaacc tgcacaatgt gcacatgtac | 26221 |
| cctaaaactt aaagtataat aataaaaaat aaaaataaaa aaataaaaag tgaaaaaaga | 26281 |
| aaaaaaaaaa aaaaagaaat ggtctgttca aatcacaaac cagattcaga aacaatagcc | 26341 |
| atacattaca ttttattaat atgtctctta aatttctttt aatctattac agtctttgga | 26401 |
| atttttatgt cttcgtttat ccttccaatt attaaaaaaa aagtattttt gtattcattg | 26461 |
| aatagacaat gcttgcagaa aagtaaaaaa aaaaaaattt agtacaaaaa ggtacatagt | 26521 |
| gagctgttcc ttagtctccc ttcccagaag caatgttacc acttttgtac aaatagtctc | 26581 |
| tgcctagaca cacatgccag tccctaaggt ggctgtaaca aggtggttaa gagtgagaac | 26641 |
| atgaattcaa attcctatta tgccactcac taagtataaa tcttggtcat ggtacatgcc | 26701 |
| tctgtgcctc agtttttaat aatggtacct acctcatagg gctgttgaga gaattaaatc | 26761 |
| agataagtgc ttaaataact attaatattt attattattc acattccctt ttggcttttt | 26821 |
| tcccaaatag cagagtggtg cacatatgtc ttcatttatt tggcttgttt tttcacctca | 26881 |
| catcacattt tgatgaataa ttccatacat gttgttatag atttgcttca ttctttgtaa | 26941 |
| tcattgacta atattccatt gtatgaatat gctactgcta aacatgtacg ttatttccaa | 27001 |
| cctcttatta tcaagaaatg ctgcaatgaa tatccttgta atactttagt ggattcatgt | 27061 |
| gcaaaaatat tcataggata aaatcctgaa agttaaattg ctgagctaaa gggtatgtgc | 27121 |
| attttaatgc tttatagatt gcccagctgc ctcaaaggag gttataacaa tttacactcc | 27181 |
| caagaaaaat gcacaagggt ccccatttcc ccatacccta gctaacacag gatattgcta | 27241 |
| aatgctttca tctttgtaaa catgatgtat tgaaaatggt atctcaaagt tttaatgtgc | 27301 |
| atttttctga ttgtgagaag ataaaggaaa tgtagtacaa ctaaacatca gcagtcaaat | 27361 |
| gacctggcca tgactcctga gtgaggacac tggtaaacac catcaggatc caaacacctc | 27421 |
| tgtatttacg aagaggatgc tccctattgg atagcactaa gcttatttca tgtatgtaca | 27481 |
| tatgtagtta gttaattaca tccagcggtg gcaaagggct tgttctgacc caatgaaact | 27541 |
| ttctctcctg gcccccttcc agcatgtggt caggagtaga gtgttgtggc catgaggcat | 27601 |
| gcatttgtac agatgactac ttactcctcc ttgaaacatt tttttccatt tgcttccctg | 27661 |
| ctgtctcact catgggtctg ctcctaattc acaaatcact cttttcccag tcttcttggt | 27721 |
| tgggttttct cctcttctgt gcttgtagac atgggggagc cccagggctt ctctcttgaa | 27781 |
| ctacagcttc tccctgggtt catctccttg ggatgtctgt tccaatgggt ttaaatacta | 27841 |
| gggctaggac tagggagagg tcattgaggc acttagcaca taaagtttaa ggaaacattc | 27901 |
| tttatcaggc ccatgcaagt gcaggactgg ccctggaggg tgactaccac cttacctttt | 27961 |
| ccaccctagg caccttgttt gccttaccct agccccagtc ctctttaaca ccccagtctt | 28021 |
| ctccctggac ctccagaaac ataaatccta tttgacattt ctacttggag gttttaaggt | 28081 |
| aactcaaacg taaaatatct aagacagaac tcttgcatca cttcccatcc tggggcccaa | 28141 |
| gcctgtctct tctactagtc tatctcagtt aacagcatca ccatttattc agttgctcag | 28201 |
| gacaaaaaat ttgaagtaat ccttgactct tctttttttt tttttgagac ggattctcac | 28261 |
| tctgttgccc aggctggagt gcagtggtgg gaccttggct cactgcaacc tctgcctcct | 28321 |
| gggttcaagc aattctcctg cctcagtctt ctgacctcgt gatccactcg cctcggcctc | 28381 |
| ccaaagtgct gggattacag gcgtgagcca ccgcacccgg cctggattct tttttttttt | 28441 |
| tttaaacaag tcctatcttc catctccaaa atgtatccca aatctgacaa cctctcccac | 28501 |
| cgtaggccag cccccatctc tcccctctga aaatagcctc ccttagatct ctggacattt | 28561 |
| gttcttcccc acccccttgt gatcactatt cagcattcag aatgatcttt taatattatg | 28621 |
| aaggagactg tgttcctctc ctacttaaaa ttctctagtg gcttcctaat aaatttagaa | 28681 |
| taaaaagcca actcctcgcc atggtcacca ggccaggatc cagtgggtac aacaatctgt | 28741 |
| tcccagcaca ataatcccac ttctgcctcc ccaccattca ccaggctcca ctgcactggc | 28801 |
| tcattcctgc ctcagttgtg cttcttttcc ctggaaagtt ctttctgtag atctttaaag | 28861 |
| ggtgatttcc ttctcaacat tcaggtgtca gctcgtttca ctttcctgac catgcccatc | 28921 |
| cactcagaga tcactcaaaa tcccattacc ctattttatt tctccatcat atgtatcact | 28981 |
| atctgaaact atcttgttgt tgatgcaggc tattttcttg accccttcat gggactccca | 29041 |
| acaggggtac cccatttact cagcctgccc tgctcaacct cttgcaggag ggagcacacg | 29101 |
| agtgaacgag tgcaggaacc agctggctgc tttagtgctg tgaggagtaa actccatgca | 29161 |
| ggccctgcag cagcaaccag gtaggggtgc ctgcaacccc agggccccag agggtgtgtt | 29221 |
| acaatgctct cgtagctctg ccatctgtgg acagcagtgt gttgtcagct cagtgggccc | 29281 |
| tttgcttcat catgtagggt ggctgccctc tgcctgtgag ggcaaagggc cagggtgaca | 29341 |
| gtctttttgg gtacccacaa tttgtgcatc ctgaattctt gtttggtgcc caagaagaat | 29401 |
| ggggtcacac agatgaactg aaggatggtg aatgcagaga attagcaatg aaagtggctc | 29461 |
| tcagcagaga gagaagctga aaagggaatg ggaagggcag gtcactctcc cctgaagtca | 29521 |
| agtcacatct ctccaatgtc cagccaccat ctctgaagtc aagtttcctc tctctgatgt | 29581 |
| ccagccactt ctcctctcta ctggctgagt ctggggtatt tataggcaga ggataggtgg | 29641 |
| tggggcaggc catacataat tttggaaaag gcaacattct attggtaaaa agacattatt | 29701 |
| cataaagaac caattgggaa agagcgggca cacagggatg gaagttctca ctttgggctg | 29761 |
| caggtttcag gcttttcagc tcaaaagtga ggtttttcca gggacctgcc ccgtctgcct | 29821 |
| aaaatttcta cacttctgtc attgataccg ctggataaca gctctcctat aaagttcagg | 29881 |
| ggcttaaaac aaaaatttac taattcaccc ggacagttct tgatagggtc tctcctaact | 29941 |
| gttgcagtta catagcaact gggttggagt catcttttct gggcttgaca tccaggacag | 30001 |
| cttcttccct tgtgtgtctg gtgcctcagt gctcctccag gcagcctttc tctccagaag | 30061 |
| agtagcctgg acttcttggc aactcaagct tccaaaagaa aaaaaagcag agactgctgg | 30121 |
| ttctcttatc aaacaggcct ggaactggca caatgtactt ctgctgccat attcaggagg | 30181 |
| tcaaagcaat cgaaggccaa tccaagttca aaggcattgg agaaaaatga gaagtgtcac | 30241 |
| ttaaatgaga agtgacacat gcgtaaaagg gggaaaagca ttgattgtgg ccatttttgg | 30301 |
| agataagcta tcacgtttat ttgtttgttt gctttctaat ggtctgtctt ctcccattag | 30361 |
| gttataagct ctgtgagaca acaggaatct tgtccatctt gttttatggc tctacttcca | 30421 |
| acacctagaa taatgcctgg cacatagtag gtgctcagtg aataacttaa gcacttgata | 30481 |
| catgtttggg gaactaaaat gaacagaatt aaacttcccc agattggtcc tgccagattt | 30541 |
| gctgatgcca agcatgctga tgcctcacca gatgaaagaa gccctaaaat gtagggtttc | 30601 |
| gctttctctg caaacaagaa aaacttgccc tgaacacaaa atctagaaat agatttggcg | 30661 |
| tgttttctac attgaaatat ttcccgtagt accagaaatt attttcccac agctttgtgc | 30721 |
| tacattaaaa tattgaagtt gactgaaaat atctccattc tttaatcttg gtgtagacta | 30781 |
| gaaacaattt ttttgtaaca aagtaaatat gaaaacttcc taatatttga actccccaga | 30841 |
| tatccccaga tatctccaaa cttaaaatat cattgcaagt taagataaat ttttttaaat | 30901 |
| gactaccgag aaaggtcatt aaaggcttgg ttattaaaat gtacagattt gggttataaa | 30961 |
| gccagaactt atttgtttaa atcattacat atgaccaagc acagaaaata aattacctca | 31021 |
| aatctcctct ttgctaattt ttactggtaa actctataaa atgatcctat ctttaaacct | 31081 |
| ttttgtaaac cccttataag ttagtaagtg agaatgtatt catcagaagg attttagtga | 31141 |
| tgtttgaaat taaaaaagag agatttgatt tttaaaatta tacttgcaga ctactgctaa | 31201 |
| tgaaacttct tctaacccta gttttgtctt atcttcagtt tttccagatt tgctcaaagc | 31261 |
| aatcccagtg agcatccacg tcaatgtcat tctcttctct gccatcctta ttgtgttaac | 31321 |
| catggtgggg acagccttct tcatgtacaa tgcttttgga aaaccttttg aaactctgca | 31381 |
| tggtccccta gggctgtacc ttttgagctt catttcaggt aagtacaaaa ttctacctct | 31441 |
| gaagacaaat gtgcttttca atatgtcaaa aagaccgtct acctaaatat aaagttataa | 31501 |
| tcttaacata tatacatgga tgcacactgt agtattatac ataataacaa aaagtgtgga | 31561 |
| aataccacac ttgctcaaca gtagggaatt caataaatac tttatggaca tctatatgaa | 31621 |
| taactgtgat gctgacatta aattatattt ttgaagatgt aatcaagagg ataaacgctt | 31681 |
| gtctcaaaaa gttacatggg aaaagcagta tgtaaactta tataaacatt gtgaacctaa | 31741 |
| tttgattata tatataaaat atagggaata tacatataaa atacatatat gtatatatgg | 31801 |
| gggctatata tatatatata tgaaagagat agatagatag atagatagat agatagatag | 31861 |
| acagacagac agacaataca gactccaatc tgttggtcgt ggttgtctct gacccatgac | 31921 |
| actatggggg acttttattt ttgctcatac ttttcaatat ttcttagtgt tcaataatgt | 31981 |
| gctattattt atacataata ataaaaataa ataaatggca tcaaaaaaga gtaaagggcc | 32041 |
| agtgttccgc ccacatatga gcagccatat tcaagcctgt agacactttg tgtagcctaa | 32101 |
| tgctaggtgt atctgggcaa ggataaactc taaagccaga aattagttca tcaataaaca | 32161 |
| tgtgctactc aatagctagg gctgatggaa aagaatataa aacccagtct gtgccaaatg | 32221 |
| gtgcttacta tctgcaagtg ggagaaggag aaagacgaga aaatgaaaaa tgtgtgtata | 32281 |
| atttatatgt agctgttctg taggagatct ctgacttcac cccattctaa ctttgcaaaa | 32341 |
| agatccaaca ctttgtcaga ttcctgggag gcaagtaatt ttattgatgg tttcatggag | 32401 |
| ggatacagaa cgataacaac tcacacaaag caaacaatgt aatgaaaatc tctattcgac | 32461 |
| tgtttctttt tctcctgaag ttgccctttg gctgccagct accaggcacc aggctcaagg | 32521 |
| tactttcttg ctcttgacac tactcccttc tctcatacaa ttcaacccca accacaaacg | 32581 |
| tgtatagatc tctctctcta taaaacaaag gcctgtagtt aacaggaggt cacttgcagt | 32641 |
| gtagcctctg ttcattgtta cttgtgcaca ctgcttaggg tctcacccca tccacattct | 32701 |
| gctaatcaca ttattcaccc atccaatgta gatctctcca gtggagattc tgctaatatt | 32761 |
| ttctttagat ttgtcacaag tatataatac agttttaaat tgtacagatg attatcctat | 32821 |
| aacagaagag tctaagcctt ttacatcttt gtatctctaa cgaaagcatt cagcatgaag | 32881 |
| ctctgtacac agcagacaat tcaatatgaa tttgctgact tgaaacagca agcctagaaa | 32941 |
| ggagatgtta acttggtcac ttagacagaa caggtttcag caatcagaat tcagatgaca | 33001 |
| tggaactggt agaacaggcg ctttgaagca ataggacatg agccagtgag gagagggatg | 33061 |
| gaatatcata aacaaaggcc aagggctttg caatcagagc tgaagagcca agagcacagg | 33121 |
| ctcagggtgt gggcagactg aatgagaaag tgattcaatc acatgtgaaa gtccagatga | 33181 |
| gaagagagag ttgggattac ttctgctcac caaacatcca aaaccaaaca ggtggatccg | 33241 |
| ggtggtgtgc tgttttactg atgaccatta cacagaattt taacagaagg aatgtaaagc | 33301 |
| agtggttctc aaactggagt tcccagatga gcagcgtctg catcatctgg aaacctgata | 33361 |
| aagcagcaaa ttctcaggcc ctaccccaga cccactgaat cagaaacttg ggggtctggg | 33421 |
| ggaagatggc catctgtatt ttaacaatct cccttcagga gattctgagg ctggctcaag | 33481 |
| tttgaactac aggtagttgg ttcaacaggt gttggtggac tgacaaacaa aaagagactc | 33541 |
| cgaggtaact ccaagatggt aatgtcagaa agcagctacc acccctaggg cttgggggaa | 33601 |
| ccaacaaaag agtttggcat tgccagaacc tagaatcttg aggagaggcc ccagagcatt | 33661 |
| gtgtctcaga ccttgaggac ttggcactgg gacaccatga ggggtttctg ggtgtgggaa | 33721 |
| agggctggaa actccccagt tgctgccacc agggagaact acaagtgaag tggaaggtgt | 33781 |
| gggcctttct cccttttctt ttctcgtctt ctctctcccc ctggtgctca tgtttgacag | 33841 |
| aaaacagctg aaaaggcaga actagtttgg ggagtcttga cctggcatca taaagcagag | 33901 |
| aaaagcaaag ctggagtgaa ggtgagacac aacagctcat tagcagcaac agccgtctag | 33961 |
| cgctccagct tctgaatgaa attctgaaga acagcgcact tggaagacaa attatttgac | 34021 |
| agttctgaca gacgaccaaa ctaacagcat ttgaaaagca agatgactca gagaatacag | 34081 |
| aatttaatcc aaatcccaga tcctatctct gcctttggcc aggcctaatg caaggagaac | 34141 |
| ctgagcacac aaatatatgc aggaatgatc aggacctgtg cctgcattct attctgtctc | 34201 |
| acccaccttc aaatttgttg taaaaacatg ggctcaataa aggtttgtga atcagggaag | 34261 |
| gaagagaagg ggagaaagga agggaaggag ccagctccag atctgtgtct tgcagaggat | 34321 |
| aaaggccagt gtttttagat cacccagtgt ttttctaagc ccccaatact tattttgaaa | 34381 |
| tatcaaaatg ttcaataact aaaaaaaaaa ccgttacaac aataaaacat gtttgagggt | 34441 |
| cagatggact ggcagtttgt gacctctggg gataaacagg tcactttgga atcacagact | 34501 |
| tcctcattcc ccttaaatct catatggtac ccagaagccc ttggaacttt ggaaggtgtt | 34561 |
| tattcacagt tgtaatgtcc atgcagaccc tggctctaag acccaattgt gtaagggtag | 34621 |
| gtttgtagcc cttatcccaa acattctaag tgtgagccaa tgcgtcacac actcagaggc | 34681 |
| cagagactgt attggggtcc tttatttcac gtacgagtca cattccatta agagacccca | 34741 |
| gaagtcagct ctcttccact gactggttct cttcccttgt ttctcttgcc aatgtgtgct | 34801 |
| gcccaggtgg cagtgctcac tgtcagcaga gaagaaaaat gctttcctcc ttggacctct | 34861 |
| tttctctttt tctcctccct actcacattc aggttcccta agcttccccg ctccttgtgc | 34921 |
| tgaagtcatt ctatggtcat ttcttcaact gtctacttcc ctgctggatg ggcacccaag | 34981 |
| acttggcatc ctggggcatg tagaaagggg aaagggaagg gaagggaaaa gaagtcctcc | 35041 |
| caattgtcta tctggacctt tccacactgc ccagagtact gctatgggca tctccttatg | 35101 |
| tctcccgatg tggtgcatgc cagaccctgc aggtagaaaa ggaaagaaag caacccattg | 35161 |
| gaccaggcca gcaaaggctt cagtcacaca gctggctcat acttatggct tcatattctg | 35221 |
| ttgcctcttg aaccagacat ttcttccact ctcataacct ccagtttagc tcgtattcct | 35281 |
| cagcattctc catgtaatat tgttgcatga aacccatgca agtcagccaa tttgctcttt | 35341 |
| ctcatcttgt catttataaa ttgatgctga gaagtctttt tcccaaggtt tttagtaata | 35401 |
| ccttcatcat cccccatagt tcattttggg cagtgattcg cttctttgac tgtacattag | 35461 |
| aatcatctga agaactttct aaaactactg atctcaggtc tcacacaaca ctaattaaat | 35521 |
| tatagtctct ggtggggtag ggcttgggca ctgctatttt accttaagct ctctggagtc | 35581 |
| attctaattt gtagccagtg ctaagggttg caatataagt gaatatattt cacgtatttg | 35641 |
| tcaaacattg actgagtgcc cattatgtgc cagccattat aataggcact ggtgatccca | 35701 |
| cagtgaatca ggcacacaat gctgtcttca cggagcttgt tgtctagtgg gagagtcaaa | 35761 |
| caaaagtgta tatcaataat taagtgatta cagattgcaa taattacaat aagggtgata | 35821 |
| aacaggttgc tataatatac aatagtattg cctttcacca gacatttctt aaagaggtaa | 35881 |
| atcatctatc agacaccttt taaaaatctc atctaatttc aaaagtgtac ataaataatt | 35941 |
| aagtgattac agtttgcaat aattacaatg agggtgataa acaagttgct atgaaagaga | 36001 |
| aagatagcag agatctggct ttgagatggt ggtcagggaa gactgctcca atagctgagt | 36061 |
| tctaaagcta agaaggaact gagaaccttc acagaacgtc ccaagtagaa gagaaagcac | 36121 |
| actgaagact ctagggaaag aggtctgctt gttgtaggaa ccgaaagaag gccaatgtgg | 36181 |
| ctaggtgctg ggtagtgagg ggaaatggca caaggaaaaa atgaggttag agagatcagt | 36241 |
| tgataccagt taatgttgga ccctaaacat taaccatggt aagtctttta gaattgattc | 36301 |
| taattgcaat gaaaaccttt tgaaagattt taaaaagata tctgatagct gatttacctc | 36361 |
| tctaagaaat gtctggtgaa agacaatacc atcacattgg agatggaaaa agatgaatgg | 36421 |
| attctaaaaa cattctgaaa gtacattcaa aatgtttttc aggtagctta tgcaactaat | 36481 |
| aaatagtggt ggcattctgg gtaagacaag ggaggagcag gcttgcagtt tagggcaaga | 36541 |
| aaggggtggg gagaagagct cagcactaaa atcatgtgtt ccatttgggg cacatcgagt | 36601 |
| ctgagttgct atgagaccac caagtggaga tgccaagtaa atagtcagtt acatgaatct | 36661 |
| ggagttcagt gaagaggtct agaagaaaga tgtatatttg ggcattattc ggatatagat | 36721 |
| attatgtaaa gcaataaaat tggatgagat cacctaggga gagaatgcac atagataaaa | 36781 |
| actgacctag gaccacttca tgtctaaaac accaaaagca atgtcaacaa aagccaaaat | 36841 |
| tgacaaatgg gatctaatta aactaaagag cttctgcaca gcaaaagaat cagagtgaac | 36901 |
| aggcaaccca caaaatggaa gaaaattttc acaacctact catctgacaa agggctaata | 36961 |
| tccagaatct acagtgaact caaacaaatt tacaagaaaa aaacaaacaa ccccatcaaa | 37021 |
| aagtgggtga aggacatgaa cagacacttc tcaaaagaag acatttatgc agccaaaaaa | 37081 |
| cacatgaaaa aatgctcacc atcactggcc atcagagaaa tgcaaatcaa aaccacaatg | 37141 |
| agataccatc tcacaccagt tcaaatggca atcattaaaa agtcaggaaa caacaggtgc | 37201 |
| tggagaggat ctggagaaat aggaacactt ttacactgtt ggtgggacta taaactagtt | 37261 |
| caaccattgg ggaagtcatt gtggcgattc ctcagggatc tagaactaga aataccattt | 37321 |
| gacccagcca tcccattact gggtatatac ccaaaggact ataaatcatg ctgctataaa | 37381 |
| gacacatgca catgtatgtt tattgcggca ctattcacaa tagcaaagac ttggaaccaa | 37441 |
| cctaaatgtc caacaatgat agactggatt aagaaaatgt ggcacatata caccatggaa | 37501 |
| tactatgcag ccataaaaaa tgatgagttc atgtcctttg tagggacatg gatgaagctg | 37561 |
| gaaaccatca tcctcagcaa actatcgcaa ggacaaaaaa ccaaacaccg catgttctca | 37621 |
| cccataggtg ggaattgaac gatgagaaca catggacacg ggaaggggaa catcacacac | 37681 |
| tggggactgt tgtggggtgg gaagaggggg gagggatagc attaggagat atacctaatg | 37741 |
| ctaaatgacg agttattggg tgcagcacac cagcatggca catgtataca tatgtaccta | 37801 |
| acctgcacat tgagcacacg taccctaaaa cttaaagtat aataataata aaataaaata | 37861 |
| aaataaaaaa acaaaaattg atgtaggacc aattcctgaa gaacactgac agttaatttt | 37921 |
| ttggtttagg aggaggagaa gccagcaaac gacactgagt agcaatatcc aaagaaaaag | 37981 |
| aggaaaaagg aaaactggga gattatgagt gtcccagagg gaatgtttca agattaccat | 38041 |
| cagcagtgag ctttgtgtaa aggtggcctc ctgtaataga ggtgcgggca ggagaaggca | 38101 |
| gaatagggaa aagggggtga aaaagcttcc ctcaagattt ataatacagt ggaagagaga | 38161 |
| gacagagaga gagaaagaga aagagagaga gagaacttaa ggaggtagag gaagagagag | 38221 |
| aaccaaaaaa gagggagctg agtatagaag caattagatt catagttttt agttgcggca | 38281 |
| gtgatatttg agtgggggcc ttttatatat tccattctag gtgtttccca gttgatggga | 38341 |
| gagggtctta cctagatctg catgtaaaag ggagtaggcc agctggcaga cttgacatgg | 38401 |
| atcagtggta gaacatccta gcagttctgt gaatactctc tgagaatgac atgaaaggta | 38461 |
| ttggttcagg ccttttggag gtgataaaaa ccaccaaaat gtggacttat tgcaaattgt | 38521 |
| atttgttacc atttgcaatg attataatta ttctcttata tataggcttt cttacatata | 38581 |
| gcttctctta cacatagcgt catgagttat gccttctctt acatatagtg tctgctatga | 38641 |
| gttatgccaa gcagggcaaa caaaattgct gcctttcttt aaaaagagga cgctcctagt | 38701 |
| atgggcctaa ttaattatga taattacagc tatggcatgg aacataagca cattcatata | 38761 |
| cacaaagaca ataaaataaa gaacagttca aaaacagaac agttacatta tatatcagtt | 38821 |
| tcagtatgaa taataccctg gactctgaaa tatgtctggg gcacattatt ctgtaattgg | 38881 |
| tggtgaaaaa aaatctgcat cttatctcta cgccaatcct tatgaaggag ctgtttttca | 38941 |
| ggagttcgag aaagagacac agggatgtcc agtcatcaaa gccgcagagc ctgaggaaga | 39001 |
| ataaaggatt tgtggcagga aaacccagta atgaatgtat tctgctagtt tctcagcata | 39061 |
| gaacttagaa aagaggccac agaaggaaaa gagaagtaat gttagacagc tgatgttggc | 39121 |
| aatgggcaaa gaatttcatt ctcatatgca gggcagtggc taaaggggca gtgttgtgag | 39181 |
| gaatcacatt ccaggtcatt catgtccagg gtgtggtagg aggactgtgt ttcatttatt | 39241 |
| ttgtacatgg cccagctatg accttgtgca aggaaatgct taatcatttt ctatcacagt | 39301 |
| tgcaaagaga ttcttatcct actcagaatg tacgtcttct cttctgtttc atttacagtc | 39361 |
| acaacccaag tccttgcttt gacctccaaa gcaccacttg atgtatccac ttcagaaaca | 39421 |
| cacacagaca gctcacctct ttctatccct taccttcctc ccctcttcac tcaaaccacc | 39481 |
| tacttccttt gcattcactc ttccttcagc ctgaaataat cttcctccaa atatctacct | 39541 |
| tctcactccc tcacttctct caagatacac ttaaatgtta tattccctat gaggcctccc | 39601 |
| ctggccatcc ctccccagcc ttcctatccc ccctctctgc tttattttat tctccttaat | 39661 |
| atatatcaca ctctgataaa ccattgaatg tacttatcaa gttattgcct ctctctccct | 39721 |
| ttccattgtc tccatcactg agagctctgt gaagaaaagg atttgtacct gtttcattta | 39781 |
| gtgctgtacc cccagttccc acaacagcgc aacaggcact caataaatag ttgttgaata | 39841 |
| agtgaataaa acagaagtag ctgcatattt tctggtaaca aatgatattc ttctgaaaat | 39901 |
| gtcatatttt cagacatatt tccgaaaata aattcaaatt agataaacat tgtattttta | 39961 |
| gaccatttct tctttgcatt aatcatcctt ctcaataata taacatttgt aaaacttagg | 40021 |
| ttagatatgg gctcttcaac tttccattac agaagataaa gtgaaaaggc tagacccaat | 40081 |
| ggtgttattc cttcatctac atctatcctt tggaaacaca tgaccaaatt gcttgccatc | 40141 |
| acaatctcaa aatctaccct ttggtattaa ctcacttcac ttgtccctct gtcccttttt | 40201 |
| agatggtagc catcggtctc tggagcagtg tagagtcaga acaacttcta tttggggaag | 40261 |
| aaatcattgc tggtgacctt actttcaatt actaactttc tagtgacatt tacataattt | 40321 |
| tagagaaaat taacacctac acttgtaaag ttgtggcttt cccacaccta tttatcatct | 40381 |
| ctcaatattc cttgaaaagg aaattatcaa tttatcattc tatattggca atgaaatgcc | 40441 |
| cctaatatct gtcacctata agacaattga agatgatgtg ttgaaagctt tctgaaaatg | 40501 |
| ctgatcatta ctttaaatgg aattgaaatt ccagtttatt atttccaaaa atatgatctt | 40561 |
| actgatcata ggataacatt tcataacatt tcagagattt cttccccttc gaggagccaa | 40621 |
| acccatagga cctctggact cccacagatc ctggcaggga gttcccactc ataaaagcac | 40681 |
| aggtgccctc agagtcattc agggatgaag aagcaaccct cattggccat gtcctacgtt | 40741 |
| ccccatatag taaggactgg aggagaccag tgctcaattc tgcagtctca gacagctgtc | 40801 |
| agaggagagt catgaatgtg cagtgtctag cacattacaa acgtttgtta attgactgat | 40861 |
| cattcatggt gtgacagccc tataactcag ctatcctatt cagtcagaaa ttaactcagt | 40921 |
| aatcaaagtc attaaaagga agaaaaaaaa aacctacagt accagacaga tggtggggaa | 40981 |
| atcagacaga tgaaaggaaa aatggctgta ggtcattgag taagacactg ggcagcaaaa | 41041 |
| cctgggcctt gtgcctggtt atactccaca ttatagctcc agcaaggttt ggcaggattt | 41101 |
| ccacagtcct ggcttattct aacctttctt gggagcagga gcagtgttgg tcagatagac | 41161 |
| agacacataa ggaatctgtc caactggcac cgtgtgaatt tgggctcttg gtgtacatgg | 41221 |
| ataactggga aaaagaggag agagacatgt aggactgatc ctaccgtttg tgaagtcttg | 41281 |
| ggcaagagta tgaatgaaaa cccacttctc ttcccctgcc tggctccact gcacacagta | 41341 |
| aagagcctca agcataggtg tgtggacatt gcaccatgta tccaagctct gaccatgcct | 41401 |
| cttgaaacag ctattcctca gccaccctct gaccatggga ggaatgaccc aggagaaatg | 41461 |
| accacatagg tcttgaaaat gggctcaggg ctatttacga agtcaattcc ggggtcccag | 41521 |
| gagtatggac taaaatgtga gtcaggcatg ccagatgggt atgttctatt gacttcaagg | 41581 |
| attcctcatg ctgtgggaag gaacctctcc agaagagaga cagagcagaa ccctctaaat | 41641 |
| gtggggcaca aagcaggagc ccctcttgct ggattcaaag ggtcatactg gaagagtgta | 41701 |
| ggttgagtct tattctcaca tcactcatat cacttacaca cttcttttat agccttagca | 41761 |
| gccatgccac aaagagaaac tcttcatgca tatcttttgg tccataagtc ataaatagtt | 41821 |
| atccttgatc ccatgtcttt tttagagcca tggacagaga gaagcaaaaa tataccaagt | 41881 |
| tcacactgag ttgtctccct tcatatctct tagcagtcac tgaaaggtta tgagactcag | 41941 |
| gctgggtttc tatcctctgt ccctgaaacg acaacgttga cctcgtgatc aaccctagaa | 42001 |
| tccagagcaa gatctcagac tgtcctctct actaccagac agcacacttt gttttggggg | 42061 |
| ctgtgtgctt gaaatattag ctatggcaaa aggctttgag tcttatgaca ccccaagtaa | 42121 |
| cttttacttt aggaatttga aatacagcct tgctgtaatg ctgtctcctt aacaaagcag | 42181 |
| tacctttgaa atatttaaca acttgaaaag gaaaccgagc ttgaattttc ctttcaggtg | 42241 |
| ctcaggaaat aatgtttcac ttctgtctga aattcaccat ctcctcagac aaagaaggct | 42301 |
| cttatggtaa aaggaatggc attttctcca caattttcga ataaaagata aagagaaaac | 42361 |
| agcactgcag cctttttgtt aggatctaac aataaagaaa taatacggtt ttgccagggg | 42421 |
| agagctctgg ttttaagctc agaatacaaa aataggctga caaaatttta caaaggaata | 42481 |
| ttctcagcta ccactctgag gatggtagaa agtgaaattt caagaaaatt atattatttg | 42541 |
| attatttgat gatgattaag ctgattggcc agtcctatgt gaaattctaa agtagaagaa | 42601 |
| atgtgatgtt gtcttttctg ctcacctctc ccctcattcc tacccccaaa tctctgcctc | 42661 |
| taccccaaac ccagcctgac ctttgggaaa ggaatggggg ctgtcacttg caccgtagct | 42721 |
| cctcctgcct gcagtactct ccccccacag tccagcctcc ctctgctcag ctaacttttc | 42781 |
| ctatagctcc ttctggctac ttctaaggaa ccttccataa tgctgcccac cctctcagta | 42841 |
| acagcccctc tactatccct tgattacatg cactgtaatt ggttagtaat tgattttatg | 42901 |
| tcctctgcca aattatactc catgagagca aaaatcatga gtattatctt taatgtttaa | 42961 |
| atctccacaa ctatccccca tataagtcta gagaataaat gaatgagtga attaatgaat | 43021 |
| agaaactcaa gccattatgt tgccactcct aggatatttg gattaacttt aactgaagag | 43081 |
| taaaaagcat ttacctgtcc taaaggagac ataaaattag tgggagagta tttggagaaa | 43141 |
| aaaaagacac tgtaatacat tcttttgtgt tgcctaccct gatgtagtca ggtgtccctg | 43201 |
| atatggggtg ggctgaggat ttgaaataaa atacctaatt tgacattgta agtggaggaa | 43261 |
| tcaggatttg aaaccaaatt ggtttgacct aaatcaagct attatgataa ggacttgcaa | 43321 |
| gaaaaaagga atccataaaa accatatgaa tagctaccaa ttagtaagca tttatatgtg | 43381 |
| tcaattacaa agccaaatgc aaatcatgct ttatttatat tagccacctt ttatagatga | 43441 |
| agaaattgag acctgaatat taaaattgcc tacttttaca tagtaagtaa aggaatcagg | 43501 |
| gtttgaaccc aaattggttt cacctaaggt agaaaaccat cccagcaagt ctcctattaa | 43561 |
| ctggaaccct attgtggtgg cctgagatat aacagtagct gtggaagcgc tgtagagtcc | 43621 |
| tggccatcct atgtgctcct gatctggtcc ctcctgccac ctgcttctgc tccctgtgcc | 43681 |
| atccacccat ctggaagtct cccagtgtcc atcttcgggg gagacactca ccagagtttc | 43741 |
| cagcttccag ccagtatgga gtgcccctgt cccacagcaa tctcaccgaa atcacagcta | 43801 |
| catctgttaa aattaggcta ccaatgagtg atagatgagg gggaaaaata ataatagtgt | 43861 |
| actaaacaaa acaaatgttt atttttctca cacataaaaa tctagaggtt gaagtccagg | 43921 |
| gctggtccag aggctccaag gatctgggat ttagactccc tctttcttgt ttttccacag | 43981 |
| catatggctt ccatttctgg ggccacattg gtccaaaatg tatgctgggg ctccagccat | 44041 |
| tgcatccata tttcagccac aggaaggagg aagtggggaa gaaaggacag gcccctaata | 44101 |
| cctgtatagt tcaagaagac tatcccgccc atacttccca accaccctta gttgaacaat | 44161 |
| gctgtcttaa ttcaagacac tcacatgtct agccaaaaat ctgaattctg ttacaaacaa | 44221 |
| ggagaataga gatgtgcgcc acctcaatac ctcatccata gctacctttt cctttgtgca | 44281 |
| gctgtggcca agtgaaagct gaaggagctg tggtaaccct tctgaaggga ggctggggcc | 44341 |
| tttcacaaga ggctgcatga ttgacattta tcctgcatgg cctgtgaagt acagagaaat | 44401 |
| attttctctt gaagccacat catagcagtg gctgctttgt agcctgattc caccattatg | 44461 |
| cctttaaagt gcctagcaat tcagccttca catcatgcaa agaggaatat ctcccagtct | 44521 |
| ttgtaagatc agcttaattc taaccacctc cttacctccc actgcactcc tacacgcaca | 44581 |
| cacaaatctt cttcactcag agcagaacca taacccaagc cctaccacct agagactgaa | 44641 |
| gaatcaggct catgattaca aatatgcaat aattttttgt gtggataatg tcaatgggga | 44701 |
| tgatggtaag agaattcctt ggtttacaca ttgaccctct tccctgtccc ttacaatcag | 44761 |
| gaaatatttg tcccaacacc ttgtttcttc tgttgcaggc tcctgtggct gtcttgtcat | 44821 |
| gatattgttt gcctctgaag tgaaaatcca tcacctctca gaaaaaattg caaattataa | 44881 |
| agaagggact tatgtctaca aaacgcaaag tgaaaaatat accacctcat tctgggtcat | 44941 |
| tttcttttgc ttttttgttc attttctgaa tgggctccta atacgacttg ctggatttca | 45001 |
| gttccctttt gcaaaatcta aagacgcaga aacaactaat gtagctgcag atctaatgta | 45061 |
| ctgaaaggca aacctttcta taattttaca agggagtaga cttgctttgg tcacttttag | 45121 |
| atgtggttaa ttttgcatat ccttttagtc tgcatatatt aaagcatcag gacccttcgt | 45181 |
| gacaatgttt acaaattacg tactaaggat acaggctgga aagtaaggga agcagaagga | 45241 |
| aggctttgaa aagttgtttt atctggtggg aaattgcttg acccaggtag tcaaaggcag | 45301 |
| ttgactagaa tcgacaaatt gttactccat atatatatat gtgtgtgtgt gtgtgtgtgt | 45361 |
| gtgtgtgtaa gatgtcttcc tatcaaaaag atatcaaagg cacatggaat atattttaat | 45421 |
| aaaaacaaat aatatctcta atatatccac acatttgttg ccagatttca gaaaactgag | 45481 |
| ctgcaatcgc tttcctaaaa cagtagtgta ttaaatgaac atctataaaa tgtatcaaca | 45541 |
| cacattttaa aaaatttgtt taaagtatac tcttaggcca ggcgtggtga ctcacacctg | 45601 |
| taattccagc acttcaggag gccaaggtgg gaagatcatt tgagttcagg agttcgagtt | 45661 |
| acagcctggg caataaagtg agaccctgtc actaacaaaa ttaaaaaata aaataaatat | 45721 |
| aaaatatagg ctttaaaaaa gcatagtctt attaaccatg tctgttggtc aaaatctgca | 45781 |
| aactctaaaa gaagaaaaga agaaaaaacc aagcttaggg tatttttcct cccgtgcctg | 45841 |
| agtcccaatt acattcacga cagtactttc aatgaacata attgttagga ccactgagga | 45901 |
| atcatgaaaa atgatctctg cttagtacat ttgatgcaaa atgacttatt aggggctgtt | 45961 |
| tttctagcta tagtgtctcg agtactaata tgcaattatg aaaattatat taaatctggg | 46021 |
| attatgacgg tatcactgta tcatcttggt cttgttctgg ctgtcaccaa gcatgaccca | 46081 |
| ggtcaacttt ttttttcccc tgaattaccc atcaaattga tctgcagctg actaaaggcc | 46141 |
| acagctgagc ctggaactga cccttccttc atcctcaacc tgctgtcctc cagaaagcac | 46201 |
| caaggaaaaa gcagagaatg acagcaaaca gatcactagg cctctgacca caggtgctga | 46261 |
| gtactcagca gccctcatat aataggtttg aaagtactcc ttaaaataaa acactgtttc | 46321 |
| cctttggaac tatttacaag gatgaaacaa ccgtatacct gagaaataac ttgctctggt | 46381 |
| gtcaattcgc tattcgccag cagacatcag aacacaccga gtttccagat gctggttttt | 46441 |
| ccccttaaat caggaaatac acctggacaa tttctagaag actacaattc agtctagcca | 46501 |
| caaaggggat tttttttttt tggtaacagg ctagagcccg gttctgtaag tctttagctg | 46561 |
| aaatggtcca gtacaaaagc actggaaatg agtgggctag gaggacaagg accgtctcct | 46621 |
| gcgtgaggag ttggttggag gtccccaagg ccaggtaccc cctgcactct tattggattc | 46681 |
| ctctctgtct tcttggagtt ttgaaaaact ccttcgaaca ccaggctttt ttctttagaa | 46741 |
| aacaagtctc caatcgttct ctgttccgta gaaagagaaa gaaaacctgg agcagctgct | 46801 |
| gaaaaatcta atgaggaact aagaggcaaa cccacca | |

A non-limiting example of a human wildtype CLRN1 genomic DNA sequence is SEQ ID NO: 9. The exons in SEQ ID NO: 9 are: nucleotide positions 1-544 (exon 1), nucleotide positions 28764-29180 (exon 2), nucleotide positions 31239-31418 (exon 3), nucleotide positions 32481-32519 (exon 4), nucleotide positions 44799-46433 (exon 5), nucleotide positions 44799-44935 (exon 6), and nucleotide positions 46128-46837 (exon 7). The introns are located between each pair of these exons in SEQ ID NO: 9, i.e., at nucleotide positions 545-28763 (intron 1), nucleotide positions 29181-31238 (intron 2), nucleotide positions 31419-32480 (intron 3), nucleotide positions 32520-44798 (intron 4), and nucleotide positions 44936-46127 (intron 7).

Mouse CLRN1 Protein Isoform 1
(SEQ ID NO: 10)
MPSQQKKIIFCMAGVLSFLCALGVVTAVGTPLWVKATILCKTGALLVNASG
KELDKFMGEMQYGLFHGEGVRQCGLGARPFRFSSRSMKERYSLYEDKGETA
VFPDLVQAIPVSIHINIILFSMILVVLTMVGTAFFMYNAFGKPFETLHGPL
GLYLVSFISGSCGCLVMILFASEVKVHRLSEKIANFKEGTYAYRTQNENYT
TSFWVVFICFFVHFLNGLLIRLAGFQFPFTKSKETETTNVASDLMY
Dog CLRN1 Protein
(SEQ ID NO: 11)
MPNQQKKVVFCTAGVLSFVCALGVVTALGTPLWIKATFLCKTGALLVNASG
QELDKFMGEMQYGLFHGEGIRQCGLGARPFRFSLFPDLLKVIPVSIHVNVI
LFSTILVVLTMVGTAFFMYNAFGKPFETLHGPLGLYLLSFISGSCGCLVMI
LFASEVKIHHLSEKIANYKEGTYAYKTQSEKYTTSFWVVFICFLVHLLNGL
LRLAGFQFPFAKSKDTETTNVAADLMY

### Vectors

The compositions provided herein include at least two (e.g., two, three, four, five, or six) nucleic acid vectors, where: each of the at least two different vectors includes a coding sequence that encodes a different portion of a CLRN1 protein, each of the encoded portions being at least 30 amino acids (e.g., between about 30 amino acids to about 202 amino acids, about 30 amino acids to about 200 amino acids, about 30 amino acids to about 180 amino acids, about 30 amino acids to about 170 amino acids, about 30 amino acids to about 160 amino acids, about 30 amino acids to about 150 amino acids, about 30 amino acids to about 140 amino acids, about 30 amino acids to about 130 amino acids, about 30 amino acids to about 120 amino acids, about 30 amino acids to about 110 amino acids, about 30 amino acids to about 100 amino acids, about 30 amino acids to about 90 amino acids, about 30 amino acids to about 80 amino acids, about 30 amino acids to about 70 amino acids, about 30 amino acids to about 60 amino acids, about 30 amino acids to about 50 amino acids, about 30 amino acids to about 40 amino acids, about 60 amino acids to about 202 amino acids, about 60 amino acids to about 200 amino acids, about 60 amino acids to about 180 amino acids, about 60 amino acids to about 170 amino acids, about 60 amino acids to about 160 amino acids, about 60 amino acids to about 150 amino acids, about 60 amino acids to about 140 amino acids, about 60 amino acids to about 130 amino acids, about 60 amino acids to about 120 amino acids, about 60 amino acids to about 110 amino acids, about 60 amino acids to about 100 amino acids, about 60 amino acids to about 90 amino acids, about 60 amino acids to about 80 amino acids, about 60 amino acids to about 70 amino acids, about 90 amino acids to about 202 amino acids, about 90 amino acids to about 200 amino acids, about 90 amino acids to about 180 amino acids, about 90 amino acids to about 170 amino acids, about 90 amino acids to about 160 amino acids, about 90 amino acids to about 150 amino acids, about 90 amino acids to about 140 amino acids, about 90 amino acids to about 130 amino acids, about 90 amino acids to about 120 amino acids, about 90 amino acids to about 110 amino acids, about 90 amino acids to about 100 amino acids, about 100 amino acids to about 202 amino acids, about 100 amino acids to about 200 amino acids, about 100 amino acids to about 180 amino acids, about 100 amino acids to about 170 amino acids, about 100 amino acids to about 160 amino acids, about 100 amino acids to about 150 amino acids, about 100 amino acids to about 140 amino acids, about 100 amino acids to about 130 amino acids, about 100 amino acids to about 120 amino acids, about 90 amino acids to about 110 amino acids, about 120 amino acids to about 202 amino acids, about 120 amino acids to about 200 amino acids, about 120 amino acids to about 180 amino acids, about 120 amino acids to about 170 amino acids, about 120 amino acids to about 160 amino acids, about 120 amino acids to about 150 amino acids, about 120 amino acids to about 140 amino acids, about 120 amino acids to about 130 amino acids, about 150 amino acids to about 202 amino acids, about 150 amino acids to about 200 amino acids, about 150 amino acids to about 180 amino acids, about 150 amino acids to about 170 amino acids, about 150 amino acids to about 160 amino acids, about 170 amino acids to about 202 amino acids, about 170 amino acids to about 200 amino acids, about 170 amino acids to about 180 amino acids, about 190 amino acids to about 202 amino acids, or about 190 amino acids to about 200 amino acids) in length.

In some embodiments of these compositions, at least one of the coding sequences includes a nucleotide sequence spanning two consecutive exons of CLRN1 genomic DNA (e.g., exons 1 and 2, or exons 5 and 6), and lacking the intronic sequence that naturally occurs between the two consecutive exons.

In some embodiments, the amino acid sequence of none of the encoded portions overlaps even in part with the amino acid sequence of a different one of the encoded portions. In some embodiments, the amino acid sequence of one or more of the encoded portions partially overlaps with the amino acid sequence of a different one of the encoded portions. In some embodiments, the amino acid sequence of each of the encoded portions partially overlaps with the amino acid sequence of a different one of the encoded portions.

In some embodiments, the overlapping amino acid sequence is between about 30 amino acid residues to about 202 amino acids (e.g., or any of the subranges of this range described herein) in length.

In some examples, the vectors include two different vectors, each of which comprises a different segment of an intron, wherein the intron includes the nucleotide sequence of an intron that is present in a CLRN1 genomic DNA (e.g., any of the exemplary introns in SEQ ID NO: 9 described herein), and wherein the two different segments overlap in sequence by at least 100 nucleotides (e.g., about 100 nucleotides to about 10,000 nucleotides, about 100 nucleotides to about 5,000 nucleotides, about 100 nucleotides to about 4,500 nucleotides, about 100 nucleotides to about 4,000 nucleotides, about 100 nucleotides to about 3,500 nucleotides, about 100 nucleotides to about 3,000 nucleotides, about 100 nucleotides to about 2,500 nucleotides, about 100 nucleotides to about 2,000 nucleotides, about 100 nucleotides to about 1,500 nucleotides, about 100 nucleotides to about 1,000 nucleotides, about 100 nucleotides to about 800 nucleotides, about 100 nucleotides to about 600 nucleotides, about 100 nucleotides to about 400 nucleotides, about 100 nucleotides to about 200 nucleotides, about 200 nucleotides to about 10,000 nucleotides, about 200 nucleotides to about 5,000 nucleotides, about 200 nucleotides to about 4,500 nucleotides, about 200 nucleotides to about 4,000 nucleotides, about 200 nucleotides to about 3,500 nucleotides, about 200 nucleotides to about 3,000 nucleotides, about 200 nucleotides to about 2,500 nucleotides, about 200 nucleotides to about 2,000 nucleotides, about 200 nucleotides to about 1,500 nucleotides, about 200 nucleotides to about 1,000 nucleotides, about 200 nucleotides to about 800 nucleotides, about 200 nucleotides to about 600 nucleotides, about 200 nucleotides to about 400 nucleotides, about 400 nucleotides to about 10,000 nucleotides, about 400 nucleotides to about 5,000 nucleotides, about 400 nucleotides to about 4,500 nucleotides, about 400 nucleotides to about 4,000 nucleotides, about 400 nucleotides to about 3,500 nucleotides, about 400 nucleotides to about 3,000 nucleotides, about 400 nucleotides to about 2,500 nucleotides, about 400 nucleotides to about 2,000 nucleotides, about 400 nucleotides to about 1,500 nucleotides, about 400 nucleotides to about 1,000 nucleotides, about 400 nucleotides to about 800 nucleotides, about 400 nucleotides to about 600 nucleotides, about 600 nucleotides to about 10,000 nucleotides, about 600 nucleotides to about 5,000 nucleotides, about 600 nucleotides to about 4,500 nucleotides, about 600 nucleotides to about 4,000 nucleotides, about 600 nucleotides to about 3,500 nucleotides, about 600 nucleotides to about 3,000 nucleotides, about 600 nucleotides to about 2,500 nucleotides, about 600 nucleotides to about 2,000 nucleotides, about 600 nucleotides to about 1,500 nucleotides, about 600 nucleotides to about 1,000 nucleotides, about 600 nucleotides to about 800 nucleotides, about 800 nucleotides to about 10,000 nucleotides, about 800 nucleotides to about 5,000 nucleotides, about 800 nucleotides to about 4,500 nucleotides, about 800 nucleotides to about 4,000 nucleotides, about 800 nucleotides to about 3,500 nucleotides, about 800 nucleotides to about 3,000 nucleotides, about 800 nucleotides to about 2,500 nucleotides, about 800 nucleotides to about 2,000 nucleotides, about 800 nucleotides to about 1,500 nucleotides, about 800 nucleotides to about 1,000 nucleotides, about 1,000 nucleotides to about 10,000 nucleotides, about 1,000 nucleotides to about 5,000 nucleotides, about 1,000 nucleotides to about 4,500 nucleotides, about 1,000 nucleotides to about 4,000 nucleotides, about 1,000 nucleotides to about 3,500 nucleotides, about 1,000 nucleotides to about 3,000 nucleotides, about 1,000 nucleotides to about 2,500 nucleotides, about 1,000 nucleotides to about 2,000 nucleotides, about 1,000 nucleotides to about 1,500 nucleotides, about 1,500 nucleotides to about 10,000 nucleotides, about 1,500 nucleotides to about 5,000 nucleotides, about 1,500 nucleotides to about 4,500 nucleotides, about 1,500 nucleotides to about 4,000 nucleotides, about 1,500 nucleotides to about 3,500 nucleotides, about 1,500 nucleotides to about 3,000 nucleotides, about 1,500 nucleotides to about 2,500 nucleotides, about 1,500 nucleotides to about 2,000 nucleotides, about 2,000 nucleotides to about 10,000 nucleotides, about 2,000 nucleotides to about 5,000 nucleotides, about 2,000 nucleotides to about 4,500 nucleotides, about 2,000 nucleotides to about 4,000 nucleotides, about 2,000 nucleotides to about 3,500 nucleotides, about 2,000 nucleotides to about 3,000 nucleotides, about 2,000 nucleotides to about 2,500 nucleotides, about 2,500 nucleotides to about 10,000 nucleotides, about 2,500 nucleotides to about 5,000 nucleotides, about 2,500 nucleotides to about 4,500 nucleotides, about 2,500 nucleotides to about 4,000 nucleotides, about 2,500 nucleotides to about 3,500 nucleotides, about 2,500 nucleotides to about 3,000 nucleotides, about 3,000 nucleotides to about 10,000 nucleotides, about 3,000 nucleotides to about 5,000 nucleotides, about 3,000 nucleotides to about 4,500 nucleotides, about 3,000 nucleotides to about 4,000 nucleotides, about 3,000 nucleotides to about 3,500 nucleotides, about 3,500 nucleotides to about 10,000 nucleotides, about 3,500 nucleotides to about 5,000 nucleotides, about 3,500 nucleotides to about 4,500 nucleotides, about 3,500 nucleotides to about 4,000 nucleotides, about 4,000 nucleotides to about 10,000 nucleotides, about 4,000 nucleotides to about 5,000 nucleotides, about 4,000 nucleotides to about 4,500 nucleotides, about 4,500 nucleotides to about 10,000 nucleotides about 4,500 nucleotides to about 5,000 nucleotides, or about 5,000 nucleotides to about 10,000 nucleotides) in length.

The overlapping nucleotide sequence in any two of the different vectors can include part or all of one or more exons of a CLRN1 gene (e.g., any one or more of the exemplary exons in SEQ ID NO: 9 described herein).

In some embodiments, the number of different vectors in the composition is two, three, four, or five. In compositions where the number of different vectors in the composition is two, the first of the two different vectors can include a coding sequence that encodes an N-terminal portion of the CLRN1 protein. In some examples, the N-terminal portion of the CLRN1 gene is between about 30 amino acids to about 202 amino acids (or any of the subranges of this range described above) in length. In some examples, the first vector further includes one or both of a promoter (e.g., any of the promoters described herein or known in the art) and a Kozak sequence (e.g., any of the exemplary Kozak sequences described herein or known in the art). In some examples, the first vector includes a promoter that is an inducible promoter, a constituitive promoter, or a tissue-specific promoter. In some examples, the second of the two different vectors includes a coding sequence that encodes a C-terminal portion of the CLRN1 protein. In some examples, the C-terminal portion of the CLRN1 protein is between 30 amino acids to about 202 amino acids (or any of the subranges of this range described above) in length. In some examples, the second vector further includes a polyadenylation signal sequence.

In some examples where the number of different vectors in the composition is two, the N-terminal portion encoded by one of the two vectors can include a portion comprising amino acid position 1 to any of the following: about amino acid position 202, about amino acid position 200, about amino acid 190, about amino acid position 180, about amino acid position 170, about amino acid position 160, about amino acid position 150, about amino acid position 140, about amino acod position 130, about amino acid position 120, about amino acid position 110, about amino acid position 100, about amino acid position 90, about amino acid position 80, about amino acid position 70, about amino acid position 60, about amino acid position 50, or about amino acid position 40 of a wildtype CLRN1 protein (e.g., SEQ ID NO: 1, 3, 5, or 7).

In some examples where the number of different vectors in the composition is two, the N-terminal portion of the precursor CLRN1 protein can include a portion comprising amino acid position 1 to amino acid position 202, amino acid position 1 to about amino acid position 200, amino acid position 1 to about amino acid position 190, amino acid position 1 to about amino acid position 180, amino acid position 1 to about amino acid position 170, amino acid position 1 to about amino acid position 160, amino acid position 1 to about amino acid position 150, amino acid position 1 to about amino acid position 140, amino acid position 1 to about amino acid position 130, amino acid position 1 to about amino acid position 120, amino acid position 1 to about amino acid position 110, amino acid position 1 to about amino acid position 100, amino acid position 1 to about amino acid position 90, amino acid position 1 to about amino acid position80, amino acid position 1 to about amino acid position 70, amino acid position 1 to about amino acid position 60, amino acid position 1 to about amino acid position 50, amino acid position 1 to about amino acid position 40, amino acid position 1 to about amino acid position 30 of a wildtype CLRN1 protein (e.g., SEQ ID NO: 1, 3, 5, or 7). As used herein, the term “vector” means a composition including a polynucleotide capable of carrying at least one exogenous nucleic acid fragment, e.g., a plasmid vector, a transposon, a cosmid, an artificial chromosome (e.g., a human artificial chromosome (HAC), a yeast artificial chromosome (YAC), a bacterial artificial chromosome (BAC), or a P1-derived artificial chromosome (PAC)), a viral vector (e.g., any adenoviral vectors (e.g., pSV or pCMV vectors) or any retroviral vectors as described herein), and any Gateway® vectors. A vector can, e.g., include sufficient cis-acting elements for expression; other elements for expression can be supplied by the host mammalian cell or in an in vitro expression system. The term “vector” includes any genetic element (e.g., a plasmid, a transposon, a cosmid, an artificial chromosome, a viral vector, etc.) that is capable of replicating when associated with the proper control elements. Thus, the term includes cloning and expression vectors, as well as viral vectors (e.g., an adeno-associated virus (AAV) vector, an adenovirus vector, a lentivirus vector, or a retrovirus vector).

Vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide. Skilled practitioners will be capable of selecting suitable vectors and mammalian cells for making any of the nucleic acids described herein.

In some embodiments, the vector is a plasmid (i.e. a circular DNA molecule that can autonomously replicate inside a cell). In some embodiments, the vector can be a cosmid (e.g., pWE and sCos series (Wahl et al. (1987), Evans et al. (1989)).

In some embodiments, the vector(s) is an artificial chromosome. An artificial chromosome is a genetically engineered chromosome that can be used as a vector to carry large DNA inserts. In some embodiments, the artificial chromosome is human artificial chromosome (HAC) (see, e.g., Kouprina et al., Expert Opin. Drug Deliv 11(4): 517-535, 2014; Basu et al., Pediatr. Clin. North Am. 53: 843-853, 2006; Ren et al., Stem. Cell Rev. 2(1):43-50, 2006; Kazuki et al., Mol. Ther. 19(9):1591-1601, 2011; Kazuki et al., Gen. Ther. 18: 384-393, 2011; and Katoh et al., Biochem. Biophys. Res. Commun. 321:280-290, 2004).

In some embodiments, the vector(s) is a yeast artificial chromosome (YAC) (see, e.g., Murray et al., Nature 305: 189-193, 1983; Ikeno et al. (1998) Nat. Biotech. 16:431-439, 1998). In some embodiments, the vector(s) is a bacterial artificial chromosome (BAC) (e.g., pBeloBAC11, pECBAC1, and pBAC108L). In some embodiments, the vector(s) is a P1-derived artificial chromosome (PAC). Examples of artificial chromosome are known in the art.

In some embodiments, the vector(s) is a viral vector (e.g., adeno-associated virus, adenovirus, lentivirus, and retrovirus). Non-limiting examples of viral vectors are described herein. In some embodiments, the vector(s) is an adeno-associated viral vector (AAV) (see, e.g., Asokan et al., Mol. Ther. 20: 699-7080, 2012). Recombinant AAV vectors or “rAAVs” are typically composed of, at a minimum, a transgene or a portion thereof and a regulatory sequence, and optionally 5′ and 3′ AAV inverted terminal repeats (ITRs). Such a recombinant AAV vector is packaged into a capsid and delivered to a selected target cell (e.g., a cochlear hair cell).

The AAV sequences of the vector typically comprise the cis-acting 5′ and 3′ ITR sequences (See, e.g., B. J. Carter, in “Handbook of Parvoviruses”, ed., P. Tijsser, CRC Press, pp. 155 168, 1990). Typical AAV ITR sequences are about 145 nucleotides in length. In some embodiments, at least 75% of a typical ITR sequence (e.g., at least 80%, at least 85%, at least 90%, or at least 95%) is incorporated into the AAV vector. The ability to modify these ITR sequences is within the skill of the art. (See, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996). In some embodiments, any of the coding sequences described herein is flanked by 5′ and 3′ AAV ITR sequences in the AAV vectors. The AAV ITR sequences may be obtained from any known AAV, including presently identified AAV types.

AAV vectors as described herein may include any of the regulatory elements described herein (e.g., one or more of a promoter, a polyadenylation (poly(A)) signal sequence, and an IRES).

In some embodiments, the AAV vector is selected from the group consisting of: an AAV1 vecotr, an AAV2 vector, an AAV3 vector, an AAV4 vector, an AAV5 vector, an AAV6 vector, an AAV7 vector, an AAV8 vector, an AAV9 vector, an AAV2.7m8 vector, an AAV8BP2 vector, and an AAV293 vector. Additional exemplary AAV vectors that can be used herein are known in the art. See, e.g., Kanaan et al., Mol. Ther. Nucleic Acids 8:184-197, 2017; Li et al., Mol. Ther. 16(7): 1252-1260; Adachi et al., Nat. Commun. 5: 3075, 2014; Isgrig et al., Nat. Commun. 10(1): 427, 2019; and Gao et al., J. Virol. 78(12): 6381-6388.

In some embodiments, an AAV vector provided herein includes or consists of a sequence that is at least 80% identical (e.g., at least 82%, at least 84%, at least 85%, at least 86%, at least 88%, at least 90%, at least 92%, at least 94%, at least 95%, at least 96%, at least 98%, at least 99%, or 100% identical) to SEQ ID NO: 40, 41, 42, 43, 44, 46, 47, 48, 49, 50, 51, 52, 53, 54, 55, 56, 57, 58, 59 or 60. In some embodiments, the vector(s) is an adenovirus (see, e.g., Dmitriev et al. (1998) J. Virol. 72: 9706-9713; and Poulin et al., J. Virol 8: 10074-10086, 2010). In some embodiments, the vector(s) is a retrovirus (see, e.g., Maier et al. (2010) Future Microbiol 5: 1507-23).

In some embodiments, the vector(s) is a lentivirus (see, e.g., Matrai et al. (2010) Mol Ther. 18: 477-490; Banasik et al. (2010) Gene Ther. 17:150-7; and Wanisch et al. (2009) Mol. Ther. 17: 1316-32). A lentiviral vector refers to a vector derived from at least a portion of a lentivirus genome, including especially a self-inactivating lentiviral vector as described in Milone et al., Mol. Ther. 17(8): 1453-1464 (2009). Non-limiting lentivirus vectors that may be used in the clinic include the LENTIVECTOR® gene delivery technology from Oxford BioMedica, the LENTIMAX™ vector system from Lentigen, and the like. Other types of lentiviral vectors are also available and would be known to one skilled in the art.

The vectors provided herein can be of different sizes. The choice of vector that is used in any of the compositions, kits, and methods described herein may depend on the size of the vector.

In some embodiments, the vector(s) is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some embodiments, the vector(s) is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some embodiments, the vector(s) is a transposon (e.g., PiggyBac™ transposon) and can include greater than 200 kb. In some examples, the vector(s) is a transposon having a total length in the range of about 1 kb to about 10 kb, about 1 kb to about 20 kb, about 1 kb to about 30 kb, about 1 kb to about 40 kb, about 1 kb to about 50 kb, about 1 kb to about 60 kb, about 1 kb to about 70 kb, about 1 kb to about 80 kb, about 1 kb to about 90 kb, about 10 kb to about 20 kb, about 10 kb to about 30 kb, about 10 kb to about 40 kb, about 10 kb to about 50 kb, about 10 kb to about 60 kb, about 10 kb to about 70 kb, about 10 kb to about 90 kb, about 10 kb to about 100 kb, about 20 kb to about 30 kb, about 20 kb to about 40 kb, about 20 kb to about 50 kb, about 20 kb to about 60 kb, about 20 kb to about 70 kb, about 20 kb to about 80 kb, about 20 kb to about 90 kb, about 20 kb to about 100 kb, about 30 kb to about 40 kb, about 30 kb to about 50 kb, about 30 kb to about 60 kb, about 30 kb to about 70 kb, about 30 kb to about 80 kb, about 30 kb to about 90 kb, about 30 kb to about 100 kb, about 40 kb to about 50 kb, about 40 kb to about 60 kb, about 40 kb to about 70 kb, about 40 kb to about 80 kb, about 40 kb to about 90 kb, about 40 kb to about 100 kb, about 50 kb to about 60 kb, about 50 kb to about 70 kb, about 50 kb to about 80 kb, about 50 kb to about 90 kb, about 50 kb to about 100 kb, about 60 kb to about 70 kb, about 60 kb to about 80 kb, about 60 kb to about 90 kb, about 60 kb to about 100 kb, about 70 kb to about 80 kb, about 70 kb to about 90 kb, about 70 kb to about 100 kb, about 80 kb to about 90 kb, about 80 kb to about 100 kb, about 90 kb to about 100 kb, about 1 kb to about 100 kb, about 100 kb to about 200 kb, about 100 kb to about 300 kb, about 100 kb to about 400 kb, or about 100 kb to about 500 kb.

In some embodiments, the vector is a cosmid and can have a total length of up to 55 kb. In some examples, the vector is a cosmid and has a total number of nucleotides of about 1 kb to about 10 kb, about 1 kb to about 20 kb, about 1 kb to about 30 kb, about 1 kb to about 40 kb, about 1 kb to about 50 kb, about 1 kb to about 55 kb, about 10 kb to about 20 kb, about 10 kb to about 30 kb, about 10 kb to about 40 kb, about 10 kb to about 50 kb, about 10 kb to about 55 kb, about 15 kb to about 55 kb, about 15 kb to about 50 kb, about 15 kb to about 40 kb, about 15 kb to about 30 kb, about 15 kb to about 20 kb, about 20 kb to about 55 kb, about 20 kb to about 50 kb, about 20 kb to about 40 kb, about 20 kb to about 30 kb, about 25 kb to about 55 kb, about 25 kb to about 50 kb, about 25 kb to about 40 kb, about 25 kb to about 30 kb, about 30 kb to about 55 kb, about 30 kb to about 50 kb, about 30 kb to about 40 kb, about 35 kb to about 55 kb, about 40 kb to about 55 kb, about 40 kb to about 50 kb, or about 45 kb to about 55 kb.

In some embodiments, the vector(s) is an artificial chromosome and can have a total number of nucleotides of about 100 kb to about 2000 kb. In some embodiments, the artificial chromosome(s) is a human artificial chromosome (HAC) and can have a total number of nucleotides in the range of about 1 kb to about 10 kb, 1 kb to about 20 kb, about 1 kb to about 30 kb, about 1 kb to about 40 kb, about 1 kb to about 50 kb, about 1 kb to about 60 kb, about 10 kb to about 20 kb, about 10 kb to about 30 kb, about 10 kb to about 40 kb, about 10 kb to about 50 kb, about 10 kb to about 60 kb, about 20 kb to about 30 kb, about 20 kb to about 40 kb, about 20 kb to about 50 kb, about 20 kb to about 60 kb, about 30 kb to about 40 kb, about 30 kb to about 50 kb, about 30 kb to about 60 kb, about 40 kb to about 50 kb, about 40 kb to about 60 kb, or about 50 kb to about 60 kb.

In some embodiments, the artificial chromosome(s) is a yeast artificial chromosome (YAC) and can have a total number of nucleotides up to 1000 kb. In some embodiments, the articial chromosome(s) is a YAC having a total number of nucleotides in the range of about 100 kb to about 1,000 kb, about 100 kb to about 900 kb, about 100 kb to about 800 kb, about 100 kb to about 700 kb, about 100 kb to about 600 kb, about 100 kb to about 500 kb, about 100 kb to about 400 kb, about 100 kb to about 300 kb, about 100 kb to about 200 kb, about 200 kb to about 1,000 kb, about 200 kb to about 900 kb, about 200 kb to about 800 kb, about 200 kb to about 700 kb, about 200 kb to about 600 kb, about 200 kb to about 500 kb, about 200 kb to about 400 kb, about 200 kb to about 300 kb, about 300 kb to about 1,000 kb, about 300 kb to about 900 kb, about 300 kb to about 800 kb, about 300 kb to about 700 kb, about 300 kb to about 600 kb, about 300 kb to about 500 kb, about 300 kb to about 400 kb, about 400 kb to about 1,000 kb, about 400 kb to about 900 kb, about 400 kb to about 800 kb, about 400 kb to about 700 kb, about 400 kb to about 600 kb, about 400 kb to about 500 kb, about 500 kb to about 1,000 kb, about 500 kb to about 900 kb, about 500 kb to about 800 kb, about 500 kb to about 700 kb, about 500 kb to about 600 kb, about 600 kb to about 1,000 kb, about 600 kb to about 900 kb, about 600 kb to about 800 kb, about 600 kb to about 700 kb, about 700 kb to about 1,000 kb, about 700 kb to about 900 kb, about 700 kb to about 800 kb, about 800 kb to about 1,000 kb, about 800 kb to about 900 kb, or about 900 kb to about 1,000 kb.

In some embodiments, the artificial chromosome(s) is a bacterial artificial chromosome (BAC) and can have a total number of nucleotides of up to 750 kb. In some embodiments, the artificial chrosome(s) is a BAC and can have a total number of nucleotides in the range of about 100 kb to about 750 kb, about 100 kb to about 700 kb, about 100 kb to about 600 kb, about 100 kb to about 500 kb, about 100 kb to about 400 kb, about 100 kb to about 300 kb, about 100 kb to about 200 kb, about 150 kb to about 750 kb, about 150 kb to about 700 kb, about 150 kb to about 600 kb, about 150 kb to about 500 kb, about 150 kb to about 400 kb, about 150 kb to about 300 kb, about 150 kb to about 200 kb, about 200 kb to about 750 kb, about 200 kb to about 700 kb, about 200 kb to about 600 kb, about 200 kb to about 500 kb, about 200 kb to about 400 kb, about 200 kb to about 300 kb, about 250 kb to about 750 kb, about 250 kb to about 700 kb, about 250 kb to about 600 kb, about 250 kb to about 500 kb, about 250 kb to about 400 kb, about 250 kb to about 300 kb, about 300 kb to about 750 kb, about 300 kb to about 700 kb, about 300 kb to about 600 kb, about 300 kb to about 500 kb, about 300 kb to about 400 kb, about 350 kb to about 750 kb, about 350 kb to about 700 kb, about 350 kb to about 600 kb, about 350 kb to about 500 kb, about 350 kb to about 400 kb, about 400 kb to about 750 kb, about 400 kb to about 700 kb, about 450 kb to about 600 kb, about 450 kb to about 500 kb, about 500 kb to about 750 kb, about 500 kb to about 700 kb, about 500 kb to about 600 kb, about 550 kb to about 750 kb, about 550 kb to about 700 kb, about 550 kb to about 600 kb, about 600 kb to about 750 kb, about 600 kb to about 700 kb, or about 650 kb to about 750 kb.

In some embodiments, the artificial chromosome(s) is a P1-derived artificial chromosome (PAC) and can have a total number of nucleotides of up to 300 kb. In some embodiments, the P1-derived artificial chromosome(s) can have a total number of nucleotides in the range of about 100 kb to about 300 kb, about 100 kb to about 200 kb, or about 200 kb to about 300 kb.

In some embodiments, the vector(s) is a viral vector and can have a total number of nucleotides of up to 10 kb. In some embodiments, the viral vector(s) can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some embodiments, the vector(s) is a lentivirus and can have a total number of nucleotides of up to 8 kb. In some examples, the lentivirus(es) can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb.

In some embodiments, the vector(s) is an adenovirus and can have a total number of nucleotides of up to 8 kb. In some embodiments, the adenovirus(es) can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kh, about 6 kb to about 8 kb, or about 7 kb to about 8 kb.

In some embodiments, the vector(s) is an adeno-associated virus (AAV vector) and can include a total number of nucleotides of up to 5 kb. In some embodiments, the AAV vector(s) can include a total number of nucleotides in the range of about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

In some embodiments, the vector(s) is a Gateway® vector and can include a total number of nucleotides of up to 5 kb. In some embodiments, each Gateway® vector(s) includes a total number of nucleotides in the range of about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

In some embodiments of any of the compositions, kits, and methods provided herein, the at least two different vectors can be substantially the same type of vector and may differ in size. In some embodiments, the at least two different vectors can be different types of vector, and may have substantially the same size or have different sizes.

In some embodiments, any of the at least two vectors can have a total number of nucleotides in the range of about 500 nucleotides to about 15,000 nucleotides, about 500 nucleotides to about 14,500 nucleotides, about 500 nucleotides to about 14,000 nucleotides, about 500 nucleotides to about 13,500 nucleotides, about 500 nucleotides to about 13,000 nucleotides, about 500 nucleotides to about 12,500 nucleotides, about 500 nucleotides to about 12,000 nucleotides, about 500 nucleotides to about 11,500 nucleotides, about 500 nucleotides to about 11,000 nucleotides, about 500 nucleotides to about 10,500 nucleotides, about 500 nucleotides to about 10,000 nucleotides, about 500 nucleotides to about 9,500 nucleotides, about 500 nucleotides to about 9,000 nucleotides, about 500 nucleotides to about 8,500 nucleotides, about 500 nucleotides to about 8,000 nucleotides, about 500 nucleotides to about 7,800 nucleotides, about 500 nucleotides to about 7,600 nucleotides, about 500 nucleotides to about 7,400 nucleotides, about 500 nucleotides to about 7,200 nucleotides, about 500 nucleotides to about 7,000 nucleotides, about 500 nucleotides to about 6,800 nucleotides, about 500 nucleotides to about 6,600 nucleotides, about 500 nucleotides to about 6,400 nucleotides, about 500 nucleotides to about 6,200 nucleotides, about 500 nucleotides to about 6,000 nucleotides, about 500 nucleotides to about 5,800 nucleotides, about 500 nucleotides to about 5,600 nucleotides, about 500 nucleotides to about 5,400 nucleotides, about 500 nucleotides to about 5,200 nucleotides, about 500 nucleotides to about 5,000 nucleotides, about 500 nucleotides to about 4,800 nucleotides, about 4,600 nucleotides, about 500 nucleotides to about 4,400 nucleotides, about 500 nucleotides to about 4,200 nucleotides, about 500 nucleotides to about 4,000 nucleotides, about 500 nucleotides to about 3,800 nucleotides, about 500 nucleotides to about 3,600 nucleotides, about 500 nucleotides to about 3,400 nucleotides, about 500 nucleotides to about 3,200 nucleotides, about 500 nucleotides to about 3,000 nucleotides, about 500 nucleotides to about 2,800 nucleotides, about 500 nucleotides to about 2,600 nucleotides, about 500 nucleotides to about 2,400 nucleotides, about 500 nucleotides to about 2,200 nucleotides, about 500 nucleotides to about 2,000 nucleotides, about 500 nucleotides to about 1,800 nucleotides, about 500 nucleotides to about 1,600 nucleotides, about 500 nucleotides to about 1,400 nucleotides, about 500 nucleotides to about 1,200 nucleotides, about 500 nucleotides to about 1,000 nucleotides, about 500 nucleotides to about 800 nucleotides, about 800 nucleotides to about 15,000 nucleotides, about 800 nucleotides to about 14,500 nucleotides, about 800 nucleotides to about 14,000 nucleotides, about 800 nucleotides to about 13,500 nucleotides, about 800 nucleotides to about 13,000 nucleotides, about 800 nucleotides to about 12,500 nucleotides, about 800 nucleotides to about 12,000 nucleotides, about 800 nucleotides to about 11,500 nucleotides, about 800 nucleotides to about 11,000 nucleotides, about 800 nucleotides to about 10,500 nucleotides, about 800 nucleotides to about 10,000 nucleotides, about 800 nucleotides to about 9,500 nucleotides, about 800 nucleotides to about 9,000 nucleotides, about 800 nucleotides to about 8,500 nucleotides, about 800 nucleotides to about 8,000 nucleotides, about 800 nucleotides to about 7,800 nucleotides, about 800 nucleotides to about 7,600 nucleotides, about 800 nucleotides to about 7,400 nucleotides, about 800 nucleotides to about 7,200 nucleotides, about 800 nucleotides to about 7,000 nucleotides, about 800 nucleotides to about 6,800 nucleotides, about 800 nucleotides to about 6,600 nucleotides, about 800 nucleotides to about 6,400 nucleotides, about 800 nucleotides to about 6,200 nucleotides, about 800 nucleotides to about 6,000 nucleotides, about 800 nucleotides to about 5,800 nucleotides, about 800 nucleotides to about 5,600 nucleotides, about 800 nucleotides to about 5,400 nucleotides, about 800 nucleotides to about 5,200 nucleotides, about 800 nucleotides to about 5,000 nucleotides, about 800 nucleotides to about 4,800 nucleotides, about 800 nucleotides to about 4,600 nucleotides, about 800 nucleotides to about 4,400 nucleotides, about 800 nucleotides to about 4,200 nucleotides, about 800 nucleotides to about 4,000 nucleotides, about 800 nucleotides to about 3,800 nucleotides, about 800 nucleotides to about 3,600 nucleotides, about 800 nucleotides to about 3,400 nucleotides, about 800 nucleotides to about 3,200 nucleotides, about 800 nucleotides to about 3,000 nucleotides, about 800 nucleotides to about 2,800 nucleotides, about 800 nucleotides to about 2,600 nucleotides, about 800 nucleotides to about 2,400 nucleotides, about 800 nucleotides to about 2,200 nucleotides, about 800 nucleotides to about 2,000 nucleotides, about 800 nucleotides to about 1,800 nucleotides, about 800 nucleotides to about 1,600 nucleotides, about 800 nucleotides to about 1,400 nucleotides, about 800 nucleotides to about 1,200 nucleotides, about 800 nucleotides to about 1,000 nucleotides, about 1,000 nucleotides to about 15,000 nucleotides, about 1,000 nucleotides to about 14,500 nucleotides, about 1,000 nucleotides to about 14,000 nucleotides, about 1,000 nucleotides to about 13,500 nucleotides, about 1,000 nucleotides to about 13,000 nucleotides, about 1,000 nucleotides to about 12,500 nucleotides, about 1,000 nucleotides to about 12,000 nucleotides, about 1,000 nucleotides to about 11,500 nucleotides, about 1,000 nucleotides to about 11,000 nucleotides, about 1,000 nucleotides to about 10,500 nucleotides, about 1,000 nucleotides to about 10,000 nucleotides, about 1,000 nucleotides to about 9,000 nucleotides, about 1,000 nucleotides to about 8,500 nucleotides, about 1,000 nucleotides to about 8,000 nucleotides, about 1,000 nucleotides to about 7,800 nucleotides, about 1,000 nucleotides to about 7,600 nucleotides, about 1,000 nucleotides to about 7,400 nucleotides, about 1,000 nucleotides to about 7,200 nucleotides, about 1,000 nucleotides to about 7,000 nucleotides, about 1,000 nucleotides to about 6,800 nucleotides, about 1,000 nucleotides to about 6,600 nucleotides, about 1,000 nucleotides to about 6,400 nucleotides, about 1,000 nucleotides to about 6,200 nucleotides, about 1,000 nucleotides to about 6,000 nucleotides, about 1,000 nucleotides to about 5,800 nucleotides, about 1,000 nucleotides to about 5,600 nucleotides, about 1,000 nucleotides to about 5,400 nucleotides, about 1,000 nucleotides to about 5,200 nucleotides, about 1,000 nucleotides to about 5,000 nucleotides, about 1,000 nucleotides to about 4,800 nucleotides, about 1,000 nucleotides to about 4,600 nucleotides, about 1,000 nucleotides to about 4,400 nucleotides, about 1,000 nucleotides to about 4,200 nucleotides, about 1,000 nucleotides to about 4,000 nucleotides, about 1,000 nucleotides to about 3,800 nucleotides, about 1,000 nucleotides to about 3,600 nucleotides, about 1,000 nucleotides to about 3,400 nucleotides, about 1,000 nucleotides to about 3,200 nucleotides, about 1,000 nucleotides to about 3,000 nucleotides, about 1,000 nucleotides to about 2,600 nucleotides, about 1,000 nucleotides to about 2,400 nucleotides, about 1,000 nucleotides to about 2,200 nucleotides, about 1,000 nucleotides to about 2,000 nucleotides, about 1,000 nucleotides to about 1,800 nucleotides, about 1,000 nucleotides to about 1,600 nucleotides, about 1,000 nucleotides to about 1,400 nucleotides, about 1,000 nucleotides to about 1,200 nucleotides, about 1,200 nucleotides to about 15,000 nucleotides, about 1,200 nucleotides to about 14,500 nucleotides, about 1,200 nucleotides to about 14,000 nucleotides, about 1,200 nucleotides to about 13,500 nucleotides, about 1,200 nucleotides to about 13,000 nucleotides, about 1,200 nucleotides to about 12,500 nucleotides, about 1,200 nucleotides to about 12,000 nucleotides, about 1,200 nucleotides to about 11,500 nucleotides, about 1,200 nucleotides to about 11,000 nucleotides, about 1,200 nucleotides to about 10,500 nucleotides, about 1,200 nucleotides to about 10,000 nucleotides, about 1,200 nucleotides to about 9,500 nucleotides, about 1,200 nucleotides to about 9,000 nucleotides, about 1,200 nucleotides to about 8,500 nucleotides, about 1,200 nucleotides to about 8,000 nucleotides, about 1,200 nucleotides to about 7,800 nucleotides, about 1,200 nucleotides to about 7,600 nucleotides, about 1,200 nucleotides to about 7,400 nucleotides, about 1,200 nucleotides to about 7,200 nucleotides, about 1,200 nucleotides to about 7,000 nucleotides, about 1,200 nucleotides to about 6,800 nucleotides, about 1,200 nucleotides to about 6,600 nucleotides, about 1,200 nucleotides to about 6,400 nucleotides, about 1,200 nucleotides to about 6,200 nucleotides, about 1,200 nucleotides to about 6,000 nucleotides, about 1,200 nucleotides to about 5,800 nucleotides, about 1,200 nucleotides to about 5,600 nucleotides, about 1,200 nucleotides to about 5,400 nucleotides, about 1,200 nucleotides to about 5,000 nucleotides, about 1,200 nucleotides to about 4,800 nucleotides, about 1,200 nucleotides to about 4,600 nucleotides, about 1,200 nucleotides to about 4,400 nucleotides, about 1,200 nucleotides to about 4,200 nucleotides, about 1,200 nucleotides to about 4,000 nucleotides, about 1,200 nucleotides to about 3,800 nucleotides, about 1,200 nucleotides to about 3,600 nucleotides, about 1,200 nucleotides to about 3,400 nucleotides, about 1,200 nucleotides to about 3,200 nucleotides, about 1,200 nucleotides to about 3,000 nucleotides, about 1,200 nucleotides to about 2,800 nucleotides, about 1,200 nucleotides to about 2,600 nucleotides, about 1,200 nucleotides to about 2,400 nucleotides, about 1,200 nucleotides to about 2,200 nucleotides, about 1,200 nucleotides to about 2,000 nucleotides, about 1,200 nucleotides to about 1,800 nucleotides, about 1,200 nucleotides to about 1,600 nucleotides, about 1,200 nucleotides to about 1,400 nucleotides, about 1,400 nucleotides to about 15,000 nucleotides, about 1,400 nucleotides to about 14,500 nucleotides, about 1,400 nucleotides to about 14,000 nucleotides, about 1,400 nucleotides to about 13,500 nucleotides, about 1,400 nucleotides to about 13,000 nucleotides, about 1,400 nucleotides to about 12,500 nucleotides, about 1,400 nucleotides to about 12,000 nucleotides, about 1,400 nucleotides to about 11,500 nucleotides, about 1,400 nucleotides to about 11,000 nucleotides, about 1,400 nucleotides to about 10,500 nucleotides, about 1,400 nucleotides to about 10,000 nucleotides, about 1,400 nucleotides to about 9,500 nucleotides, about 1,400 nucleotides to about 9,000 nucleotides, about 1,400 nucleotides to about 8,500 nucleotides, about 1,400 nucleotides to about 8,000 nucleotides, about 1,400 nucleotides to about 7,800 nucleotides, about 1,400 nucleotides to about 7,600 nucleotides, about 1,400 nucleotides to about 7,400 nucleotides, about 1,400 nucleotides to about 7,200 nucleotides, about 1,400 nucleotides to about 7,000 nucleotides, about 1,400 nucleotides to about 6,800 nucleotides, about 1,400 nucleotides to about 6,600 nucleotides, about 1,400 nucleotides to about 6,400 nucleotides, about 1,400 nucleotides to about 6,200 nucleotides, about 1,400 nucleotides to about 6,000 nucleotides, about 1,400 nucleotides to about 5,800 nucleotides, about 1,400 nucleotides to about 5,600 nucleotides, about 1,400 nucleotides to about 5,400 nucleotides, about 1,400 nucleotides to about 5,200 nucleotides, about 1,400 nucleotides to about 5,000 nucleotides, about 1,400 nucleotides to about 4,800 nucleotides, about 1,400 nucleotides to about 4,600 nucleotides, about 1,400 nucleotides to about 4,400 nucleotides, about 1,400 nucleotides to about 4,200 nucleotides, about 1,400 nucleotides to about 4,000 nucleotides, about 1,400 nucleotides to about 3,800 nucleotides, about 1,400 nucleotides to about 3,600 nucleotides, about 1,400 nucleotides to about 3,400 nucleotides, about 1,400 nucleotides to about 3,200 nucleotides, about 1,400 nucleotides to about 3,000 nucleotides, about 1,400 nucleotides to about 2,600 nucleotides, about 1,400 nucleotides to about 2,400 nucleotides, about 1,400 nucleotides to about 2,200 nucleotides, about 1,400 nucleotides to about 2,000 nucleotides, about 1,400 nucleotides to about 1,800 nucleotides, about 1,400 nucleotides to about 1,600 nucleotides, about 1,600 nucleotides to about 15,000 nucleotides, about 1,600 nucleotides to about 14,500 nucleotides, about 1,600 nucleotides to about 14,000 nucleotides, about 1,600 nucleotides to about 13,500 nucleotides, about 1,600 nucleotides to about 13,000 nucleotides, about 1,600 nucleotides to about 12,500 nucleotides, about 1,600 nucleotides to about 12,000 nucleotides, about 1,600 nucleotides to about 11,500 nucleotides, about 1,600 nucleotides to about 11,000 nucleotides, about 1,600 nucleotides to about 10,500 nucleotides, about 1,600 nucleotides to about 10,000 nucleotides, about 1,600 nucleotides to about 9,500 nucleotides, about 1,600 nucleotides to about 9,000 nucleotides, about 1,600 nucleotides to about 8,500 nucleotides, about 1,600 nucleotides to about 8,000 nucleotides, about 1,600 nucleotides to about 7,800 nucleotides, about 1,600 nucleotides to about 7,600 nucleotides, about 1,600 nucleotides to about 7,400 nucleotides, about 1,600 nucleotides to about 7,200 nucleotides, about 1,600 nucleotides to about 7,000 nucleotides, about 1,600 nucleotides to about 6,800 nucleotides, about 1,600 nucleotides to about 6,400 nucleotides, about 1,600 nucleotides to about 6,200 nucleotides, about 1,600 nucleotides to about 6,000 nucleotides, about 1,600 nucleotides to about 5,800 nucleotides, about 1,600 nucleotides to about 5,600 nucleotides, about 1,600 nucleotides to about 5,400 nucleotides, about 1,600 nucleotides to about 5,200 nucleotides, about 1,600 nucleotides to about 5,000 nucleotides, about 1,600 nucleotides to about 4,800 nucleotides, about 1,600 nucleotides to about 4,600 nucleotides, about 1,600 nucleotides to about 4,400 nucleotides, about 1,600 nucleotides to about 4,200 nucleotides, about 1,600 nucleotides to about 4,000 nucleotides, about 1,600 nucleotides to about 3,800 nucleotides, about 1,600 nucleotides to about 3,600 nucleotides, about 1,600 nucleotides to about 3,400 nucleotides, about 1,600 nucleotides to about 3,200 nucleotides, about 1,600 nucleotides to about 3,000 nucleotides, about 1,600 nucleotides to about 2,800 nucleotides, about 1,600 nucleotides to about 2,600 nucleotides, about 1,600 nucleotides to about 2,400 nucleotides, about 1,600 nucleotides to about 2,200 nucleotides, about 1,600 nucleotides to about 2,000 nucleotides, about 1,600 nucleotides to about 1,800 nucleotides, about 1,800 nucleotides to about 15,000 nucleotides, about 1,800 nucleotides to about 14,500 nucleotides, about 1,800 nucleotides to about 14,000 nucleotides, about 1,800 nucleotides to about 13,500 nucleotides, about 1,800 nucleotides to about 13,000 nucleotides, about 1,800 nucleotides to about 12,500 nucleotides, about 1,800 nucleotides to about 12,000 nucleotides, about 1,800 nucleotides to about 11,500 nucleotides, about 1,800 nucleotides to about 11,000 nucleotides, about 1,800 nucleotides to about 10,500 nucleotides, about 1,800 nucleotides to about 10,000 nucleotides, about 1,800 nucleotides to about 9,500 nucleotides, about 1,800 nucleotides to about 9,000 nucleotides, about 1,800 nucleotides to about 8,500 nucleotides, about 1,800 nucleotides to about 8,000 nucleotides, about 1,800 nucleotides to about 7,800 nucleotides, about 1,800 nucleotides to about 7,600 nucleotides, about 1,800 nucleotides to about 7,400 nucleotides, about 1,800 nucleotides to about 7,200 nucleotides, about 1,800 nucleotides to about 7,000 nucleotides, about 1,800 nucleotides to about 6,800 nucleotides, about 1,800 nucleotides to about 6,600 nucleotides, about 1,800 nucleotides to about 6,400 nucleotides, about 1,800 nucleotides to about 6,200 nucleotides, about 1,800 nucleotides to about 6,000 nucleotides, about 1,800 nucleotides to about 5,800 nucleotides, about 1,800 nucleotides to about 5,600 nucleotides, about 1,800 nucleotides to about 5,400 nucleotides, about 1,800 nucleotides to about 5,200 nucleotides, about 1,800 nucleotides to about 5,000 nucleotides, about 1,800 nucleotides to about 4,800 nucleotides, about 1,800 nucleotides to about 4,600 nucleotides, about 1,800 nucleotides to about 4,400 nucleotides, about 1,800 nucleotides to about 4,200 nucleotides, about 1,800 nucleotides to about 4,000 nucleotides, about 1,800 nucleotides to about 3,800 nucleotides, about 1,800 nucleotides to about 3,600 nucleotides, about 1,800 nucleotides to about 3,400 nucleotides, about 1,800 nucleotides to about 3,200 nucleotides, about 1,800 nucleotides to about 3,000 nucleotides, about 1,800 nucleotides to about 2,800 nucleotides, about 1,800 nucleotides to about 2,600 nucleotides, about 1,800 nucleotides to about 2,400 nucleotides, about 1,800 nucleotides to about 2,200 nucleotides, about 1,800 nucleotides to about 2,000 nucleotides, about 2,000 nucleotides to about 15,000 nucleotides, about 2,000 nucleotides to about 14,500 nucleotides, about 2,000 nucleotides to about 14,000 nucleotides, about 2,000 nucleotides to about 13,500 nucleotides, about 2,000 nucleotides to about 13,000 nucleotides, about 2,000 nucleotides to about 12,500 nucleotides, about 2,000 nucleotides to about 12,000 nucleotides, about 2,000 nucleotides to about 11,500 nucleotides, about 2,000 nucleotides to about 11,000 nucleotides, about 2,000 nucleotides to about 10,500 nucleotides, about 2,000 nucleotides to about 10,000 nucleotides, about 2,000 nucleotides to about 9,500 nucleotides, about 2,000 nucleotides to about 9,000 nucleotides, about 2,000 nucleotides to about 8,500 nucleotides, about 2,000 nucleotides to about 8,000 nucleotides, about 2,000 nucleotides to about 7,800 nucleotides, about 2,000 nucleotides to about 7,600 nucleotides, about 2,000 nucleotides to about 7,400 nucleotides, about 2,000 nucleotides to about 7,200 nucleotides, about 2,000 nucleotides to about 7,000 nucleotides, about 2,000 nucleotides to about 6,800 nucleotides, about 2,000 nucleotides to about 6,600 nucleotides, about 2,000 nucleotides to about 6,400 nucleotides, about 2,000 nucleotides to about 6,200 nucleotides, about 2,000 nucleotides to about 6,000 nucleotides, about 2,000 nucleotides to about 5,800 nucleotides, about 2,000 nucleotides to about 5,600 nucleotides, about 2,000 nucleotides to about 5,400 nucleotides, about 2,000 nucleotides to about 5,200 nucleotides, about 2,000 nucleotides to about 5,000 nucleotides, about 2,000 nucleotides to about 4,800 nucleotides, about 2,000 nucleotides to about 4,600 nucleotides, about 2,000 nucleotides to about 4,400 nucleotides, about 2,000 nucleotides to about 4,200 nucleotides, about 2,000 nucleotides to about 4,000 nucleotides, about 2,000 nucleotides to about 3,800 nucleotides, about 2,000 nucleotides to about 3,600 nucleotides, about 2,000 nucleotides to about 3,400 nucleotides, about 2,000 nucleotides to about 3,200 nucleotides, about 2,000 nucleotides to about 3,000 nucleotides, about 2,000 nucleotides to about 2,800 nucleotides, about 2,000 nucleotides to about 2,600 nucleotides, about 2,000 nucleotides to about 2,400 nucleotides, about 2,000 nucleotides to about 2,200 nucleotides, about 2,200 nucleotides to about 15,000 nucleotides, about 2,200 nucleotides to about 14,500 nucleotides, about 2,200 nucleotides to about 14,000 nucleotides, about 2,200 nucleotides to about 13,500 nucleotides, about 2,200 nucleotides to about 13,000 nucleotides, about 2,200 nucleotides to about 12,500 nucleotides, about 2,200 nucleotides to about 12,000 nucleotides, about 2,200 nucleotides to about 11,500 nucleotides, about 2,200 nucleotides to about 11,000 nucleotides, about 2,200 nucleotides to about 10,500 nucleotides, about 2,200 nucleotides to about 10,000 nucleotides, about 9,500 nucleotides, about 9,000 nucleotides, about 8,500 nucleotides, about 8,000 nucleotides, about 7,800 nucleotides, about 7,600 nucleotides, about 7,400 nucleotides, about 7,200 nucleotides, about 7,000 nucleotides, about 6,800 nucleotides, about 6,600 nucleotides, about 6,400 nucleotides, about 6,200 nucleotides, about 6,000 nucleotides, about 5,800 nucleotides, about 5,600 nucleotides, about 5,400 nucleotides, about 5,200 nucleotides, about 5,000 nucleotides, about 4,800 nucleotides, about 4,600 nucleotides, about 4,400 nucleotides, about 4,200 nucleotides, about 4,000 nucleotides, about 3,800 nucleotides, about 3,600 nucleotides, about 3,400 nucleotides, about 3,200 nucleotides, about 3,000 nucleotides, about 2,800 nucleotides, about 2,600 nucleotides, about 2,400 nucleotides, about 2,400 nucleotides to about 15,000 nucleotides, about 2,400 nucleotides to about 14,500 nucleotides, about 2,400 nucleotides to about 14,000 nucleotides, about 2,400 nucleotides to about 13,500 nucleotides, about 2,400 nucleotides to about 13,000 nucleotides, about 2,400 nucleotides to about 12,500 nucleotides, about 2,400 nucleotides to about 12,000 nucleotides, about 2,400 nucleotides to about 11,500 nucleotides, about 2,400 nucleotides to about 11,000 nucleotides, about 2,400 nucleotides to about 10,500 nucleotides, about 2,400 nucleotides to about 10,000 nucleotides, about 2,400 nucleotides to about 9,500 nucleotides, about 2,400 nucleotides to about 9,000 nucleotides, about 2,400 nucleotides to about 8,500 nucleotides, about 2,400 nucleotides to about 8,000 nucleotides, about 2,400 nucleotides to about 7,800 nucleotides, about 2,400 nucleotides to about 7,600 nucleotides, about 2,400 nucleotides to about 7,400 nucleotides, about 2,400 nucleotides to about 7,200 nucleotides, about 2,400 nucleotides to about 7,000 nucleotides, about 2,400 nucleotides to about 6,800 nucleotides, about 2,400 nucleotides to about 6,600 nucleotides, about 2,400 nucleotides to about 6,400 nucleotides, about 2,400 nucleotides to about 6,200 nucleotides, about 2,400 nucleotides to about 6,000 nucleotides, about 2,400 nucleotides to about 5,800 nucleotides, about 2,400 nucleotides to about 5,600 nucleotides, about 2,400 nucleotides to about 5,400 nucleotides, about 2,400 nucleotides to about 5,200 nucleotides, about 2,400 nucleotides to about 5,000 nucleotides, about 2,400 nucleotides to about 4,800 nucleotides, about 2,400 nucleotides to about 4,600 nucleotides, about 2,400 nucleotides to about 4,400 nucleotides, about 2,400 nucleotides to about 4,200 nucleotides, about 2,400 nucleotides to about 4,000 nucleotides, about 2,400 nucleotides to about 3,800 nucleotides, about 2,400 nucleotides to about 3,600 nucleotides, about 2,400 nucleotides to about 3,400 nucleotides, about 2,400 nucleotides to about 3,200 nucleotides, about 2,400 nucleotides to about 3,000 nucleotides, about 2,400 nucleotides to about 2,800 nucleotides, about 2,400 nucleotides to about 2,600 nucleotides, about 2,600 nucleotides to about 15,000 nucleotides, about 2,600 nucleotides to about 14,500 nucleotides, about 2,600 nucleotides to about 14,000 nucleotides, about 2,600 nucleotides to about 13,500 nucleotides, about 2,600 nucleotides to about 13,000 nucleotides, about 2,600 nucleotides to about 12,500 nucleotides, about 2,600 nucleotides to about 12,000 nucleotides, about 2,600 nucleotides to about 11,500 nucleotides, about 2,600 nucleotides to about 11,000 nucleotides, about 2,600 nucleotides to about 10,500 nucleotides, about 2,600 nucleotides to about 10,000 nucleotides, about 2,600 nucleotides to about 9,500 nucleotides, about 2,600 nucleotides to about 9,000 nucleotides, about 2,600 nucleotides to about 8,500 nucleotides, about 2,600 nucleotides to about 8,000 nucleotides, about 2,600 nucleotides to about 7,800 nucleotides, about 2,600 nucleotides to about 7,600 nucleotides, about 2,600 nucleotides to about 7,400 nucleotides, about 2,600 nucleotides to about 7,200 nucleotides, about 2,600 nucleotides to about 7,000 nucleotides, about 2,600 nucleotides to about 6,800 nucleotides, about 2,600 nucleotides to about 6,600 nucleotides, about 2,600 nucleotides to about 6,400 nucleotides, about 2,600 nucleotides to about 6,200 nucleotides, about 2,600 nucleotides to about 6,000 nucleotides, about 2,600 nucleotides to about 5,800 nucleotides, about 2,600 nucleotides to about 5,600 nucleotides, about 2,600 nucleotides to about 5,400 nucleotides, about 2,600 nucleotides to about 5,200 nucleotides, about 2,600 nucleotides to about 5,000 nucleotides, about 2,600 nucleotides to about 4,800 nucleotides, about 2,600 nucleotides to about 4,600 nucleotides, about 2,600 nucleotides to about 4,400 nucleotides, about 2,600 nucleotides to about 4,200 nucleotides, about 2,600 nucleotides to about 4,000 nucleotides, about 2,600 nucleotides to about 3,800 nucleotides, about 2,600 nucleotides to about 3,600 nucleotides, about 2,600 nucleotides to about 3,400 nucleotides, about 2,600 nucleotides to about 3,200 nucleotides, about 2,600 nucleotides to about 3,000 nucleotides, about 2,600 nucleotides to about 2,800 nucleotides, about 2,800 nucleotides to about 15,000 nucleotides, about 2,800 nucleotides to about 14,500 nucleotides, about 2,800 nucleotides to about 14,000 nucleotides, about 2,800 nucleotides to about 13,500 nucleotides, about 2,800 nucleotides to about 13,000 nucleotides, about 2,800 nucleotides to about 12,500 nucleotides, about 2,800 nucleotides to about 12,000 nucleotides, about 2,800 nucleotides to about 11,500 nucleotides, about 2,800 nucleotides to about 11,000 nucleotides, about 2,800 nucleotides to about 10,500 nucleotides, about 2,800 nucleotides to about 10,000 nucleotides, about 2,800 nucleotides to about 9,500 nucleotides, about 2,800 nucleotides to about 9,000 nucleotides, about 2,800 nucleotides to about 8,500 nucleotides, about 2,800 nucleotides to about 8,000 nucleotides, about 2,800 nucleotides to about 7,800 nucleotides, about 2,800 nucleotides to about 7,600 nucleotides, about 2,800 nucleotides to about 7,400 nucleotides, about 2,800 nucleotides to about 7,200 nucleotides, about 2,800 nucleotides to about 7,000 nucleotides, about 2,800 nucleotides to about 6,800 nucleotides, about 2,800 nucleotides to about 6,600 nucleotides, about 2,800 nucleotides to about 6,400 nucleotides, about 2,800 nucleotides to about 6,200 nucleotides, about 2,800 nucleotides to about 6,000 nucleotides, about 2,800 nucleotides to about 5,800 nucleotides, about 2,800 nucleotides to about 5,600 nucleotides, about 2,800 nucleotides to about 5,400 nucleotides, about 2,800 nucleotides to about 5,200 nucleotides, about 2,800 nucleotides to about 5,000 nucleotides, about 2,800 nucleotides to about 4,800 nucleotides, about 2,800 nucleotides to about 4,600 nucleotides, about 2,800 nucleotides to about 4,400 nucleotides, about 2,800 nucleotides to about 4,200 nucleotides, about 2,800 nucleotides to about 4,000 nucleotides, about 2,800 nucleotides to about 3,800 nucleotides, about 2,800 nucleotides to about 3,600 nucleotides, about 2,800 nucleotides to about 3,400 nucleotides, about 2,800 nucleotides to about 3,200 nucleotides, about 2,800 nucleotides to about 3,000 nucleotides, about 3,000 nucleotides to about 15,000 nucleotides, about 3,000 nucleotides to about 14,500 nucleotides, about 3,000 nucleotides to about 14,000 nucleotides, about 3,000 nucleotides to about 13,500 nucleotides, about 3,000 nucleotides to about 13,000 nucleotides, about 3,000 nucleotides to about 12,500 nucleotides, about 3,000 nucleotides to about 12,000 nucleotides, about 3,000 nucleotides to about 11,500 nucleotides, about 3,000 nucleotides to about 11,000 nucleotides, about 3,000 nucleotides to about 10,500 nucleotides, about 3,000 nucleotides to about 10,000 nucleotides, about 3,000 nucleotides to about 9,500 nucleotides, about 3,000 nucleotides to about 9,000 nucleotides, about 3,000 nucleotides to about 8,500 nucleotides, about 3,000 nucleotides to about 8,000 nucleotides, about 3,000 nucleotides to about 7,800 nucleotides, about 3,000 nucleotides to about 7,600 nucleotides, about 3,000 nucleotides to about 7,400 nucleotides, about 3,000 nucleotides to about 7,200 nucleotides, about 3,000 nucleotides to about 7,000 nucleotides, about 3,000 nucleotides to about 6,800 nucleotides, about 3,000 nucleotides to about 6,600 nucleotides, about 3,000 nucleotides to about 6,400 nucleotides, about 3,000 nucleotides to about 6,200 nucleotides, about 3,000 nucleotides to about 6,000 nucleotides, about 3,000 nucleotides to about 5,800 nucleotides, about 3,000 nucleotides to about 5,600 nucleotides, about 3,000 nucleotides to about 5,400 nucleotides, about 3,000 nucleotides to about 5,200 nucleotides, about 3,000 nucleotides to about 5,000 nucleotides, about 3,000 nucleotides to about 4,800 nucleotides, about 3,000 nucleotides to about 4,600 nucleotides, about 3,000 nucleotides to about 4,400 nucleotides, about 3,000 nucleotides to about 4,200 nucleotides, about 3,000 nucleotides to about 4,000 nucleotides, about 3,000 nucleotides to about 3,800 nucleotides, about 3,000 nucleotides to about 3,600 nucleotides, about 3,000 nucleotides to about 3,400 nucleotides, about 3,000 nucleotides to about 3,200 nucleotides, about 3,200 nucleotides to about 15,000 nucleotides, about 3,200 nucleotides to about 14,500 nucleotides, about 3,200 nucleotides to about 14,000 nucleotides, about 3,200 nucleotides to about 13,500 nucleotides, about 3,200 nucleotides to about 13,000 nucleotides, about 3,200 nucleotides to about 12,500 nucleotides, about 3,200 nucleotides to about 12,000 nucleotides, about 3,200 nucleotides to about 11,500 nucleotides, about 3,200 nucleotides to about 11,000 nucleotides, about 3,200 nucleotides to about 10,500 nucleotides, about 3,200 nucleotides to about 10,000 nucleotides, about 3,200 nucleotides to about 9,500 nucleotides, about 3,200 nucleotides to about 9,000 nucleotides, about 3,200 nucleotides to about 8,500 nucleotides, about 3,200 nucleotides to about 8,000 nucleotides, about 3,200 nucleotides to about 7,800 nucleotides, about 3,200 nucleotides to about 7,600 nucleotides, about 3,200 nucleotides to about 7,400 nucleotides, about 3,200 nucleotides to about 7,200 nucleotides, about 3,200 nucleotides to about 7,000 nucleotides, about 3,200 nucleotides to about 6,800 nucleotides, about 3,200 nucleotides to about 6,600 nucleotides, about 3,200 nucleotides to about 6,400 nucleotides, about 3,200 nucleotides to about 6,200 nucleotides, about 3,200 nucleotides to about 6,000 nucleotides, about 3,200 nucleotides to about 5,800 nucleotides, about 3,200 nucleotides to about 5,600 nucleotides, about 3,200 nucleotides to about 5,400 nucleotides, about 3,200 nucleotides to about 5,200 nucleotides, about 3,200 nucleotides to about 5,000 nucleotides, about 3,200 nucleotides to about 4,800 nucleotides, about 3,200 nucleotides to about 4,600 nucleotides, about 3,200 nucleotides to about 4,400 nucleotides, about 3,200 nucleotides to about 4,200 nucleotides, about 3,200 nucleotides to about 4,000 nucleotides, about 3,200 nucleotides to about 3,800 nucleotides, about 3,200 nucleotides to about 3,600 nucleotides, about 3,200 nucleotides to about 3,400 nucleotides, about 3,400 nucleotides to about 15,000 nucleotides, about 3,400 nucleotides to about 14,500 nucleotides, about 3,400 nucleotides to about 14,000 nucleotides, about 3,400 nucleotides to about 13,500 nucleotides, about 3,400 nucleotides to about 13,000 nucleotides, about 3,400 nucleotides to about 12,500 nucleotides, about 3,400 nucleotides to about 12,000 nucleotides, about 3,400 nucleotides to about 11,500 nucleotides, about 3,400 nucleotides to about 11,000 nucleotides, about 3,400 nucleotides to about 10,500 nucleotides, about 3,400 nucleotides to about 10,000 nucleotides, about 3,400 nucleotides to about 9,500 nucleotides, about 3,400 nucleotides to about 9,000 nucleotides, about 3,400 nucleotides to about 8,500 nucleotides, about 3,400 nucleotides to about 8,000 nucleotides, about 3,400 nucleotides to about 7,800 nucleotides, about 3,400 nucleotides to about 7,600 nucleotides, about 3,400 nucleotides to about 7,400 nucleotides, about 3,400 nucleotides to about 7,200 nucleotides, about 3,400 nucleotides to about 7,000 nucleotides, about 3,400 nucleotides to about 6,800 nucleotides, about 3,400 nucleotides to about 6,600 nucleotides, about 3,400 nucleotides to about 6,400 nucleotides, about 3,400 nucleotides to about 6,200 nucleotides, about 3,400 nucleotides to about 6,000 nucleotides, about 3,400 nucleotides to about 5,800 nucleotides, about 3,400 nucleotides to about 5,600 nucleotides, about 3,400 nucleotides to about 5,400 nucleotides, about 3,400 nucleotides to about 5,200 nucleotides, about 3,400 nucleotides to about 5,000 nucleotides, about 3,400 nucleotides to about 4,800 nucleotides, about 3,400 nucleotides to about 4,600 nucleotides, about 3,400 nucleotides to about 4,400 nucleotides, about 3,400 nucleotides to about 4,200 nucleotides, about 3,400 nucleotides to about 4,000 nucleotides, about 3,400 nucleotides to about 3,800 nucleotides, about 3,400 nucleotides to about 3,600 nucleotides, about 3,600 nucleotides to about 15,000 nucleotides, about 3,600 nucleotides to about 14,500 nucleotides, about 3,600 nucleotides to about 14,000 nucleotides, about 3,600 nucleotides to about 13,500 nucleotides, about 3,600 nucleotides to about 13,000 nucleotides, about 3,600 nucleotides to about 12,500 nucleotides, about 3,600 nucleotides to about 12,000 nucleotides, about 3,600 nucleotides to about 11,500 nucleotides, about 3,600 nucleotides to about 11,000 nucleotides, about 3,600 nucleotides to about 10,500 nucleotides, about 3,600 nucleotides to about 10,000 nucleotides, about 3,600 nucleotides to about 9,500 nucleotides, about 3,600 nucleotides to about 9,000 nucleotides, about 3,600 nucleotides to about 8,500 nucleotides, about 3,600 nucleotides to about 8,000 nucleotides, about 3,600 nucleotides to about 7,800 nucleotides, about 3,600 nucleotides to about 7,600 nucleotides, about 3,600 nucleotides to about 7,400 nucleotides, about 3,600 nucleotides to about 7,200 nucleotides, about 3,600 nucleotides to about 7,000 nucleotides, about 3,600 nucleotides to about 6,800 nucleotides, about 3,600 nucleotides to about 6,600 nucleotides, about 3,600 nucleotides to about 6,400 nucleotides, about 3,600 nucleotides to about 6,200 nucleotides, about 3,600 nucleotides to about 6,000 nucleotides, about 3,600 nucleotides to about 5,800 nucleotides, about 3,600 nucleotides to about 5,600 nucleotides, about 3,600 nucleotides to about 5,400 nucleotides, about 3,600 nucleotides to about 5,200 nucleotides, about 3,600 nucleotides to about 5,000 nucleotides, about 3,600 nucleotides to about 4,800 nucleotides, about 3,600 nucleotides to about 4,600 nucleotides, about 3,600 nucleotides to about 4,400 nucleotides, about 3,600 nucleotides to about 4,200 nucleotides, about 3,600 nucleotides to about 4,000 nucleotides, about 3,600 nucleotides to about 3,800 nucleotides, about 3,800 nucleotides to about 15,000 nucleotides, about 3,800 nucleotides to about 14,500 nucleotides, about 3,800 nucleotides to about 14,000 nucleotides, about 3,800 nucleotides to about 13,500 nucleotides, about 3,800 nucleotides to about 13,000 nucleotides, about 3,800 nucleotides to about 12,500 nucleotides, about 3,800 nucleotides to about 12,000 nucleotides, about 3,800 nucleotides to about 11,500 nucleotides, about 3,800 nucleotides to about 11,000 nucleotides, about 3,800 nucleotides to about 10,500 nucleotides, about 3,800 nucleotides to about 10,000 nucleotides, about 3,800 nucleotides to about 9,500 nucleotides, about 3,800 nucleotides to about 9,000 nucleotides, about 3,800 nucleotides to about 8,500 nucleotides, about 3,800 nucleotides to about 8,000 nucleotides, about 3,800 nucleotides to about 7,800 nucleotides, about 3,800 nucleotides to about 7,600 nucleotides, about 3,800 nucleotides to about 7,400 nucleotides, about 3,800 nucleotides to about 7,200 nucleotides, about 3,800 nucleotides to about 7,000 nucleotides, about 3,800 nucleotides to about 6,800 nucleotides, about 3,800 nucleotides to about 6,600 nucleotides, about 3,800 nucleotides to about 6,400 nucleotides, about 3,800 nucleotides to about 6,200 nucleotides, about 3,800 nucleotides to about 6,000 nucleotides, about 3,800 nucleotides to about 5,800 nucleotides, about 3,800 nucleotides to about 5,600 nucleotides, about 3,800 nucleotides to about 5,400 nucleotides, about 3,800 nucleotides to about 5,200 nucleotides, about 3,800 nucleotides to about 5,000 nucleotides, about 3,800 nucleotides to about 4,800 nucleotides, about 3,800 nucleotides to about 4,600 nucleotides, about 3,800 nucleotides to about 4,200 nucleotides, about 3,800 nucleotides to about 4,000 nucleotides, about 4,000 nucleotides to about 15,000 nucleotides, about 4,000 nucleotides to about 14,500 nucleotides, about 4,000 nucleotides to about 14,000 nucleotides, about 4,000 nucleotides to about 13,500 nucleotides, about 4,000 nucleotides to about 13,000 nucleotides, about 4,000 nucleotides to about 12,500 nucleotides, about 4,000 nucleotides to about 12,000 nucleotides, about 4,000 nucleotides to about 11,500 nucleotides, about 4,000 nucleotides to about 11,000 nucleotides, about 4,000 nucleotides to about 10,500 nucleotides, about 4,000 nucleotides to about 10,000 nucleotides, about 4,000 nucleotides to about 9,500 nucleotides, about 4,000 nucleotides to about 9,000 nucleotides, about 4,000 nucleotides to about 8,500 nucleotides, about 4,000 nucleotides to about 8,000 nucleotides, about 4,000 nucleotides to about 7,800 nucleotides, about 4,000 nucleotides to about 7,600 nucleotides, about 4,000 nucleotides to about 7,400 nucleotides, about 4,000 nucleotides to about 7,200 nucleotides, about 4,000 nucleotides to about 7,000 nucleotides, about 4,000 nucleotides to about 6,800 nucleotides, about 4,000 nucleotides to about 6,600 nucleotides, about 4,000 nucleotides to about 6,400 nucleotides, about 4,000 nucleotides to about 6,200 nucleotides, about 4,000 nucleotides to about 6,000 nucleotides, about 4,000 nucleotides to about 5,800 nucleotides, about 4,000 nucleotides to about 5,600 nucleotides, about 4,000 nucleotides to about 5,400 nucleotides, about 4,000 nucleotides to about 5,200 nucleotides, about 4,000 nucleotides to about 5,000 nucleotides, about 4,000 nucleotides to about 4,800 nucleotides, about 4,000 nucleotides to about 4,600 nucleotides, about 4,000 nucleotides to about 4,400 nucleotides, about 4,000 nucleotides to about 4,200 nucleotides, about 4,200 nucleotides to about 15,000 nucleotides, about 4,200 nucleotides to about 14,500 nucleotides, about 4,200 nucleotides to about 14,000 nucleotides, about 4,200 nucleotides to about 13,500 nucleotides, about 4,200 nucleotides to about 13,000 nucleotides, about 4,200 nucleotides to about 12,500 nucleotides, about 4,200 nucleotides to about 12,000 nucleotides, about 4,200 nucleotides to about 11,500 nucleotides, about 4,200 nucleotides to about 11,000 nucleotides, about 4,200 nucleotides to about 10,500 nucleotides, about 4,200 nucleotides to about 10,000 nucleotides, about 4,200 nucleotides to about 9,500 nucleotides, about 4,200 nucleotides to about 9,000 nucleotides, about 4,200 nucleotides to about 8,500 nucleotides, about 4,200 nucleotides to about 8,000 nucleotides, about 4,200 nucleotides to about 7,800 nucleotides, about 4,200 nucleotides to about 7,600 nucleotides, about 4,200 nucleotides to about 7,400 nucleotides, about 4,200 nucleotides to about 7,200 nucleotides, about 4,200 nucleotides to about 7,000 nucleotides, about 4,200 nucleotides to about 6,800 nucleotides, about 4,200 nucleotides to about 6,600 nucleotides, about 4,200 nucleotides to about 6,400 nucleotides, about 4,200 nucleotides to about 6,200 nucleotides, about 4,200 nucleotides to about 6,000 nucleotides, about 4,200 nucleotides to about 5,800 nucleotides, about 4,200 nucleotides to about 5,600 nucleotides, about 4,200 nucleotides to about 5,400 nucleotides, about 4,200 nucleotides to about 5,200 nucleotides, about 4,200 nucleotides to about 5,000 nucleotides, about 4,200 nucleotides to about 4,800 nucleotides, about 4,200 nucleotides to about 4,600 nucleotides, about 4,200 nucleotides to about 4,400 nucleotides, about 4,400 nucleotides to about 15,000 nucleotides, about 4,400 nucleotides to about 14,500 nucleotides, about 4,400 nucleotides to about 14,000 nucleotides, about 4,400 nucleotides to about 13,500 nucleotides, about 4,400 nucleotides to about 13,000 nucleotides, about 4,400 nucleotides to about 12,500 nucleotides, about 4,400 nucleotides to about 12,000 nucleotides, about 4,400 nucleotides to about 11,500 nucleotides, about 4,400 nucleotides to about 11,000 nucleotides, about 4,400 nucleotides to about 10,500 nucleotides, about 4,400 nucleotides to about 10,000 nucleotides, about 4,400 nucleotides to about 9,500 nucleotides, about 4,400 nucleotides to about 9,000 nucleotides, about 4,400 nucleotides to about 8,500 nucleotides, about 4,400 nucleotides to about 8,000 nucleotides, about 4,400 nucleotides to about 7,800 nucleotides, about 4,400 nucleotides to about 7,600 nucleotides, about 4,400 nucleotides to about 7,400 nucleotides, about 4,400 nucleotides to about 7,200 nucleotides, about 4,400 nucleotides to about 7,000 nucleotides, about 4,400 nucleotides to about 6,800 nucleotides, about 4,400 nucleotides to about 6,600 nucleotides, about 4,400 nucleotides to about 6,400 nucleotides, about 4,400 nucleotides to about 6,200 nucleotides, about 4,400 nucleotides to about 6,000 nucleotides, about 4,400 nucleotides to about 5,800 nucleotides, about 4,400 nucleotides to about 5,600 nucleotides, about 4,400 nucleotides to about 5,400 nucleotides, about 4,400 nucleotides to about 5,200 nucleotides, about 4,400 nucleotides to about 5,000 nucleotides, about 4,400 nucleotides to about 4,800 nucleotides, about 4,400 nucleotides to about 4,600 nucleotides, about 4,600 nucleotides to about 15,000 nucleotides, about 4,600 nucleotides to about 14,500 nucleotides, about 4,600 nucleotides to about 14,000 nucleotides, about 4,600 nucleotides to about 13,500 nucleotides, about 4,600 nucleotides to about 13,000 nucleotides, about 4,600 nucleotides to about 12,500 nucleotides, about 4,600 nucleotides to about 12,000 nucleotides, about 4,600 nucleotides to about 11,500 nucleotides, about 4,600 nucleotides to about 11,000 nucleotides, about 4,600 nucleotides to about 10,500 nucleotides, about 4,600 nucleotides to about 10,000 nucleotides, about 4,600 nucleotides to about 9,500 nucleotides, about 4,600 nucleotides to about 9,000 nucleotides, about 4,600 nucleotides to about 8,500 nucleotides, about 4,600 nucleotides to about 8,000 nucleotides, about 4,600 nucleotides to about 7,800 nucleotides, about 4,600 nucleotides to about 7,600 nucleotides, about 4,600 nucleotides to about 7,400 nucleotides, about 4,600 nucleotides to about 7,200 nucleotides, about 4,600 nucleotides to about 7,000 nucleotides, about 4,600 nucleotides to about 6,800 nucleotides, about 4,600 nucleotides to about 6,600 nucleotides, about 4,600 nucleotides to about 6,400 nucleotides, about 4,600 nucleotides to about 6,200 nucleotides, about 4,600 nucleotides to about 6,000 nucleotides, about 4,600 nucleotides to about 5,800 nucleotides, about 4,600 nucleotides to about 5,600 nucleotides, about 4,600 nucleotides to about 5,400 nucleotides, about 4,600 nucleotides to about 5,200 nucleotides, about 4,600 nucleotides to about 5,000 nucleotides, about 4,600 nucleotides to about 4,800 nucleotides, about 4,800 nucleotides to about 15,000 nucleotides, about 4,800 nucleotides to about 14,500 nucleotides, about 4,800 nucleotides to about 14,000 nucleotides, about 4,800 nucleotides to about 13,500 nucleotides, about 4,800 nucleotides to about 13,000 nucleotides, about 4,800 nucleotides to about 12,500 nucleotides, about 4,800 nucleotides to about 12,000 nucleotides, about 4,800 nucleotides to about 11,500 nucleotides, about 4,800 nucleotides to about 11,000 nucleotides, about 4,800 nucleotides to about 10,500 nucleotides, about 4,800 nucleotides to about 10,000 nucleotides, about 4,800 nucleotides to about 9,500 nucleotides, about 4,800 nucleotides to about 9,000 nucleotides, about 4,800 nucleotides to about 8,500 nucleotides, about 4,800 nucleotides to about 8,000 nucleotides, about 4,800 nucleotides to about 7,800 nucleotides, about 4,800 nucleotides to about 7,600 nucleotides, about 4,800 nucleotides to about 7,400 nucleotides, about 4,800 nucleotides to about 7,200 nucleotides, about 4,800 nucleotides to about 7,000 nucleotides, about 4,800 nucleotides to about 6,800 nucleotides, about 4,800 nucleotides to about 6,600 nucleotides, about 4,800 nucleotides to about 6,400 nucleotides, about 4,800 nucleotides to about 6,200 nucleotides, about 4,800 nucleotides to about 6,000 nucleotides, about 4,800 nucleotides to about 5,800 nucleotides, about 4,800 nucleotides to about 5,600 nucleotides, about 4,800 nucleotides to about 5,400 nucleotides, about 4,800 nucleotides to about 5,200 nucleotides, about 4,800 nucleotides to about 5,000 nucleotides, about 5,000 nucleotides to about 15,000 nucleotides, about 5,000 nucleotides to about 14,500 nucleotides, about 5,000 nucleotides to about 14,000 nucleotides, about 5,000 nucleotides to about 13,500 nucleotides, about 5,000 nucleotides to about 13,000 nucleotides, about 5,000 nucleotides to about 12,500 nucleotides, about 5,000 nucleotides to about 12,000 nucleotides, about 5,000 nucleotides to about 11,500 nucleotides, about 5,000 nucleotides to about 11,000 nucleotides, about 5,000 nucleotides to about 10,500 nucleotides, about 5,000 nucleotides to about 10,000 nucleotides, about 5,000 nucleotides to about 9,500 nucleotides, about 5,000 nucleotides to about 9,000 nucleotides, about 5,000 nucleotides to about 8,500 nucleotides, about 5,000 nucleotides to about 8,000 nucleotides, about 5,000 nucleotides to about 7,800 nucleotides, about 5,000 nucleotides to about 7,600 nucleotides, about 5,000 nucleotides to about 7,400 nucleotides, about 5,000 nucleotides to about 7,200 nucleotides, about 5,000 nucleotides to about 7,000 nucleotides, about 5,000 nucleotides to about 6,800 nucleotides, about 5,000 nucleotides to about 6,600 nucleotides, about 5,000 nucleotides to about 6,400 nucleotides, about 5,000 nucleotides to about 6,200 nucleotides, about 5,000 nucleotides to about 6,000 nucleotides, about 5,000 nucleotides to about 5,800 nucleotides, about 5,000 nucleotides to about 5,600 nucleotides, about 5,000 nucleotides to about 5,400 nucleotides, about 5,000 nucleotides to about 5,200 nucleotides, about 5,200 nucleotides to about 15,000 nucleotides, about 5,200 nucleotides to about 14,500 nucleotides, about 5,200 nucleotides to about 14,000 nucleotides, about 5,200 nucleotides to about 13,500 nucleotides, about 5,200 nucleotides to about 13,000 nucleotides, about 5,200 nucleotides to about 12,500 nucleotides, about 5,200 nucleotides to about 12,000 nucleotides, about 5,200 nucleotides to about 11,500 nucleotides, about 5,200 nucleotides to about 11,000 nucleotides, about 5,200 nucleotides to about 10,500 nucleotides, about 5,200 nucleotides to about 10,000 nucleotides, about 5,200 nucleotides to about 9,500 nucleotides, about 5,200 nucleotides to about 9,000 nucleotides, about 5,200 nucleotides to about 8,500 nucleotides, about 5,200 nucleotides to about 8,000 nucleotides, about 5,200 nucleotides to about 7,800 nucleotides, about 5,200 nucleotides to about 7,600 nucleotides, about 5,200 nucleotides to about 7,400 nucleotides, about 5,200 nucleotides to about 7,200 nucleotides, about 5,200 nucleotides to about 7,000 nucleotides, about 5,200 nucleotides to about 6,800 nucleotides, about 5,200 nucleotides to about 6,600 nucleotides, about 5,200 nucleotides to about 6,400 nucleotides, about 5,200 nucleotides to about 6,200 nucleotides, about 5,200 nucleotides to about 6,000 nucleotides, about 5,200 nucleotides to about 5,800 nucleotides, about 5,200 nucleotides to about 5,600 nucleotides, about 5,200 nucleotides to about 5,400 nucleotides, about 5,400 nucleotides to about 15,000 nucleotides, about 5,400 nucleotides to about 14,500 nucleotides, about 5,400 nucleotides to about 14,000 nucleotides, about 5,400 nucleotides to about 13,500 nucleotides, about 5,400 nucleotides to about 13,000 nucleotides, about 5,400 nucleotides to about 12,500 nucleotides, about 5,400 nucleotides to about 12,000 nucleotides, about 5,400 nucleotides to about 11,500 nucleotides, about 5,400 nucleotides to about 11,000 nucleotides, about 5,400 nucleotides to about 10,500 nucleotides, about 5,400 nucleotides to about 10,000 nucleotides, about 5,400 nucleotides to about 9,500 nucleotides, about 5,400 nucleotides to about 9,000 nucleotides, about 5,400 nucleotides to about 8,500 nucleotides, about 5,400 nucleotides to about 8,000 nucleotides, about 5,400 nucleotides to about 7,800 nucleotides, about 5,400 nucleotides to about 7,600 nucleotides, about 5,400 nucleotides to about 7,400 nucleotides, about 5,400 nucleotides to about 7,200 nucleotides, about 5,400 nucleotides to about 7,000 nucleotides, about 5,400 nucleotides to about 6,800 nucleotides, about 5,400 nucleotides to about 6,600 nucleotides, about 5,400 nucleotides to about 6,400 nucleotides, about 5,400 nucleotides to about 6,200 nucleotides, about 5,400 nucleotides to about 6,000 nucleotides, about 5,400 nucleotides to about 5,800 nucleotides, about 5,400 nucleotides to about 5,600 nucleotides, about 5,600 nucleotides to about 15,000 nucleotides, about 5,600 nucleotides to about 14,500 nucleotides, about 5,600 nucleotides to about 14,000 nucleotides, about 5,600 nucleotides to about 13,500 nucleotides, about 5,600 nucleotides to about 13,000 nucleotides, about 5,600 nucleotides to about 12,500 nucleotides, about 5,600 nucleotides to about 12,000 nucleotides, about 5,600 nucleotides to about 11,500 nucleotides, about 5,600 nucleotides to about 11,000 nucleotides, about 5,600 nucleotides to about 10,500 nucleotides, about 5,600 nucleotides to about 10,000 nucleotides, about 5,600 nucleotides to about 9,500 nucleotides, about 5,600 nucleotides to about 9,000 nucleotides, about 5,600 nucleotides to about 8,500 nucleotides, about 5,600 nucleotides to about 8,000 nucleotides, about 5,600 nucleotides to about 7,800 nucleotides, about 5,600 nucleotides to about 7,600 nucleotides, about 5,600 nucleotides to about 7,400 nucleotides, about 5,600 nucleotides to about 7,200 nucleotides, about 5,600 nucleotides to about 7,000 nucleotides, about 5,600 nucleotides to about 6,800 nucleotides, about 5,600 nucleotides to about 6,600 nucleotides, about 5,600 nucleotides to about 6,400 nucleotides, about 5,600 nucleotides to about 6,200 nucleotides, about 5,600 nucleotides to about 6,000 nucleotides, about 5,600 nucleotides to about 5,800 nucleotides, about 5,800 nucleotides to about 15,000 nucleotides, about 5,800 nucleotides to about 14,500 nucleotides, about 5,800 nucleotides to about 14,000 nucleotides, about 5,800 nucleotides to about 13,500 nucleotides, about 5,800 nucleotides to about 13,000 nucleotides, about 5,800 nucleotides to about 12,500 nucleotides, about 5,800 nucleotides to about 12,000 nucleotides, about 5,800 nucleotides to about 11,500 nucleotides, about 5,800 nucleotides to about 11,000 nucleotides, about 5,800 nucleotides to about 10,500 nucleotides, about 5,800 nucleotides to about 10,000 nucleotides, about 5,800 nucleotides to about 9,500 nucleotides, about 5,800 nucleotides to about 9,000 nucleotides, about 5,800 nucleotides to about 8,500 nucleotides, about 5,800 nucleotides to about 8,000 nucleotides, about 5,800 nucleotides to about 7,800 nucleotides, about 5,800 nucleotides to about 7,600 nucleotides, about 5,800 nucleotides to about 7,400 nucleotides, about 5,800 nucleotides to about 7,200 nucleotides, about 5,800 nucleotides to about 7,000 nucleotides, about 5,800 nucleotides to about 6,800 nucleotides, about 5,800 nucleotides to about 6,600 nucleotides, about 5,800 nucleotides to about 6,400 nucleotides, about 5,800 nucleotides to about 6,200 nucleotides, about 5,800 nucleotides to about 6,000 nucleotides, about 6,000 nucleotides to about 15,000 nucleotides, about 6,000 nucleotides to about 14,500 nucleotides, about 6,000 nucleotides to about 14,000 nucleotides, about 6,000 nucleotides to about 13,500 nucleotides, about 6,000 nucleotides to about 13,000 nucleotides, about 6,000 nucleotides to about 12,500 nucleotides, about 6,000 nucleotides to about 12,000 nucleotides, about 6,000 nucleotides to about 11,500 nucleotides, about 6,000 nucleotides to about 11,000 nucleotides, about 6,000 nucleotides to about 10,500 nucleotides, about 6,000 nucleotides to about 10,000 nucleotides, about 6,000 nucleotides to about 9,500 nucleotides, about 6,000 nucleotides to about 9,000 nucleotides, about 6,000 nucleotides to about 8,500 nucleotides, about 6,000 nucleotides to about 8,000 nucleotides, about 6,000 nucleotides to about 7,800 nucleotides, about 6,000 nucleotides to about 7,600 nucleotides, about 6,000 nucleotides to about 7,400 nucleotides, about 6,000 nucleotides to about 7,200 nucleotides, about 6,000 nucleotides to about 7,000 nucleotides, about 6,000 nucleotides to about 6,800 nucleotides, about 6,000 nucleotides to about 6,600 nucleotides, about 6,000 nucleotides to about 6,400 nucleotides, about 6,000 nucleotides to about 6,200 nucleotides, about 6,200 nucleotides to about 15,000 nucleotides, about 6,200 nucleotides to about 14,500 nucleotides, about 6,200 nucleotides to about 14,000 nucleotides, about 6,200 nucleotides to about 13,500 nucleotides, about 6,200 nucleotides to about 13,000 nucleotides, about 6,200 nucleotides to about 12,500 nucleotides, about 6,200 nucleotides to about 12,000 nucleotides, about 6,200 nucleotides to about 11,500 nucleotides, about 6,200 nucleotides to about 11,000 nucleotides, about 6,200 nucleotides to about 10,500 nucleotides, about 6,200 nucleotides to about 10,000 nucleotides, about 6,200 nucleotides to about 9,000 nucleotides, about 6,200 nucleotides to about 8,500 nucleotides, about 6,200 nucleotides to about 8,000 nucleotides, about 6,200 nucleotides to about 7,800 nucleotides, about 6,200 nucleotides to about 7,600 nucleotides, about 6,200 nucleotides to about 7,400 nucleotides, about 6,200 nucleotides to about 7,200 nucleotides, about 6,200 nucleotides to about 7,000 nucleotides, about 6,200 nucleotides to about 6,800 nucleotides, about 6,200 nucleotides to about 6,600 nucleotides, about 6,200 nucleotides to about 6,400 nucleotides, about 6,400 nucleotides to about 15,000 nucleotides, about 6,400 nucleotides to about 14,500 nucleotides, about 6,400 nucleotides to about 14,000 nucleotides, about 6,400 nucleotides to about 13,500 nucleotides, about 6,400 nucleotides to about 13,000 nucleotides, about 6,400 nucleotides to about 12,500 nucleotides, about 6,400 nucleotides to about 12,000 nucleotides, about 6,400 nucleotides to about 11,500 nucleotides, about 6,400 nucleotides to about 11,000 nucleotides, about 6,400 nucleotides to about 10,500 nucleotides, about 6,400 nucleotides to about 10,000 nucleotides, about 6,400 nucleotides to about 9,500 nucleotides, about 6,400 nucleotides to about 9,000 nucleotides, about 6,400 nucleotides to about 8,500 nucleotides, about 6,400 nucleotides to about 8,000 nucleotides, about 6,400 nucleotides to about 7,800 nucleotides, about 6,400 nucleotides to about 7,600 nucleotides, about 6,400 nucleotides to about 7,400 nucleotides, about 6,400 nucleotides to about 7,200 nucleotides, about 6,400 nucleotides to about 7,000 nucleotides, about 6,400 nucleotides to about 6,800 nucleotides, about 6,400 nucleotides to about 6,600 nucleotides, about 6,600 nucleotides to about 15,000 nucleotides, about 6,600 nucleotides to about 14,500 nucleotides, about 6,600 nucleotides to about 14,000 nucleotides, about 6,600 nucleotides to about 13,500 nucleotides, about 6,600 nucleotides to about 13,000 nucleotides, about 6,600 nucleotides to about 12,500 nucleotides, about 6,600 nucleotides to about 12,000 nucleotides, about 6,600 nucleotides to about 11,500 nucleotides, about 6,600 nucleotides to about 11,000 nucleotides, about 6,600 nucleotides to about 10,500 nucleotides, about 6,600 nucleotides to about 10,000 nucleotides, about 6,600 nucleotides to about 9,500 nucleotides, about 6,600 nucleotides to about 9,000 nucleotides, about 6,600 nucleotides to about 8,500 nucleotides, about 6,600 nucleotides to about 8,000 nucleotides, about 6,600 nucleotides to about 7,800 nucleotides, about 6,600 nucleotides to about 7,600 nucleotides, about 6,600 nucleotides to about 7,400 nucleotides, about 6,600 nucleotides to about 7,200 nucleotides, about 6,600 nucleotides to about 7,000 nucleotides, about 6,600 nucleotides to about 6,800 nucleotides, about 6,800 nucleotides to about 15,000 nucleotides, about 6,800 nucleotides to about 14,500 nucleotides, about 6,800 nucleotides to about 14,000 nucleotides, about 6,800 nucleotides to about 13,500 nucleotides, about 6,800 nucleotides to about 13,000 nucleotides, about 6,800 nucleotides to about 12,500 nucleotides, about 6,800 nucleotides to about 12,000 nucleotides, about 6,800 nucleotides to about 11,500 nucleotides, about 6,800 nucleotides to about 11,000 nucleotides, about 6,800 nucleotides to about 10,500 nucleotides, about 6,800 nucleotides to about 10,000 nucleotides, about 6,800 nucleotides to about 9,500 nucleotides, about 6,800 nucleotides to about 9,000 nucleotides, about 6,800 nucleotides to about 8,500 nucleotides, about 6,800 nucleotides to about 8,000 nucleotides, about 6,800 nucleotides to about 7,800 nucleotides, about 6,800 nucleotides to about 7,600 nucleotides, about 6,800 nucleotides to about 7,400 nucleotides, about 6,800 nucleotides to about 7,200 nucleotides, about 6,800 nucleotides to about 7,000 nucleotides, about 7,000 nucleotides to about 15,000 nucleotides, about 7,000 nucleotides to about 14,500 nucleotides, about 7,000 nucleotides to about 14,000 nucleotides, about 7,000 nucleotides to about 13,500 nucleotides, about 7,000 nucleotides to about 13,000 nucleotides, about 7,000 nucleotides to about 12,500 nucleotides, about 7,000 nucleotides to about 12,000 nucleotides, about 7,000 nucleotides to about 11,500 nucleotides, about 7,000 nucleotides to about 11,000 nucleotides, about 7,000 nucleotides to about 10,500 nucleotides, about 7,000 nucleotides to about 10,000 nucleotides, about 7,000 nucleotides to about 9,500 nucleotides, about 7,000 nucleotides to about 9,000 nucleotides, about 7,000 nucleotides to about 8,500 nucleotides, about 7,000 nucleotides to about 8,000 nucleotides, about 7,000 nucleotides to about 7,800 nucleotides, about 7,000 nucleotides to about 7,600 nucleotides, about 7,000 nucleotides to about 7,400 nucleotides, about 7,000 nucleotides to about 7,200 nucleotides, about 7,200 nucleotides to about 15,000 nucleotides, about 7,200 nucleotides to about 14,500 nucleotides, about 7,200 nucleotides to about 14,000 nucleotides, about 7,200 nucleotides to about 13,500 nucleotides, about 7,200 nucleotides to about 13,000 nucleotides, about 7,200 nucleotides to about 12,500 nucleotides, about 7,200 nucleotides to about 12,000 nucleotides, about 7,200 nucleotides to about 11,500 nucleotides, about 7,200 nucleotides to about 11,000 nucleotides, about 7,200 nucleotides to about 10,500 nucleotides, about 7,200 nucleotides to about 10,000 nucleotides, about 7,200 nucleotides to about 9,500 nucleotides, about 7,200 nucleotides to about 9,000 nucleotides, about 7,200 nucleotides to about 8,500 nucleotides, about 7,200 nucleotides to about 8,000 nucleotides, about 7,200 nucleotides to about 7,800 nucleotides, about 7,200 nucleotides to about 7,600 nucleotides, about 7,200 nucleotides to about 7,400 nucleotides, about 7,400 nucleotides to about 15,000 nucleotides, about 7,400 nucleotides to about 14,500 nucleotides, about 7,400 nucleotides to about 14,000 nucleotides, about 7,400 nucleotides to about 13,500 nucleotides, about 7,400 nucleotides to about 13,000 nucleotides, about 7,400 nucleotides to about 12,500 nucleotides, about 7,400 nucleotides to about 12,000 nucleotides, about 7,400 nucleotides to about 11,500 nucleotides, about 7,400 nucleotides to about 11,000 nucleotides, about 7,400 nucleotides to about 10,500 nucleotides, about 7,400 nucleotides to about 10,000 nucleotides, about 7,400 nucleotides to about 9,500 nucleotides, about 7,400 nucleotides to about 9,000 nucleotides, about 7,400 nucleotides to about 8,500 nucleotides, about 7,400 nucleotides to about 8,000 nucleotides, about 7,400 nucleotides to about 7,800 nucleotides, about 7,400 nucleotides to about 7,600 nucleotides, about 7,600 nucleotides to about 15,000 nucleotides, about 7,600 nucleotides to about 14,500 nucleotides, about 7,600 nucleotides to about 14,000 nucleotides, about 7,600 nucleotides to about 13,500 nucleotides, about 7,600 nucleotides to about 13,000 nucleotides, about 7,600 nucleotides to about 12,500 nucleotides, about 7,600 nucleotides to about 12,000 nucleotides, about 7,600 nucleotides to about 11,500 nucleotides, about 7,600 nucleotides to about 11,000 nucleotides, about 7,600 nucleotides to about 10,500 nucleotides, about 7,600 nucleotides to about 10,000 nucleotides, about 7,600 nucleotides to about 9,500 nucleotides, about 7,600 nucleotides to about 9,000 nucleotides, about 7,600 nucleotides to about 8,500 nucleotides, about 7,600 nucleotides to about 8,000 nucleotides, about 7,600 nucleotides to about 7,800 nucleotides, about 7,800 nucleotides to about 15,000 nucleotides, about 7,800 nucleotides to about 14,500 nucleotides, about 7,800 nucleotides to about 14,000 nucleotides, about 7,800 nucleotides to about 13,500 nucleotides, about 7,800 nucleotides to about 13,000 nucleotides, about 7,800 nucleotides to about 12,500 nucleotides, about 7,800 nucleotides to about 12,000 nucleotides, about 7,800 nucleotides to about 11,500 nucleotides, about 7,800 nucleotides to about 11,000 nucleotides, about 7,800 nucleotides to about 10,500 nucleotides, about 7,800 nucleotides to about 10,000 nucleotides, about 7,800 nucleotides to about 9,500 nucleotides, about 7,800 nucleotides to about 9,000 nucleotides, about 7,800 nucleotides to about 8,500 nucleotides, about 7,800 nucleotides to about 8,000 nucleotides, about 8,000 nucleotides to about 15,000 nucleotides, about 8,000 nucleotides to about 14,500 nucleotides, about 8,000 nucleotides to about 14,000 nucleotides, about 8,000 nucleotides to about 13,500 nucleotides, about 8,000 nucleotides to about 13,000 nucleotides, about 8,000 nucleotides to about 12,500 nucleotides, about 8,000 nucleotides to about 12,000 nucleotides, about 8,000 nucleotides to about 11,500 nucleotides, about 8,000 nucleotides to about 11,000 nucleotides, about 8,000 nucleotides to about 10,500 nucleotides, about 8,000 nucleotides to about 10,000 nucleotides, about 8,000 nucleotides to about 9,500 nucleotides, about 8,000 nucleotides to about 9,000 nucleotides, about 8,000 nucleotides to about 8,500 nucleotides, about 8,500 nucleotides to about 15,000 nucleotides, about 8,500 nucleotides to about 14,500 nucleotides, about 8,500 nucleotides to about 14,000 nucleotides, about 8,500 nucleotides to about 13,500 nucleotides, about 8,500 nucleotides to about 13,000 nucleotides, about 8,500 nucleotides to about 12,500 nucleotides, about 8,500 nucleotides to about 12,000 nucleotides, about 8,500 nucleotides to about 11,500 nucleotides, about 8,500 nucleotides to about 11,000 nucleotides, about 8,500 nucleotides to about 10,500 nucleotides, about 8,500 nucleotides to about 10,000 nucleotides, about 8,500 nucleotides to about 9,500 nucleotides, about 8,500 nucleotides to about 9,000 nucleotides, about 9,000 nucleotides to about 15,000 nucleotides, about 9,000 nucleotides to about 14,500 nucleotides, about 9,000 nucleotides to about 14,000 nucleotides, about 9,000 nucleotides to about 13,500 nucleotides, about 9,000 nucleotides to about 13,000 nucleotides, about 9,000 nucleotides to about 12,500 nucleotides, about 9,000 nucleotides to about 12,000 nucleotides, about 9,000 nucleotides to about 11,500 nucleotides, about 9,000 nucleotides to about 11,000 nucleotides, about 9,000 nucleotides to about 10,500 nucleotides, about 9,000 nucleotides to about 10,000 nucleotides, about 9,000 nucleotides to about 9,500 nucleotides, about 9,500 nucleotides to about 10,000 nucleotides, about 10,000 nucleotides to about 15,000 nucleotides, about 10,000 nucleotides to about 14,500 nucleotides, about 10,000 nucleotides to about 14,000 nucleotides, about 10,000 nucleotides to about 13,500 nucleotides, about 10,000 nucleotides to about 13,000 nucleotides, about 10,000 nucleotides to about 12,500 nucleotides, about 10,000 nucleotides to about 12,000 nucleotides, about 10,000 nucleotides to about 11,500 nucleotides, about 10,000 nucleotides to about 11,000 nucleotides, about 10,000 nucleotides to about 10,500 nucleotides, about 10,500 nucleotides to about 15,000 nucleotides, about 10,500 nucleotides to about 14,500 nucleotides, about 10,500 nucleotides to about 14,000 nucleotides, about 10,500 nucleotides to about 13,500 nucleotides, about 10,500 nucleotides to about 13,000 nucleotides, about 10,500 nucleotides to about 12,500 nucleotides, about 10,500 nucleotides to about 12,000 nucleotides, about 10,500 nucleotides to about 11,500 nucleotides, about 10,500 nucleotides to about 11,000 nucleotides, about 11,000 nucleotides to about 15,000 nucleotides, about 11,000 nucleotides to about 14,500 nucleotides, about 11,000 nucleotides to about 14,000 nucleotides, about 11,000 nucleotides to about 13,500 nucleotides, about 11,000 nucleotides to about 13,000 nucleotides, about 11,000 nucleotides to about 12,500 nucleotides, about 11,000 nucleotides to about 12,000 nucleotides, about 11,000 nucleotides to about 11,500 nucleotides, about 11,500 nucleotides to about 15,000 nucleotides, about 11,500 nucleotides to about 14,500 nucleotides, about 11,500 nucleotides to about 14,000 nucleotides, about 11,500 nucleotides to about 13,500 nucleotides, about 11,500 nucleotides to about 13,000 nucleotides, about 11,500 nucleotides to about 12,500 nucleotides, about 11,500 nucleotides to about 12,000 nucleotides, about 12,000 nucleotides to about 15,000 nucleotides, about 12,000 nucleotides to about 14,500 nucleotides, about 12,000 nucleotides to about 14,000 nucleotides, about 12,000 nucleotides to about 13,500 nucleotides, about 12,000 nucleotides to about 13,000 nucleotides, about 12,000 nucleotides to about 12,500 nucleotides, about 12,500 nucleotides to about 15,000 nucleotides, about 12,500 nucleotides to about 14,500 nucleotides, about 12,500 nucleotides to about 14,000 nucleotides, about 12,500 nucleotides to about 13,500 nucleotides, about 12,500 nucleotides to about 13,000 nucleotides, about 13,000 nucleotides to about 15,000 nucleotides, about 13,000 nucleotides to about 14,500 nucleotides, about 13,000 nucleotides to about 14,000 nucleotides, about 13,000 nucleotides to about 13,500 nucleotides, about 13,500 nucleotides to about 15,000 nucleotides, about 13,500 nucleotides to about 14,500 nucleotides, about 13,500 nucleotides to about 14,000 nucleotides, about 14,000 nucleotides to about 15,000 nucleotides, about 14,000 nucleotides to about 14,500 nucleotides, or about 14,500 nucleotides to about 15,000 nucleotides (inclusive).

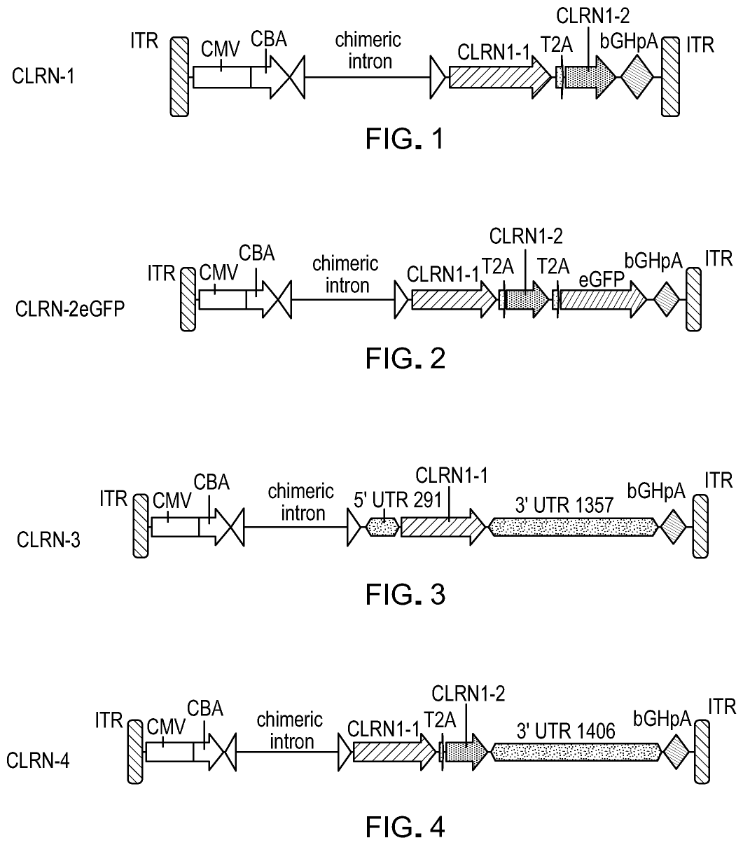

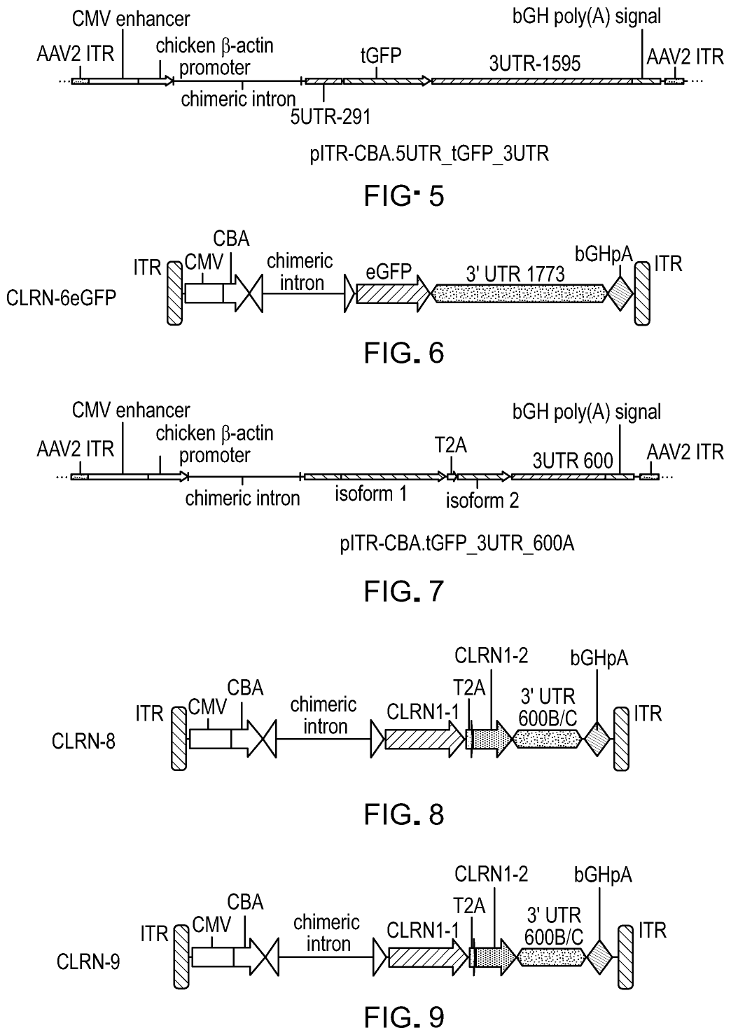

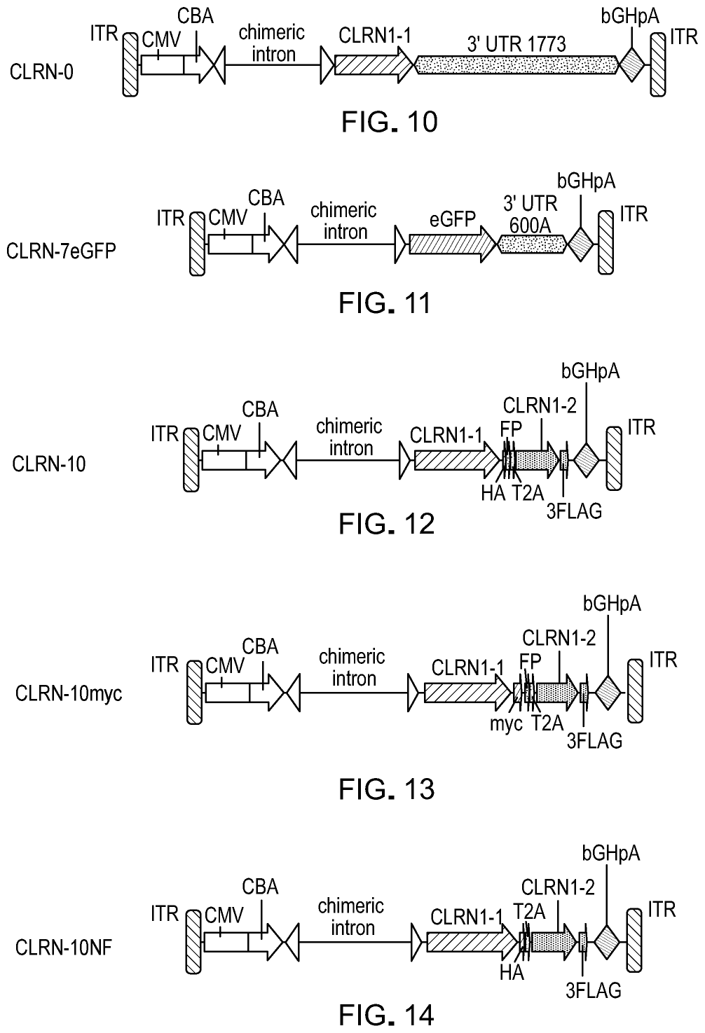

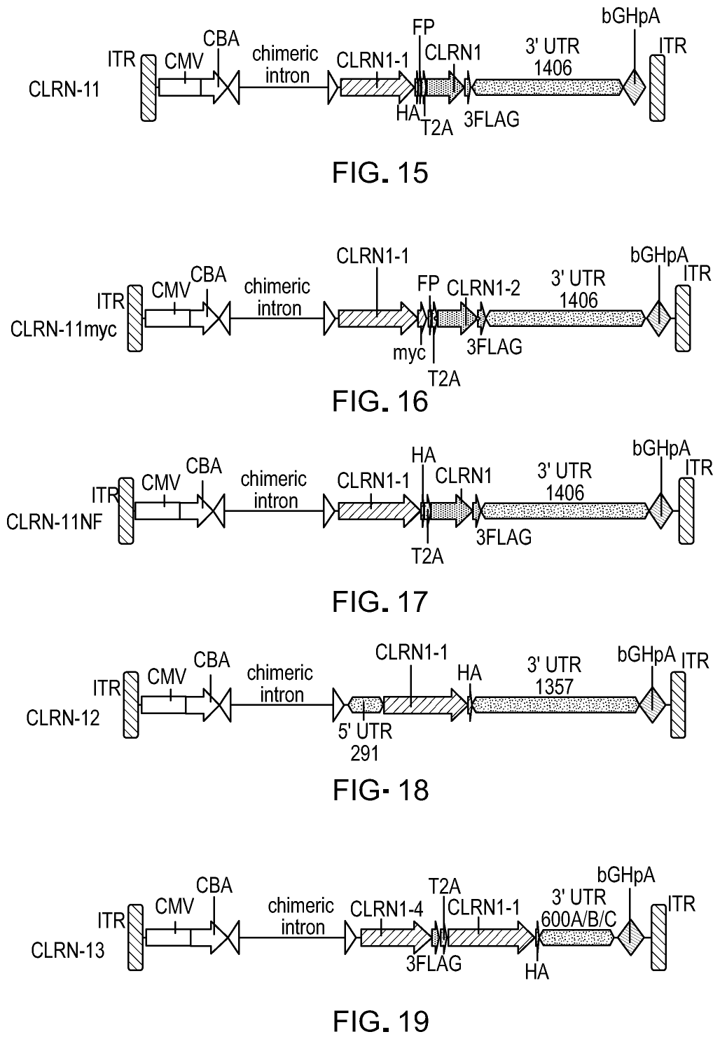

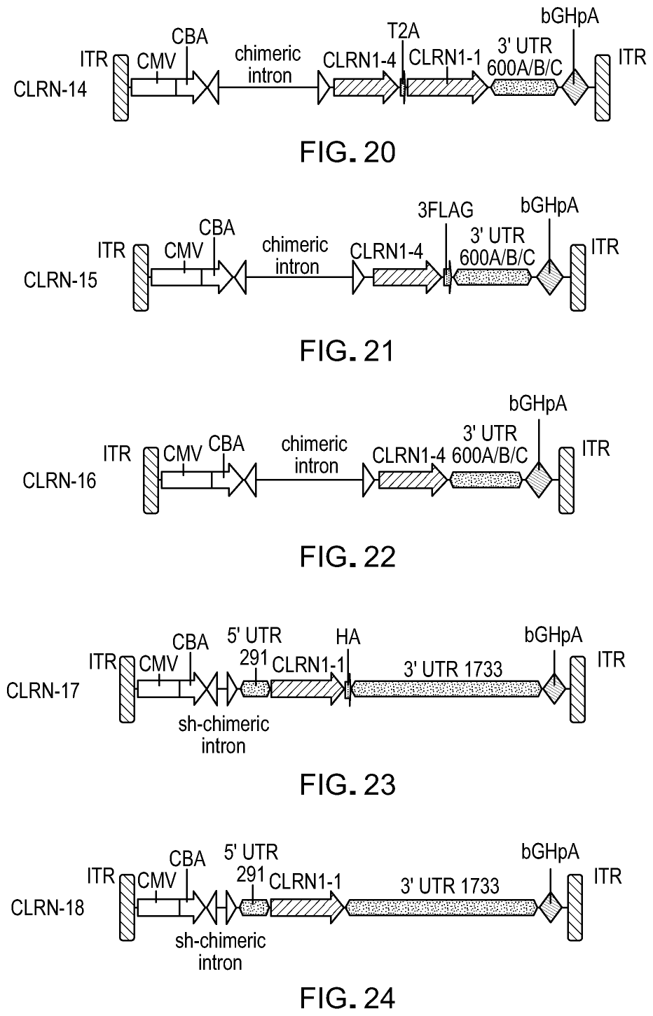

Provided herein are exemplary vectors that can be used in any of the compositions and methods described herein. See, e.g., FIGS. 1-24.

A variety of different methods known in the art can be used to introduce any of vectors disclosed herein into a mammalian cell (e.g., a cochlear inner hair cell, a cochlear outer hair cell, a retinal cell). Non-limiting examples of methods for introducing nucleic acid into a mammalian cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection.

Skilled practitioners will appreciate that any of the vectors described herein can be introduced into a mammalian cell by, for example, lipofection, and can be stably integrated into an endogenous gene locus (e.g., a CLRN1 gene locus). In some embodiments, the vectors provided herein stably integrate into an endogenous defective CLRN1 gene locus, and thereby replace the defective CLRN1 gene with a nucleic acid encoding a functioning (e.g., wildtype) CLRN1 protein.

Various molecular biology techniques that can be used to introduce a mutation(s) and/or a deletion(s) into an endogenous gene are also known in the art. Non-limiting examples of such techniques include site-directed mutagenesis, CRISPR (e.g., CRISPR/Cas9-induced knock-in mutations and CRISPR/Cas9-induced knock-out mutations), and TALENs. These methods can be used to correct the sequence of a defective endogenous gene present in a chromosome of a target cell.

Any of the vectors described herein can further include a control sequence, e.g., a control sequence selected from the group of a transcription initiation sequence, a transcription termination sequence, a promoter sequence, an enhancer sequence, an RNA splicing sequence, a polyadenylation (polyA) signal, and a Kozak consensus sequence. Non-limiting examples of these control sequences are described herein. In some embodiments, a promoter can be a native promoter, a constitutive promoter, an inducible promoter, and/or a tissue-specific promoter.

### Promoters

The term “promoter” means a DNA sequence recognized by enzymes/proteins in a mammalian cell required to initiate the transcription of a specific gene (e.g., a CLRN1 gene). A promoter typically refers to, e.g., a nucleotide sequence to which an RNA polymerase and/or any associated factor binds and at which transcription is initiated. Non-limiting examples of promoters are described herein. Additional examples of promoters are known in the art.

In some embodiments, a vector encoding an N-terminal portion of a CLRN1 protein (e.g., a human CLRN1 protein) can include a promoter and/or an enhancer. The vector encoding the N-terminal portion of the CLRN1 protein can include any of the promoters and/or enhancers described herein or known in the art.

In some embodiments, the promoter is an inducible promoter, a constitutive promoter, a mammalian cell promoter, a viral promoter, a chimeric promoter, an engineered promoter, a tissue-specific promoter, or any other type of promoter known in the art. In some embodiments, the promoter is a RNA polymerase II promoter, such as a mammalian RNA polymerase II promoter. In some embodiments, the promoter is a RNA polymerase III promoter, including, but not limited to, a H1 promoter, a human U6 promoter, a mouse U6 promoter, or a swine U6 promoter. The promoter will generally be one that is able to promote transcription in an inner hair cell In some examples, the promoter is a cochlea-specific promoter or a cochlea-oriented promoter.

A variety of promoters are known in the art that can be used herein. Non-limiting examples of promoters that can be used herein include: human EF1a, human cytomegalovirus (CMV) (U.S. Pat. No. 5,168,062), human ubiquitin C (UBC), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g. human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2κb, HSP70, proliferin, tumor necrosis factor, thyroid stimulating hormone a gene, immunoglobulin light chain, T-cell receptor, HLA DQα and DQβ, interleukin-2 receptor, MHC class II, MHC class II HLA-DRα, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, and Gibbon Ape Leukemia Virus (GALV) promoters. Additional examples of promoters are known in the art. See, e.g., Lodish, Molecular Cell Biology, Freeman and Company, New York 2007. In some embodiments, the promoter is the CMV immediate early promoter. In some embodiments, the promoter is a CAG promoter or a CAG/CBA promoter. In some embodiments, the promoter is a CBA promoter, e.g., a CBA promoter comprising or consisting of SEQ ID NO: 18.

The term “constitutive” promoter refers to a nucleotide sequence that, when operably linked with a nucleic acid encoding a protein (e.g., a CLRN1 protein), causes RNA to be transcribed from the nucleic acid in a mammalian cell under most or all physiological conditions.

Examples of constitutive promoters include, without limitation, the retroviral Rous sarcoma virus (RSV) LTR promoter, the cytomegalovirus (CMV) promoter (see, e.g., Boshart et al, Cell 41:521-530, 1985), the SV40 promoter, the dihydrofolate reductase promoter, the beta-actin promoter, the phosphoglycerol kinase (PGK) promoter, and the EF1-alpha promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Additional examples of inducible promoters are known in the art.

Examples of inducible promoters regulated by exogenously supplied compounds include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. U.S.A. 93:3346-3351, 1996), the tetracycline-repressible system (Gossen et al, Proc. Natl. Acad. Sci. U.S.A. 89:5547-5551, 1992), the tetracycline-inducible system (Gossen et al, Science 268:1766-1769, 1995, see also Harvey et al, Curr. Opin. Chem. Biol. 2:512-518, 1998), the RU486-inducible system (Wang et al, Nat. Biotech. 15:239-243, 1997) and Wang et al, Gene Ther. 4:432-441, 1997), and the rapamycin-inducible system (Magari et al. J. Clin. Invest. 100:2865-2872, 1997).

The term “tissue-specific” promoter refers to a promoter that is active only in certain specific cell types and/or tissues (e.g., transcription of a specific gene occurs only within cells expressing transcription regulatory proteins that bind to the tissue-specific promoter).

In some embodiments, the regulatory sequences impart tissue-specific gene expression capabilities. In some cases, the tissue-specific regulatory sequences bind tissue-specific transcription factors that induce transcription in a tissue-specific manner.

Exemplary tissue-specific promoters include but are not limited to the following: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, an alpha-myosin heavy chain (a-MHC) promoter, and a cardiac Troponin T (cTnT) promoter. Additional exemplary promoters include Beta-actin promoter, hepatitis B virus core promoter (Sandig et al., Gene Ther. 3:1002-1009, 1996), alpha-fetoprotein (AFP) promoter (Arbuthnot et al., Hum. Gene Ther. 7:1503-1514, 1996), bone osteocalcin promoter (Stein et al., Mol. Biol. Rep. 24:185-196, 1997); bone sialoprotein promoter (Chen et al., J. Bone Miner. Res. 11:654-664, 1996), CD2 promoter (Hansal et al., J. Immunol. 161:1063-1068, 1998); immunoglobulin heavy chain promoter; T cell receptor alpha-chain promoter, neuronal such as neuron-specific enolase (NSE) promoter (Andersen et al., Cell. Mol. Neurobiol. 13:503-515, 1993), neurofilament light-chain gene promoter (Piccioli et al., Proc. Natl. Acad. Sci. U.S.A. 88:5611-5615, 1991), and the neuron-specific vgf gene promoter (Piccioli et al., Neuron 15:373-384, 1995).

In some embodiments, the tissue-specific promoter is a cochlea-specific promoter. In some embodiments, the tissue-specific promoter is a cochlear hair cell-specific promoter. Non-limiting examples of cochlear hair cell-specific promoters include but are not limited to: a ATOH1 promoter, a POU4F3 promoter, a LHX3 promoter, a MYO7A promoter, a MYO6 promoter, a α9ACHR promoter, and a α10ACHR promoter. In some embodiments, the promoter is an cochlear hair cell-specific promoter such as a PRESTIN promoter or an ONCOMOD promoter. See, e.g., Zheng et al., Nature 405:149-155, 2000; Tian et al. Dev. Dyn. 231:199-203, 2004; and Ryan et al., Adv. Otorhinolaryngol. 66: 99-115, 2009.

### Enhancers

In some instances, a vector can include an enhancer sequence. The term “enhancer” refers to a nucleotide sequence that can increase the level of transcription of a nucleic acid encoding a protein of interest (e.g., a CLRN1 protein). Enhancer sequences (50-1500 basepairs in length) generally increase the level of transcription by providing additional binding sites for transcription-associated proteins (e.g., transcription factors). In some embodiments, an enhancer sequence is found within an intronic sequence. Unlike promoter sequences, enhancer sequences can act at much larger distance away from the transcription start site (e.g., as compared to a promoter). Non-limiting examples of enhancers include a RSV enhancer, a CMV enhancer, and a SV40 enhancer. In some embodiments, the CMV enhancer sequence comprises or consists of SEQ ID NO: 17.

### Poly(A) Signal

In some embodiments, any of the vectors provided herein can include a polyadenylation (poly(A)) signal sequence. Most nascent eukaryotic mRNAs possess a poly(A) tail at their 3′ end which is added during a complex process that includes cleavage of the primary transcript and a coupled polyadenylation reaction driven by the poly(A) signal sequence (see, e.g., Proudfoot et al., Cell 108:501-512, 2002). The poly(A) tail confers mRNA stability and transferability (Molecular Biology of the Cell, Third Edition by B. Alberts et al., Garland Publishing, 1994). In some embodiments, the poly(A) signal sequence is positioned 3′ to the nucleic acid sequence encoding the C-terminus of the CLRN1 protein.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In higher eukaryotes, the poly(A) tail is added onto transcripts that contain a specific sequence, the polyadenylation (or poly(A)) signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but also can occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used, including those derived from bovine growth hormone (bgh) (Woychik et al., Proc. Natl. Acad. Sci. U.S.A. 81(13):3944-3948, 1984; U.S. Pat. No. 5,122,458), mouse-β-globin, mouse-α-globin (Orkin et al., EMBO J. 4(2):453-456, 1985; Thein et al., Blood 71(2):313-319, 1988), human collagen, polyoma virus (Batt et al., Mol. Cell Biol. 15(9):4783-4790, 1995), the Herpes simplex virus thymidine kinase gene (HSV TK), IgG heavy-chain gene polyadenylation signal (US 2006/0040354), human growth hormone (hGH) (Szymanski et al., Mol. Therapy 15(7):1340-1347, 2007), the group consisting of SV40 poly(A) site, such as the SV40 late and early poly(A) site (Schek et al., Mol. Cell Biol. 12(12):5386-5393, 1992).

The poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414).

In some embodiments, the poly(A) signal sequence can be a synthetic polyadenylation site (see, e.g., the pCl-neo expression vector of Promega that is based on Levitt el al, Genes Dev. 3(7):1019-1025, 1989). In some embodiments, the poly(A) signal sequence is the polyadenylation signal of bovine growth hormone (CTGTGCCTTCTAGTTGCCAGCCATCTGTTGTTTGCCCCTCCCCCGTGCCTTCC TTGACCCTGGAAGGTGCCACTCCCACTGTCCTTTCCTAATAAAATGAGGAAAT TGCATCGCATTGTCTGAGTAGGTGTCATTCTATTCTGGGGGGTGGGGTGGGGC AGGACAGCAAGGGGGAGGATTGGGAAGACAATAGCAGGCATGCTGGGGATG CGGTGGGCTCTATGG (SEQ ID NO: 20)). In some embodiments, the poly(A) signal sequence is the polyadenylation signal of soluble neuropilin-1 (sNRP) (AAATAAAATACGAAATG (SEQ ID NO: 21)) (see, e.g., WO 05/073384). Additional examples of poly(A) signal sequences are known in the art.

### Internal Ribosome Entry Site (IRES)

In some embodiments, a vector encoding the C-terminal portion of the CLRN1 protein can include a polynucleotide internal ribosome entry site (IRES). An IRES sequence is used to produce more than one polypeptide from a single gene transcript. An IRES forms a complex secondary structure that allows translation initiation to occur from any position with an mRNA immediately downstream from where the IRES is located (see, e.g., Pelletier and Sonenberg, Mol. Cell. Biol. 8(3):1103-1112, 1988).

There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV). See e.g., Alberts, Molecular Biology of the Cell, Garland Science, 2002; and Hellen et al., Genes Dev. 15(13):1593-612, 2001.

In some embodiments, the IRES sequence that is incorporated into the vector that encodes the C-terminal portion of a CLRN1 protein is the foot and mouth diseause virus (FMDV) 2A sequence. The Foot and Mouth Disease Virus 2A sequence is a small peptide (approximately 18 amino acids in length) that has been shown to mediate the cleavage of polyproteins (Ryan, M D et al., EMBO 4:928-933, 1994; Mattion et al., J. Virology 70:8124-8127, 1996; Furler et al., Gene Therapy 8:864-873, 2001; and Halpin et al., Plant Journal 4:453-459, 1999). The cleavage activity of the 2A sequence has previously been demonstrated in artificial systems including plasmids and gene therapy vectors (AAV and retroviruses) (Ryan et al., EMBO 4:928-933, 1994; Mattion et al., J. Virology 70:8124-8127, 1996; Furler et al., Gene Therapy 8:864-873, 2001; and Halpin et al., Plant Journal 4:453-459, 1999; de Felipe et al., Gene Therapy 6:198-208, 1999; de Felipe et al., Human Gene Therapy 11:1921-1931, 2000; and Klump et al., Gene Therapy 8:811-817, 2001).

### Reporter Sequences

Any of the vectors provided herein can optionally include a sequence encoding a reporter protein (“a reporter sequence”). Non-limiting examples of reporter sequences include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. When associated with regulatory elements which drive their expression, the reporter sequence can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some embodiments, the reporter sequence is tGFP (SEQ ID NO: 19). In some embodiments, the reporter sequence is the LacZ gene, and the presence of a vector carrying the LacZ gene in a mammalian cell (e.g., a cochlear hair cell, an ocular cell, such as a retinal cell) is detected by assays for beta-galactosidase activity. When the reporter is a fluorescent protein (e.g., green fluorescent protein) or luciferase, the presence of a vector carrying the fluorescent protein or luciferase in a mammalian cell (e.g., a cochlear hair cell, an ocular cell, such as a retinal cell) may be measured by fluorescent techniques (e.g., fluorescent microscopy or FACS) or light production in a luminometer (e.g., a spectrophotometer or an IVIS imaging instrument). In some embodiments, the reporter sequence can be used to verify the tissue-specific targeting capabilities and tissue-specific promoter regulatory activity of any of the vectors described herein.

### Flanking Regions Untranslated Regions (UTRs)

In some embodiments, any of the vectors described herein (e.g., any of the at least two different vectors) can include an untranslated region, such as a 5′ UTR or a 3′ UTR.

Untranslated regions (UTRs) of a gene are transcribed but not translated. The 5′ UTR starts at the transcription start site and continues to the start codon but does not include the start codon. The 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into any of the vectors, compositions, kits, or methods as described herein to enhance the expression of a CLRN1 protein.

Natural 5′ UTRs include a sequence that plays a role in translation initiation. They harbor signatures like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. The 5′ UTRs have also been known to form secondary structures that are involved in elongation factor binding.

In some embodiments, a 5′ UTR is included in any of the vectors described herein. Non-limiting examples of 5′ UTRs, including those from the following genes: albumin, serum amyloid A, Apolipoprotein A/B/E, transferrin, alpha fetoprotein, erythropoietin, and Factor VIII, can be used to enhance expression of a nucleic acid molecule, such as a mRNA.

In some embodiments, a 5′ UTR from a mRNA that is transcribed by a cell in the cochlea or retina can be included in any of the vectors, compositions, kits, and methods described herein.

3′ UTRs are known to have stretches of adenosines and uridines (in the RNA form) or thymidines (in the DNA form) embedded in them. These AU-rich signatures are particularly prevalent in genes with high rates of turnover. Based on their sequence features and functional properties, the AU-rich elements (AREs) can be separated into three classes (Chen et al., Mol. Cell. Biol. 15:5777-5788, 1995; Chen et al., Mol. Cell Biol. 15:2010-2018, 1995): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif. Two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

An exemplary human wildtype 5′ UTR is or includes the sequence of SEQ ID NO: 12 or SEQ ID NO: 13. An exemplary human wildtype 5′ UTR is or includes the sequence of SEQ ID NO: 14 or SEQ ID NO: 15.

In some embodiments of any of the compositions described herein, a 5′ untranslated region (UTR), a 3′ UTR, or both are included in a vector (e.g., any of the vectors described herein). For example, any of the 5′-UTRs described herein can be operatively linked to the start codon in any of the coding sequences described herein. For example, any of the 3′-UTR's can be operately linked to the 3′-terminal codon (last codon) in any of the coding sequences described herein.

In some embodiments of any of the compositions described herein, the 5′ UTR comprises at least 10 contiguous (e.g., at least 15 contiguous, at least 20 contiguous, at least 25 contiguous, at least 30 contiguous, at least 35 contiguous, at least 40 contiguous, at least 45 contiguous, at least 50 contiguous, at least 55 contiguous, at least 60 contiguous, at least 65 contiguous, at least 70 contiguous, at least 75 contiguous, at least 80 contiguous, at least 85 contiguous, at least 90 contiguous, at least 100 contiguous, at least 105 contiguous, at least 110 contiguous, at least 115 contiguous, at least 120 contiguous, at least 125 contiguous, at least 130 contiguous, at least 135 contiguous, at least 140 contiguous, at least 145 contiguous, at least 150 contiguous, at least 155 contiguous, at least 160 contiguous, at least 165 contiguous, at least 170 contiguous, at least 175 contiguous, at least 180 contiguous, at least 185 contiguous, at least 190 contiguous, at least 195 contiguous, at least 200 contiguous, at least 205 contiguous, at least 210 contiguous, at least 215 contiguous, at least 220 contiguous, at least 225 contiguous, at least 230 contiguous, at least 235 contiguous, at least 240 contiguous, at least 245 contiguous, at least 250 contiguous, at least 255 contiguous, or at least 260 contiguous) nucleotides from anywhere within SEQ ID NO: 12 or SEQ ID NO: 13.

For example, a 5′ UTR can include or consist of one or more of: nucleotide positions 1 to 291, nucleotide positions 1 to 290, nucleotide positions 1 to 280, nucleotide positions 1 to 270, nucleotide positions 1 to 260, nucleotide positions 1 to 250, nucleotide positions 1 to 240, nucleotide positions 1 to 230, nucleotide positions 1 to 220, nucleotide positions 1 to 210, nucleotide positions 1 to 200, nucleotide positions 1 to 190, nucleotide positions 1 to 180, nucleotide positions 1 to 170, nucleotide positions 1 to 160, nucleotide positions 1 to 150, nucleotide positions 1 to 140, nucleotide positions 1 to 130, nucleotide positions 1 to 120, nucleotide positions 1 to 110, nucleotide positions 1 to 100, nucleotide positions 1 to 90, nucleotide positions 1 to 80, nucleotide positions 1 to 70, nucleotide positions 1 to 60, nucleotide positions 1 to 50, nucleotide positions 1 to 40, nucleotide positions 1 to 30, nucleotide positions 1 to 20, nucleotide positions 1 to 10, nucleotide positions 10 to 291, nucleotide positions 10 to 290, nucleotide positions 10 to 280, nucleotide positions 10 to 270, nucleotide positions 10 to 260, nucleotide positions 10 to 250, nucleotide positions 10 to 240, nucleotide positions 10 to 230, nucleotide positions 10 to 220, nucleotide positions 10 to 210, nucleotide positions 10 to 200, nucleotide positions 10 to 190, nucleotide positions 10 to 180, nucleotide positions 10 to 170, nucleotide positions 10 to 160, nucleotide positions 10 to 150, nucleotide positions 10 to 140, nucleotide positions 10 to 130, nucleotide positions 10 to 120, nucleotide positions 10 to 110, nucleotide positions 10 to 100, nucleotide positions 10 to 90, nucleotide positions 10 to 80, nucleotide positions 10 to 70, nucleotide positions 10 to 60, nucleotide positions 10 to 50, nucleotide positions 10 to 40, nucleotide positions 10 to 30, nucleotide positions 10 to 20, nucleotide positions 20 to 291, nucleotide positions 20 to 290, nucleotide positions 20 to 280, nucleotide positions 20 to 270, nucleotide positions 20 to 260, nucleotide positions 20 to 250, nucleotide positions 20 to 240, nucleotide positions 20 to 230, nucleotide positions 20 to 220, nucleotide positions 20 to 210, nucleotide positions 20 to 200, nucleotide positions 20 to 190, nucleotide positions 20 to 180, nucleotide positions 20 to 170, nucleotide positions 20 to 160, nucleotide positions 20 to 150, nucleotide positions 20 to 140, nucleotide positions 20 to 130, nucleotide positions 20 to 120, nucleotide positions 20 to 110, nucleotide positions 20 to 100, nucleotide positions 20 to 90, nucleotide positions 20 to 80, nucleotide positions 20 to 70, nucleotide positions 20 to 60, nucleotide positions 20 to 50, nucleotide positions 20 to 40, nucleotide positions 20 to 30, nucleotide positions 30 to 291, nucleotide positions 30 to 290, nucleotide positions 30 to 280, nucleotide positions 30 to 270, nucleotide positions 30 to 260, nucleotide positions 30 to 250, nucleotide positions 30 to 240, nucleotide positions 30 to 230, nucleotide positions 30 to 220, nucleotide positions 30 to 210, nucleotide positions 30 to 200, nucleotide positions 30 to 190, nucleotide positions 30 to 180, nucleotide positions 30 to 170, nucleotide positions 30 to 160, nucleotide positions 30 to 150, nucleotide positions 30 to 140, nucleotide positions 30 to 130, nucleotide positions 30 to 120, nucleotide positions 30 to 110, nucleotide positions 30 to 100, nucleotide positions 30 to 90, nucleotide positions 30 to 80, nucleotide positions 30 to 70, nucleotide positions 30 to 60, nucleotide positions 30 to 50, nucleotide positions 30 to 40, nucleotide positions 40 to 291, nucleotide positions 40 to 290, nucleotide positions 40 to 280, nucleotide positions 40 to 270, nucleotide positions 40 to 260, nucleotide positions 40 to 250, nucleotide positions 40 to 240, nucleotide positions 40 to 230, nucleotide positions 40 to 220, nucleotide positions 40 to 210, nucleotide positions 40 to 200, nucleotide positions 40 to 190, nucleotide positions 40 to 180, nucleotide positions 40 to 170, nucleotide positions 40 to 160, nucleotide positions 40 to 150, nucleotide positions 40 to 140, nucleotide positions 40 to 130, nucleotide positions 40 to 120, nucleotide positions 40 to 110, nucleotide positions 40 to 100, nucleotide positions 40 to 90, nucleotide positions 40 to 80, nucleotide positions 40 to 70, nucleotide positions 40 to 60, nucleotide positions 40 to 50, nucleotide positions 50 to 291, nucleotide positions 50 to 290, nucleotide positions 50 to 280, nucleotide positions 50 to 270, nucleotide positions 50 to 260, nucleotide positions 50 to 250, nucleotide positions 50 to 240, nucleotide positions 50 to 230, nucleotide positions 50 to 220, nucleotide positions 50 to 210, nucleotide positions 50 to 200, nucleotide positions 50 to 190, nucleotide positions 50 to 180, nucleotide positions 50 to 170, nucleotide positions 50 to 160, nucleotide positions 50 to 150, nucleotide positions 50 to 140, nucleotide positions 50 to 130, nucleotide positions 50 to 120, nucleotide positions 50 to 110, nucleotide positions 50 to 100, nucleotide positions 50 to 90, nucleotide positions 50 to 80, nucleotide positions 50 to 70, nucleotide positions 50 to 60, nucleotide positions 60 to 291, nucleotide positions 60 to 290, nucleotide positions 60 to 280, nucleotide positions 60 to 270, nucleotide positions 60 to 260, nucleotide positions 60 to 250, nucleotide positions 60 to 240, nucleotide positions 60 to 230, nucleotide positions 60 to 220, nucleotide positions 60 to 210, nucleotide positions 60 to 200, nucleotide positions 60 to 190, nucleotide positions 60 to 180, nucleotide positions 60 to 170, nucleotide positions 60 to 160, nucleotide positions 60 to 150, nucleotide positions 60 to 140, nucleotide positions 60 to 130, nucleotide positions 60 to 120, nucleotide positions 60 to 110, nucleotide positions 60 to 100, nucleotide positions 60 to 90, nucleotide positions 60 to 80, nucleotide positions 60 to 70, nucleotide positions 70 to 291, nucleotide positions 70 to 290, nucleotide positions 70 to 280, nucleotide positions 70 to 270, nucleotide positions 70 to 260, nucleotide positions 70 to 250, nucleotide positions 70 to 240, nucleotide positions 70 to 230, nucleotide positions 70 to 220, nucleotide positions 70 to 210, nucleotide positions 70 to 200, nucleotide positions 70 to 190, nucleotide positions 70 to 180, nucleotide positions 70 to 170, nucleotide positions 70 to 160, nucleotide positions 70 to 150, nucleotide positions 70 to 140, nucleotide positions 70 to 130, nucleotide positions 70 to 120, nucleotide positions 70 to 110, nucleotide positions 70 to 100, nucleotide positions 70 to 90, nucleotide positions 70 to 80, nucleotide positions 80 to 291, nucleotide positions 80 to 290, nucleotide positions 80 to 280, nucleotide positions 80 to 270, nucleotide positions 80 to 260, nucleotide positions 80 to 250, nucleotide positions 80 to 240, nucleotide positions 80 to 230, nucleotide positions 80 to 220, nucleotide positions 80 to 210, nucleotide positions 80 to 200, nucleotide positions 80 to 190, nucleotide positions 80 to 180, nucleotide positions 80 to 170, nucleotide positions 80 to 160, nucleotide positions 80 to 150, nucleotide positions 80 to 140, nucleotide positions 80 to 130, nucleotide positions 80 to 120, nucleotide positions 80 to 110, nucleotide positions 80 to 100, nucleotide positions 80 to 90, nucleotide positions 90 to 291, nucleotide positions 90 to 290, nucleotide positions 90 to 280, nucleotide positions 90 to 270, nucleotide positions 90 to 260, nucleotide positions 90 to 250, nucleotide positions 90 to 240, nucleotide positions 90 to 230, nucleotide positions 90 to 220, nucleotide positions 90 to 210, nucleotide positions 90 to 200, nucleotide positions 90 to 190, nucleotide positions 90 to 180, nucleotide positions 90 to 170, nucleotide positions 90 to 160, nucleotide positions 90 to 150, nucleotide positions 90 to 140, nucleotide positions 90 to 130, nucleotide positions 90 to 120, nucleotide positions 90 to 110, nucleotide positions 90 to 100, nucleotide positions 100 to 291, nucleotide positions 100 to 290, nucleotide positions 100 to 280, nucleotide positions 100 to 270, nucleotide positions 100 to 260, nucleotide positions 100 to 250, nucleotide positions 100 to 240, nucleotide positions 100 to 230, nucleotide positions 100 to 220, nucleotide positions 100 to 210, nucleotide positions 100 to 200, nucleotide positions 100 to 190, nucleotide positions 100 to 180, nucleotide positions 100 to 170, nucleotide positions 100 to 160, nucleotide positions 100 to 150, nucleotide positions 100 to 140, nucleotide positions 100 to 130, nucleotide positions 100 to 120, nucleotide positions 100 to 110, nucleotide positions 110 to 291, nucleotide positions 110 to 290, nucleotide positions 110 to 280, nucleotide positions 110 to 270, nucleotide positions 110 to 260, nucleotide positions 110 to 250, nucleotide positions 110 to 240, nucleotide positions 110 to 230, nucleotide positions 110 to 220, nucleotide positions 110 to 210, nucleotide positions 110 to 200, nucleotide positions 110 to 190, nucleotide positions 110 to 180, nucleotide positions 110 to 170, nucleotide positions 110 to 160, nucleotide positions 110 to 150, nucleotide positions 110 to 140, nucleotide positions 110 to 130, nucleotide positions 110 to 120, nucleotide positions 120 to 291, nucleotide positions 120 to 290, nucleotide positions 120 to 280, nucleotide positions 120 to 270, nucleotide positions 120 to 260, nucleotide positions 120 to 250, nucleotide positions 120 to 240, nucleotide positions 120 to 230, nucleotide positions 120 to 220, nucleotide positions 120 to 210, nucleotide positions 120 to 200, nucleotide positions 120 to 190, nucleotide positions 120 to 180, nucleotide positions 120 to 170, nucleotide positions 120 to 160, nucleotide positions 120 to 150, nucleotide positions 120 to 140, nucleotide positions 120 to 130, nucleotide positions 130 to 291, nucleotide positions 130 to 290, nucleotide positions 130 to 280, nucleotide positions 130 to 270, nucleotide positions 130 to 260, nucleotide positions 130 to 250, nucleotide positions 130 to 240, nucleotide positions 130 to 230, nucleotide positions 130 to 220, nucleotide positions 130 to 210, nucleotide positions 130 to 200, nucleotide positions 130 to 190, nucleotide positions 130 to 180, nucleotide positions 130 to 170, nucleotide positions 130 to 160, nucleotide positions 130 to 150, nucleotide positions 130 to 140, nucleotide positions 140 to 291, nucleotide positions 140 to 290, nucleotide positions 140 to 280, nucleotide positions 140 to 270, nucleotide positions 140 to 260, nucleotide positions 140 to 250, nucleotide positions 140 to 240, nucleotide positions 140 to 230, nucleotide positions 140 to 220, nucleotide positions 140 to 210, nucleotide positions 140 to 200, nucleotide positions 140 to 190, nucleotide positions 140 to 180, nucleotide positions 140 to 170, nucleotide positions 140 to 160, nucleotide positions 140 to 150, nucleotide positions 150 to 291, nucleotide positions 150 to 290, nucleotide positions 150 to 280, nucleotide positions 150 to 270, nucleotide positions 150 to 260, nucleotide positions 150 to 250, nucleotide positions 150 to 240, nucleotide positions 150 to 230, nucleotide positions 150 to 220, nucleotide positions 150 to 210, nucleotide positions 150 to 200, nucleotide positions 150 to 190, nucleotide positions 150 to 180, nucleotide positions 150 to 170, nucleotide positions 150 to 160, nucleotide positions 160 to 291, nucleotide positions 160 to 290, nucleotide positions 160 to 280, nucleotide positions 160 to 270, nucleotide positions 160 to 260, nucleotide positions 160 to 250, nucleotide positions 160 to 240, nucleotide positions 160 to 230, nucleotide positions 160 to 220, nucleotide positions 160 to 210, nucleotide positions 160 to 200, nucleotide positions 160 to 190, nucleotide positions 160 to 180, nucleotide positions 160 to 170, nucleotide positions 170 to 291, nucleotide positions 170 to 290, nucleotide positions 170 to 280, nucleotide positions 170 to 270, nucleotide positions 170 to 260, nucleotide positions 170 to 250, nucleotide positions 170 to 240, nucleotide positions 170 to 230, nucleotide positions 170 to 220, nucleotide positions 170 to 210, nucleotide positions 170 to 200, nucleotide positions 170 to 190, nucleotide positions 170 to 180, nucleotide positions 180 to 291, nucleotide positions 180 to 290, nucleotide positions 180 to 280, nucleotide positions 180 to 270, nucleotide positions 180 to 260, nucleotide positions 180 to 250, nucleotide positions 180 to 240, nucleotide positions 180 to 230, nucleotide positions 180 to 220, nucleotide positions 180 to 210, nucleotide positions 180 to 200, nucleotide positions 180 to 190, nucleotide positions 190 to 291, nucleotide positions 190 to 290, nucleotide positions 190 to 280, nucleotide positions 190 to 270, nucleotide positions 190 to 260, nucleotide positions 190 to 250, nucleotide positions 190 to 240, nucleotide positions 190 to 230, nucleotide positions 190 to 220, nucleotide positions 190 to 210, nucleotide positions 190 to 200, nucleotide positions 200 to 291, nucleotide positions 200 to 290, nucleotide positions 200 to 280, nucleotide positions 200 to 270, nucleotide positions 200 to 260, nucleotide positions 200 to 250, nucleotide positions 200 to 240, nucleotide positions 200 to 230, nucleotide positions 200 to 220, nucleotide positions 200 to 210, nucleotide positions 210 to 291, nucleotide positions 210 to 290, nucleotide positions 210 to 280, nucleotide positions 210 to 270, nucleotide positions 210 to 260, nucleotide positions 210 to 250, nucleotide positions 210 to 240, nucleotide positions 210 to 230, nucleotide positions 210 to 220, nucleotide positions 220 to 291, nucleotide positions 220 to 290, nucleotide positions 220 to 280, nucleotide positions 220 to 270, nucleotide positions 220 to 260, nucleotide positions 220 to 250, nucleotide positions 220 to 240, nucleotide positions 220 to 230, nucleotide positions 230 to 291, nucleotide positions 230 to 290, nucleotide positions 230 to 280, nucleotide positions 230 to 270, nucleotide positions 230 to 260, nucleotide positions 230 to 250, nucleotide positions 230 to 240, nucleotide positions 240 to 291, nucleotide positions 240 to 290, nucleotide positions 240 to 280, nucleotide positions 240 to 270, nucleotide positions 240 to 260, nucleotide positions 240 to 250, nucleotide positions 250 to 291, nucleotide positions 250 to 290, nucleotide positions 250 to 280, nucleotide positions 250 to 270, nucleotide positions 250 to 260, nucleotide positions 260 to 291, nucleotide positions 260 to 290, nucleotide positions 260 to 280, nucleotide positions 260 to 270, nucleotide positions 270 to 291, nucleotide positions 270 to 290, nucleotide positions 270 to 280, nucleotide positions 280 to 291, or nucleotide positions 280 to 290, of SEQ ID NO: 12 or 13.

In some embodiments of any of the compositions described herein, the 5′ UTR comprises a sequence that is at least 70% (e.g., at least 75%, at least 80%, at least 85%, at least 90%, at least 91%, at least 92%, at least 93%, at least 94%, at least 95%, at least 96%, at least 97%, at least 98%, or at least 99%) identical to SEQ ID NO: 12 or 13. In some embodiments of any of the compositions described herein, the 3′ UTR comprises at least 10 contiguous (e.g., at least 15 contiguous, at least 20 contiguous, at least 25 contiguous, at least 30 contiguous, at least 35 contiguous, at least 40 contiguous, at least 45 contiguous, at least 50 contiguous, at least 55 contiguous, at least 60 contiguous, at least 65 contiguous, at least 70 contiguous, at least 75 contiguous, at least 80 contiguous, at least 85 contiguous, at least 90 contiguous, at least 100 contiguous, at least 105 contiguous, at least 110 contiguous, at least 115 contiguous, at least 120 contiguous, at least 125 contiguous, at least 130 contiguous, at least 135 contiguous, at least 140 contiguous, at least 145 contiguous, at least 150 contiguous, at least 155 contiguous, at least 160 contiguous, at least 165 contiguous, at least 170 contiguous, at least 175 contiguous, at least 180 contiguous, at least 185 contiguous, at least 190 contiguous, at least 195 contiguous, at least 200 contiguous, at least 205 contiguous, at least 210 contiguous, at least 215 contiguous, at least 220 contiguous, at least 225 contiguous, at least 230 contiguous, at least 235 contiguous, at least 240 contiguous, at least 245 contiguous, at least 250 contiguous, at least 255 contiguous, at least 260 contiguous, at least 265 contiguous, at least 270 contiguous, at least 275 contiguous, at least 280 contiguous, at least 285 contiguous, at least 290 contiguous, at least 295 contiguous, at least 300 contiguous, at least 305 contiguous, at least 310 contiguous, at least 315 contiguous, at least 320 contiguous, at least 325 contiguous, at least 330 contiguous, at least 335 contiguous, at least 340 contiguous, at least 345 contiguous, at least 350 contiguous, at least 355 contiguous, at least 360 contiguous, at least 365 contiguous, at least 370 contiguous, at least 375 contiguous, at least 380 contiguous, at least 385 contiguous, at least 390 contiguous, at least 395 contiguous, at least 400 contiguous, at least 450 contiguous, at least 500 contiguous, at least 550 contiguous, at least 600 contiguous, at least 650 contiguous, at least 700 contiguous, at least 750 contiguous, at least 800 contiguous, at least 850 contiguous, at least 900 contiguous, at least 950 contiguous, at least 1000 contiguous, at least 1050 contiguous, at least 1100 contiguous, at least 1150 contiguous, at least 1200 contiguous, at least 1250 contiguous, at least 1300 contiguous, at least 1350 contiguous, at least 1400 contiguous, at least 1450 contiguous, at least 1500 contiguous, at least 1550 contiguous, at least 1600 contiguous, at least 1650 contiguous, at least 1700 contiguous, or at least 1750 contiguous) nucleotides from anywhere within SEQ ID NO: 14 or 15.

For example, a 5′ UTR can include or consist of one or more of: nucleotide positions 1 to 1773, nucleotide positions 1 to 1770, nucleotide positions 1 to 1750, nucleotide positions 1 to 1700, nucleotide positions 1 to 1650, nucleotide positions 1 to 1600, nucleotide positions 1 to 1550, nucleotide positions 1 to 1500, nucleotide positions 1 to 1450, nucleotide positions 1 to 1400, nucleotide positions 1 to 1350, nucleotide positions 1 to 1300, nucleotide positions 1 to 1250, nucleotide positions 1 to 1200, nucleotide positions 1 to 1150, nucleotide positions 1 to 1100, nucleotide positions 1 to 1050, nucleotide positions 1 to 1000, nucleotide positions 1 to 950, nucleotide positions 1 to 900, nucleotide positions 1 to 850, nucleotide positions 1 to 800, nucleotide positions 1 to 750, nucleotide positions 1 to 700, nucleotides positions 1 to 650, nucleotide positions 1 to 600, nucleotide positions 1 to 550, nucleotide positions 1 to 500, nucleotide positions 1 to 450, nucleotide positions 1 to 400, nucleotide positions 1 to 350, nucleotide positions 1 to 300, nucleotide positions 1 to 250, nucleotide positions 1 to 200, nucleotide positions 1 to 150, nucleotide positions 1 to 100, nucleotide positions 1 to 50, nucleotide position 1 to 25, nucleotide positions 25 to 1773, nucleotide positions 25 to 1770, nucleotide positions 25 to 1750, nucleotide positions 25 to 1700, nucleotide positions 25 to 1650, nucleotide positions 25 to 1600, nucleotide positions 25 to 1550, nucleotide positions 25 to 1500, nucleotide positions 25 to 1450, nucleotide positions 25 to 1400, nucleotide positions 25 to 1350, nucleotide positions 25 to 1300, nucleotide positions 25 to 1250, nucleotide positions 25 to 1200, nucleotide positions 25 to 1150, nucleotide positions 25 to 1100, nucleotide positions 25 to 1050, nucleotide positions 25 to 1000, nucleotide positions 25 to 950, nucleotide positions 25 to 900, nucleotide positions 25 to 850, nucleotide positions 25 to 800, nucleotide positions 25 to 750, nucleotide positions 25 to 700, nucleotides positions 25 to 650, nucleotide positions 25 to 600, nucleotide positions 25 to 550, nucleotide positions 25 to 500, nucleotide positions 25 to 450, nucleotide positions 25 to 400, nucleotide positions 25 to 350, nucleotide positions 25 to 300, nucleotide positions 25 to 250, nucleotide positions 25 to 200, nucleotide positions 25 to 150, nucleotide positions 25 to 100, nucleotide positions 25 to 50, nucleotide positions 50 to 1773, nucleotide positions 50 to 1770, nucleotide positions 50 to 1750, nucleotide positions 50 to 1700, nucleotide positions 50 to 1650, nucleotide positions 50 to 1600, nucleotide positions 50 to 1550, nucleotide positions 50 to 1500, nucleotide positions 50 to 1450, nucleotide positions 50 to 1400, nucleotide positions 50 to 1350, nucleotide positions 50 to 1300, nucleotide positions 50 to 1250, nucleotide positions 50 to 1200, nucleotide positions 50 to 1150, nucleotide positions 50 to 1100, nucleotide positions 50 to 1050, nucleotide positions 50 to 1000, nucleotide positions 50 to 950, nucleotide positions 50 to 900, nucleotide positions 50 to 850, nucleotide positions 50 to 800, nucleotide positions 50 to 750, nucleotide positions 50 to 700, nucleotides positions 50 to 650, nucleotide positions 50 to 600, nucleotide positions 50 to 550, nucleotide positions 50 to 500, nucleotide positions 50 to 450, nucleotide positions 50 to 400, nucleotide positions 50 to 350, nucleotide positions 50 to 300, nucleotide positions 50 to 250, nucleotide positions 50 to 200, nucleotide positions 50 to 150, nucleotide positions 50 to 100, nucleotide positions 100 to 1773, nucleotide positions 100 to 1770, nucleotide positions 100 to 1750, nucleotide positions 100 to 1700, nucleotide positions 100 to 1650, nucleotide positions 100 to 1600, nucleotide positions 100 to 1550, nucleotide positions 100 to 1500, nucleotide positions 100 to 1450, nucleotide positions 100 to 1400, nucleotide positions 100 to 1350, nucleotide positions 100 to 1300, nucleotide positions 100 to 1250, nucleotide positions 100 to 1200, nucleotide positions 100 to 1150, nucleotide positions 100 to 1100, nucleotide positions 100 to 1050, nucleotide positions 100 to 1000, nucleotide positions 100 to 950, nucleotide positions 100 to 900, nucleotide positions 100 to 850, nucleotide positions 100 to 800, nucleotide positions 100 to 750, nucleotide positions 100 to 700, nucleotides positions 100 to 650, nucleotide positions 100 to 600, nucleotide positions 100 to 550, nucleotide positions 100 to 500, nucleotide positions 100 to 450, nucleotide positions 100 to 400, nucleotide positions 100 to 350, nucleotide positions 100 to 300, nucleotide positions 100 to 250, nucleotide positions 100 to 200, nucleotide positions 100 to 150, nucleotide positions 150 to 1773, nucleotide positions 150 to 1770, nucleotide positions 150 to 1750, nucleotide positions 150 to 1700, nucleotide positions 150 to 1650, nucleotide positions 150 to 1600, nucleotide positions 150 to 1550, nucleotide positions 150 to 1500, nucleotide positions 150 to 1450, nucleotide positions 150 to 1400, nucleotide positions 150 to 1350, nucleotide positions 150 to 1300, nucleotide positions 150 to 1250, nucleotide positions 150 to 1200, nucleotide positions 150 to 1150, nucleotide positions 150 to 1100, nucleotide positions 150 to 1050, nucleotide positions 150 to 1000, nucleotide positions 150 to 950, nucleotide positions 150 to 900, nucleotide positions 150 to 850, nucleotide positions 150 to 800, nucleotide positions 150 to 750, nucleotide positions 150 to 700, nucleotides positions 150 to 650, nucleotide positions 150 to 600, nucleotide positions 150 to 550, nucleotide positions 150 to 500, nucleotide positions 150 to 450, nucleotide positions 150 to 400, nucleotide positions 150 to 350, nucleotide positions 150 to 300, nucleotide positions 150 to 250, nucleotide positions 150 to 200, nucleotide positions 200 to 1773, nucleotide positions 200 to 1770, nucleotide positions 200 to 1750, nucleotide positions 200 to 1700, nucleotide positions 200 to 1650, nucleotide positions 200 to 1600, nucleotide positions 200 to 1550, nucleotide positions 200 to 1500, nucleotide positions 200 to 1450, nucleotide positions 200 to 1400, nucleotide positions 200 to 1350, nucleotide positions 200 to 1300, nucleotide positions 200 to 1250, nucleotide positions 200 to 1200, nucleotide positions 200 to 1150, nucleotide positions 200 to 1100, nucleotide positions 200 to 1050, nucleotide positions 200 to 1000, nucleotide positions 200 to 950, nucleotide positions 200 to 900, nucleotide positions 200 to 850, nucleotide positions 200 to 800, nucleotide positions 200 to 750, nucleotide positions 200 to 700, nucleotides positions 200 to 650, nucleotide positions 200 to 600, nucleotide positions 200 to 550, nucleotide positions 200 to 500, nucleotide positions 200 to 450, nucleotide positions 200 to 400, nucleotide positions 200 to 350, nucleotide positions 200 to 300, nucleotide positions 200 to 250, nucleotide positions 250 to 1773, nucleotide positions 250 to 1770, nucleotide positions 250 to 1750, nucleotide positions 250 to 1700, nucleotide positions 250 to 1650, nucleotide positions 250 to 1600, nucleotide positions 250 to 1550, nucleotide positions 250 to 1500, nucleotide positions 250 to 1450, nucleotide positions 250 to 1400, nucleotide positions 250 to 1350, nucleotide positions 250 to 1300, nucleotide positions 250 to 1250, nucleotide positions 250 to 1200, nucleotide positions 250 to 1150, nucleotide positions 250 to 1100, nucleotide positions 250 to 1050, nucleotide positions 250 to 1000, nucleotide positions 250 to 950, nucleotide positions 250 to 900, nucleotide positions 250 to 850, nucleotide positions 250 to 800, nucleotide positions 250 to 750, nucleotide positions 250 to 700, nucleotides positions 250 to 650, nucleotide positions 250 to 600, nucleotide positions 250 to 550, nucleotide positions 250 to 500, nucleotide positions 250 to 450, nucleotide positions 250 to 400, nucleotide positions 250 to 350, nucleotide positions 250 to 300, nucleotide positions 300 to 1773, nucleotide positions 300 to 1770, nucleotide positions 300 to 1750, nucleotide positions 300 to 1700, nucleotide positions 300 to 1650, nucleotide positions 300 to 1600, nucleotide positions 300 to 1550, nucleotide positions 300 to 1500, nucleotide positions 300 to 1450, nucleotide positions 300 to 1400, nucleotide positions 300 to 1350, nucleotide positions 300 to 1300, nucleotide positions 300 to 1250, nucleotide positions 300 to 1200, nucleotide positions 300 to 1150, nucleotide positions 300 to 1100, nucleotide positions 300 to 1050, nucleotide positions 300 to 1000, nucleotide positions 300 to 950, nucleotide positions 300 to 900, nucleotide positions 300 to 850, nucleotide positions 300 to 800, nucleotide positions 300 to 750, nucleotide positions 300 to 700, nucleotides positions 300 to 650, nucleotide positions 300 to 600, nucleotide positions 300 to 550, nucleotide positions 300 to 500, nucleotide positions 300 to 450, nucleotide positions 300 to 400, nucleotide positions 300 to 350, nucleotide positions 350 to 1773, nucleotide positions 350 to 1770, nucleotide positions 350 to 1750, nucleotide positions 350 to 1700, nucleotide positions 350 to 1650, nucleotide positions 350 to 1600, nucleotide positions 350 to 1550, nucleotide positions 350 to 1500, nucleotide positions 350 to 1450, nucleotide positions 350 to 1400, nucleotide positions 350 to 1350, nucleotide positions 350 to 1300, nucleotide positions 350 to 1250, nucleotide positions 350 to 1200, nucleotide positions 350 to 1150, nucleotide positions 350 to 1100, nucleotide positions 350 to 1050, nucleotide positions 350 to 1000, nucleotide positions 350 to 950, nucleotide positions 350 to 900, nucleotide positions 350 to 850, nucleotide positions 350 to 800, nucleotide positions 350 to 750, nucleotide positions 350 to 700, nucleotides positions 350 to 650, nucleotide positions 350 to 600, nucleotide positions 350 to 550, nucleotide positions 350 to 500, nucleotide positions 350 to 450, nucleotide positions 350 to 400, nucleotide positions 400 to 1773, nucleotide positions 400 to 1770, nucleotide positions 400 to 1750, nucleotide positions 400 to 1700, nucleotide positions 400 to 1650, nucleotide positions 400 to 1600, nucleotide positions 400 to 1550, nucleotide positions 400 to 1500, nucleotide positions 400 to 1450, nucleotide positions 400 to 1400, nucleotide positions 400 to 1350, nucleotide positions 400 to 1300, nucleotide positions 400 to 1250, nucleotide positions 400 to 1200, nucleotide positions 400 to 1150, nucleotide positions 400 to 1100, nucleotide positions 400 to 1050, nucleotide positions 400 to 1000, nucleotide positions 400 to 950, nucleotide positions 400 to 900, nucleotide positions 400 to 850, nucleotide positions 400 to 800, nucleotide positions 400 to 750, nucleotide positions 400 to 700, nucleotides positions 400 to 650, nucleotide positions 400 to 600, nucleotide positions 400 to 550, nucleotide positions 400 to 500, nucleotide positions 400 to 450, nucleotide positions 450 to 1773, nucleotide positions 450 to 1770, nucleotide positions 450 to 1750, nucleotide positions 450 to 1700, nucleotide positions 450 to 1650, nucleotide positions 450 to 1600, nucleotide positions 450 to 1550, nucleotide positions 450 to 1500, nucleotide positions 450 to 1450, nucleotide positions 450 to 1400, nucleotide positions 450 to 1350, nucleotide positions 450 to 1300, nucleotide positions 450 to 1250, nucleotide positions 450 to 1200, nucleotide positions 450 to 1150, nucleotide positions 450 to 1100, nucleotide positions 450 to 1050, nucleotide positions 450 to 1000, nucleotide positions 450 to 950, nucleotide positions 450 to 900, nucleotide positions 450 to 850, nucleotide positions 450 to 800, nucleotide positions 450 to 750, nucleotide positions 450 to 700, nucleotides positions 450 to 650, nucleotide positions 450 to 600, nucleotide positions 450 to 550, nucleotide positions 450 to 500, nucleotide positions 500 to 1773, nucleotide positions 500 to 1770, nucleotide positions 500 to 1750, nucleotide positions 500 to 1700, nucleotide positions 500 to 1650, nucleotide positions 500 to 1600, nucleotide positions 500 to 1550, nucleotide positions 500 to 1500, nucleotide positions 500 to 1450, nucleotide positions 500 to 1400, nucleotide positions 500 to 1350, nucleotide positions 500 to 1300, nucleotide positions 500 to 1250, nucleotide positions 500 to 1200, nucleotide positions 500 to 1150, nucleotide positions 500 to 1100, nucleotide positions 500 to 1050, nucleotide positions 500 to 1000, nucleotide positions 500 to 950, nucleotide positions 500 to 900, nucleotide positions 500 to 850, nucleotide positions 500 to 800, nucleotide positions 500 to 750, nucleotide positions 500 to 700, nucleotides positions 500 to 650, nucleotide positions 500 to 600, nucleotide positions 500 to 550, nucleotide positions 550 to 1773, nucleotide positions 550 to 1770, nucleotide positions 550 to 1750, nucleotide positions 550 to 1700, nucleotide positions 550 to 1650, nucleotide positions 550 to 1600, nucleotide positions 550 to 1550, nucleotide positions 550 to 1500, nucleotide positions 550 to 1450, nucleotide positions 550 to 1400, nucleotide positions 550 to 1350, nucleotide positions 550 to 1300, nucleotide positions 550 to 1250, nucleotide positions 550 to 1200, nucleotide positions 550 to 1150, nucleotide positions 550 to 1100, nucleotide positions 550 to 1050, nucleotide positions 550 to 1000, nucleotide positions 550 to 950, nucleotide positions 550 to 900, nucleotide positions 550 to 850, nucleotide positions 550 to 800, nucleotide positions 550 to 750, nucleotide positions 550 to 700, nucleotides positions 550 to 650, nucleotide positions 550 to 600, nucleotide positions 600 to 1773, nucleotide positions 600 to 1770, nucleotide positions 600 to 1750, nucleotide positions 600 to 1700, nucleotide positions 600 to 1650, nucleotide positions 600 to 1600, nucleotide positions 600 to 1550, nucleotide positions 600 to 1500, nucleotide positions 600 to 1450, nucleotide positions 600 to 1400, nucleotide positions 600 to 1350, nucleotide positions 600 to 1300, nucleotide positions 600 to 1250, nucleotide positions 600 to 1200, nucleotide positions 600 to 1150, nucleotide positions 600 to 1100, nucleotide positions 600 to 1050, nucleotide positions 600 to 1000, nucleotide positions 600 to 950, nucleotide positions 600 to 900, nucleotide positions 600 to 850, nucleotide positions 600 to 800, nucleotide positions 600 to 750, nucleotide positions 600 to 700, nucleotides positions 600 to 650, nucleotide positions 650 to 1773, nucleotide positions 650 to 1770, nucleotide positions 650 to 1750, nucleotide positions 650 to 1700, nucleotide positions 650 to 1650, nucleotide positions 650 to 1600, nucleotide positions 650 to 1550, nucleotide positions 650 to 1500, nucleotide positions 650 to 1450, nucleotide positions 650 to 1400, nucleotide positions 650 to 1350, nucleotide positions 650 to 1300, nucleotide positions 650 to 1250, nucleotide positions 650 to 1200, nucleotide positions 650 to 1150, nucleotide positions 650 to 1100, nucleotide positions 650 to 1050, nucleotide positions 650 to 1000, nucleotide positions 650 to 950, nucleotide positions 650 to 900, nucleotide positions 650 to 850, nucleotide positions 650 to 800, nucleotide positions 650 to 750, nucleotide positions 650 to 700, nucleotide positions 700 to 1773, nucleotide positions 700 to 1770, nucleotide positions 700 to 1750, nucleotide positions 700 to 1700, nucleotide positions 700 to 1650, nucleotide positions 700 to 1600, nucleotide positions 700 to 1550, nucleotide positions 700 to 1500, nucleotide positions 700 to 1450, nucleotide positions 700 to 1400, nucleotide positions 700 to 1350, nucleotide positions 700 to 1300, nucleotide positions 700 to 1250, nucleotide positions 700 to 1200, nucleotide positions 700 to 1150, nucleotide positions 700 to 1100, nucleotide positions 700 to 1050, nucleotide positions 700 to 1000, nucleotide positions 700 to 950, nucleotide positions 700 to 900, nucleotide positions 700 to 850, nucleotide positions 700 to 800, nucleotide positions 700 to 750, nucleotide positions 750 to 1773, nucleotide positions 750 to 1770, nucleotide positions 750 to 1750, nucleotide positions 750 to 1700, nucleotide positions 750 to 1650, nucleotide positions 750 to 1600, nucleotide positions 750 to 1550, nucleotide positions 750 to 1500, nucleotide positions 750 to 1450, nucleotide positions 750 to 1400, nucleotide positions 750 to 1350, nucleotide positions 750 to 1300, nucleotide positions 750 to 1250, nucleotide positions 750 to 1200, nucleotide positions 750 to 1150, nucleotide positions 750 to 1100, nucleotide positions 750 to 1050, nucleotide positions 750 to 1000, nucleotide positions 750 to 950, nucleotide positions 750 to 900, nucleotide positions 750 to 850, nucleotide positions 750 to 800, nucleotide positions 800 to 1773, nucleotide positions 800 to 1770, nucleotide positions 800 to 1750, nucleotide positions 800 to 1700, nucleotide positions 800 to 1650, nucleotide positions 800 to 1600, nucleotide positions 800 to 1550, nucleotide positions 800 to 1500, nucleotide positions 800 to 1450, nucleotide positions 800 to 1400, nucleotide positions 800 to 1350, nucleotide positions 800 to 1300, nucleotide positions 800 to 1250, nucleotide positions 800 to 1200, nucleotide positions 800 to 1150, nucleotide positions 800 to 1100, nucleotide positions 800 to 1050, nucleotide positions 800 to 1000, nucleotide positions 800 to 950, nucleotide positions 800 to 900, nucleotide positions 800 to 850, nucleotide positions 850 to 1773, nucleotide positions 850 to 1770, nucleotide positions 850 to 1750, nucleotide positions 850 to 1700, nucleotide positions 850 to 1650, nucleotide positions 850 to 1600, nucleotide positions 850 to 1550, nucleotide positions 850 to 1500, nucleotide positions 850 to 1450, nucleotide positions 850 to 1400, nucleotide positions 850 to 1350, nucleotide positions 850 to 1300, nucleotide positions 850 to 1250, nucleotide positions 850 to 1200, nucleotide positions 850 to 1150, nucleotide positions 850 to 1100, nucleotide positions 850 to 1050, nucleotide positions 850 to 1000, nucleotide positions 850 to 950, nucleotide positions 850 to 900, nucleotide positions 900 to 1773, nucleotide positions 900 to 1770, nucleotide positions 900 to 1750, nucleotide positions 900 to 1700, nucleotide positions 900 to 1650, nucleotide positions 900 to 1600, nucleotide positions 900 to 1550, nucleotide positions 900 to 1500, nucleotide positions 900 to 1450, nucleotide positions 900 to 1400, nucleotide positions 900 to 1350, nucleotide positions 900 to 1300, nucleotide positions 900 to 1250, nucleotide positions 900 to 1200, nucleotide positions 900 to 1150, nucleotide positions 900 to 1100, nucleotide positions 900 to 1050, nucleotide positions 900 to 1000, nucleotide positions 900 to 950, nucleotide positions 950 to 1773, nucleotide positions 950 to 1770, nucleotide positions 950 to 1750, nucleotide positions 950 to 1700, nucleotide positions 950 to 1650, nucleotide positions 950 to 1600, nucleotide positions 950 to 1550, nucleotide positions 950 to 1500, nucleotide positions 950 to 1450, nucleotide positions 950 to 1400, nucleotide positions 950 to 1350, nucleotide positions 950 to 1300, nucleotide positions 950 to 1250, nucleotide positions 950 to 1200, nucleotide positions 950 to 1150, nucleotide positions 950 to 1100, nucleotide positions 950 to 1050, nucleotide positions 950 to 1000, nucleotide positions 1000 to 1773, nucleotide positions 1000 to 1770, nucleotide positions 1000 to 1750, nucleotide positions 1000 to 1700, nucleotide positions 1000 to 1650, nucleotide positions 1000 to 1600, nucleotide positions 1000 to 1550, nucleotide positions 1000 to 1500, nucleotide positions 1000 to 1450, nucleotide positions 1000 to 1400, nucleotide positions 1000 to 1350, nucleotide positions 1000 to 1300, nucleotide positions 1000 to 1250, nucleotide positions 1000 to 1200, nucleotide positions 1000 to 1150, nucleotide positions 1000 to 1100, nucleotide positions 1000 to 1050, nucleotide positions 1050 to 1773, nucleotide positions 1050 to 1770, nucleotide positions 1050 to 1750, nucleotide positions 1050 to 1700, nucleotide positions 1050 to 1650, nucleotide positions 1050 to 1600, nucleotide positions 1050 to 1550, nucleotide positions 1050 to 1500, nucleotide positions 1050 to 1450, nucleotide positions 1050 to 1400, nucleotide positions 1050 to 1350, nucleotide positions 1050 to 1300, nucleotide positions 1050 to 1250, nucleotide positions 1050 to 1200, nucleotide positions 1050 to 1150, nucleotide positions 1050 to 1100, nucleotide positions 1100 to 1773, nucleotide positions 1100 to 1770, nucleotide positions 1100 to 1750, nucleotide positions 1100 to 1700, nucleotide positions 1100 to 1650, nucleotide positions 1100 to 1600, nucleotide positions 1100 to 1550, nucleotide positions 1100 to 1500, nucleotide positions 1100 to 1450, nucleotide positions 1100 to 1400, nucleotide positions 1100 to 1350, nucleotide positions 1100 to 1300, nucleotide positions 1100 to 1250, nucleotide positions 1100 to 1200, nucleotide positions 1100 to 1150, nucleotide positions 1150 to 1773, nucleotide positions 1150 to 1770, nucleotide positions 1150 to 1750, nucleotide positions 1150 to 1700, nucleotide positions 1150 to 1650, nucleotide positions 1150 to 1600, nucleotide positions 1150 to 1550, nucleotide positions 1150 to 1500, nucleotide positions 1150 to 1450, nucleotide positions 1150 to 1400, nucleotide positions 1150 to 1350, nucleotide positions 1150 to 1300, nucleotide positions 1150 to 1250, nucleotide positions 1150 to 1200, nucleotide positions 1200 to 1773, nucleotide positions 1200 to 1770, nucleotide positions 1200 to 1750, nucleotide positions 1200 to 1700, nucleotide positions 1200 to 1650, nucleotide positions 1200 to 1600, nucleotide positions 1200 to 1550, nucleotide positions 1200 to 1500, nucleotide positions 1200 to 1450, nucleotide positions 1200 to 1400, nucleotide positions 1200 to 1350, nucleotide positions 1200 to 1300, nucleotide positions 1200 to 1250, nucleotide positions 1250 to 1773, nucleotide positions 1250 to 1770, nucleotide positions 1250 to 1750, nucleotide positions 1250 to 1700, nucleotide positions 1250 to 1650, nucleotide positions 1250 to 1600, nucleotide positions 1250 to 1550, nucleotide positions 1250 to 1500, nucleotide positions 1250 to 1450, nucleotide positions 1250 to 1400, nucleotide positions 1250 to 1350, nucleotide positions 1250 to 1300, nucleotide positions 1300 to 1773, nucleotide positions 1300 to 1770, nucleotide positions 1300 to 1750, nucleotide positions 1300 to 1700, nucleotide positions 1300 to 1650, nucleotide positions 1300 to 1600, nucleotide positions 1300 to 1550, nucleotide positions 1300 to 1500, nucleotide positions 1300 to 1450, nucleotide positions 1300 to 1400, nucleotide positions 1300 to 1350, nucleotide positions 1350 to 1773, nucleotide positions 1350 to 1770, nucleotide positions 1350 to 1750, nucleotide positions 1350 to 1700, nucleotide positions 1350 to 1650, nucleotide positions 1350 to 1600, nucleotide positions 1350 to 1550, nucleotide positions 1350 to 1500, nucleotide positions 1350 to 1450, nucleotide positions 1350 to 1400, nucleotide positions 1400 to 1773, nucleotide positions 1400 to 1770, nucleotide positions 1400 to 1750, nucleotide positions 1400 to 1700, nucleotide positions 1400 to 1650, nucleotide positions 1400 to 1600, nucleotide positions 1400 to 1550, nucleotide positions 1400 to 1500, nucleotide positions 1400 to 1450, nucleotide positions 1450 to 1773, nucleotide positions 1450 to 1770, nucleotide positions 1450 to 1750, nucleotide positions 1450 to 1700, nucleotide positions 1450 to 1650, nucleotide positions 1450 to 1600, nucleotide positions 1450 to 1550, nucleotide positions 1450 to 1500, nucleotide positions 1500 to 1773, nucleotide positions 1500 to 1770, nucleotide positions 1500 to 1750, nucleotide positions 1500 to 1700, nucleotide positions 1500 to 1650, nucleotide positions 1500 to 1600, nucleotide positions 1500 to 1550, nucleotide positions 1550 to 1773, nucleotide positions 1550 to 1770, nucleotide positions 1550 to 1750, nucleotide positions 1550 to 1700, nucleotide positions 1550 to 1650, nucleotide positions 1550 to 1600, nucleotide positions 1600 to 1773, nucleotide positions 1600 to 1770, nucleotide positions 1600 to 1750, nucleotide positions 1600 to 1700, nucleotide positions 1600 to 1650, nucleotide positions 1650 to 1773, nucleotide positions 1650 to 1770, nucleotide positions 1650 to 1750, nucleotide positions 1650 to 1700, nucleotide positions 1700 to 1773, nucleotide positions 1700 to 1770, nucleotide positions 1700 to 1750, nucleotide positions 1750 to 1773, or nucleotide positions 1750 to 1770, of SEQ ID NO: 14 or 15.

In some embodiments of any of the compositions described herein, the 3′ UTR comprises a sequence that is at least 70% (e.g., at least 75%, at least 80%, at least 85%, at least 90%, at least 91%, at least 92%, at least 93%, at least 94%, at least 95%, at least 96%, at least 97%, at least 98%, or at least 99%) identical to SEQ ID NO: 14 or 15.

Human 5′ UTR of CLRN1
(SEQ ID NO: 12)
AGGAGATACTTGAAGGCAGTTTGAAAGACTTGTTTTACAGATTCTTAGTCC
AAAGATTTCCAATTAGGGAGAAGAAGCAGCAGAAAAGGAGAAAAGCCAAGT
ATGAGTGATGATGAGGCCTTCATCTACTGACATTTAACCTGGCGAGAACCG
TCGATGGTGAAGTTGCCTTTTCAGCTGGGAGCTGTCCGTTCAGCTTCCGTA
ATAAATGCAGTCAAAGAGGCAGTCCCTTCCCATTGCTCACAAAGGTCTTGT
TTTTGAACCTCGCCCTCACAGAAGCCGTTTCTCATC
Human 5′ UTR of CLRN1
(SEQ ID NO: 13)
CCTGCAGGCAGCTGCGCGCTCGCTCGCTCACTGAGGCCGCCCGGGCGTCGG
GCGACCTTTGGTCGCCCGGCCTCAGTGAGCGAGCGAGCGCGCAGAGAGGGA
GTGGCCAACTCCATCACTAGGGGTTCCT
Human 3′ UTR of CLRN1
(SEQ ID NO: 14)
AGGAACCCCTAGTGATGGAGTTGGCCACTCCCTCTCTGCGCGCTCGCTCGC
TCACTGAGGCCGGGCGACCAAAGGTCGCCCGACGCCCGGGCTTTGCCCGGG
CGGCCTCAGTGAGCGAGCGAGCGCGCAGCTGCCTGCAGG
Human 3′ UTR of CLRN1
(SEQ ID NO: 15)
AAGGCAAACCTTTCTATAATTTTACAAGGGAGTAGACTTGCTTTGGTCACT
TTTAGATGTGGTTAATTTTGCATATCCTTTTAGTCTGCATATATTAAAGCA
TCAGGACCCTTCGTGACAATGTTTACAAATTACGTACTAAGGATACAGGCT
GGAAAGTAAGGGAAGCAGAAGGAAGGCTTTGAAAAGTTGTTTTATCTGGTG
GGAAATTGCTTGACCCAGGTAGTCAAAGGCAGTTGACTAGAATCGACAAAT
TGTTACTCCATATATATATATGTGTGTGTGTGTGTGTGTGTGTGTGTGTAA
GATGTCTTCCTATCAAAAAGATATCAAAGGCACATGGAATATATTTTAATA
AAAACAAATAATATCTCTAATATATCCACACATTTGTTGCCAGATTTCAGA
AAACTGAGCTGCAATCGCTTTCCTAAAACAGTAGTGTATTAAATGAACATC
TATAAAATGTATCAACACACATTTTAAAAAATTTGTTTAAAGTATACTCTT
AGGCCAGGCGTGGTGACTCACACCTGTAATTCCAGCACTTCAGGAGGCCAA
GGTGGGAAGATCATTTGAGTTCAGGAGTTCGAGTTACAGCCTGGGCAATAA
AGTGAGACCCTGTCACTAACAAAATTAAAAAATAAAATAAATATAAAATAT
AGGCTTTAAAAAAGCATAGTCTTATTAACCATGTCTGTTGGTCAAAATCTG
CAAACTCTAAAAGAAGAAAAGAAGAAAAAACCAAGCTTAGGGTATTTTTCC
TCCCGTGCCTGAGTCCCAATTACATTCACGACAGTACTTTCAATGAACATA
ATTGTTAGGACCACTGAGGAATCATGAAAAATGATCTCTGCTTAGTACATT
TGATGCAAAATGACTTATTAGGGGCTGTTTTTCTAGCTATAGTGTCTCGAG
TACTAATATGCAATTATGAAAATTATATTAAATCTGGGATTATGACGGTAT
CACTGTATCATCTTGGTCTTGTTCTGGCTGTCACCAAGCATGACCCAGGTC
AACTTTTTTTTTCCCCTGAATTACCCATCAAATTGATCTGCAGCTGACTAA
AGGCCACAGCTGAGCCTGGAACTGACCCTTCCTTCATCCTCAACCTGCTGT
CCTCCAGAAAGCACCAAGGAAAAAGCAGAGAATGACAGCAAACAGATCACT
AGGCCTCTGACCACAGGTGCTGAGTACTCAGCAGCCCTCATATAATAGGTT
TGAAAGTACTCCTTAAAATAAAACACTGTTTCCCTTTGGAACTATTTACAA
GGATGAAACAACCGTATACCTGAGAAATAACTTGCTCTGGTGTCAATTCGC
TATTCGCCAGCAGACATCAGAACACACCGAGTTTCCAGATGCTGGTTTTTC
CCCTTAAATCAGGAAATACACCTGGACAATTTCTAGAAGACTACAATTCAG
TCTAGCCACAAAGGGGATTTTTTTTTTTTGGTAACAGGCTAGAGCCCGGTT
CTGTAAGTCTTTAGCTGAAATGGTCCAGTACAAAAGCACTGGAAATGAGTG
GGCTAGGAGGACAAGGACCGTCTCCTGCGTGAGGAGTTGGTTGGAGGTCCC
CAAGGCCAGGTACCCCCTGCACTCTTATTGGATTCCTCTCTGTCTTCTTGG
AGTTTTGAAAAACTCCTTCGAACACCAGGCTTTTTTCTTTAGAAAACAAGT
CTCCAATCGTTCTCTGTTCCGTAGAAAGAGAAAGAAAACCTGGAGCAGCTG
CTGAAAAATCTAATGAGGAACTAAGAGGCAAACCCACCA

In some embodiments, the introduction, removal, or modification of 3′ UTR AREs can be used to modulate the stability of an mRNA encoding a CLRN1 protein. In other embodiments, AREs can be removed or mutated to increase the intracellular stability and thus increase translation and production of a CLRN1 protein.

In other embodiments, non-ARE sequences may be incorporated into the 5′ or 3′ UTRs. In some embodiments, introns or portions of intron sequences may be incorporated into the flanking regions of the polynucleotides in any of the vectors, compositions, kits, and methods provided herein. Incorporation of intronic sequences may increase protein production as well as mRNA levels.

In some embodiments of any of the vectors described herein, the vector includes a chimeric intron sequence (SEQ ID NO: 16).

### Mammalian Cells

Also provided herein is a cell (e.g., a mammalian cell) that includes any of the nucleic acids, vectors (e.g., at least two different vectors described herein), or compositions described herein. Skilled practitioners will appreciate that the nucleic acids and vectors described herein can be introduced into any mammalian cell. Non-limiting examples of vectors and methods for introducing vectors into mammalian cells are described herein.

In some embodiments, the cell is a human cell, a mouse cell, a porcine cell, a rabbit cell, a dog cell, a cat cell, a rat cell, or a non-human primate cell. In some embodiments, the cell is a specialized cell of the cochlea. In some embodiments, the cell is a cochlear hair cell, such as a cochlear inner hair cell or a cochlear out hair cell. In some embodiments, the cell is an ocular cell (e.g. a retinal cell, a retinal ganglion cell, an amacrine cell, a hortizontal cell, a bipolar cell, a photoreceptor cell).

In some embodiments, the mammalian cell is in vitro. In some embodiments, the mammalian cell is present in a mammal. In some embodiments, the mammalian cell is an autologous cell obtained from a subject and cultured ex vivo.

### Methods

Also provided herein are methods that include: introducing into a cochlea of a mammal (e.g., a human) a therapeutically effective amount of any of the compositions described herein.

Also provided herein are methods of increasing expression of a full-length CLRN1 protein in a mammalian cell, that include: introducing any of the compositions described herein into the mammalian cell.

Also provided herein are methods of increasing expression of a full-length CLRN1 protein in an inner hair cell, an outer hair cell, or both, in a cochlea of a mammal (e.g., a human) that include: introducing into the cochlea of the mammal a therapeutically effective amount of any of the compositions described herein.

Also provided herein are methods of increasing expression of a full-length CLRN1 protein in an eye of a mammal (e.g., a human) that include: intraocularly administering to the eye of the mammal a therapeutically effective amount of any of the compositions described herein.

Also provided herein are methods of treating hearing loss in a subject identified as having a defective CLRN1 gene that include: administering a therapeutically effective amount of any of the compositions described herein into the cochlea of the subject.

Also provided herein are methods of treating vision loss in a subject identified as having a defective CLRN1 gene that include: administering a therapeutically effective amount of any of the compositions described herein into the eye of the subject.

In some embodiments of any of these methods, the mammal has been previously identified as having a defective CLRN1 gene (e.g., a CLRN1 gene having a mutation that results in a decrease in the expression and/or activity of a CLRN1 protein encoded by the gene). Some embodiments of any of these methods further include, prior to the introducing or administering step, determining that the subject has a defective CLRN1 gene. Some embodiments of any of these methods can further include detecting a mutation in a CLRN1 gene in a subject. Some embodiments of any of the methods can further include identifying or diagnosing a subject as having hearing loss and/or vision loss.

In some embodiments of any of these methods, two or more doses of any of the compositions described herein are introduced or administered into the cochlea of the mammal or subject. Some embodiments of any of these methods can include introducing or administering a first dose of the composition into the cochlea of the mammal or subject, assessing hearing function of the mammal or subject following the introducing or the administering of the first dose, and administering an additional dose of the composition into the cochlea of the mammal or subject found not to have a hearing function within a normal range (e.g., as determined using any test for hearing known in the art).

In some embodiments of any of the methods described herein, the composition can be formulated for intra-cochlear administration. In some embodiments of any of the methods described herein, the compositions described herein can be administered via intra-cochlear administration or local administration. In some embodiments of any of the methods described herein, the compositions are administered through the use of a medical device (e.g., any of the exemplary medical devices described herein).

In some embodiments, intra-cochlear administration can be performed using any of the methods described herein or known in the art. For example, a composition can be administered or introduced into the cochlea using the following surgical technique: first using visualization with a 0 degree, 2.5-mm rigid endoscope, the external auditory canal is cleared and a round knife is used to sharply delineate an approximately 5-mm tympanomeatal flap. The tympanomeatal flap is then elevated and the middle ear is entered posteriorly. The chorda tympani nerve is identified and divided, and a currette is used to remove the scutal bone, exposing the round window membrane. To enhance apical distribution of the administered or introduced composition, a surgical laser may be used to make a small 2-mm fenestration in the oval window to allow for perilymph displacement during trans-round window membrane infusion of the composition. The microinfusion device is then primed and brought into the surgical field. The device is maneuvered to the round window, and the tip is seated within the bony round window overhang to allow for penetration of the membrane by the microneedle(s). The footpedal is engaged to allow for a measured, steady infusion of the composition. The device is then withdrawn and the round window and stapes foot plate are sealed with a gelfoam patch.

In some embodiments of any of these methods, two or more doses of any of the compositions described herein are introduced or administered into the eye of the mammal or subject. Some embodiments of any of these methods can include introducing or administering a first dose of the composition into the eye (e.g., intraocular space) of the mammal or subject, assessing hearing function of the mammal or subject following the introducing or the administering of the first dose, and administering an additional dose of the composition into the eye of the mammal or subject found not to have a vision within a normal range (e.g., as determined using any test for vision known in the art).

In some embodiments of any of the methods described herein, the composition can be formulated for intra-ocular administration. In some embodiments of any of the methods described herein, the compositions described herein can be administered via intra-ocular administration or local administration.

In some embodiments, intra-ocular administration can be performed using any of the methods described herein or known in the art.

In some embodiments of any of the methods described herein, the subject or mammal is a rodent, a non-human primate, or a human. In some embodiments of any of the methods described herein, the subject or mammal is an adult, a teenager, a juvenile, a child, a toddler, an infant, or a newborn. In some embodiments of any of the methods described herein, the subject or mammal is 1-5, 1-10, 1-20, 1-30, 1-40, 1-50, 1-60, 1-70, 1-80, 1-90, 1-100, 1-110, 2-5, 2-10, 10-20, 20-30, 30-40, 40-50, 50-60, 60-70, 70-80, 80-90, 90-100, 100-110, 10-30, 10-40, 10-50, 10-60, 10-70, 10-80, 10-90, 10-100, 10-110, 20-40, 20-50, 20-60, 20-70, 20-80, 20-90, 20-100, 20-110, 30-50, 30-60, 30-70, 30-80, 30-90, 30-100, 40-60, 40-70, 40-80, 40-90, 40-100, 50-70, 50-80, 50-90, 50-100, 60-80, 60-90, 60-100, 70-90, 70-100, 70-110, 80-100, 80-110, or 90-110 years of age. In some embodiments of any of the methods described herein, the subject or mammal is 1, 2, 3, 4, 5, 6, 7, 8, 9, 10, or 11 months of age.

In some embodiments of any of the methods described herein, the subject or mammal has or is at risk of developing hearing loss and/or vision loss (e.g., Usher syndrome type III, retinitis pigmentosa). In some embodiments of any of the methods described herein, the subject or mammal has been previously identified as having a mutation in a CLRN1 gene. In some embodiments of any of the methods described herein, the subject or mammal has any of the mutations in a CLRN1 gene that are described herein or are known in the art to be associated with hearing loss and/or vision loss.

In some embodiments of any of the methods described herein, the subject or mammal has been identified as being a carrier of a mutation in a CLRN1 gene (e.g., via genetic testing). In some embodiments of any of the methods described herein, the subject or human has been identified as having a mutation in a CLRN1 gene and has been diagnosed with hearing loss and/or vision loss (e.g., Usher syndrome type III, retinitis pigmentosa). In some embodiments of any of the methods described herein, the subject or human has been identified as having hearing loss and/or vision loss (e.g., Usher syndrome type III, retinitis pigmentosa).

In some embodiments, successful treatment of hearing loss (e.g., Usher syndrome type III) can be determined in a subject using any of the conventional functional hearing tests known in the art. Non-limiting examples of functional hearing tests are various types of audiometric assays (e.g., pure-tone testing, speech testing, test of the middle ear, auditory brainstem response, and otoacoustic emissions).

In some embodiments, successful treatment of vision loss can be determined in a subject using any of the conventional functional vision tests known in the art. Non-limiting examples of functional retinal and vision tests are acuity testing, intraocular pressure (IOP) testing, and an electroretinogram (ERG).

Also provided herein are methods of increasing expression of an active CLRN1 protein (e.g., a full-length CLRN1 protein) in a mammalian cell that include introducing any of the compositions described herein into the mammalian cell. In some embodiments of these methods, the mammalian cell is a cochlear hair cell (e.g., an inner hair cell, an outer hair cell) or an ocular cell (e.g., a retinal cell). In some embodiments of these methods, the mammalian cell is a human cell (e.g., a human cochlear hair cell). In some embodiments of these methods, the mammalian cell is in vitro. In some embodiments of these methods, the mammalian cell is in a mammal. In some embodiments of these methods, the mammalian cell is originally obtained from a mammal and is cultured ex vivo. In some embodiments, the mammalian cell has previously been determined to have a defective CLRN1 gene.

Methods for introducing any of the compositions described herein into a mammalian cell are known in the art (e.g., via lipofection or through the use of a viral vector, e.g., any of the viral vectors described herein).

An increase in expression of an active CLRN1 protein (e.g., a full-length CLRN1 protein) as described herein is, e.g., as compared to a control or to the level of expression of an active CLRN1 protein (e.g., a full-length CLRN1 protein) prior to the introduction of the vector(s).

Methods of detecting expression and/or activity of CLRN1 are known in the art. In some embodiments, the level of expression of a CLRN1 protein can be detected directly (e.g., detecting CLRN1 protein or detecting CLRN1 mRNA). Non-limiting examples of techniques that can be used to detect expression and/or activity of CLRN1 directly include: real-time PCR, Western blotting, immunoprecipitation, immunohistochemistry, or immunofluorescence. In some embodiments, expression of a CLRN1 protein can be detected indirectly (e.g., through functional hearing tests, functional retinal and vision tests).

### Pharmaceutical Compositions and Kits

In some embodiments, any of the compositions described herein can further include one or more agents that promote the entry of a nucleic acid or any of the vectors described herein into a mammalian cell (e.g., a liposome or cationic lipid). In some embodiments, any of the vectors described herein can be formulated using natural and/or synthetic polymers. Non-limiting examples of polymers that may be included in any of the compositions described herein can include, but are not limited to, DYNAMIC POLYCONJUGATE® (Arrowhead Research Corp., Pasadena, Calif.), formulations from Mirus Bio (Madison, Wis.) and Roche Madison (Madison, Wis.), PhaseRX polymer formulations such as, without limitation, SMARTT POLYMER TECHNOLOGY® (PhaseRX, Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly (lactic-co-glycolic acid) (PLGA) polymers, RONDEL™ (RNAi/Oligonucleotide Nanoparticle Delivery) polymers (Arrowhead Research Corporation, Pasadena, Calif.), and pH responsive co-block polymers, such as, but not limited to, those produced by PhaseRX (Seattle, Wash.). Many of these polymers have demonstrated efficacy in delivering oligonucleotides in vivo into a mammalian cell (see, e.g., deFougerolles, Human Gene Ther. 19:125-132, 2008; Rozema et al., Proc. Natl. Acad. Sci. U.S.A. 104:12982-12887, 2007; Rozema et al., Proc. Natl. Acad. Sci. U.S.A. 104:12982-12887, 2007; Hu-Lieskovan et al., Cancer Res. 65:8984-8982, 2005; Heidel et al., Proc. Natl. Acad. Sci. U.S.A. 104:5715-5721, 2007).

Any of the compositions described herein can be, e.g., a pharmaceutical composition. A pharmaceutical composition can include any of the compositions described herein and one or more pharmaceutically or physiologically acceptable carriers, diluents, or excipients. Such compositions may comprise one or more buffers, such as neutral-buffered saline, phosphate-buffered saline, and the like; one or more carbohydrates, such as glucose, mannose, sucrose, and dextran; mannitol; one or more proteins, polypeptides, or amino acids, such as glycine; one or more antioxidants; one or more chelating agents, such as EDTA or glutathione; and/or one or more preservatives.

In some embodiments, the composition includes a pharmaceutically acceptable carrier (e.g., phosphate buffered saline, saline, or bacteriostatic water). Upon formulation, solutions will be administered in a manner compatible with the dosage formulation and in such amount as is therapeutically effective. The formulations are easily administered in a variety of dosage forms such as injectable solutions, injectable gels, drug-release capsules, and the like.

As used herein, the term “pharmaceutically acceptable carrier” includes solvents, dispersion media, coatings, antibacterial agents, antifungal agents, and the like that are compatible with pharmaceutical administration. Supplementary active compounds can also be incorporated into any of the compostions described herein.

In some embodiments, a single dose of any of the compositions described herein can include a total sum amount of the at least two different vectors of at least 1 ng, at least 2 ng, at least 4 ng, about 6 ng, about 8 ng, at least 10 ng, at least 20 ng, at least 30 ng, at least 40 ng, at least 50 ng, at least 60 ng, at least 70 ng, at least 80 ng, at least 90 ng, at least 100 ng, at least 200 ng, at least 300 ng, at least 400 ng, at least 500 ng, at least 1 μg, at least 2 μg, at least 4 μg, at least 6 μg, at least 8 μg, at least 10 μg, at least 12 μg, at least 14 μg, at least 16 μg, at least 18 μg, at least 20 μg, at least 22 μg, at least 24 μg, at least 26 μg, at least 28 μg, at least 30 μg at least 32 μg, at least 34 μg, at least 36 μg, at least 38 μg, at least 40 μg, at least 42 μg, at least 44 μg, at least 46 μg, at least 48 μg, at least 50 μg, at least 52 μg, at least 54 μg, at least 56 μg, at least 58 μg, at least 60 μg, at least 62 μg, at least 64 μg, at least 66 μg, at least 68 μg, at least 70 μg, at least 72 μg, at least 74 μg, at least 76 μg, at least 78 μg, at least 80 μg, at least 82 μg, at least 84 μg, at least 86 μg, at least 88 μg, at least 90 μg, at least 92 μg, at least 94 μg, at least 96 μg, at least 98 μg, at least 100 μg, at least 102 μg, at least 104 μg, at least 106 μg, at least 108 μg, at least 110 μg, at least 112 μg, at least 114 μg, at least 116 μg, at least 118 μg, at least 120 μg, at least 122 μg, at least 124 μg, at least 126 μg, at least 128 μg, at least 130 μg at least 132 μg, at least 134 μg, at least 136 μg, at least 138 μg, at least 140 μg, at least 142 μg, at least 144 μg, at least 146 μg, at least 148 μg, at least 150 μg, at least 152 μg, at least 154 μg, at least 156 μg, at least 158 μg, at least 160 μg, at least 162 μg, at least 164 μg, at least 166 μg, at least 168 μg, at least 170 μg, at least 172 μg, at least 174 μg, at least 176 μg, at least 178 μg, at least 180 μg, at least 182 μg, at least 184 μg, at least 186 μg, at least 188 μg, at least 190 μg, at least 192 μg, at least 194 μg, at least 196 μg, at least 198 μg, or at least 200 μg, e.g., in a buffered solution.

The compositions provided herein can be, e.g., formulated to be compatible with their intended route of administration. A non-limiting example of an intended route of administration is local administration (e.g., intra-cochlear administration).

In some embodiments, the therapeutic compositions are formulated to include a lipid nanoparticle. In some embodiments, the therapeutic compositions are formulated to include a polymeric nanoparticle. In some embodiments, the therapeutic compositions are formulated to comprise a mini-circle DNA. In some embodiments, the therapeutic compositions are formulated to comprise a CELiD DNA. In some embodiments, the therapeutic compositions are formulated to comprise a synthetic perilymph solution. An exemplary synthetic perilymph solution includes 20-200 mM NaCl; 1-5 mM KCl; 0.1-10 mM CaCl₂; 1-10 mM glucose; 2-50 mM HEPES, having a pH of between about 6 and about 9.

Also provided are kits including any of the compositions described herein. In some embodiments, a kit can include a solid composition (e.g., a lyophilized composition including the at least two different vectors described herein) and a liquid for solubilizing the lyophilized composition. In some embodiments, a kit can include a pre-loaded syringe including any of the compositions described herein.

In some embodiments, the kit includes a vial comprising any of the compositions described herein (e.g., formulated as an aqueous composition, e.g., an aqueous pharmaceutical composition).

In some embodiments, the kits can include instructions for performing any of the methods described herein.

### Devices and Surgical Methods

Provided herein are therapeutic delivery systems for treating hearing loss and/or vision loss (e.g., Usher syndrome type III, retinitis pigmentosa). In one aspect, the therapeutic delivery systems include i) a medical device capable of creating one or a plurality of incisions in a round window membrane of an inner ear of a human subject in need thereof, and ii) an effective dose of a composition (e.g., any of the compositions described herein). In some embodiments, the medical device includes a plurality of micro-needles.

Also provided herein are surgical methods for treatment of hearing loss (e.g., Usher syndrome type III). In some embodiments, the methods include the steps of: introducing into a cochlea of a human subject a first incision at a first incision point; and administering intra-cochlearly a therapeutically effective amount of any of the compositions provided herein. In some embodiments, the composition is administered to the subject at the first incision point. In some embodiments, the composition is administered to the subject into or through the first incision.

In some embodiments of any of the methods described herein, any of the compositions described herein is administered to the subject into or through the cochlea oval window membrane. In some embodiments of any of the methods described herein, any of the compositions described herein is administered to the subject into or through the cochlea round window membrane. In some embodiments of any of the methods described herein, the composition is administered using a medical device capable of creating a plurality of incisions in the round window membrane. In some embodiments, the medical device includes a plurality of micro-needles. In some embodiments, the medical device includes a plurality of micro-needles including a generally circular first aspect, where each micro-needle has a diameter of at least about 10 microns. In some embodiments, the medical device includes a base and/or a reservoir capable of holding the composition. In some embodiments, the medical device includes a plurality of hollow micro-needles individually including a lumen capable of transferring the composition. In some embodiments, the medical device includes a means for generating at least a partial vacuum.

Also provided herein are surgical methods for treatment of vision loss (e.g., retinitis pigmentosa). In some embodiments, the methods include the steps of: administering intra-ocularly a therapeutically effective amount of any of the compositions provided herein.

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations that become evident as a result of the teaching provided herein.

Without further description, it is believed that one of ordinary skill in the art can, using the preceding description and the following illustrative examples, make and utilize the compounds of the present invention and practice the claimed methods. The following working examples specifically point out various aspects of the present invention, and are not to be construed as limiting in any way the remainder of the disclosure.

### EXAMPLES

### Example 1: Construction of Viral Vectors

Recombinant AAV is generated by transfection with an adenovirus-free method as used by Xiao et al. J. Virol. 73(5):3994-4003, 1999. The cis plasmids with AAV ITRs, the trans plasmid with AAV Rep and Cap genes, and a helper plasmid with an essential region from an adenovirus genome are co-transfected in 293 cells in a ratio of 1:1:2. The AAV vectors used here express human CLRN1 or mouse CLRN1 under multiple dual vector strategies using the constructs described below. AAV serotypes 1, 2, 3, 4, 5, 6, 7, 8, 9, rh8, rh10, rh39, rh43, and Anc80 are each prepared to encapsulate three sets of CLRN1 constructs to test (i) a concatemerization-transplicing strategy, (ii) a hybrid intronic-homologous recombination-transplicing strategy, and (iii) an exonic homologous recombination strategy, as summarized by Pryadkina et al., Meth. Clin. Devel. 2:15009, 2015.

### Example 2: Generating and Purifying Viral Particles

Recombinant AAV-1 is produced using a triple transfection protocol and purified by two sequential cesium chloride (CsCl) density gradients, as described by Pryadkina et al., Mol. Ther. 2:15009, 2015. At the end of second centrifugation, 11 fractions of 500 μl are recovered from the CsCl density gradient tube and purified through dialysis in 1×PBS. The fractions are analyzed by dot blot to determine those containing rAAV genomes. The viral genome number (vg) of each preparation is determined by a quantitative real-time PCR-based titration method using primers and probe corresponding to the ITR region of the AAV vector genome (Bartoli et al. Gene. Ther. 13:20-28, 2006).

### Example 3: Formulation of Viral Particles

AAV produced at a titer of 1e14 vg/mL is prepared at dilutions of 3.2e13, 1.0e13, 3.2e12, 1.0e12 vg/mL in artificial perilymph. Artificial perilymph is prepared by combining the following reagents: NaCl, 120 mM; KCl, 3.5 mM; CaCl₂, 1.5 mM; glucose, 5.5 mM; HEPES, 20 mM. The artificial perilymph is titrated with NaOH to adjust its pH to 7.5 (total Na⁺ concentration of 130 mM) (Chen et al., J. Controlled Rd. 110:1-19, 2005).

### Example 4: Device Description

The AAV-CLRN1 formulation is delivered to the cochlea using a specialized microcatheter designed for consistent and safe penetration of the round window membrane (RWM). The microcatheter is shaped such that the surgeon performing the delivery procedure can enter the middle ear cavity via the external auditory canal and contact the end of the microcatheter with the RWM. The distal end of the microcatheter is comprised of at least one microneedle with diameter between 10 and 1,000 microns, which produces perforations in the RWM that are sufficient to allow AAV-CLRN1 to enter the cochlear perilymph of the scala tympani at a rate of approximately 1 uL/min, but small enough to heal without surgical repair. The remaining portion of the microcatheter, proximal to the microneedle(s), is loaded with the AAV-CLRN1/artificial perilymph formulation at a titer of approximately 1e13 vg/mL. The proximal end of the microcatheter is connected to a micromanipulator that allows for precise, low volume infusions of approximately 1 μL/min.

### Example 5: Animal Model 1A: Surgical Method in Aged Mice

AAV-CLRN1 prepared in artificial perilymph is administered to the scala tympani in mice as described by Shu et al. (Human Gene Therapy, doi:10.1089/hum.2016.053, June 2016). Six-week-old male mice are anesthetized using an intraperitoneal injection of xylazine (20 mg/kg) and ketamine (100 mg/kg). Body temperature is maintained at 37° C. using an electric heating pad. An incision is made from the right post-auricular region and the tympanic bulla is exposed. The bulla is perforated with a surgical needle and the small hole is expanded to provide access to the cochlea. The bone of the cochlear lateral wall of the scala tympani is thinned with a dental drill so that the membranous lateral wall is left intact. A Nanoliter Microinjection System in conjunction with glass micropipette is used to deliver a total of approximately 300 nL of AAV-CLRN1 in artificial perilymph to the scala tympani at a rate of 2 nL/second. The glass micropipette is left in place for 5 minutes post-injection. Following cochleostomy and injection, the opening in the tympanic bulla is sealed with dental cement, and the muscle and skin are sutured. The mice are allowed to awaken from anesthesia and their pain is controlled with 0.15 mg/kg buprenorphine hydrochloride for 3 days.

### Example 6: Animal Model 2: Reciprocating Micropump in Guinea Pig

### Surgical Procedure

AAV-CLRN1 prepared in artificial perilymph is administered to guinea pigs to assess distribution and toxicity following intracochlear delivery with a reciprocating micropump as descrbied by Tandon et al., Lab Chip, DOI: 10.1039/c51c01396h, 2015. Male guinea pigs weighing approximately 350 g each (n=16) are anesthetized with a combination of pentobarbital sodium (Nembutal; 25 mg kg-1, injected intraperitoneally), fentanyl (0.2 mg kg-1, intramuscularly), and haloperidol (10 mg kg-1, intramuscularly). Lidocaine with epinephrine is given subcutaneously at the incision site as a topical anesthetic. Using a dorsal approach, a 5 mm diameter hole is made in the bulla and a cochleostomy is created approximately 0.5 mm distal to the round window membrane. The cannula of the micropump (described below) is inserted into the cochleostomy, threaded into the cochlea 3 mm apically, and glued to the bulla with a common cyanoacrylate glue. For compound action potential (CAP) measurements, a perfluoroalkoxy-alkane-insulated silver wire electrode (203 μm uncoated diameter) is inserted near the round window niche and glued to the bulla.

Procedures for measurement of distortion product otoacoustic emissions (DPOAEs) and CAPs are performed as previously described in Tandon et al. Biomed Microdevices 17:3-21, 2015. DPOAEs are measured before and after the cochleostomy procedure at the characteristic frequencies: 32, 24, 16, 12, 8, 5.6, 4, and 2.78 kHz in order to monitor any damage that occurs as a result of the surgery.

AAV-CLRN1 at a maximum titer of 1e14 vg/mL is administered to the guinea pig using a micropump as described by Tandon et al. Lab Chip, DOI: 10.1039/c51c01396h, 2015. The micropump system has 4 selectable ports. These ports are connected to: (i) a large fluidic capacitor used for artificial perilymph storage; (ii) an outlet that connects to the cochlea; (iii) the outlet from an integrated AAV-CLRN1 reservoir; (iv) the inlet to the integrated AAV-CLRN1 reservoir. Each port is fluidically connected to a central pump chamber, and each is individually addressed with a valve. The sequence of events for reciprocating AAV-CLRN1 delivery is as follows: (i) an internal AAV-CLRN1-refresh loop is run, transferring AAV-CLRN1 from the AAV-CLRN1 reservoir into the main infuse-withdraw line; (ii) AAV-CLRN1 is infused into the cochlea and some artificial perilymph is drained from the artificial perilymph storage capacitor; (iii) the first two steps can be repeated several times for additional doses; (iv) after the AAV-CLRN1 has been allowed to diffuse for some time, a volume of perilymph is withdrawn from the cochlea that is equal to the volume infused in steps (i)-(iii), refilling the artificial perilymph storage capacitor. This process results in net delivery of drug with zero net fluid volume added to the cochlea.

The fluidic capacitors in the micropump are cylindrical chambers whose ceilings are a thin (25.4 μm), flexible, polyimide membrane. The pump chamber has a diameter of 3.5 mm, the fluidic storage capacitor has a diameter of 14 mm, and all of the remaining capacitors have diameters of 4 mm. The same membrane is deflected to block flow at each of the valves. The valve chambers have diameters of 3.1 mm. The serpentine channel that comprises the drug reservoir has a square cross section of width 762 μm and a length of 410 mm for a total volume of 238 μL. All of the other microchannels in the pump have a width of 400 μm and a height of 254 μm.

### Acute Drug Delivery in Guinea Pigs

The micropump is loaded with AAV-CLRN1 and artificial perilymph, and the cannula inserted into a cochleostomy made in the region of the cochlea between the locations with characteristic frequency sensitivity of 24 and 32 kHz, and threaded apically 3 mm, terminating in the 12-16 kHz region. Baseline DPOAE and CAP hearing tests are performed prior to the start of AAV-CLRN1/artificial perilymph infusion. The pump is then activated and approximately 1 μL of artificial perilymph is infused every 5 min until a total of approximately 10 μL of artificial perilymph is delivered to the cochlea. After a 20 min wait time, approximately 10 μL of perilymph is withdrawn from the cochlea. AAV-CLRN1 delivery is then initiated at a rate of approximately 1 μL every 5 min until a total of approximately 10 μL of fluid delivered.

Animals are sacrificed at 1 week, 1 month, 3 months, and 6 months post-treatment (n=4 per group) and their cochleae extracted. Extent of AAV transduction and CLRN1 expression along the organ of Corti is assessed via immunostaining with anti-CLRN1 antibodies. Antibodies against markers for hair cells (Myo7a) and supporting cells (Sox2) are used to quantify IHCs, OHCs, supporting cells and stereocilia morphology. Annexin V staining is used to assess evidence of apoptosis in cells along the cochlear sensory epithelium.

### Example 7: Animal Model 3: Sheep

AAV-CLRN1 prepared in artificial perilymph is administered to juvenile sheep to assess distribution and toxicity following delivery to the cochlea via trans-RWM infusion. Baseline auditory brainstem response (ABR) and distortion product optoacoustic emissions (DPOAEs) are measured in female sheep at 3 months of age (n=40), bilaterally, to assess pre-treatment inner hair cell (IHC) and outer hair cell (OHC) function. Following baseline ABR and DPOAE measurements, 20 uL of AAV1-CLRN1 at titers of 1.0e14, 3.2e13, 1.0e13 and 3.2e12 vg/mL is injected into the left scala tympani of the sheep (n=10 per group). Each animal's right ear is left as an untreated control. ABR and DPOAE measurements are taken again bilaterally 1, 5 and 10 days following the surgical procedure. At 6 months post-procedure, additional bilateral ABR and DPOAE measurements are taken from all animals, and the animals are subsequently sacrificed and their cochleae removed.

In half of the sacrificed animals (n=5 from each of the dose cohorts), immunostaining is performed to identify hair cell structures and to assess CLRN1 protein expression along the cochlear sensory epithelium. Antibodies against markers for hair cells (Myo7a), supporting cells (Sox2) and CLRN1 are used as described previously (Duncker et al. 2013, J Neurosci 33(22):9508-9519). At the basal, middle and apical turns of the organ of corti, total numbers of hair cells and those hair cells expressing CLRN1 are counted within 200 um regions.

In the remaining half of the sacrificed animals (remaining 5 animals from each dose cohort), cochlear tissue samples are collected from the same basal, middle and apical regions as described above, and assayed for CLRN1 mRNA transcript.

### Example 8: Human Clinical Example (Pediatric Treatment)

The patient is put under general anesthesia. The surgeon approaches the tympanic membrane from external auditory canal, makes a small incision at the inferior edge of the external auditory canal where it meets the tympani membrane, and lifts the tympanic membrane as a flap to expose the middle ear space. A surgical laser is used to make a small opening (approximately 2 mm) in the stapes footplate. The surgeon then penetrates the round window membrane with a microcatheter loaded with a solution of AAV-CLRN1 prepared in artificial perilymph at a titer of 1e13 vg/mL. The microcatheter is connected to a micromanipulator that infuses approximately 20 uL of the AAV-CLRN1 solution at a rate of approximately 1 uL/min. At the conclusion of the AAV-CLRN1 infusion, the surgeon withdraws the microcatheter and patches the holes in the stapes foot plate and RWM with a gel foam patch. The procedure concludes with replacement of the tympanic membrane flap.

### Example 9: Non-Invasive Prenatal Testing of Maternal Blood to Detect CLRN1 Mutation

Maternal blood samples (20-40 mL) are collected into Cell-free DNA tubes. At least 7 mL of plasma is isolated from each sample via a double centrifugation protocol of 2,000 g for 20 minutes, followed by 3,220 g for 30 minutes, with supernatant transfer following the first spin. cfDNA is isolated from 7-20 mL plasma using a QIAGEN QIAmp® Circulating Nuclei Acid kit and eluted in 45 μL TE buffer. Pure maternal genomic DNA is isolated from the buffy coat obtained following the first centrifugation.

By combining thermodynamic modeling of the assays to select probes with minimized likelihood of probe-probe interaction with amplification approaches described previously (Stiller et al., Genome Res. 19(10):1843-1848, 2009), multiplexing of 11,000 assays can be achieved. Maternal cfDNA and maternal genomic DNA samples are pre-amplified for 15 cycles using 11,000 target-specific assays and an aliquot is transferred to a second PCR reaction of 15 cycles using nested primers. Samples are prepared for sequencing by adding barcoded tags in a third 12-cycle round of PCR. The amplicons are then sequenced using an Illumina HiSeq sequencer. Genome sequence alignment is performed using commercially available software.

### Example 10: Adenovirus (AAV) Trans-Splicing Strategy

At least two different nucleic acid vectors (e.g., AAV vectors) can be used to reconstitute an active CLRN1 gene (e.g., a full-length CLRN1 gene) within a cell following intermolecular concatamerization and trans-splicing. See, e.g., Yan et al., Proc. Natl. Acad. Sci. U.S.A. 97:12; 6716-6721, 2000, incorporated in its entirety herein.

In some examples, two different nucleic acid vectors will be used. A first nucleic acid vector can include a promoter (e.g., any of the promoters described herein), a first coding sequence that encodes an N-terminal portion of a CLRN1 protein positioned 3′ of the promoter (e.g., any of the sizes of a portion of a CLRN1 protein described herein and/or any of the N-terminal portions of a CLRN1 protein described herein), and a splice donor sequence positioned at the 3′ end of the first coding sequence. A second nucleic acid vector can include a splice acceptor sequence, a second coding sequence that encodes a C-terminal portion of a CLRN1 protein (i.e., the entire portion of the CLRN1 protein that is not included in the N-terminal portion) positioned at the 3′ end of the splice acceptor sequence (e.g., any of the sizes of a portion of a CLRN1 protein described herein and/or any of the C-terminal portions of a CLRN1 protein described herein), and a polyadenylation sequence at the 3′ end of the second coding sequence (e.g., any of the polyadenylation sequences described herein). In some embodiments, each of the encoded portions is at least 30 amino acid residues in length (e.g., at least 50 amino acids, at least 75 amino acids, or at least 100 amino acids in length), the amino acid sequence of each of the encoded portions does not overlap with the sequence of the other encoded portion, and no single vector of the two different vectors encodes an active CLRN1 protein (e.g., a full-length CLRN1 protein). When introduced into a mammalian cell (e.g., any of the mammalian cells described herein) splicing occurs between the splice donor sequence and the splice acceptor sequence, thereby forming a recombined nucleic acid that encodes an active CLRN1 protein (e.g., a full-length CLRN1 protein).

In another example, three different nucleic acid vectors can be used. A first nucleic acid vector can include a portion of a promoter sequence (e.g., any of the promoter sequences described herein), a first coding sequence of a CLRN1 gene that encodes a first portion of a CLRN1 protein (e.g., any of the CLRN1 coding sequences described herein) positioned 3′ of the promoter, and a first splice donor sequence positioned at the 3′ end of the first coding sequence. A second nucleic acid vector can include a first splice acceptor sequence, a second coding sequence of a CLRN1 gene that encodes a second portion of a CLRN1 protein positioned at the 3′ end of the first splice acceptor sequence, and a second splice donor sequence positioned at the 3′ end of the second coding sequence (e.g., any of the splicedonor sequences described herein). A feature of the second nucleic acid vector will be that self-splicing cannot occur (i.e., splicing will not occur between the second splice donor sequence and the first splice acceptor sequence of the second nucleic acid vector). In some embodiments, the splice donor sequence of the first nucleic acid vector and the second splice donor sequence of the second nucleic acid vector are the same (e.g., any of the splice donor sequences described herein or known in the art). In some embodiments, the first splice donor sequence of the first nucleic acid vector and the second splice donor sequence of the second nucleic acid vector are different (e.g., any of the splice donor sequences described herein or known in the art). A third nucleic acid vector will include a second splice acceptor sequence, a third coding sequence of a CLRN1 gene that encodes a third portion of a CLRN1 protein positioned at the 3′ end of the second splice acceptor sequence, and a polyadenylation sequence positioned at the 3′ end of the third coding sequence (e.g., any of the polyadenylation sequences described herein). In such methods where three nucleic acid vectors are used, the first splice donor sequence and the first splice acceptor sequence can assemble together (recombine) and the second splice donor sequence and the second splice acceptor sequence can assemble together (recombine), and the portion of CLRN1 protein encoded by the first, second, and third coding sequences do not overlap, and when introduced into a mammalian cell (e.g., any of the mammalian cells described herein), splicing occurs between the first splice donor sequence and the first splice acceptor sequence, and between the second splice donor sequence and the second splice acceptor sequence, to form a recombined nucleic acid that encodes an active CLRN1 protein (e.g., a full-length CLRN1 protein). Based on the strategies provided above, one skilled in the art would understand how to develop a strategy using four, five, or six different nucleic acid vectors.

In any of the examples of these methods, none of the amino acid sequences of the encoded portions overlap with any other encoded portion, and no single vector encodes an active CLRN1 protein (e.g., a full-length CLRN1 protein).

Each of the at least two different vectors includes a coding sequence that encodes a different portion of a CLRN1 protein, and each of the encoded portions can be at least 30 amino acids (e.g., between about 30 amino acids to about 1200 amino acids, or any of the other subranges of this range described herein).

In some embodiments, each of the coding sequences can include at least one exon and at least one intron of SEQ ID NO: 9 (e.g., at least two exons and at least one intron, at least two exons and at least two introns, at least three exons and at least one intron, at least three exons and at least two introns, or at least three exons and at least three introns). In some embodiments, each of the at least two different vectors includes a coding sequence that encodes a different portion of a CLRN1 protein, and each of the encoded portions can encode up to 80% of the amino acid sequence of SEQ ID NO: 1 (e.g., up to 10%, up to 20%, up to 30%, up to 40%, up to 50%, up to 60%, or up to 70% of SEQ ID NO: 1) such that each of the encoded portions is non-overlapping. In some embodiments, each of the at least two different vectors includes a coding sequence that encodes a different portion of a CLRN1 protein, each of the encoded portions encoding up to 80% of the amino acid sequence of SEQ ID NO: 3 (e.g., up to 10%, up to 20%, up to 30%, up to 40%, up to 50%, up to 60%, or up to 70% of SEQ ID NO: 3), provided that each of the encoded portions is non-overlapping with any other. In some embodiments, each of the at least two different vectors includes a coding sequence that encodes a different portion of a CLRN1 protein, each of the encoded portions encoding up to 80% of the amino acid sequence of SEQ ID NO: 5 (e.g., up to 10%, up to 20%, up to 30%, up to 40%, up to 50%, up to 60%, or up to 70% of SEQ ID NO: 5), provided that each of the encoded portions is non-overlapping with any other. In some embodiments, each of the at least two different vectors includes a coding sequence that encodes a different portion of a CLRN1 protein, each of the encoded portions encoding up to 80% of the amino acid sequence of SEQ ID NO: 7 (e.g., up to 10%, up to 20%, up to 30%, up to 40%, up to 50%, up to 60%, or up to 70% of SEQ ID NO: 7), provided that each of the encoded portions is non-overlapping with any other.

Each of the at least two nucleic acid vectors may further include an inverted terminal repeat (ITR) to allow head-to-tail recombination. The ITR will be subsequently removed via splicing. For example, the ITR could be a palindromic double-D ITR as described in Yan et al., Proc. Natl. Acad. Sci. U.S.A. 97(12):6716-6721, 2000, incorporated in its entirety herein, or an AAV serotype-2 ITR as described in Gosh et al., Mol. Ther. 16:124-130, 2008, and Gosh et al., Human Gene Ther. 22: 77-83, 2011. Non-limiting examples of splice acceptor and/or donor sequences are known in the art. See, e.g., Reich et al., Human Gene Ther. 14(1):37-44, 2003, and Lai et al. (2005) Nat. Biotechnol. 23(11):1435-1439, 2005, 2005. The splice donor and acceptor sequences can be any endogenous intron splice donor/acceptor sequence of a gene (e.g., a CLRN1 gene). For example, the splice donor sequence can be: 5′-GTAAGTATCAAGGTTACAAGACAGGTTTAAGGAGACCAATAGA AACTGGGCTTGTCGAGACAGAGAAGACTCTTGCGTTTCT-3′ (SEQ ID NO: 22) and the splice acceptor sequence can be 5′-GATAGGCACCTATTGGTCTTACTG ACATCCACTTTGCCTTTCTCTCCACAG-3′ (SEQ ID NO: 23) (see, e.g., Trapani et al., EMBO Mol. Med. 6(2):194-211, 2014). Methods of evaluating splicing and splicing efficiency are known in the art (see, e.g., Lai et al., Nat. Biotechnol. 23(11): 1435-1439, 2005).

### Example 11: Hybrid Vector Trans-Splicing Strategy Using an Alkaline Phosphatase (AP) Highly Recombinogenic Exogenous Gene Region

At least two (e.g., two, three, four, five, or six) different nucleic acid vectors (e.g., AAV vectors) can also be used in any of the methods described herein to reconstitute an active CLRN1 gene (e.g., a full-length CLRN1 gene) within a cell following intermolecular concatamerization, marker gene-mediated recombination, and trans-splicing. This strategy is a hybrid strategy as it will include homologous recombination and/or trans-splicing. See, e.g., Gosh et al., Mol. Ther. 16: 124-130, 2008; Gosh et al., Human Gene Ther. 22: 77-83, 2011; and Duan et al., Mol. Ther. 4: 383-391, 2001, each incorporated in its entirety herein. As used herein, a detectable marker gene can be a highly recombinogenic DNA sequence that will allow for coding sequence-independent recombination. An non-limiting example of a detectable marker gene is an alkaline phosphatase (AP) gene. For example, the detectable marker gene can be the middle one-third of the human placental AP complementary DNA, which is 872 bp in length (see, e.g., Gosh et al., 2008). At least two different nucleic acid vectors will contain a detectable marker gene (e.g., any of the detectable marker genes described herein). Since the hybrid vector will be constructed based on a trans-splicing vector as described in Example 10, an active CLRN1 gene (e.g., a full-length CLRN1 gene) may be reconstituted using either ITR-mediated recombination and trans-splicing or detectable marker gene-mediated (e.g., AP-gene mediated) recombination and trans-splicing. After trans-splicing, an active CLRN1 gene (e.g., a full-length CLRN1 gene) will be reconstituted in the genomic DNA of a mammalian cell (e.g., any mammalian cell described herein).

In one example, two different nucleic acid vectors will be used. A first nucleic acid vector can include a promoter (e.g., any of the promoters described herein), a first coding sequence that encodes an N-terminal portion of a CLRN1 protein positioned 3′ of the promoter (e.g., any of the sizes of a portion of a CLRN1 protein described herein and/or any of the N-terminal portions of a CLRN1 protein described herein), a splice donor sequence positioned at the 3′ end of the first coding sequence, and a first detectable marker gene positioned 3′ of the splice donor sequence. A second nucleic acid vector can include a second detectable marker gene, a splice acceptor sequence positioned 3′ of the second detectable marker gene, a second coding sequence that encodes a C-terminal portion of a CLRN1 protein positioned at the 3′ end of the splice acceptor sequence (e.g., any of the sizes of a portion of a CLRN1 protein described herein and/or any of the C-terminal portions of a CLRN1 protein described herein), and a polyadenylation sequence at the 3′ end of the second coding sequence (e.g., any of the polyadenylation sequences described herein). In some embodiments, each of the encoded portions is at least 30 amino acid residues in length (e.g., at least 50 amino acids, at least 75 amino acids, or at least 100 amino acids in length), the amino acid sequences of the encoded portions do not overlap, and no single vector of the two different vectors encodes an active CLRN1 protein (e.g., a full-length CLRN1 protein). When introduced into a mammalian cell (e.g., any of the mammalian cells described herein) splicing occurs between the splice donor sequence and the splice acceptor sequence, thereby forming a recombined nucleic acid that encodes an active CLRN1 protein (e.g., a full-length CLRN1 protein).

In another example, three different nucleic acid vectors can be used. A first nucleic acid vector can include a portion of promoter sequence (e.g., any of the promoter sequences described herein), a first coding sequence of a CLRN1 gene that encodes a first portion of a CLRN1 protein (e.g., any of the CLRN1 coding sequences described herein) positioned 3′ of the promoter, a first splice donor sequence positioned at the 3′ end of the first coding sequence, and a first detectable marker gene. A second nucleic acid vector can include a second detectable marker gene, a first splice acceptor sequence positioned 3′ of the second detectable marker gene, a second coding sequence of a CLRN1 gene that encodes a second portion of a CLRN1 protein positioned at the 3′ end of the first splice acceptor sequence, a second splice donor sequence positioned at the 3′ end of the second coding sequence (e.g., any of the splice donor sequences described herein), and a third detectable marker gene. A feature of the second nucleic acid vector will be that self-splicing cannot occur (i.e., splicing will not occur between the second splice donor sequence and the first splice acceptor sequence of the second nucleic acid vector). In some embodiments, the splice donor sequence of the first nucleic acid vector and the second splice donor sequence of the second nucleic acid vector are the same (e.g., any of the splice donor sequences described herein or known in the art). In some embodiments, the first splice donor sequence of the first nucleic acid vector and the second splice donor sequence of the second nucleic acid vector are different (e.g., any of the splice donor sequences described herein or known in the art). A third nucleic acid vector can include a fourth detectable marker gene, a second splice acceptor sequence positioned 3′ of the fourth detectable marker gene, a third coding sequence of a CLRN1 gene that encodes a third portion of a CLRN1 protein positioned at the 3′ end of the second splice acceptor sequence, and a polyadenylation sequence positioned at the 3′ end of the third coding sequence (e.g., any of the polyadenylation sequences described herein). In such methods where three nucleic acid vectors are used, the first splice donor sequence and the first splice acceptor sequence can assemble together (recombine) and the second splice donor sequence and the second splice acceptor sequence can assemble together (recombine), and the portions of CLRN1 protein encoded by the first, second, and third coding sequences do not overlap with each other, and when introduced into a mammalian cell (e.g., any of the mammalian cells described herein), splicing occurs between the first splice donor sequence and the first splice acceptor sequence, and between the second splice donor sequence and the second splice acceptor sequence, to form a recombined nucleic acid that encodes an active CLRN1 protein (e.g., a full-length CLRN1 protein). As can be appreciated in the art, when three nucleic acid vectors are used, two of the at least two different nucleic acid vectors can include a detectable marker gene (e.g., an AP marker gene) and one of the at least two different nucleic acid vectors may include a splice acceptor sequence that is complementary to a splice donor sequence in a nucleic acid vector that includes a detectable marker gene. For example, in some embodiments, the first and second nucleic acid vectors can include a detectable marker gene (e.g., an AP marker gene), and the third nucleic acid vector will include a splice acceptor sequence that is complementary to the splice donor sequence in the second nucleic acid vector, and the third nucleic acid vector will not include a detectable marker gene (e.g., an AP marker gene). In other examples, the second and third nucleic acid vector can include a detectable marker gene (e.g., an AP marker gene), and the first nucleic acid vector will include a splice donor sequence that is complementary to the splice acceptor sequence in the second nucleic acid vector and the first nucleic acid vector will not include a detectable marker gene (e.g., an AP marker gene).

Based on the strategies provided above, one skilled in the art would understand how to develop a strategy using four, five, or six vectors.

The CLRN1 coding sequences provided in the at least two nucleic acid vectors (e.g., two, three, four, five or six) will not be overlapping. Each of the at least two different vectors can include a coding sequence that encodes a different portion of a CLRN1 protein, each of the encoded portions being, e.g., at least 30 amino acids (e.g., about 30 amino acids to about 1600 amino acids, or any of the other subranges of this range described herein).

In some embodiments, each of the at least two different vectors includes a coding sequence that encodes a different portion of a CLRN1 protein, each of the encoded portions encoding at least one exon and at least one intron of SEQ ID NO: 9 (e.g., at least two exons and at least one intron, at least two exons and at least two introns, at least three exons at least one intron, at least three exons and at least two introns, or at least three exons and at least three introns). In some embodiments, each of the at least two different vectors include a coding sequence that encodes a different portion of a CLRN1 protein, each of the encoded portions encoding up to 80% of SEQ ID NO: 1 (e.g., up to 10%, up to 20%, up to 30%, up to 40%, up to 50%, up to 60%, up to 70% of SEQ ID NO: 1), provided that each of the encoded portions is non-overlapping with any other. In some embodiments, each of the at least two different vectors include a coding sequence that encodes a different portion of a CLRN1 protein, each of the encoded portions encoding up to 80% of SEQ ID NO: 3 (e.g., up to 10%, up to 20%, up to 30%, up to 40%, up to 50%, up to 60%, up to 70% of SEQ ID NO: 3), provided that each of the encoded portions is non-overlapping with any other. In some embodiments, each of the at least two different vectors include a coding sequence that encodes a different portion of a CLRN1 protein, each of the encoded portions encoding up to 80% of SEQ ID NO: 5 (e.g., up to 10%, up to 20%, up to 30%, up to 40%, up to 50%, up to 60%, up to 70% of SEQ ID NO: 5), provided that each of the encoded portions is non-overlapping with any other. In some embodiments, each of the at least two different vectors include a coding sequence that encodes a different portion of a CLRN1 protein, each of the encoded portions encoding up to 80% of SEQ ID NO: 7 (e.g., up to 10%, up to 20%, up to 30%, up to 40%, up to 50%, up to 60%, up to 70% of SEQ ID NO: 7), provided that each of the encoded portions is non-overlapping with any other.

As described in Example 10, each of the at least two nucleic acid vectors may further include an inverted terminal repeat (ITR) to allow head-to-tail recombination. The ITR will be subsequently removed via splicing. Examples of ITRs and splice acceptor sequences and/or splice donor sequences are known in the art and have been described in Example 10.

### Example 12: Hybrid Vector Trans-Splicing Strategy Using a F1 Phage Highly Recombinogenic Exogenous Gene Region (AK)

At least two (e.g., two, three, four, five, or six) different nucleic acid vectors (e.g., AAV vectors) can also be used in any of the methods described herein to reconstitute an active CLRN1 gene (e.g., a full-length CLRN1 gene) within a cell following intermolecular concatamerization, marker gene-mediated recombination, and trans-splicing. This strategy is a hybrid strategy as it will include homologous recombination and/or trans-splicing. See, e.g., Trapani et al., EMBO Mol. Med. 6(2):194-211, 2014, incorporated in its entirety herein. As used herein, an F1 phage recombinogenic region (AK) will be used to allow coding sequence-independent recombination. The F1 phage recombinogenic region may be a 77 bp recombinogenic region from the F1 phage genome as described in Trapani et al. (2014). At least two different nucleic acid vectors will contain an F1 phage recombinogenic region. Since the hybrid vector will be constructed based on a trans-splicing vector as described in Example 10, a nucleic acid encoding an active CLRN1 protein (e.g., a full-length CLRN1 protein) may be generated using F1 phage recombinogenic region-induced recombination and trans-splicing. After trans-splicing, a nucleic acid encoding an active CLRN1 protein (e.g., a full-length CLRN1 protein) will be generated in a mammalian cell (e.g., any of the mammalian cells described herein).

In one example, two different nucleic acid vectors will be used. A first nucleic acid vector can include a promoter (e.g., any of the promoters described herein), a first coding sequence that encodes an N-terminal portion of a CLRN1 protein positioned 3′ of the promoter (e.g., any of the sizes of a portion of a CLRN1 protein described herein and/or any of the N-terminal portions of a CLRN1 protein described herein), a splice donor sequence positioned at the 3′ end of the first coding sequence, and an F1 phage recombinogenic region positioned 3′ of the splice donor sequence. A second nucleic acid vector can include an F1 phage recombinogenic region, a splice acceptor sequence positioned 3′ of the F1 phage recombinogenic region, a second coding sequence that encodes a C-terminal portion of a CLRN1 protein positioned at the 3′ end of the splice acceptor sequence (e.g., any of the sizes of a portion of a CLRN1 protein described herein and/or any of the C-terminal portions of a CLRN1 protein described herein), and a polyadenylation sequence at the 3′ end of the second coding sequence (e.g., any of the polyadenylation sequences described herein). In some embodiments, each of the encoded portions is at least 30 amino acid residues in length (e.g., at least 50 amino acids, at least 75 amino acids, or at least 100 amino acids in length), the amino acid sequence of each of the encoded portions do not overlap, and no single vector of the two different vectors encodes an active CLRN1 protein (e.g., a full-length CLRN1 protein). When introduced into a mammalian cell (e.g., any of the mammalian cells described herein) splicing occurs between the splice donor sequence and the splice acceptor sequence, thereby forming a recombined nucleic acid that encodes an active CLRN1 protein (e.g., a full-length CLRN1 protein).

In another example, three different nucleic acid vectors will be used. A first nucleic acid vector can include a promoter sequence (e.g., any of the promoter sequences described herein), a first coding sequence that encodes a first portion of a CLRN1 protein (e.g., any of the CLRN1 coding sequences described herein) positioned 5′ of the promoter, a first splice donor sequence positioned at the 3′ end of the first coding sequence, and an F1 phage recombinogenic region. A second nucleic acid vector can include an F1 phage recombinogenic region, a first splice acceptor sequence positioned 3′ of the F1 phage recombinogenic region, a second coding sequence that encodes a second portion of a CLRN1 protein positioned at the 3′ end of the first splice acceptor sequence, a second splice donor sequence positioned at the 3′ end of the second coding sequence (e.g., any of the splice donor sequences described herein), and an F1 phage recombinogenic region. A feature of the second nucleic acid vector will be that self-splicing cannot occur (i.e., splicing will not occur between the second splice donor sequence and the first splice acceptor sequence of the second nucleic acid vector). In some embodiments, the splice donor sequence of the first nucleic acid vector and the second splice donor sequence of the second nucleic acid vector are the same (e.g., any of the splice donor sequences described herein or known in the art). In some embodiments, the first splice donor sequence of the first nucleic acid vector and the second splice donor sequence of the second nucleic acid vector are different (e.g., any of the splice donor sequences described herein or known in the art). A third nucleic acid vector can include an F1 phage recombinogenic region, a second splice acceptor sequence positioned 3′ of the F1 phage recombinogenic region, a third coding sequence that encodes a third portion of a CLRN1 protein positioned at the 3′ end of the second splice acceptor sequence, and a polyadenylation sequence positioned at the 3′ end of the third coding sequence (e.g., any of the polyadenylation sequences described herein). In such methods where three nucleic acid vectors are used, the first splice donor sequence and the first splice acceptor sequence can assemble together (recombine) and the second splice donor sequence and the second splice acceptor sequence can assemble together (recombine), and the portion of CLRN1 protein encoded by the first, second, and third coding sequences do not overlap, and when introduced into a mammalian cell (e.g., any of the mammalian cells described herein), splicing occurs between the first splice donor sequence and the first splice acceptor sequence, and between the second splice donor sequence and the second splice acceptor sequence, to form a recombined nucleic acid that encodes an active CLRN1 protein (e.g., a full-length CLRN1 protein). As can be appreciated in the art when three nucleic acid vectors are used, two of the different nucleic acid vectors can include an F1 phage recombinogenic region and one of the different nucleic acid vectors may include a splice acceptor sequence that is complementary to a splice donor sequence in a nucleic acid vector that includes an F1 phage recombinogenic region. For example, in some embodiments, the first and second nucleic acid vectors can include an F1 phage recombinogenic region, and the third nucleic acid vector will include a splice acceptor sequence that is complementary to the splice donor sequence in the second nucleic acid vector, and the third nucleic acid vector will not include an F1 phage recombinogenic region (e.g., an AP marker gene). In other examples, the second and third nucleic acid vector can include an F1 phage recombinogenic region and the first nucleic acid vector will include a splice donor sequence that is complementary to the splice acceptor sequence in the second nucleic acid vector and the first nucleic acid vector will not include an F1 phage recombinogenic region. Based on the strategies provided above, one skilled in the art would understand how to develop a strategy using four, five, or six vectors.

The CLRN1 coding sequences provided in each of the at least two nucleic acid vectors (e.g., two, three, four, five or six) will not be overlapping. Each of the at least two different vectors include a coding sequence that encodes a different portion of a CLRN1 protein, each of the encoded portions being at least 30 amino acids (e.g., about 30 amino acids to about 1600 amino acids, or any of the subranges of this range described herein).

In some embodiments, each of the at least two different vectors include a coding sequence that encodes a different portion of a CLRN1 protein, each of the encoded portions encoding at least one exon and at least one intron of SEQ ID NO: 9 (e.g., at least two exons and at least one intron, at least two exons and at least two introns, at least three exons and at least one intron, at least three exons and at least two introns, or at least three exons and at least three introns). In some embodiments, each of the at least two different vectors includes a coding sequence that encodes a different portion of a CLRN1 protein, each of the encoded portions encoding up to 80% of SEQ ID NO: 1 (e.g., up to 10%, up to 20%, up to 30%, up to 40%, up to 50%, up to 60%, or up to 70% of SEQ ID NO: 1), provided that each of the encoded portions is non-overlapping. In some embodiments, each of the at least two different vectors include a coding sequence that encodes a different portion of a CLRN1 protein, each of the encoded portions encoding up to 80% of SEQ ID NO: 3 (e.g., up to 10%, up to 20%, up to 30%, up to 40%, up to 50%, up to 60%, or up to 70% of SEQ ID NO: 3), provided that each of the encoded portions is non-overlapping. In some embodiments, each of the at least two different vectors include a coding sequence that encodes a different portion of a CLRN1 protein, each of the encoded portions encoding up to 80% of SEQ ID NO: 5 (e.g., up to 10%, up to 20%, up to 30%, up to 40%, up to 50%, up to 60%, or up to 70% of SEQ ID NO: 5), provided that each of the encoded portions is non-overlapping. In some embodiments, each of the at least two different vectors include a coding sequence that encodes a different portion of a CLRN1 protein, each of the encoded portions encoding up to 80% of SEQ ID NO: 7 (e.g., up to 10%, up to 20%, up to 30%, up to 40%, up to 50%, up to 60%, or up to 70% of SEQ ID NO: 7), provided that each of the encoded portions is non-overlapping.

As described in Example 10, each of the at least two nucleic acid vectors may further include an inverted terminal repeat (ITR) to allow head-to-tail recombination. The ITR will be subsequently removed via splicing. Examples of ITRs and splice acceptor sequences and/or splice donor sequences are known in the art and have been described in Example 10.

### Example 13. Hybrid Vector Trans-Splicing Strategy Using Two CLRN1 Isoforms

At least two different nucleic acid vectors (e.g., AAV vectors) can be used to reconstitute an active CLRN1 gene (e.g., a full-length CLRN1 gene) within a cell following intermolecular concatamerization and trans-splicing. See, e.g., Yan et al., Proc. Natl. Acad. Sci. U.S.A. 97:12; 6716-6721, 2000, incorporated in its entirety herein.

In some examples, two different nucleic acid vectors will be used. A first nucleic acid vector can include a promoter (e.g., any of the promoters described herein), a first coding sequence that encodes an N-terminal portion of a CLRN1 protein positioned 3′ of the promoter (e.g., any of the sizes of a portion of a CLRN1 protein described herein and/or any of the N-terminal portions of a CLRN1 protein described herein), and a splice donor sequence positioned at the 3′ end of the first coding sequence.

A second nucleic acid vector can include a splice acceptor sequence, a second coding sequence that encodes a C-terminal portion of a CLRN1 protein (i.e., the entire portion of the CLRN1 protein that is not included in the N-terminal portion) positioned at the 3′ end of the splice acceptor sequence (e.g., any of the sizes of a portion of a CLRN1 protein described herein and/or any of the C-terminal portions of a CLRN1 protein described herein), and a polyadenylation signal sequence at the 3′ end of the second coding sequence (e.g., any of the polyadenylation seqences described herein).

In some embodiments, each of the encoded portions is at least 30 amino acid residues in length (e.g., at least 50 amino acids, at least 75 amino acids, or at least 100 amino acids in length), the amino acid sequences of the two encoded portions do not overlap with each other; and no single vector of the two different vectors encodes an active CLRN1 protein (e.g., a full-length CLRN1 protein).

In some embodiments, each of the at least two different vectors includes a coding sequence that encodes a different portion of a first isoform of the CLRN1 protein (e.g., SEQ ID NO: 3). In some embodiments, each of the at least two different vectors includes a coding sequence that encodes a different portion of a CLRN1 protein, each of the encoded portions encoding up to 80% of the amino acid sequence of SEQ ID NO: 3 (e.g., up to 10%, up to 20%, up to 30%, up to 40%, up to 50%, up to 60%, or up to 70% of SEQ ID NO: 3), provided that each of the encoded portions is non-overlapping with any other.

In some embodiments, one of the at least two different nucleic acid vectors further includes a sequence that encodes a second isoform of the CLRN1 protein (e.g., SEQ ID NO: 5). In some embodiments, each of the at least two different vectors includes a coding sequence that encodes a different portion of a CLRN1 protein, each of the encoded portions encoding up to 80% of the amino acid sequence of SEQ ID NO: 5 (e.g., up to 10%, up to 20%, up to 30%, up to 40%, up to 50%, up to 60%, or up to 70% of SEQ ID NO: 5), provided that each of the encoded portions is non-overlapping with any other.

In some embodiments, each of the at least two different vectors includes a coding sequence that encodes a different potion of a second isoform of the CLRN1 protein. In some embodiments, one of the at least two different nucleic acid vectors further incudes a sequence that encodes a first isoform of the CLRN1 protein.

When introduced into a mammalian cell (e.g., any of the mammalian cells described herein), splicing occurs between the splice donor sequence and the splice acceptor sequence, thereby forming a recombined nucleic acid that encodes an active CLRN1 protein (e.g., a full-length CLRN1 protein).

Non-limiting examples of such vectors are shown in FIGS. 1, 2, 4, 7-10, and 12-24.

### Example 14. CLRN1 Expression in HEK293FT Cells

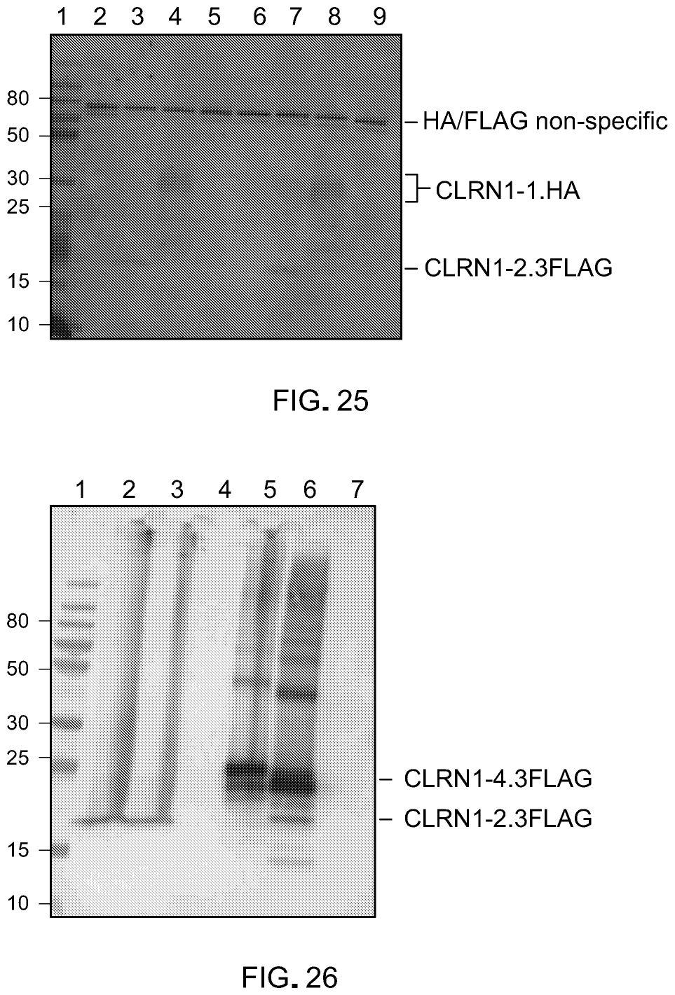

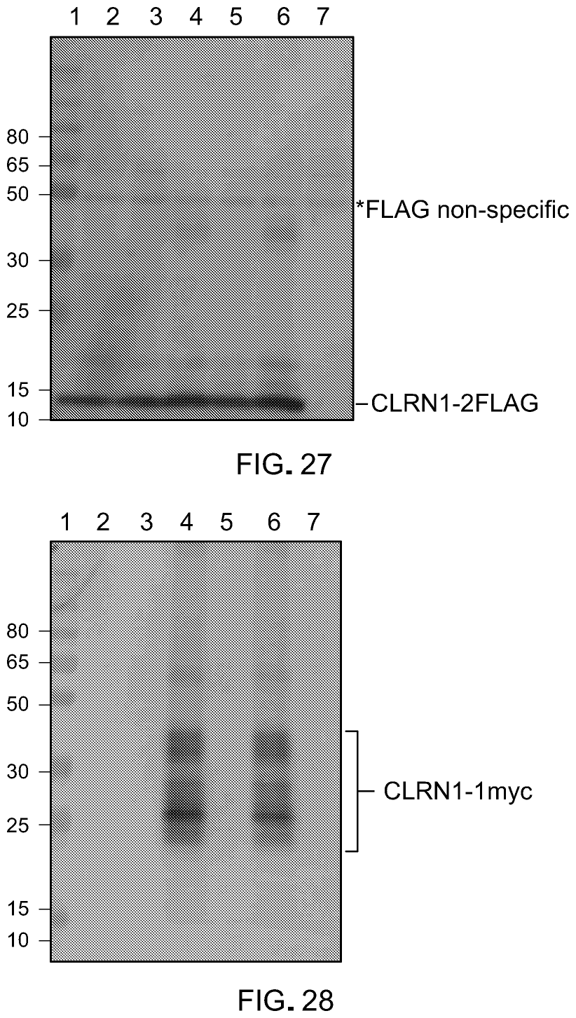

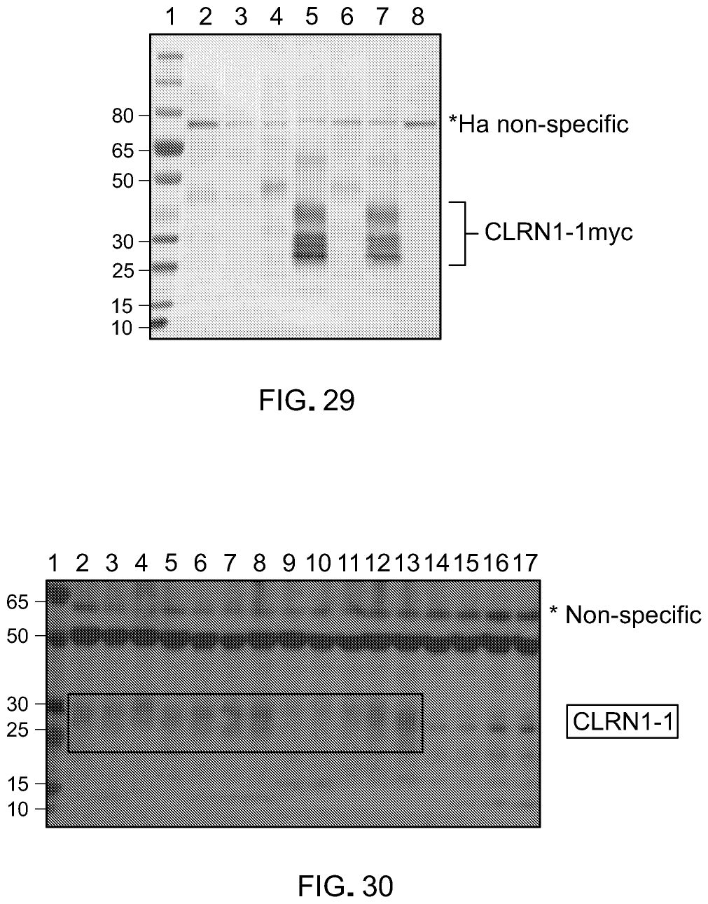

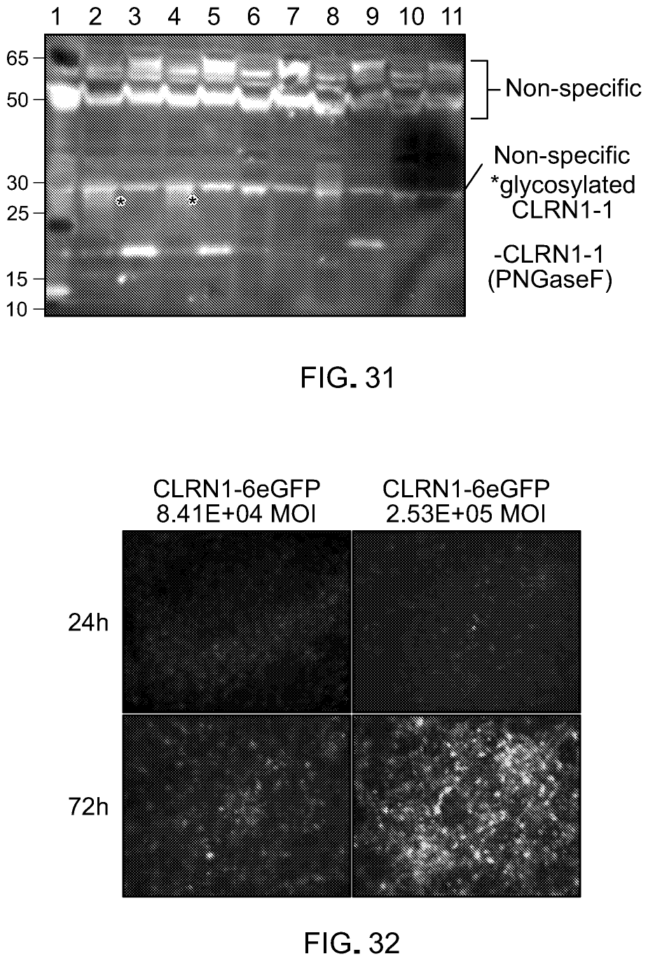

HEK293FT cells were transfected with exemplary CLRN vectors. 48 hours post-transfection, HEK293FT cell lysates were prepared and CLRN1 protein expression was determined by Western blot. As shown in FIGS. 25 to 31, CLRN1 protein was detected in all tested samples. This result confirmed that CLRN1 protein can be expressed by exemplary CLRN1 vectors described herein.

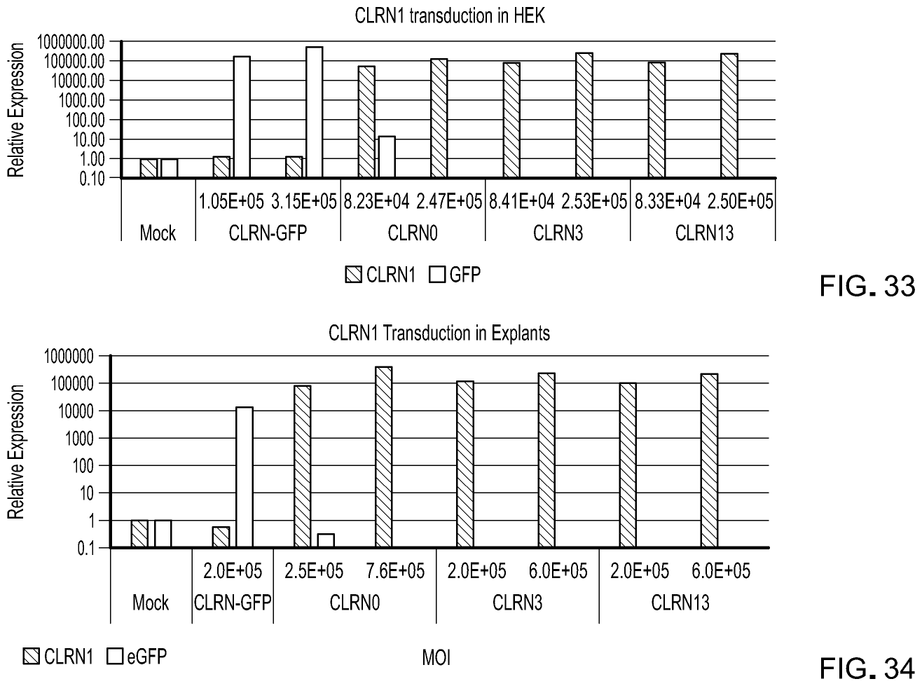

FIG. 32 showed that HEK293FT cells transfected with CLRN1-6eGFP vector expressed high levels of GFP as soon as 72 hours post-transfection. At 24 hours post-transfection, few HEK293FT cells expressed GFP following transfection with CLRN1-e6GFP vector at MOI 8.41E+04 and 2.53E+05. At 72 hours post-transfection, most HEK293FT cells transfected with CLRN1-e6GFP vector at MOI 2.53E+05 expressed GFP, while some HEK293FT cells transfected with CLRN1-e6GFP vector at MOI 8.41E+04 expressed GFP. As shown in FIG. 33, CLRN1 was expressed at high levels in HEK293FT cells transfected with CLRN-0 vector, CLRN-3 vector, and CLRN-13 vector.

### Example 15. CLRN1 Expression P2 Cochlear Mouse Explants

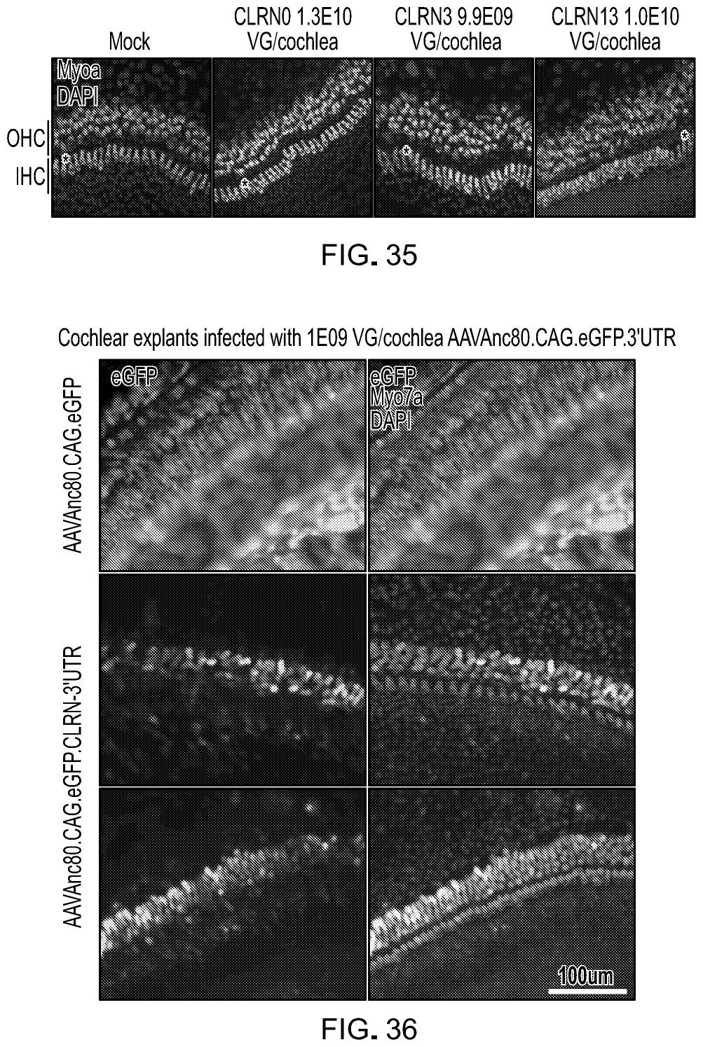

P2 cochlear explants from WT mice were infected 16 hours after plating and were harvested for RNA and immunofluorescence 72 hours after infection. As shown in FIG. 34, CLRN1 was efficiently expressed in cochlear explants. As shown in FIG. 35, outher hair cells (OHC) and inner hair cells (IHC) of P2 cochlear explants express Myo7a when transfected with CLRN-0 vector (1.3E10 VG/cochlea), CLRN-3 (9.9E09 VG/cochlea) and CLRN-13 (1.0E10 VG/cochlea). Transfection with either vector did not disrupt the structural integrity of OHCs or IHCs of the cochlea. Thus, FIG. 35 shows lack of toxicity of CLRN1 constructs with viable and organized outer hair cells (OHC), inner hair cells (IHC) and stereociliary bundles. As shown in FIG. 36, eGFP expression with CLRN1-3′UTR appeared to specify the transduction compared to CAG promoter alone. Cochlear explants infected with 1E09 AAV/Anc80.CAG.eGFP expressed Myo7a in the inner hair cells. Cochlear explants infected with 1E09 AAV/Anc80.CAG.eGFP.CLRN-3′UTR expressed My07a in both the OHC and IHC. Higher transduction and co-expression of CLRN1 was seen in OHCs of cochlear explants infected with AAV/Anc80.CAG.eGFP.CLRN-3′UTR. GFP expression in cochlear explants infected with AAV/Anc80.CAG.eGFP.CLRN-3′UTR was restricted to OHCs.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. Section headings and any descriptions of materials, methods, and examples are illustrative only and not intended to be limiting.

| SEQUENCE LISTING | |
|---|---|
| - Human Full-length Wildtype CLRN1 Protein Isoform D | |
| SEQ ID NO: 1 | |
| MPSQQKKIIFCMAGVFSFACALGVVTALGTPLWIKATVLCKTGALLVNASGQELDKFMGEMQYGLFHGEGV | |
| RQCGLGARPFRFSFFPDLLKAIPVSIHVNVILFSAILIVLTMVGTAFFMYNAFGKPFETLHGPLGLYLLSF | |
| ISVALWLPATRHQAQGSCGCLVMILFASEVKIHHLSEKIANYKEGTYVYKTQSEKYTTSFWVIFFCFFVHF | |
| LNGLLIRLAGFQFPFAKSKDAETTNVAADLMY | |
| - Human Wildtype CLRN1 Isoform D cDNA | |
| SEQ ID NO: 2 | |
| atgccaagc caacagaaga aaatcatttt ttgcatggcc ggagtgttca gttttgcatg | |
| tgccctcgga gttgtgacag ccttggggac accgttgtgg atcaaagcca ctgtcctctg | |
| caaaacggga gctctgctcg tcaatgcctc agggcaggag ctggacaagt ttatgggtga | |
| aatgcagtac gggcttttcc acggagaggg tgtgaggcag tgtgggttgg gagcaaggcc | |
| ctttcggttc tcattttttc cagatttgct caaagcaatc ccagtgagca tccacgtcaa | |
| tgtcattctc ttctctgcca tccttattgt gttaaccatg gtggggacag ccttcttcat | |
| gtacaatgct tttggaaaac cttttgaaac tctgcatggt cccctagggc tgtacctttt | |
| gagcttcatt tcagttgccc tttggctgcc agctaccagg caccaggctc aaggctcctg | |
| tggctgtctt gtcatgatat tgtttgcctc tgaagtgaaa atccatcacc tctcagaaaa | |
| aattgcaaat tataaagaag ggacttatgt ctacaaaacg caaagtgaaa aatataccac | |
| ctcattctgg gtcattttct tttgcttttt tgttcatttt ctgaatgggc tcctaatacg | |
| acttgctgga tttcagttcc cttttgcaaa atctaaagac gcagaaacaa ctaatgtagc | |
| tgcagatcta atgtactga | |
| - Human Full-length Wildtype CLRN1 Protein Isoform A | |
| SEQ ID NO: 3 | |
| MPSQQKKIIFCMAGVFSFACALGVVTALGTPLWIKATVLCKTGALLVNASGQELDKFMGEMQYGLFHGEGV | |
| RQCGLGARPFRFSFFPDLLKAIPVSIHVNVILFSAILIVLTMVGTAFFMYNAFGKPFETLHGPLGLYLLSF | |
| ISGSCGCLVMILFASEVKIHHLSEKIANYKEGTYVYKTQSEKYTTSFWVIFFCFFVHFLNGLLIRLAGFQF | |
| PFAKS KDAETTNVAADLMY | |
| - Human Wildtype CLRN1 Isoform A cDNA | |
| SEQ ID NO: 4 | |
| atgccaagc caacagaaga aaatcatttt ttgcatggcc ggagtgttca gttttgcatg | |
| tgccctcgga gttgtgacag ccttggggac accgttgtgg atcaaagcca ctgtcctctg | |
| caaaacggga gctctgctcg tcaatgcctc agggcaggag ctggacaagt ttatgggtga | |
| aatgcagtac gggcttttcc acggagaggg tgtgaggcag tgtgggttgg gagcaaggcc | |
| ctttcggttc tcattttttc cagatttgct caaagcaatc ccagtgagca tccacgtcaa | |
| tgtcattctc ttctctgcca tccttattgt gttaaccatg gtggggacag ccttcttcat | |
| gtacaatgct tttggaaaac cttttgaaac tctgcatggt cccctagggc tgtacctttt | |
| gagcttcatt tcaggctcct gtggctgtct tgtcatgata ttgtttgcct ctgaagtgaa | |
| aatccatcac ctctcagaaa aaattgcaaa ttataaagaa gggacttatg tctacaaaac | |
| gcaaagtgaa aaatatacca cctcattctg ggtcattttc ttttgctttt ttgttcattt | |
| tctgaatggg ctcctaatac gacttgctgg atttcagttc ccttttgcaa aatctaaaga | |
| cgcagaaaca actaatgtag ctgcagatct aatgtactga | |
| - Human Full-length Wildtype CLRN1 Protein Isoform C | |
| SEQ ID NO: 5 | |
| MQALQQQPVFPDLLKAIPVSIHVNVILFSAILIVLTMVGTAFFMYNAFGKPFETLHGPLGLYLLSFISGSC | |
| GCLVMILFASEVKIHHLSEKIANYKEGTYVY KTQSEKYTTSFWLTKGHS | |
| - Human Wildtype CLRN1 Isoform C cDNA | |
| SEQ ID NO: 6 | |
| atgcaggc cctgcagcag caaccagttt ttccagattt gctcaaagca atcccagtga | |
| gcatccacgt caatgtcatt ctcttctctg ccatccttat tgtgttaacc atggtgggga | |
| cagccttctt catgtacaat gcttttggaa aaccttttga aactctgcat ggtcccctag | |
| ggctgtacct tttgagcttc atttcaggct cctgtggctg tcttgtcatg atattgtttg | |
| cctctgaagt gaaaatccat cacctctcag aaaaaattgc aaattataaa gaagggactt | |
| atgtctacaa aacgcaaagt gaaaaatata ccacctcatt ctggctgact aaaggccaca gctga | |
| - Human Full-length Wildtype CLRN1 Protein Isoform E | |
| SEQ ID NO: 7 | |
| MPSQQKKIIFCMAGVFSFACALGVVTALGTPLWIKATVLCKTGALLVNASGQELDKFMGEMQYGLFHGEGV | |
| RQCGLGARPFRFSCYFLDPFMGLPTGVPHLLSLPCSTSCRREHTSERVQEPAGCFSAVRSKLHAGPAAATS | |
| FSRFAQSNPSEHPRQCHS LLCHPYCVNHGGDSLLHVQCFWKTF | |
| - Human Wildtype CLRN1 Isoform E cDNA | |
| SEQ ID NO: 8 | |
| atgccaagc caacagaaga aaatcatttt ttgcatggcc ggagtgttca gttttgcatg | |
| tgccctcgga gttgtgacag ccttggggac accgttgtgg atcaaagcca ctgtcctctg | |
| caaaacggga gctctgctcg tcaatgcctc agggcaggag ctggacaagt ttatgggtga | |
| aatgcagtac gggcttttcc acggagaggg tgtgaggcag tgtgggttgg gagcaaggcc | |
| ctttcggttc tcatgctatt ttcttgaccc cttcatggga ctcccaacag gggtacccca | |
| tttactcagc ctgccctgct caacctcttg caggagggag cacacgagtg aacgagtgca | |
| ggaaccagct ggctgcttta gtgctgtgag gagtaaactc catgcaggcc ctgcagcagc | |
| aaccagtttt tccagatttg ctcaaagcaa tcccagtgag catccacgtc aatgtcattc | |
| tcttctctgc catccttatt gtgttaacca tggtggggac agccttcttc atgtacaatg | |
| cttttggaaa accttttga | |
| - Human Wildtype CLRN1 Genomic sequence | |
| SEQ ID NO: 9 | |
| aggagatact tgaaggcagt ttgaaagact tgttttacag attcttagtc caaagatttc | 61 |
| caattaggga gaagaagcag cagaaaagga gaaaagccaa gtatgagtga tgatgaggcc | 121 |
| ttcatctact gacatttaac ctggcgagaa ccgtcgatgg tgaagttgcc ttttcagctg | 181 |
| ggagctgtcc gttcagcttc cgtaataaat gcagtcaaag aggcagtccc ttcccattgc | 241 |
| tcacaaaggt cttgtttttg aacctcgccc tcacagaagc cgtttctcat catgccaagc | 301 |
| caacagaaga aaatcatttt ttgcatggcc ggagtgttca gttttgcatg tgccctcgga | 361 |
| gttgtgacag ccttggggac accgttgtgg atcaaagcca ctgtcctctg caaaacggga | 421 |
| gctctgctcg tcaatgcctc agggcaggag ctggacaagt ttatgggtga aatgcagtac | 481 |
| gggcttttcc acggagaggg tgtgaggcag tgtgggttgg gagcaaggcc ctttcggttc | 541 |
| tcatgtaagt agcaattgca tttgagttat ttaatgcttt aggcagactc ttcccagtgt | 601 |
| tgcgaggaat tatatttgag aattttgccg tgtttactgc aggacttttt aaatcggtgt | 661 |
| gaaccatatg aaaaacctat gactctgagc aatttcttct tcctagtttt tattatttta | 721 |
| tacttgcttt ttattataat atagagttaa ttcattgtta cataattaag gtttttggaa | 781 |
| atattggcaa ttaagatgct taagtattaa tatttatgta aaaaattatg gagtcttttt | 841 |
| aaaaaagtaa acttggggaa ataggaaagc tgtaaagaat gatctttatg ctttttgttc | 901 |
| tttataaaaa gaaccaaggt catgggctcc gtatttaacc aggttgccac ctttctcatg | 961 |
| attttgtttc ctgctcccca ctccctccca ttattcctgc taagaccttt cctgctgcta | 1021 |
| aatattcagt tttcattttt aactaatttg gaatcatttg gctatagaaa tttaaaatga | 1081 |
| tctgctgtgc taactgggaa agaaatggat gcctatttag tatagaacat tttaaactga | 1141 |
| ttgacctgca aatcatgtag agaatatgag agagattttc ttgttgtgat ttttgtgaaa | 1201 |
| tggaagtgta atccacagta tttataacct gtttatctta agaagagaat ttttaaaaat | 1261 |
| taccatgtga ataggcaact cattaaatga aaattaatag gaagtcattt gttatatctc | 1321 |
| ttacaacaca cattcagaag ttattattat ttcagaaggg ctggtttgga acaaccttat | 1381 |
| gaagacacag tcagtaaatt actgcataaa tcactcttca ggaaaggagg ttaccaactg | 1441 |
| aagcatttaa aatgaattat tattttgccc aggttttttt tttctttcta gtataggtag | 1501 |
| aaggctaaat taattgaatt tattattaac atatgcagtg cctaattaaa tttcagtgct | 1561 |
| ggtctattta tatttctgca acattcctta tatcttctta gcagtcattg gacaccaacc | 1621 |
| ttcagctcac ataggttact aagtgatatg aattttcata gggctccaga aaatttccaa | 1681 |
| gaattggttg ttagcttttt aattgatgaa gtggatacca gttcttttca ctgaatggct | 1741 |
| tttattcatt aaggtaatgg ggctgttaga gttgcttagt tttcctgggg aaggggaagg | 1801 |
| aagaaaacaa agcagaatgt catgtgatat gcaactgtat taaaaaaccg aaaaggaaaa | 1861 |
| aagttgagag agatgattta accgtgagtc accggcagcc aaagcgtgag taaagcttct | 1921 |
| cacagatgaa tttagacaaa agcggagaag gtactggtga attttctgga gcctttacat | 1981 |
| tttctacagt gaaatggaga taaactttac tcatgccata ggacatgttt caaaacaata | 2041 |
| ataagatgtt ttctgaacac ttactacata ctaagcactt tatatgcttt gtctcattta | 2101 |
| atccttacac agccacattc ttctggggtt tagcgaatga tttttgtggt tgtgtcctat | 2161 |
| gcttgtcctg tctaaggatg aagttgttct aattgggtgc ccctcctttt gctttctgtg | 2221 |
| aggacttgca gaactggtgg ggtttaaaca gtaccctcac ttatctcaca gaatttcatt | 2281 |
| agctcccaga tacccctgac attctccccc tagcctagtg aagaaaatct tccatttact | 2341 |
| tgttcattct gcagtgacag ctccatcaat atacaataga ctatacatat taagtgtact | 2401 |
| gtatatacta tacatgttaa aaatctcatt cattttggtg aggcccagct aagaatactt | 2461 |
| acagtagagc tttttttttt ttcctaagca taaaagtatc tttttcaatg cagcatgaga | 2521 |
| cagagttggg aaaaccaaaa taaatagatc caatggactc cccaaagagg ataatattca | 2581 |
| tttaaataaa cacccctctc agtgttaaaa ctttctaatc aacatgcctt tgggacacat | 2641 |
| tgcaccctca aagtttacac tcccattgca acgcagcttt gtggttcacg ttttttccat | 2701 |
| tcagaatgtc attaccctgt caatgatgtt tcatcaacgt ttgcttggat gagaatcctc | 2761 |
| tgatattctt cctgatagaa atgtataagc cctgttcata taaatgaata aaagatctaa | 2821 |
| ccttactttc tcagtagtgg cttccttgga gcaaaaagca gggacctcca gagagctcag | 2881 |
| gtggatgact cttttctgtt tcttccagag ctcaacttac aattagtgca caattcattt | 2941 |
| cccagaatgt cttctttctt attgtgcctt tagaaagtta ttaagcaaac atttgaattc | 3001 |
| acagaatctt accagtgtaa gaggaatgga aaaggtaact tatcaaggta acaatcactt | 3061 |
| cgtggccagt tttttcggct cactgcaact acccctcctg ggttcaagcg attctcctgc | 3121 |
| ctcagcctcc caagtagctg ggactacagg cgtgcaccac catgcccagc taattttttt | 3181 |
| tttgtatttt tagtaaagac agggtttcac catgtgggcc aggctggtct catggcaagt | 3241 |
| tttctttgtg ttgtcatgtt attatcaatt aataggaatt tatatttcag ttctgttagg | 3301 |
| tggataaaca ctattttgca tacctaaatg tttcatttat atcagcactg gccaataaaa | 3361 |
| atatactata agcaggccgg gtgcagtggc tcacgcctgt aatcccaact tttgggaggc | 3421 |
| caacactttg ggaggacaca gggtcaggag atcgagacca tcctggctaa cctggtgaaa | 3481 |
| tcccgtctct actaaaaata caaaaaatta gccgggcgtg gcgggggcgc ctgtagtccc | 3541 |
| agctacttgg gaggctgagg caggagaatg gcacgaaccc gggaggtgga gcttgcagtg | 3601 |
| agccgagatc tcgccactgc actccagcct tggtgacaga gcaagactct gtctcaaaaa | 3661 |
| aaaaaaaaaa aaaaaaaaaa aaaaaatata tatatatata tatatattac aagccacaag | 3721 |
| ccacatatgt acttttaaat gtcctagtag ccatattaga aaacaaaagt aaaaaggaat | 3781 |
| agatgaaatt aattttaatg atttttttaa acccaagata tccaaaatat tatcatttta | 3841 |
| acatataatt aaaaatttat tgagatgttt tacattgttt ttttttttct tgacgctgtc | 3901 |
| ttggaaatct agagtgtatt ttacatttac attacatcta attcagtcta gccacatttc | 3961 |
| acatgctcat ttttttgtgt gtggttttat ggagcagaga gtttaatagg caagaaagaa | 4021 |
| aagagaaggc agaagaaaat ggctcccctg tacagagacg cgggggtggg gcgctccaaa | 4081 |
| gccaaaagag gaggtcccta agtatggtag acaccagcca ggaatatatg cagtgtctgg | 4141 |
| aggaggggat gtctgatttg catagggtca catgctcatt tttatggcta ctgtattggt | 4201 |
| cagtacagat ttagatgggg atttgctctg aacaagttgg tgattgatgg tgttcatatt | 4261 |
| ttaattgaat ttttcctggg ttctgctgta cacttgtatg tgtgttagtt tcatgtgcaa | 4321 |
| tgcttgtgtc acttttaaaa cccagatata tggcataaca tgagaatgaa aaatggacca | 4381 |
| gaaaaatagt ttggcaatgt agtcatgttt gttcctatta aatgttccct attgaccact | 4441 |
| ctatctcttt taattataac aagaatctgc cctgccagca tgcccagtta cgctgggaaa | 4501 |
| acttctgcct catttactct ggctgattct catccactta tgggtcagtg gttcattttc | 4561 |
| tagaggtcac cagcattcat acctagcata caatttcatt cattataatg aaggaatgtt | 4621 |
| ttcccttcaa agagacacaa ctagtgggct taatttttct tgatatgtca cctgtaaaat | 4681 |
| tttaatgatg atgtttaaac tctaaatgta gccatcaaga caaaaactgc aaattttgag | 4741 |
| cctcagtgtg tgtgggtggg tttctgtttc ggtaatttga aacattgcag aatatcatca | 4801 |
| aaatatgata cccaagaatc atatggtatc aatcattcct agatatactg atctattcat | 4861 |
| tgccaagata gttcaatgag ctggcaaaaa catatggaat tattttctta aaatgtgaaa | 4921 |
| aataaaattt aaccaatcat gtatcacagc ttgcaacttt agtcatactt tgaaaagcat | 4981 |
| tttaatttgg acctcatgat tgaaaattta taaaaagctg aacagaaatt agttcacttc | 5041 |
| atattttaga aaagcagagt ttctttacta aatgaggcat ttgacccaaa ttggagagaa | 5101 |
| aatgttgaaa ctacttctgt gagcaagcag gtggcttctc aaacacatgt tgggatgaaa | 5161 |
| tggttgggcc tcagggtctc agtgcctgtc actgagagtt ggcactctct atctccatgg | 5221 |
| tctcctccaa gtgtgactct tgtctcttgc tgacctgacc tgccccaagt gactcactgg | 5281 |
| tcatgaccct gcacaccttg cgtctctcct atcaccctgc cgatggcaga gctacaaagg | 5341 |
| tctttgatgt agctctgtct gatatcctgt gtttccccct atggtctgtg tggaagcagg | 5401 |
| tatgggggtg tgtaaagggg aagcctatga agttcatctg caaagactac ctggttaggg | 5461 |
| gaggagagga agaagctata tgcaccattt caccagcaag catgggctct tctgcctttt | 5521 |
| agcttagggg tcctgttgtc tagtctcact cacctattaa aacagtccag caaatagagg | 5581 |
| ttttgtttac ctcccattaa aaaaaaagca attaatggaa tagaagataa taatgtatga | 5641 |
| gaagcactat tgtgaaagaa aaaccttcaa cttctctcag ccaaataatt gcttcctcct | 5701 |
| ctgtccttcc cagaccttga tgtttgctct attatttcaa aacaactata ttataaatat | 5761 |
| ttgagaatgt gtatttccct gcaaggagat cttaatcccc aagaaggcag gagctgtgta | 5821 |
| ttattcatcc cagtgctcct tcaaaccagg gcctcagaca gtgcatggcc caaagaggta | 5881 |
| cttaataaac gtttggtaaa ctgacactat tgaaattaag caacctggat ttgaggtggg | 5941 |
| tctctgccac tcacaagaga ttacgctttg agaaaattcc atcacttcat tgattttcag | 6001 |
| ttcttgcatc tgtatatggg agacgatact aggtgatttc tgacatctct caccgtttaa | 6061 |
| atgctctgtg atctatacaa cgaggggctc gctgttctag acaagttcct tccagcttta | 6121 |
| cagttgcata acccttctaa tcttagtcac atgatgactc cactgacaga tttttggcca | 6181 |
| ccatcattag acatgctgag ttacgtgtgc ctttgctctg atcctcaaaa ctcatgattt | 6241 |
| ttaaagtttt ctgaaatatc taccatttat caggatccag atggatttca tgaccaaagt | 6301 |
| ggatgtttct tttctctccc attacaatct tttacttttt gtgtgggaaa ttgcatgtta | 6361 |
| aagaaaggga aattgaagaa tgggatgctt tggaattctg gcaagatgga ttagtgggtt | 6421 |
| ccagaaagta ggggcagcca caaataccga aataaatgag atcgtattat tgagaaagca | 6481 |
| caaatggaag aaggtcaaaa gcaagaagaa gctgacatcc tgcttcctcc aatttttgct | 6541 |
| ttctctgttt ttccaagaaa ctcctcttcc aagccttgct gaaaaactcc actttcctaa | 6601 |
| atctaacttc ttaaactgat aatggcaaga agttaggaat gaccaatatg tttaacactc | 6661 |
| caagagtatt tgttttgttt tgttttgaga ctgggtctca ctctgtcgtc cagcctggag | 6721 |
| tgcagtggtg tgatcacagc tcactgcagc cttgacctct cctgcccaag gaatcctccc | 6781 |
| acctcagcct cctgagtaga agggaccaca ggcatgtgcc actacacctg gctaactttt | 6841 |
| aaaaaatttt tttgtagaga tggggttgcc caggcaggtc tcaaactgct gggctcaagc | 6901 |
| aatcctcctg cattggcctc ccaaagtgct ggaattatgg gcataagcca ctacgcctga | 6961 |
| cctctccaag ggcattcttt acccagaaga ggaacttggc agaacttatc ctccaattgg | 7021 |
| tgaggaatat ggagaaaatg actttaagca aaggaacttc tggttctgcc tacctaatcc | 7081 |
| agaaaaagaa gttttatttc tcccttcccc tagtaactat cttcccatat tcacataaaa | 7141 |
| aagtacagaa tcaacattgt tcaagaatta taattttact tgtaagcaca tgtgcacacg | 7201 |
| cacacccata taccttcctt ccctttaaat catcccacac cctaatagta gtaaaatcat | 7261 |
| tgacccgagc atacctggga gaggaagagg agtctgacag gggcaggttc taagtggcac | 7321 |
| tcctggaact taaccctggt gtatatgaac tttacctatt gaaggatgac tcctcaactg | 7381 |
| ttctcacaat ttgctgctct gctttctttt ctaatttctg aaggtgactc atcttcccca | 7441 |
| aggactttca gacttctcag aagaaaaaaa tattgggtgg gtctctgcca ctggcaaaag | 7501 |
| attagacttt gagaatcata aaagtatatc agtatatact cattaatatt gaattactat | 7561 |
| aattaatatt atgatattga tataatgata gaatgatatt gataaaagca atattcaata | 7621 |
| atgaatatta tttcagctgc ccacttattg ggtgcctcat aggtgccagg cattttgtat | 7681 |
| gtattatcta caacccttac atgggacata ttatgatgct gtttctcttg aagaaatatg | 7741 |
| gaaactggaa acagagaggt caccacaatt ttccaaagtc acatagctaa taggtagcag | 7801 |
| acttgggatt caaattcata tgcatatggt aaatcatgct cttcctctgc tacattttgc | 7861 |
| ccccttagaa tatgaaaaag ggatacaaag agatgaagaa aatatgtaag attatccttc | 7921 |
| aatttcacta tcttttttaa agtttttttt attatacttt aaattctgaa atacatgtgc | 7981 |
| agaacatgca ggtttgttac ataggtatac acgtgccatg atggtttgtt gcacccatca | 8041 |
| acctgtcatc tacattaggt atttttccta atgctatccc tcccctagtc ccccacccac | 8101 |
| cgacaagccc cggtgtgtga tattcccctc cctgtgtcca tgtgttctca ttgttcaact | 8161 |
| cccacttatg agtgagaaca tgcggtgttt ggttttctgt tcctgcgtca gtttgctgag | 8221 |
| aatgatggtt tccagcttca ttcatgttcc tgcaaaaggc atgaactcat tttatggctg | 8281 |
| cataccattc tatggtatac atgtgccaca ttttcttaat ctagtctatc attgatgggc | 8341 |
| atttggctta gttccaagtc cttgctattg tgaacagtgc tgcaataaac atatgtgggc | 8401 |
| atatgtcttt atagtagaat gatttataat cctttgggta tagacccagt aatgggattg | 8461 |
| ctgggtcaaa tggcatttct ggttctaggt ccttcaggaa ttgccacact gtcttccaca | 8521 |
| atagctgaac tagtttacac tcccaccaac agtgtaaaag cgttcctatt tctccacatc | 8581 |
| ctctccaaca tctgttgctt cctgactttt taatgattgc cattctaatt ggagtgagat | 8641 |
| ggtatctcag tgtggttttg attagcattt ctttaatgac aagtgatgat gagctttttt | 8701 |
| tcatgtttgt tggccgtata aatgtcttct tttgagaagt gtccgttcat atcctttgcc | 8761 |
| cactttttaa cggggttttt tcttgtaaat ttgtttaact tccttgtaga ttctggatat | 8821 |
| tagtcctttg tcagatgggt agattgcaaa aattttcttc cattctgcag gttgcccgtt | 8881 |
| cactctgata atagtttctt ttgctgcgca gaagtttttt tagtttaatt agatcccatt | 8941 |
| tgtcaatttt ggcttttgtt gccattgctt ttggtgttta gtcatgaagt ctttgcccac | 9001 |
| gcctatgtcc tgaatggtaa tgcctaggtt ttcttctagg atttttatgg ttttaggtct | 9061 |
| tatgtttaaa tctttaatcc atcttgagtt aatttttgta taaggtataa ggaaggggtc | 9121 |
| cagtttagtt ttctgcatat ggctagccag ttttcccaac accatttatt aaatagggaa | 9181 |
| tcctttcccc gttgcttgtt tttgtcaggt ttgtcaaaga gcagatggtt gtagatgtgt | 9241 |
| ggcattattt ctgaggcctc tgttctgttt cattggtctc tgtatctgtt ttgatacaag | 9301 |
| taccatgctg ttttggttac tgtagacttg tagtataatt tgaagtcagg tagcgtaata | 9361 |
| cctccagttt tgttcttttt gcttaggatt gtcttgacta ttcaggctct tttttggttc | 9421 |
| catatgaaat ttaaagtagt tttttctaat tctgtgaaga aagtcaatgg tagcttgatg | 9481 |
| ggaaaagcat tgaatctata agttactttg ggcagtatgg ccattttcat gatattaatt | 9541 |
| cttcctatcc gtgagcatgg aatgtttttc catttgtttg tgtcctttct tatttccttg | 9601 |
| agcagtagtt tgtagttctc cttgaagagg tccatcacat cccttgtaag ttgtattcct | 9661 |
| aggtattttg ttctctttgt agcaattgtg aatggaagtt cactcataag tttgctctct | 9721 |
| gtttgtctgt tattggtgta taggaatgct tgtgattttt gcacattgat tttgtatcct | 9781 |
| gagactttgc cgaagttgct tatcagctta aggagatttt gggctgagac gacagggttt | 9841 |
| tctaaatata caatcatctc atctgcaaac agagacaatt tgacttcctc tcttcctatt | 9901 |
| tgaatacgct ttatttcttt ctcttgcctg tttgccctgg ccagaacttc caatactgtg | 9961 |
| ttgaatagga gtgttcacca cctattttaa gaatagtatt gaagcctcac aaaagctggt | 10021 |
| tctcatgtaa ccatctgaga atatttggcc ttatgacttg aattcattca ttgccttttt | 10081 |
| atttcacatt ttagtgatcc tgatgtctaa atcttaatct ttgatccttg caaggtaaaa | 10141 |
| tagccaagtc aagcctgttt aataatattg gttgaggaag tcacatgctt atgatcaatc | 10201 |
| tttgggttat gtaattatat taccttaatg ttggcagttt aggtgtaagg cagagatatc | 10261 |
| tgatcacatg tgtggttagc taatttaaga tcactgccaa ctaaaatctt catggtatga | 10321 |
| tcttcaaagt tagctacttt gaccacagca atgatttcac cacagcaatt aacaaaatgg | 10381 |
| cagactcttt cctgaggtgg catgaacagt tttaaaacaa agtcaaggac caaaagaaaa | 10441 |
| gcaggcacat ggcatttgat tcatttcaaa aactagtatt gtattaagag ccaaggggat | 10501 |
| agaattgtag catcaattaa aatcttgttt gaaaaaaaat aaaaacaaac gtccattttt | 10561 |
| atctctcaaa tatattaggg ttttcataaa gttataggtg tatttttaaa aaaacaaaac | 10621 |
| tcatatacat tagactgaaa aatttgcctg tcatctcatc atgcagctaa atgcaattgt | 10681 |
| ctatggcgag atacactctt attagaggta tgatagcact aactaatagc aaactttgta | 10741 |
| cctggtagtc taatttatgc agggttcata tttcgctccc tctcagcatg ctgtaactgt | 10801 |
| ggcaaagcca ttctcaggag ttattacccc aacataatca tacccctgtg gattaggagc | 10861 |
| agttaaatgg gtcctgttat cagagacaca tatgtgccac agccgctgtc atccctaagc | 10921 |
| caccgtgggt gattaagact cactgatggg actacctctg aataggcttg agtgaggtgg | 10981 |
| atacacttca gctgagagaa attcaggtaa ggggctgaga aaatcagatt ttgaatggtt | 11041 |
| ttatcatacc atcaggtctc cttttaagtg ctggggtcat ggatttcatt caacctgacc | 11101 |
| acatagcctt tggaagcttg gctcaatacc tgagtgtgag attatgggtg atattaaagg | 11161 |
| agatgaatgc attgagctga tgtcagagaa tgtcttttac tggattttca taatgactgg | 11221 |
| ctgcagatgg gctgagggga aagtcagatc aagagcattt ctgtaagaag aaagaaatct | 11281 |
| tccctcttat tctctttcaa gaaaatgaat agctgagcac caagaggcca aatacttttt | 11341 |
| taaaaaacac atccttttat gtagagaagg acaggttgag acgaaaaaca ggacctctga | 11401 |
| aactgtcttt atagctttaa tctaggaaga aattgcggca ccattgctga catcattatc | 11461 |
| agaggctgcc ttagttctga gagcttcaca gatggccttt tctgcatttt acatctggcc | 11521 |
| agatgaaggc aaaaaggttg atgaaaccaa aactattaga tcagtggtcc ataactctgg | 11581 |
| ctgcacttta gaatcatctg gggagtcctt aaaactactg atcctgaggc tccatcccag | 11641 |
| accaattgaa tcaggatctc tgggggtggg acctgagcat tggaatgctt taaaagttcc | 11701 |
| tcagggactc taatgtgcag ccaaggctga gaatgactga ggtggatggt ggccaagaca | 11761 |
| ggtgaggcca agagttagaa gcccttactg ttagggaagg cagaagccac aaggaggagg | 11821 |
| ggagggggaa ggagcagtat ttagcatttc ttccacacag ttggggggtt ttctgatcaa | 11881 |
| aattaggctg ggatctttcg cttccatttt catgaaggtc ttatgctttg tccacagcca | 11941 |
| cctggcctca gggcaatgag caatctgatt gatgaatttt cagtaagaaa ctgagcacac | 12001 |
| ttggctctca gccccagtgg cttcccctgt ttccaaatct gcccaccagt tacaggagcc | 12061 |
| tgctcaccaa ctggttgggt taaagaagtc ggctctgtcc ttggcaggga ggccttcagc | 12121 |
| tgtctggccc tgtctgtgac tgcgtgggtg aagctgccta atttggggaa cttgatggaa | 12181 |
| gatctaagct atgttctcta acagttttat cagaaataaa gttaactttt gacctccatc | 12241 |
| tgcctgtctc ctgtggaagg gctctgctcc ttccaaagag actctcaggg gttctcctta | 12301 |
| gaggtgtgtt atcagtccaa agatcatttt agaccagcca ttacagagga tgtccaagaa | 12361 |
| attgcacaag ggaaatagaa gtaagaatga gagcaatata tcagatagta aggaaagtat | 12421 |
| gtgactggtt tcaaataagt aattaaccat aaatccaaag ttcctgcatt agttatagca | 12481 |
| atagtcatcc acctggtcag gaaaaaaaaa gtaaaagact gagctgcaag atagaaagtt | 12541 |
| tgcctgaatt ccacgtactt caaggactac tatggaggat tccttggtcc caatgcagag | 12601 |
| acatgtactc tgaccaccca tgggccaaat ccctcttacc caccctcagt gattcctcct | 12661 |
| gggacctcac tacattgagt tcttacattc cccactcttt tcgggggaaa tataaccctt | 12721 |
| ctgctcttct catgatgtta aaaatattta gacaattact taaaatttac aacaatcttc | 12781 |
| cacataaatg atctcactta tgtttcttgt gagctctgtg atttatttct attatcatct | 12841 |
| atgctgatag ttaaggaaac tgagatatcg agaccaacat gcatagtaaa tggcaaaacc | 12901 |
| agatgaattt taaactttcc taactccaaa agccacatcc tgcccaaccc gccatgctgc | 12961 |
| cgctcagtta atgcctggct gtttgtctcc ccattggccc ctctacccat tgtgctttga | 13021 |
| tggcacactg tattccaact gcctgagact cctggttaat gccattacgt acagacttag | 13081 |
| gttgaattta ctaggatttt taagatttgt aggataagag atatgactgt tagactggaa | 13141 |
| tcagcaatag ataaaaggtt aacaaagttt cagaataaaa tataatagaa aaccccagca | 13201 |
| gatagaagta aaatgaatgg taagacaact gaaatgaaag ccatacattt agaaatatca | 13261 |
| aataaaacac agattaatgg cagacaataa aggaacatac ttagcagtta gtaaaaacac | 13321 |
| tttttacctt attcttatta ccctccagta accttttttt tttttttttt tgagacaaag | 13381 |
| tctcgctgtg tcgtccaggc tggagggtag tgatgtgatc tcggctcact gcaacttctg | 13441 |
| cctcttgggc tgaagagatt ctcctgcccc agcctcctga gtagctggga ctataggggc | 13501 |
| ccatcactgc acctggctaa tttttgtatt tttagtagag acggggtttc accatgttgg | 13561 |
| ccaggctggt cttgaactcc tggcctcagg tgatccaccc gcctcagcct cccaaagtgc | 13621 |
| tgagattaca agcgtgagcc accaagcccg gctgtaacct attgaaaata acattacttg | 13681 |
| acatgtgaga caaattattt gtaagttaaa gagtttatgt gccttcaatg ctcccaccct | 13741 |
| tccctcccct aaaaagatta ttgagtgccc atgatgtgcc taagccttct ggtagactct | 13801 |
| gagaatgtga agagagttag aggatacttg ttcaacaacc cagaatctag cagaagtgtt | 13861 |
| ttgaaatgac cagccacttg ggagagctat gctagcatat cattcaggat gggctgggtc | 13921 |
| atgttttaat aacaagtaag tttgaacttt ataggtttga aacaaaaaag atgtgtttct | 13981 |
| tgctcacagt tcatattttt tggagattgg ctggagcttc attccaggat actgccacca | 14041 |
| tctggaatgt tgacagttac cgtaacagag aaagagttgt ggaggctgtc acactggcaa | 14101 |
| ttaaattcgc tgacctggaa ctgacacatg ccacctccac ttatatttta ttggccaagg | 14161 |
| cttgttacac agccacatct aacttcagag aggtcaagat gagcaaatcc taccacgagc | 14221 |
| tatagatgga tgagtagcac tcattattat cacaattatt ttatttgtta taaaaactcc | 14281 |
| aagaaggaga ggtactctat gtgaggaata ggcataggaa aatcagaggg aagttcttaa | 14341 |
| agatgagcta gatttcacta gatggcattg aagaaacatt ccaagtaaat gaacagcata | 14401 |
| agcaaatgca tgaagaactt attgtggtcc tagtacagac gatgcgtgag agtgtggagg | 14461 |
| agggaaaagg taagactgga gaggaaggca ggaaccagaa caggacagac tgtgttcact | 14521 |
| gtcaggcagt taagtctatc ttgtaggcaa catgaagcct tttaaggaag gcaagcagct | 14581 |
| atgtgacagg acagaagatg gattggaaga aaatcaaaag agaagtgggg accagtcata | 14641 |
| aggctcctcc aatatttggg gatccaaacc aaagcactgg cagagaaata gaaaggaagg | 14701 |
| gaaatatttc caaaatattc aaaacagaaa ccaagagaac ttgatgacag aagatgaacc | 14761 |
| caggtttcta ctgaatggac agttgttaca ttctctgaga taaggaatac agaaggaaaa | 14821 |
| agttgagagg gaatatgaaa ttatttttaa tcatgtttaa tttgatcact tgtggaacat | 14881 |
| caaccagaga tgtccatcag ttacaccaat ttgtaggttt tgagagaggc ctgaggtaga | 14941 |
| ggcagagaag tgggaatcag cagattagcg gcagtagttg gaaccataac tgaagatgag | 15001 |
| atttcccagg gaggggagtt gaacaggaag gtagacaaag gctgagtgca gtggctcata | 15061 |
| cctgtaatcc cagcactttg ggaggctgca gtgggtagat cacaaacaag atcaggagtt | 15121 |
| caagaccagc ctggccaaca tggtgaaacc ccgtctctac taaaaaaaca caaaaattag | 15181 |
| ccgggcgtgg tggcacacac ttgtaatccc agcctcagga ggctgaggca ggagaattgc | 15241 |
| ttgaacccgg gaggcagagg ttgcagtgag ccaagatcgt gccattgcac tccagcctgg | 15301 |
| gcgacagagc gagactccgt ccccaccgac cccctaaaaa agaaagtaga caaagatgtg | 15361 |
| tcctagaaaa cactaatatg aatgggtaga gaggggagac ttgtcaagga gatcgagggg | 15421 |
| agagtcagtg aggtgaaacc ttagaggata gaccttcacc aaggaagtaa cagtcaacag | 15481 |
| gcctaaatgc cacagagatg tcaaatgaga gacactggaa atggttcttt gaatattaca | 15541 |
| gccagaacat cacaggagac catttccaaa gcagtcttga tgaagtggtg gggaagggag | 15601 |
| gtccctgaag gagctaagga gggactggga gatcatgacc cagagataag tgttgcaggt | 15661 |
| ataaaaggga aagactgaga tcaggaaata gccacaggat ccctaaggcc aggcttcggg | 15721 |
| tggggtggtg tgtgtgtgtg tgtgtgtgtg tgtgtgtgtg tataaaatgg gaagaccttg | 15781 |
| agtataattc tgtactaaga agatagagca gtagagagga aaaggctgaa gacagatggg | 15841 |
| agggtaaata gtgaattaga aaggtctgtg caaagatgag aaaggatgag tctgaaagca | 15901 |
| cagggagaag ggccagcctt ggacaggaga aaacatttct ttcactaaga ataaagggaa | 15961 |
| ggttgggtat acaccacaaa gaaggtgtag atgggtggca atggagttct gtcaagttga | 16021 |
| gggcattcca tgatagcctc acctttctct gtgaaatgag agagtttagt ttacaagata | 16081 |
| tttctgagca cttcataagc caggttcata gactgaaaga agtggaggtg gagtgtgata | 16141 |
| ggtccttaag aataggggaa gtttggaata tctgacaagg gacagagagg aaaaaactag | 16201 |
| aaaaggcttt gcagaatgtg ggcccacaga tcagaggcta aggggtcccc atttgtgcag | 16261 |
| gagagtaagg gcagggaggc aaggctcaca gcccaaaata tagagcccct caccaaatga | 16321 |
| ctgcaggagg gcagctttcc tatgagagca tccctatcac tgttttcact ccgagtcatt | 16381 |
| aacttacgac ttactcagct ctgtttcgta atagcagact cgagtaatga gggtatgaca | 16441 |
| gcctctctct gcatgccaag gtatgcagcg tggatttcct ttttcgcttt ctctctcctg | 16501 |
| tggcttaggt gccttctgtt ctgctaccag gatagagaac ccagtgacta gtttcttcta | 16561 |
| gctctctttt tctgactagg tatcttgtca gaaatttctg cttaccagac ttcatggaga | 16621 |
| gggaatcaag ctttgaatca gggttgaaaa agtagagctt aatatatata ttacaaaatg | 16681 |
| ccactcacgt tcttgaggtt accttgtatc tataccacaa ctagcattct tttagaaagc | 16741 |
| accattaccg aagtaatccc tttcctggga attcacccaa aaaggttatt cccacttatc | 16801 |
| ccccatctcc aaaataaaaa agaaaaatgt gtgtgcttag agatgttcct ggaagcatga | 16861 |
| gctgtaatac tgaaccaata gaagacgaac taacagattg cagggcatcc gtttggcaaa | 16921 |
| aaacttatgc agttatctaa atgatagtta tgaagacaat atgtataaca cattatattc | 16981 |
| tgagtgaaaa gaacagaagg tgatttcaaa actgcattgg gataatagta atataggaaa | 17041 |
| tagtgagatg aacaaagatt tcaaaggaac aaaaataaag aaaaattttg ctttttatat | 17101 |
| tggtaggcgt atgggtgaaa ttccaatttt aattttaatt tcatagttat aaaattgttc | 17161 |
| ataaaaaaag gtcccccata gacagttggg ctttgggaca aactaacaga aacagagtag | 17221 |
| gaagaaaatt catcttcctt caatcccctt tctctgctta aaacaaaaca aaaaagagct | 17281 |
| ttgtcatgtt caggtgtgca acgaattctt tttccaaatc tggaacttta catctgctat | 17341 |
| taaacaggag tcagtttcca tgtaacatgt tgacaatccc ccaagtgtgt tggaataatt | 17401 |
| ttttttaatg aggagatttg aaattccatt tcaattgcca acctgcctct ttcaacttct | 17461 |
| aaaaacaaag taaaacaaaa caaaaacaca ctgggtccta tcaccccctc ttgctactac | 17521 |
| tattttatct ccattgccct gaattctttc caaacttctt tccacccagc tttgatttgt | 17581 |
| tttcagtcgg gtttattgag gcataattta cgtacagtaa aattcatcct tcttagattt | 17641 |
| agaggtctat gcattttgac taatgcatat tggcttataa tgactaccac aacaaagata | 17701 |
| tagaacacac ccatcactcc cgggttctcc ctgtcccttt tttggtccat ctcctctcct | 17761 |
| acccccaacc ccttggcaac cactgatctg ttttctgtcc ttatcatttt gctttttcca | 17821 |
| ggatgttgta tataaggaat catgcagcat gcagcctctc gagtctgact tcttccagtt | 17881 |
| agcacagtta tttaagatct atccgagtta ttgtgagtag cagcatcttt tttattgctg | 17941 |
| actagtattt catcacatgg atgggccaca acttgtttat ctgttcacct gtcaatggat | 18001 |
| actaagttgt ttccagtttt tggcaaatat gaataaagta aacatttgca tacagatttt | 18061 |
| tgtgtggaca catgttttca attctcttag gtaaatactg agcaatggga ttgctgggtt | 18121 |
| atatgttaag tctatgttca gttttctaag ttctgaaaca attggatatc tatatgcaaa | 18181 |
| aatacaaaat taaccttgac tcaaacttgt accatacaca aaaaataacc tgaaagagat | 18241 |
| cacaggtgta aatgtaaacc tagaactaaa aacttcaagg agagaaacat agcagaaaat | 18301 |
| atttgtgacc ttgcattagg caaagacttc ttagatttga catttgaaac atgatacata | 18361 |
| aaaaatcttg ataaattgga gttcataaaa ataagaacta ctcttcaaaa gacactgata | 18421 |
| agagaatgaa aatacaagcc acagacagag aaaatatttg tgaatttctt atctgataaa | 18481 |
| gggtttgtat ccagcatgca taaagacttt tcaaaactca ataataaaca atccaataag | 18541 |
| aaatgcacaa aagatacaaa cacatcatcg aaaatgacct atgaaaggca aataagccca | 18601 |
| caaaaaaatt ctcaacatca ttagacactt gggaaatgca aattaaaaca acactgagat | 18661 |
| aaactacata tctattaaat ggctaccact ttaaaaacct gtcaagtgcc agccagaatg | 18721 |
| tggaacaagt aggactctct tacattgcta gtgggaaggt gaatggtaca gccactttgg | 18781 |
| aaaacactcc gcagcttctt atagttacac ataggacctg ggggtagagg atggattgac | 18841 |
| tgtaaagggg caaaacttcc tggtaggggt ggggaaagtt tttagaaggg tgaaattgtt | 18901 |
| ctatatcttg attattgtgg tggttacaca actgtctgca tttgtcaaaa ctcacagaac | 18961 |
| tgtacactaa aaaggtatat ttttatgctc tgctaatttt actttaatct taaaaatagg | 19021 |
| aaggaaaaaa taaaatcaat gccactgtgc gactttgggc aagttacttc acttctctgt | 19081 |
| gccttggtct tttgaaatct atacattaag gataaaataa taccttcctc atactgttag | 19141 |
| aattaaatgt gctaatttat gttttatata tataaagtac ttggccgggt gtggtgactc | 19201 |
| acacctgtaa tcccagcact gtgggaggcc gaggtgggca gatcacttga ggacaggagt | 19261 |
| tcaagactag cctggtcaac atggtgaaac cctgtctcta ctaaaaatac aaaaattagc | 19321 |
| tgggcttggt ggtgcgtgct tgtaatccca gctacttgag tggctgaggt gggaggatca | 19381 |
| cttgaactcc agaggtggag gctgcagtgg gctccaccca ctgcctgggt gacagagcta | 19441 |
| gactccatct cgaagaaaaa ataagtactt gaaacatagc aaatgtttta taattattgg | 19501 |
| cttttttttc ttattgttat tacttgcatt attgctgttt gaagaagttt ggtgataatg | 19561 |
| gagagaaaag ggcaattagg ggtctgggat ggtttaagta tgaggagacc gagacacatt | 19621 |
| gactcagagt gaagaaatca aagataagag aatgaaagaa agggagggta attctgaacg | 19681 |
| cacagacaaa gttatgttac taacgtggca tcggctgtgt gttgtaataa ataactccct | 19741 |
| ttcacttgtc aatagctaat caaatacttt ctggagacca gaagtgactt gctggatcaa | 19801 |
| tgacaactcc tccacagatc aaaatgttca aatccttttt ctgtgttgta atcctaaatc | 19861 |
| taaaagaaca gagagaccaa gcaaatctac ctcccaacat cattaaagtg acaactctca | 19921 |
| gtatttattt gaatggtctg ctctcagctt caaccaagga aaagtcaaat tagtgttggt | 19981 |
| cagaaaacag aagggtgtta cgagagttct ggctggtcat tacagacttg gggatttttg | 20041 |
| attaaaagaa gaagaagaag aagaaacctg ataaagtgta aatatagcaa gcagggatta | 20101 |
| gtgtcctgct gggtcatgtt ctcacaacag tgagaatttc agagatttca taagaattaa | 20161 |
| actgctccac atgacaattt attttacctt ctggcttttc caggaggcaa atcagtgcaa | 20221 |
| cttctttctg cctttgtttc aatttggtaa caaccctcaa ttttaggaca ggctaaacct | 20281 |
| agccacccta tcagagatga tgaagtagcc atctttttaa caggtgggga gatgaatgga | 20341 |
| atcagggttt gtttgtttgt ttgtttaata actgctagta aaaaccaagt caatagctga | 20401 |
| ctgagtgtaa gggaggctcc agaaggcagg ttattgtagt atagatgtga ctcgacttat | 20461 |
| gatgatgtta cttcccgata aacccgtcat aagttgaaat atcgttaaat tgaaaatgct | 20521 |
| tttaatacac cgaatctacc gaacatcata gcttagctca gcctacgtta aatgtgctca | 20581 |
| ggacgcacat tgcctacagc tgagccaaat cacctggcaa cacaggacac tgtagagtat | 20641 |
| cggttgctgg cccttgtgat gctgtgactg actgggagct gcgcttagtg cctctaccca | 20701 |
| gcattgagag tttcttatcg cttattacta gcctgggaaa agaccaaaat tcaaaactca | 20761 |
| aagtgcggtt tctaccgaat gcttataact ttcagaccat catgatgttg aaaaatcgaa | 20821 |
| ccatcgtagg ttgggatcca tcctataaga cgagctacac tgccggaagt gtaagactgc | 20881 |
| tatgctgccg gaagatgggg catagtggac aactgcaagt cctgacaaca ggaggtcagc | 20941 |
| atctgcgacc tttaacatcc acattgacac taccacagtc ttccaaacag agctgatgat | 21001 |
| atagtttgga tgtcgtccct gcccaaatct catgtcgaat cgtaatcccc agtgttggag | 21061 |
| gtggggcctg gtgggaggtg attgggtcat gggggcagag ttcttatgaa tggtttagca | 21121 |
| cggtcccccc ttggtactgt atagtgagtg agttctcatg cgatctggtt gtttaaaagt | 21181 |
| gtgtggcacc tcccctctct ctctttctcc tactctggcc atgtgaagtg ttggctcccg | 21241 |
| ctttgccttc caccatgatt gttaaattcc cagaggtctc cctagaagct aatgctgcca | 21301 |
| cgtacagcct ggagaactgt gagccaatta aacctcatct ctttttaaat tacccagtct | 21361 |
| caggccgggc gcggtgactg acacctgtaa tctcagcact ttgggaggct caggcaggaa | 21421 |
| gatgatttga ggtcaggagt tcgagaccag cctggccaac atggtgaaac cccatctcta | 21481 |
| ctgaaaatat aaaaattagc caggcatggt ggcgggtgcc tgtaatccca gctacttggg | 21541 |
| aggctgaggc aggagaatcg cttgaacctg ggagtcagag gttgcagaga gccaaaatgg | 21601 |
| agccactgta ctccagcctg ggcaatggag tgagaccctg tctcaaaaaa tatatatata | 21661 |
| ttacccagac tcaggtattt ctttacctga gactatgaga gaatggacta atatagctga | 21721 |
| agaattttat tttattttta aaaaactttt acgtttgggg gtacctgtaa aagtctgtta | 21781 |
| cataggtaaa ctcctgtcat gaggatttgt tgtacagatt ctttcctgct cctccccctc | 21841 |
| ctcccaccct ccatcctcaa gaagatccca gtgtctgttg tttccttctt tgtgttcgta | 21901 |
| agttctcatc atgtagctcc cacgtataag tgagaacatg cagtatttgg ttttctgtcc | 21961 |
| ctgtgttagt ttgctaagga tgatagcctc caactccatc tatcttcctg caaaagacat | 22021 |
| gatctcattc atttttattg ctgcatagta ttccatggtg tatatgtacc acattttctt | 22081 |
| tatccagtct gtcattgatg ggcatttagg ttgattctgt gtcttcagaa ttgtgaatag | 22141 |
| tgctgcaacg aacattcgtg tgcttgtgtc tttatagtag aatgatttct attcttctgg | 22201 |
| tagtaatggg attgctgggt caaatggtcg ttctgctttt agctctttgc agaatcacca | 22261 |
| tactgctttc cacagtggtt gaactaattt acactcccac taacagtgta taagtgttcc | 22321 |
| cttttctctg caaccttgcc agcctctgtt atcttttgac tttttaataa aaaccattct | 22381 |
| aattagtgtg atggtatttc attgttgttt tgatttgcat ttctctaatg atcagtgatg | 22441 |
| ttgagctttt tttcatgttc gttggctgca ggtacatctt cttttgaaaa gtgtctgctc | 22501 |
| atgtcctttg cccacttttt aatggggttg tttttctctt gtaaatttaa gttcctcata | 22561 |
| gatgctgggt attagacctt tgtcagatgt atagcttgca aatattttct cccattctgt | 22621 |
| aggttgtctg cttactcttt tgattgtttc ttttaccatg cagaagctcc taagtttaat | 22681 |
| tagatcccat ttgtcaattt ttgcttttgt tgcaattgct tttggtgtct ttgtcatgaa | 22741 |
| atctttgcca ggtcctatgt ccagaatgat attgcctagg ttgtcttcta gggtttttat | 22801 |
| agttttgggt tttacattta aatctttaat ccatcttgag ttgatttttg tgtttggtgt | 22861 |
| aaggaagggg tccagtttca atattctgca tatggctagc cagttatccc agcattattt | 22921 |
| attgagtaag gagtatctcc tccgttgctt gtttttccca ggtttgttga agatcagatg | 22981 |
| gttgtaggtg tgtggcctta ttttggggct ctctatcctg ttcatttggt ctatgtgcct | 23041 |
| gtttttgtac cagtaccatt ctgttttggt tactgtagcc ctatagcata tttcaaagtt | 23101 |
| gggtaacatg atgcctccag ctttattctt tttgcttaga attaccttgg ccatttgggc | 23161 |
| tctttttggt accatatgaa gtttaaaata gttttttttc tagttatgtg aagaatgtcg | 23221 |
| ttggtaattt gataggaata acatgtaatg atattgattc ttcctatcca tgagcatggg | 23281 |
| atgtttttcc atttgtttgt gtcttctctg atttcttcaa gcagtgtttt gtaactcata | 23341 |
| ttgtagagat tattcacctc cttgcttagc tgtattccta ggtattgtat tctttctgta | 23401 |
| gtaattgtga atgggattgc ttttctgatt tggccctcag cttggtattg ttggtgtata | 23461 |
| ggaatgctag tgattttttg tatcctgaga ctttgctgaa gttatttatc agctgaagga | 23521 |
| gcttttgggc tgagactagg gggtttttta gatatagaat catgttctct gcaaacagat | 23581 |
| ttagtttgac ttcctctctt cctacttgga tgccctttat ttctttctct tgcctgattt | 23641 |
| ccctggccag gacttccagt accatgttga ataggcgtgg tgagagaggg cattcttgtc | 23701 |
| ttgtgccagt tttcagggag aatgcttcca ccttttgccc attcagtacg atgtttgtgg | 23761 |
| tggtttgtca tatatggcta ttattatttt gaggtgtgtt cctttaatac ctagtttatt | 23821 |
| gacagttttt aacatgaagc agtgtttaat tttattaaaa gtcttttctg cctctgttga | 23881 |
| gatagtcatg tggcttttgt ctttagttct gtttatgtga tgaatcacat ttgttgattt | 23941 |
| ccttatgttg gaccaacctt gcatcccagg gatgaagcct acttgattgt ggtggtttag | 24001 |
| ctttttgata tactactgga ttcagtttgc aagtattttg ttgaggattt ttgcattgat | 24061 |
| gttcatcacg gatatcggcc tgaagtttct ttttttgttg tgtctctgtc aggttttggt | 24121 |
| atcagaatga tgctggcctc ctagaatgag ttggggagga gttcctcctc ctcaattttt | 24181 |
| ttggaatagg ttctgtagga atggtaccag ctcttcttta tacatctggt agagtttggc | 24241 |
| tgtgaagcca tcaggtcctg ggatttttta gttggtaggg tatttattac tgattcccta | 24301 |
| aatagaccga taatgatttt agaagtggag tcggtttttt cctggtccag tcttgggaag | 24361 |
| gtgtatgtat ccaggaattt atttagctct tctaggtttt ctagtttgtg tgcatatggg | 24421 |
| tgttcatagt agtttctgat ggttgttttt atttccgtgg gatcagtggt aacattctct | 24481 |
| tcatcatttc tttttttttt tttttttttt ttttttgaga cggagtctcg ctctgtcgcc | 24541 |
| caggctggag tgcagtggcg cgatctcggc tcactgcaag ctccgcctcc cgggttcacg | 24601 |
| ccattctcct gcctcagcct cccgagtagc tgggactaca ggcgcccgct accacgtccg | 24661 |
| gctaattttt tgtattttta gtagagacgg ggtttcaccg tgttagccag gatggtctcg | 24721 |
| atctcctgac ctcgtgatcc gcccgcctcg gcctcccaaa gtgctgggat tacaggcgtg | 24781 |
| agccaccgcg cccggcccat ttctaattgt gtttatttga atcctctctc ttctcttctt | 24841 |
| tattaggcta gctagtggcc tatctatctt attaattttt tcaaaaaacc agctcctgga | 24901 |
| tttcttgatc ttttgaatgg tttttcatgt atcaatcctt cagttcagct ctgattttgg | 24961 |
| ttatttcttg tcttgtgcta gctttggggt tgacttgttc ttgcttctct aattctttca | 25021 |
| gttctgatgt tagtttgtta gtttgagatc taactttttg atgtggacat ttagtgctat | 25081 |
| aaatttaact cttaacactg ccttagctgt gtcccagaga gtctggtatg ttgtatcttt | 25141 |
| gttctcatta gtttgaaaaa acttcttgat ttctgtctta atttaattat ttatccaaag | 25201 |
| tcattcagga acatgttgtt taatttccat gtaattgcat ggttttgagc gattttctta | 25261 |
| gtcttgactt ctatttttat tgtaccgtgg tctgagggtg tttgatatga ctttggttct | 25321 |
| tttgcatttg ctgaggattg ttttatgtcc aattatgtgg ttgattttag agtatgtgcc | 25381 |
| atgtggtgat gagaagaatg tatattctgt tggttttggg tacagagttc tgtagaggtc | 25441 |
| tattagatcc atttggtcca atgttgagtt cagatcctga atatctttgc taatttcctg | 25501 |
| cctccatgat ctaatactgt cagtaaagca ctgaagtctc ctactactat tgtgtgggag | 25561 |
| tctatgtctc tttataggtc tctaagaact tgctttatga atctgggtgc ttctgtgttg | 25621 |
| gatgcatata tatttaggat agttagatct tcttgttgaa ttgaaccctt taccattatg | 25681 |
| taacgccctt ctttgtcttt ttttttcttt gttggtttga agtcttcttt gtctgaaatt | 25741 |
| aggattgcaa cccctgcttt tttctgtttt ctgtttgctt ggtagatttt cctccatccc | 25801 |
| tttattttgg acctatgggt gtccttacat attttatctt tatctatcca tccagccatc | 25861 |
| cagccatcca ttcatccgta tcatttttaa ccaataagga cttttaaaag cgcaaccaca | 25921 |
| acaccattaa cataaccaat aaaatctata acaatgataa aatatcatct aatactcagt | 25981 |
| ccatgtccaa ttttccctcg ctatctcaaa atcgtcttct tagaaatggt ctgttcaaat | 26041 |
| gagatcacat ggacagagga aggcgaacct cacactgtgg ggactgttgt ggggtggggg | 26101 |
| gaggggggag ggatagcatt gggagatata cctaatgcta gatgacaagt tagtgggtgc | 26161 |
| agcgcaccag catggcacat gtatatgtat gtaactaacc tgcacaatgt gcacatgtac | 26221 |
| cctaaaactt aaagtataat aataaaaaat aaaaataaaa aaataaaaag tgaaaaaaga | 26281 |
| aaaaaaaaaa aaaaagaaat ggtctgttca aatcacaaac cagattcaga aacaatagcc | 26341 |
| atacattaca ttttattaat atgtctctta aatttctttt aatctattac agtctttgga | 26401 |
| atttttatgt cttcgtttat ccttccaatt attaaaaaaa aagtattttt gtattcattg | 26461 |
| aatagacaat gcttgcagaa aagtaaaaaa aaaaaaattt agtacaaaaa ggtacatagt | 26521 |
| gagctgttcc ttagtctccc ttcccagaag caatgttacc acttttgtac aaatagtctc | 26581 |
| tgcctagaca cacatgccag tccctaaggt ggctgtaaca aggtggttaa gagtgagaac | 26641 |
| atgaattcaa attcctatta tgccactcac taagtataaa tcttggtcat ggtacatgcc | 26701 |
| tctgtgcctc agtttttaat aatggtacct acctcatagg gctgttgaga gaattaaatc | 26761 |
| agataagtgc ttaaataact attaatattt attattattc acattccctt ttggcttttt | 26821 |
| tcccaaatag cagagtggtg cacatatgtc ttcatttatt tggcttgttt tttcacctca | 26881 |
| catcacattt tgatgaataa ttccatacat gttgttatag atttgcttca ttctttgtaa | 26941 |
| tcattgacta atattccatt gtatgaatat gctactgcta aacatgtacg ttatttccaa | 27001 |
| cctcttatta tcaagaaatg ctgcaatgaa tatccttgta atactttagt ggattcatgt | 27061 |
| gcaaaaatat tcataggata aaatcctgaa agttaaattg ctgagctaaa gggtatgtgc | 27121 |
| attttaatgc tttatagatt gcccagctgc ctcaaaggag gttataacaa tttacactcc | 27181 |
| caagaaaaat gcacaagggt ccccatttcc ccatacccta gctaacacag gatattgcta | 27241 |
| aatgctttca tctttgtaaa catgatgtat tgaaaatggt atctcaaagt tttaatgtgc | 27301 |
| atttttctga ttgtgagaag ataaaggaaa tgtagtacaa ctaaacatca gcagtcaaat | 27361 |
| gacctggcca tgactcctga gtgaggacac tggtaaacac catcaggatc caaacacctc | 27421 |
| tgtatttacg aagaggatgc tccctattgg atagcactaa gcttatttca tgtatgtaca | 27481 |
| tatgtagtta gttaattaca tccagcggtg gcaaagggct tgttctgacc caatgaaact | 27541 |
| ttctctcctg gcccccttcc agcatgtggt caggagtaga gtgttgtggc catgaggcat | 27601 |
| gcatttgtac agatgactac ttactcctcc ttgaaacatt tttttccatt tgcttccctg | 27661 |
| ctgtctcact catgggtctg ctcctaattc acaaatcact cttttcccag tcttcttggt | 27721 |
| tgggttttct cctcttctgt gcttgtagac atgggggagc cccagggctt ctctcttgaa | 27781 |
| ctacagcttc tccctgggtt catctccttg ggatgtctgt tccaatgggt ttaaatacta | 27841 |
| gggctaggac tagggagagg tcattgaggc acttagcaca taaagtttaa ggaaacattc | 27901 |
| tttatcaggc ccatgcaagt gcaggactgg ccctggaggg tgactaccac cttacctttt | 27961 |
| ccaccctagg caccttgttt gccttaccct agccccagtc ctctttaaca ccccagtctt | 28021 |
| ctccctggac ctccagaaac ataaatccta tttgacattt ctacttggag gttttaaggt | 28081 |
| aactcaaacg taaaatatct aagacagaac tcttgcatca cttcccatcc tggggcccaa | 28141 |
| gcctgtctct tctactagtc tatctcagtt aacagcatca ccatttattc agttgctcag | 28201 |
| gacaaaaaat ttgaagtaat ccttgactct tctttttttt tttttgagac ggattctcac | 28261 |
| tctgttgccc aggctggagt gcagtggtgg gaccttggct cactgcaacc tctgcctcct | 28321 |
| gggttcaagc aattctcctg cctcagtctt ctgacctcgt gatccactcg cctcggcctc | 28381 |
| ccaaagtgct gggattacag gcgtgagcca ccgcacccgg cctggattct tttttttttt | 28441 |
| tttaaacaag tcctatcttc catctccaaa atgtatccca aatctgacaa cctctcccac | 28501 |
| cgtaggccag cccccatctc tcccctctga aaatagcctc ccttagatct ctggacattt | 28561 |
| gttcttcccc acccccttgt gatcactatt cagcattcag aatgatcttt taatattatg | 28621 |
| aaggagactg tgttcctctc ctacttaaaa ttctctagtg gcttcctaat aaatttagaa | 28681 |
| taaaaagcca actcctcgcc atggtcacca ggccaggatc cagtgggtac aacaatctgt | 28741 |
| tcccagcaca ataatcccac ttctgcctcc ccaccattca ccaggctcca ctgcactggc | 28801 |
| tcattcctgc ctcagttgtg cttcttttcc ctggaaagtt ctttctgtag atctttaaag | 28861 |
| ggtgatttcc ttctcaacat tcaggtgtca gctcgtttca ctttcctgac catgcccatc | 28921 |
| cactcagaga tcactcaaaa tcccattacc ctattttatt tctccatcat atgtatcact | 28981 |
| atctgaaact atcttgttgt tgatgcaggc tattttcttg accccttcat gggactccca | 29041 |
| acaggggtac cccatttact cagcctgccc tgctcaacct cttgcaggag ggagcacacg | 29101 |
| agtgaacgag tgcaggaacc agctggctgc tttagtgctg tgaggagtaa actccatgca | 29161 |
| ggccctgcag cagcaaccag gtaggggtgc ctgcaacccc agggccccag agggtgtgtt | 29221 |
| acaatgctct cgtagctctg ccatctgtgg acagcagtgt gttgtcagct cagtgggccc | 29281 |
| tttgcttcat catgtagggt ggctgccctc tgcctgtgag ggcaaagggc cagggtgaca | 29341 |
| gtctttttgg gtacccacaa tttgtgcatc ctgaattctt gtttggtgcc caagaagaat | 29401 |
| ggggtcacac agatgaactg aaggatggtg aatgcagaga attagcaatg aaagtggctc | 29461 |
| tcagcagaga gagaagctga aaagggaatg ggaagggcag gtcactctcc cctgaagtca | 29521 |
| agtcacatct ctccaatgtc cagccaccat ctctgaagtc aagtttcctc tctctgatgt | 29581 |
| ccagccactt ctcctctcta ctggctgagt ctggggtatt tataggcaga ggataggtgg | 29641 |
| tggggcaggc catacataat tttggaaaag gcaacattct attggtaaaa agacattatt | 29701 |
| cataaagaac caattgggaa agagcgggca cacagggatg gaagttctca ctttgggctg | 29761 |
| caggtttcag gcttttcagc tcaaaagtga ggtttttcca gggacctgcc ccgtctgcct | 29821 |
| aaaatttcta cacttctgtc attgataccg ctggataaca gctctcctat aaagttcagg | 29881 |
| ggcttaaaac aaaaatttac taattcaccc ggacagttct tgatagggtc tctcctaact | 29941 |
| gttgcagtta catagcaact gggttggagt catcttttct gggcttgaca tccaggacag | 30001 |
| cttcttccct tgtgtgtctg gtgcctcagt gctcctccag gcagcctttc tctccagaag | 30061 |
| agtagcctgg acttcttggc aactcaagct tccaaaagaa aaaaaagcag agactgctgg | 30121 |
| ttctcttatc aaacaggcct ggaactggca caatgtactt ctgctgccat attcaggagg | 30181 |
| tcaaagcaat cgaaggccaa tccaagttca aaggcattgg agaaaaatga gaagtgtcac | 30241 |
| ttaaatgaga agtgacacat gcgtaaaagg gggaaaagca ttgattgtgg ccatttttgg | 30301 |
| agataagcta tcacgtttat ttgtttgttt gctttctaat ggtctgtctt ctcccattag | 30361 |
| gttataagct ctgtgagaca acaggaatct tgtccatctt gttttatggc tctacttcca | 30421 |
| acacctagaa taatgcctgg cacatagtag gtgctcagtg aataacttaa gcacttgata | 30481 |
| catgtttggg gaactaaaat gaacagaatt aaacttcccc agattggtcc tgccagattt | 30541 |
| gctgatgcca agcatgctga tgcctcacca gatgaaagaa gccctaaaat gtagggtttc | 30601 |
| gctttctctg caaacaagaa aaacttgccc tgaacacaaa atctagaaat agatttggcg | 30661 |
| tgttttctac attgaaatat ttcccgtagt accagaaatt attttcccac agctttgtgc | 30721 |
| tacattaaaa tattgaagtt gactgaaaat atctccattc tttaatcttg gtgtagacta | 30781 |
| gaaacaattt ttttgtaaca aagtaaatat gaaaacttcc taatatttga actccccaga | 30841 |
| tatccccaga tatctccaaa cttaaaatat cattgcaagt taagataaat ttttttaaat | 30901 |
| gactaccgag aaaggtcatt aaaggcttgg ttattaaaat gtacagattt gggttataaa | 30961 |
| gccagaactt atttgtttaa atcattacat atgaccaagc acagaaaata aattacctca | 31021 |
| aatctcctct ttgctaattt ttactggtaa actctataaa atgatcctat ctttaaacct | 31081 |
| ttttgtaaac cccttataag ttagtaagtg agaatgtatt catcagaagg attttagtga | 31141 |
| tgtttgaaat taaaaaagag agatttgatt tttaaaatta tacttgcaga ctactgctaa | 31201 |
| tgaaacttct tctaacccta gttttgtctt atcttcagtt tttccagatt tgctcaaagc | 31261 |
| aatcccagtg agcatccacg tcaatgtcat tctcttctct gccatcctta ttgtgttaac | 31321 |
| catggtgggg acagccttct tcatgtacaa tgcttttgga aaaccttttg aaactctgca | 31381 |
| tggtccccta gggctgtacc ttttgagctt catttcaggt aagtacaaaa ttctacctct | 31441 |
| gaagacaaat gtgcttttca atatgtcaaa aagaccgtct acctaaatat aaagttataa | 31501 |
| tcttaacata tatacatgga tgcacactgt agtattatac ataataacaa aaagtgtgga | 31561 |
| aataccacac ttgctcaaca gtagggaatt caataaatac tttatggaca tctatatgaa | 31621 |
| taactgtgat gctgacatta aattatattt ttgaagatgt aatcaagagg ataaacgctt | 31681 |
| gtctcaaaaa gttacatggg aaaagcagta tgtaaactta tataaacatt gtgaacctaa | 31741 |
| tttgattata tatataaaat atagggaata tacatataaa atacatatat gtatatatgg | 31801 |
| gggctatata tatatatata tgaaagagat agatagatag atagatagat agatagatag | 31861 |
| acagacagac agacaataca gactccaatc tgttggtcgt ggttgtctct gacccatgac | 31921 |
| actatggggg acttttattt ttgctcatac ttttcaatat ttcttagtgt tcaataatgt | 31981 |
| gctattattt atacataata ataaaaataa ataaatggca tcaaaaaaga gtaaagggcc | 32041 |
| agtgttccgc ccacatatga gcagccatat tcaagcctgt agacactttg tgtagcctaa | 32101 |
| tgctaggtgt atctgggcaa ggataaactc taaagccaga aattagttca tcaataaaca | 32161 |
| tgtgctactc aatagctagg gctgatggaa aagaatataa aacccagtct gtgccaaatg | 32221 |
| gtgcttacta tctgcaagtg ggagaaggag aaagacgaga aaatgaaaaa tgtgtgtata | 32281 |
| atttatatgt agctgttctg taggagatct ctgacttcac cccattctaa ctttgcaaaa | 32341 |
| agatccaaca ctttgtcaga ttcctgggag gcaagtaatt ttattgatgg tttcatggag | 32401 |
| ggatacagaa cgataacaac tcacacaaag caaacaatgt aatgaaaatc tctattcgac | 32461 |
| tgtttctttt tctcctgaag ttgccctttg gctgccagct accaggcacc aggctcaagg | 32521 |
| tactttcttg ctcttgacac tactcccttc tctcatacaa ttcaacccca accacaaacg | 32581 |
| tgtatagatc tctctctcta taaaacaaag gcctgtagtt aacaggaggt cacttgcagt | 32641 |
| gtagcctctg ttcattgtta cttgtgcaca ctgcttaggg tctcacccca tccacattct | 32701 |
| gctaatcaca ttattcaccc atccaatgta gatctctcca gtggagattc tgctaatatt | 32761 |
| ttctttagat ttgtcacaag tatataatac agttttaaat tgtacagatg attatcctat | 32821 |
| aacagaagag tctaagcctt ttacatcttt gtatctctaa cgaaagcatt cagcatgaag | 32881 |
| ctctgtacac agcagacaat tcaatatgaa tttgctgact tgaaacagca agcctagaaa | 32941 |
| ggagatgtta acttggtcac ttagacagaa caggtttcag caatcagaat tcagatgaca | 33001 |
| tggaactggt agaacaggcg ctttgaagca ataggacatg agccagtgag gagagggatg | 33061 |
| gaatatcata aacaaaggcc aagggctttg caatcagagc tgaagagcca agagcacagg | 33121 |
| ctcagggtgt gggcagactg aatgagaaag tgattcaatc acatgtgaaa gtccagatga | 33181 |
| gaagagagag ttgggattac ttctgctcac caaacatcca aaaccaaaca ggtggatccg | 33241 |
| ggtggtgtgc tgttttactg atgaccatta cacagaattt taacagaagg aatgtaaagc | 33301 |
| agtggttctc aaactggagt tcccagatga gcagcgtctg catcatctgg aaacctgata | 33361 |
| aagcagcaaa ttctcaggcc ctaccccaga cccactgaat cagaaacttg ggggtctggg | 33421 |
| ggaagatggc catctgtatt ttaacaatct cccttcagga gattctgagg ctggctcaag | 33481 |
| tttgaactac aggtagttgg ttcaacaggt gttggtggac tgacaaacaa aaagagactc | 33541 |
| cgaggtaact ccaagatggt aatgtcagaa agcagctacc acccctaggg cttgggggaa | 33601 |
| ccaacaaaag agtttggcat tgccagaacc tagaatcttg aggagaggcc ccagagcatt | 33661 |
| gtgtctcaga ccttgaggac ttggcactgg gacaccatga ggggtttctg ggtgtgggaa | 33721 |
| agggctggaa actccccagt tgctgccacc agggagaact acaagtgaag tggaaggtgt | 33781 |
| gggcctttct cccttttctt ttctcgtctt ctctctcccc ctggtgctca tgtttgacag | 33841 |
| aaaacagctg aaaaggcaga actagtttgg ggagtcttga cctggcatca taaagcagag | 33901 |
| aaaagcaaag ctggagtgaa ggtgagacac aacagctcat tagcagcaac agccgtctag | 33961 |
| cgctccagct tctgaatgaa attctgaaga acagcgcact tggaagacaa attatttgac | 34021 |
| agttctgaca gacgaccaaa ctaacagcat ttgaaaagca agatgactca gagaatacag | 34081 |
| aatttaatcc aaatcccaga tcctatctct gcctttggcc aggcctaatg caaggagaac | 34141 |
| ctgagcacac aaatatatgc aggaatgatc aggacctgtg cctgcattct attctgtctc | 34201 |
| acccaccttc aaatttgttg taaaaacatg ggctcaataa aggtttgtga atcagggaag | 34261 |
| gaagagaagg ggagaaagga agggaaggag ccagctccag atctgtgtct tgcagaggat | 34321 |
| aaaggccagt gtttttagat cacccagtgt ttttctaagc ccccaatact tattttgaaa | 34381 |
| tatcaaaatg ttcaataact aaaaaaaaaa ccgttacaac aataaaacat gtttgagggt | 34441 |
| cagatggact ggcagtttgt gacctctggg gataaacagg tcactttgga atcacagact | 34501 |
| tcctcattcc ccttaaatct catatggtac ccagaagccc ttggaacttt ggaaggtgtt | 34561 |
| tattcacagt tgtaatgtcc atgcagaccc tggctctaag acccaattgt gtaagggtag | 34621 |
| gtttgtagcc cttatcccaa acattctaag tgtgagccaa tgcgtcacac actcagaggc | 34681 |
| cagagactgt attggggtcc tttatttcac gtacgagtca cattccatta agagacccca | 34741 |
| gaagtcagct ctcttccact gactggttct cttcccttgt ttctcttgcc aatgtgtgct | 34801 |
| gcccaggtgg cagtgctcac tgtcagcaga gaagaaaaat gctttcctcc ttggacctct | 34861 |
| tttctctttt tctcctccct actcacattc aggttcccta agcttccccg ctccttgtgc | 34921 |
| tgaagtcatt ctatggtcat ttcttcaact gtctacttcc ctgctggatg ggcacccaag | 34981 |
| acttggcatc ctggggcatg tagaaagggg aaagggaagg gaagggaaaa gaagtcctcc | 35041 |
| caattgtcta tctggacctt tccacactgc ccagagtact gctatgggca tctccttatg | 35101 |
| tctcccgatg tggtgcatgc cagaccctgc aggtagaaaa ggaaagaaag caacccattg | 35161 |
| gaccaggcca gcaaaggctt cagtcacaca gctggctcat acttatggct tcatattctg | 35221 |
| ttgcctcttg aaccagacat ttcttccact ctcataacct ccagtttagc tcgtattcct | 35281 |
| cagcattctc catgtaatat tgttgcatga aacccatgca agtcagccaa tttgctcttt | 35341 |
| ctcatcttgt catttataaa ttgatgctga gaagtctttt tcccaaggtt tttagtaata | 35401 |
| ccttcatcat cccccatagt tcattttggg cagtgattcg cttctttgac tgtacattag | 35461 |
| aatcatctga agaactttct aaaactactg atctcaggtc tcacacaaca ctaattaaat | 35521 |
| tatagtctct ggtggggtag ggcttgggca ctgctatttt accttaagct ctctggagtc | 35581 |
| attctaattt gtagccagtg ctaagggttg caatataagt gaatatattt cacgtatttg | 35641 |
| tcaaacattg actgagtgcc cattatgtgc cagccattat aataggcact ggtgatccca | 35701 |
| cagtgaatca ggcacacaat gctgtcttca cggagcttgt tgtctagtgg gagagtcaaa | 35761 |
| caaaagtgta tatcaataat taagtgatta cagattgcaa taattacaat aagggtgata | 35821 |
| aacaggttgc tataatatac aatagtattg cctttcacca gacatttctt aaagaggtaa | 35881 |
| atcatctatc agacaccttt taaaaatctc atctaatttc aaaagtgtac ataaataatt | 35941 |
| aagtgattac agtttgcaat aattacaatg agggtgataa acaagttgct atgaaagaga | 36001 |
| aagatagcag agatctggct ttgagatggt ggtcagggaa gactgctcca atagctgagt | 36061 |
| tctaaagcta agaaggaact gagaaccttc acagaacgtc ccaagtagaa gagaaagcac | 36121 |
| actgaagact ctagggaaag aggtctgctt gttgtaggaa ccgaaagaag gccaatgtgg | 36181 |
| ctaggtgctg ggtagtgagg ggaaatggca caaggaaaaa atgaggttag agagatcagt | 36241 |
| tgataccagt taatgttgga ccctaaacat taaccatggt aagtctttta gaattgattc | 36301 |
| taattgcaat gaaaaccttt tgaaagattt taaaaagata tctgatagct gatttacctc | 36361 |
| tctaagaaat gtctggtgaa agacaatacc atcacattgg agatggaaaa agatgaatgg | 36421 |
| attctaaaaa cattctgaaa gtacattcaa aatgtttttc aggtagctta tgcaactaat | 36481 |
| aaatagtggt ggcattctgg gtaagacaag ggaggagcag gcttgcagtt tagggcaaga | 36541 |
| aaggggtggg gagaagagct cagcactaaa atcatgtgtt ccatttgggg cacatcgagt | 36601 |
| ctgagttgct atgagaccac caagtggaga tgccaagtaa atagtcagtt acatgaatct | 36661 |
| ggagttcagt gaagaggtct agaagaaaga tgtatatttg ggcattattc ggatatagat | 36721 |
| attatgtaaa gcaataaaat tggatgagat cacctaggga gagaatgcac atagataaaa | 36781 |
| actgacctag gaccacttca tgtctaaaac accaaaagca atgtcaacaa aagccaaaat | 36841 |
| tgacaaatgg gatctaatta aactaaagag cttctgcaca gcaaaagaat cagagtgaac | 36901 |
| aggcaaccca caaaatggaa gaaaattttc acaacctact catctgacaa agggctaata | 36961 |
| tccagaatct acagtgaact caaacaaatt tacaagaaaa aaacaaacaa ccccatcaaa | 37021 |
| aagtgggtga aggacatgaa cagacacttc tcaaaagaag acatttatgc agccaaaaaa | 37081 |
| cacatgaaaa aatgctcacc atcactggcc atcagagaaa tgcaaatcaa aaccacaatg | 37141 |
| agataccatc tcacaccagt tcaaatggca atcattaaaa agtcaggaaa caacaggtgc | 37201 |
| tggagaggat ctggagaaat aggaacactt ttacactgtt ggtgggacta taaactagtt | 37261 |
| caaccattgg ggaagtcatt gtggcgattc ctcagggatc tagaactaga aataccattt | 37321 |
| gacccagcca tcccattact gggtatatac ccaaaggact ataaatcatg ctgctataaa | 37381 |
| gacacatgca catgtatgtt tattgcggca ctattcacaa tagcaaagac ttggaaccaa | 37441 |
| cctaaatgtc caacaatgat agactggatt aagaaaatgt ggcacatata caccatggaa | 37501 |
| tactatgcag ccataaaaaa tgatgagttc atgtcctttg tagggacatg gatgaagctg | 37561 |
| gaaaccatca tcctcagcaa actatcgcaa ggacaaaaaa ccaaacaccg catgttctca | 37621 |
| cccataggtg ggaattgaac gatgagaaca catggacacg ggaaggggaa catcacacac | 37681 |
| tggggactgt tgtggggtgg gaagaggggg gagggatagc attaggagat atacctaatg | 37741 |
| ctaaatgacg agttattggg tgcagcacac cagcatggca catgtataca tatgtaccta | 37801 |
| acctgcacat tgagcacacg taccctaaaa cttaaagtat aataataata aaataaaata | 37861 |
| aaataaaaaa acaaaaattg atgtaggacc aattcctgaa gaacactgac agttaatttt | 37921 |
| ttggtttagg aggaggagaa gccagcaaac gacactgagt agcaatatcc aaagaaaaag | 37981 |
| aggaaaaagg aaaactggga gattatgagt gtcccagagg gaatgtttca agattaccat | 38041 |
| cagcagtgag ctttgtgtaa aggtggcctc ctgtaataga ggtgcgggca ggagaaggca | 38101 |
| gaatagggaa aagggggtga aaaagcttcc ctcaagattt ataatacagt ggaagagaga | 38161 |
| gacagagaga gagaaagaga aagagagaga gagaacttaa ggaggtagag gaagagagag | 38221 |
| aaccaaaaaa gagggagctg agtatagaag caattagatt catagttttt agttgcggca | 38281 |
| gtgatatttg agtgggggcc ttttatatat tccattctag gtgtttccca gttgatggga | 38341 |
| gagggtctta cctagatctg catgtaaaag ggagtaggcc agctggcaga cttgacatgg | 38401 |
| atcagtggta gaacatccta gcagttctgt gaatactctc tgagaatgac atgaaaggta | 38461 |
| ttggttcagg ccttttggag gtgataaaaa ccaccaaaat gtggacttat tgcaaattgt | 38521 |
| atttgttacc atttgcaatg attataatta ttctcttata tataggcttt cttacatata | 38581 |
| gcttctctta cacatagcgt catgagttat gccttctctt acatatagtg tctgctatga | 38641 |
| gttatgccaa gcagggcaaa caaaattgct gcctttcttt aaaaagagga cgctcctagt | 38701 |
| atgggcctaa ttaattatga taattacagc tatggcatgg aacataagca cattcatata | 38761 |
| cacaaagaca ataaaataaa gaacagttca aaaacagaac agttacatta tatatcagtt | 38821 |
| tcagtatgaa taataccctg gactctgaaa tatgtctggg gcacattatt ctgtaattgg | 38881 |
| tggtgaaaaa aaatctgcat cttatctcta cgccaatcct tatgaaggag ctgtttttca | 38941 |
| ggagttcgag aaagagacac agggatgtcc agtcatcaaa gccgcagagc ctgaggaaga | 39001 |
| ataaaggatt tgtggcagga aaacccagta atgaatgtat tctgctagtt tctcagcata | 39061 |
| gaacttagaa aagaggccac agaaggaaaa gagaagtaat gttagacagc tgatgttggc | 39121 |
| aatgggcaaa gaatttcatt ctcatatgca gggcagtggc taaaggggca gtgttgtgag | 39181 |
| gaatcacatt ccaggtcatt catgtccagg gtgtggtagg aggactgtgt ttcatttatt | 39241 |
| ttgtacatgg cccagctatg accttgtgca aggaaatgct taatcatttt ctatcacagt | 39301 |
| tgcaaagaga ttcttatcct actcagaatg tacgtcttct cttctgtttc atttacagtc | 39361 |
| acaacccaag tccttgcttt gacctccaaa gcaccacttg atgtatccac ttcagaaaca | 39421 |
| cacacagaca gctcacctct ttctatccct taccttcctc ccctcttcac tcaaaccacc | 39481 |
| tacttccttt gcattcactc ttccttcagc ctgaaataat cttcctccaa atatctacct | 39541 |
| tctcactccc tcacttctct caagatacac ttaaatgtta tattccctat gaggcctccc | 39601 |
| ctggccatcc ctccccagcc ttcctatccc ccctctctgc tttattttat tctccttaat | 39661 |
| atatatcaca ctctgataaa ccattgaatg tacttatcaa gttattgcct ctctctccct | 39721 |
| ttccattgtc tccatcactg agagctctgt gaagaaaagg atttgtacct gtttcattta | 39781 |
| gtgctgtacc cccagttccc acaacagcgc aacaggcact caataaatag ttgttgaata | 39841 |
| agtgaataaa acagaagtag ctgcatattt tctggtaaca aatgatattc ttctgaaaat | 39901 |
| gtcatatttt cagacatatt tccgaaaata aattcaaatt agataaacat tgtattttta | 39961 |
| gaccatttct tctttgcatt aatcatcctt ctcaataata taacatttgt aaaacttagg | 40021 |
| ttagatatgg gctcttcaac tttccattac agaagataaa gtgaaaaggc tagacccaat | 40081 |
| ggtgttattc cttcatctac atctatcctt tggaaacaca tgaccaaatt gcttgccatc | 40141 |
| acaatctcaa aatctaccct ttggtattaa ctcacttcac ttgtccctct gtcccttttt | 40201 |
| agatggtagc catcggtctc tggagcagtg tagagtcaga acaacttcta tttggggaag | 40261 |
| aaatcattgc tggtgacctt actttcaatt actaactttc tagtgacatt tacataattt | 40321 |
| tagagaaaat taacacctac acttgtaaag ttgtggcttt cccacaccta tttatcatct | 40381 |
| ctcaatattc cttgaaaagg aaattatcaa tttatcattc tatattggca atgaaatgcc | 40441 |
| cctaatatct gtcacctata agacaattga agatgatgtg ttgaaagctt tctgaaaatg | 40501 |
| ctgatcatta ctttaaatgg aattgaaatt ccagtttatt atttccaaaa atatgatctt | 40561 |
| actgatcata ggataacatt tcataacatt tcagagattt cttccccttc gaggagccaa | 40621 |
| acccatagga cctctggact cccacagatc ctggcaggga gttcccactc ataaaagcac | 40681 |
| aggtgccctc agagtcattc agggatgaag aagcaaccct cattggccat gtcctacgtt | 40741 |
| ccccatatag taaggactgg aggagaccag tgctcaattc tgcagtctca gacagctgtc | 40801 |
| agaggagagt catgaatgtg cagtgtctag cacattacaa acgtttgtta attgactgat | 40861 |
| cattcatggt gtgacagccc tataactcag ctatcctatt cagtcagaaa ttaactcagt | 40921 |
| aatcaaagtc attaaaagga agaaaaaaaa aacctacagt accagacaga tggtggggaa | 40981 |
| atcagacaga tgaaaggaaa aatggctgta ggtcattgag taagacactg ggcagcaaaa | 41041 |
| cctgggcctt gtgcctggtt atactccaca ttatagctcc agcaaggttt ggcaggattt | 41101 |
| ccacagtcct ggcttattct aacctttctt gggagcagga gcagtgttgg tcagatagac | 41161 |
| agacacataa ggaatctgtc caactggcac cgtgtgaatt tgggctcttg gtgtacatgg | 41221 |
| ataactggga aaaagaggag agagacatgt aggactgatc ctaccgtttg tgaagtcttg | 41281 |
| ggcaagagta tgaatgaaaa cccacttctc ttcccctgcc tggctccact gcacacagta | 41341 |
| aagagcctca agcataggtg tgtggacatt gcaccatgta tccaagctct gaccatgcct | 41401 |
| cttgaaacag ctattcctca gccaccctct gaccatggga ggaatgaccc aggagaaatg | 41461 |
| accacatagg tcttgaaaat gggctcaggg ctatttacga agtcaattcc ggggtcccag | 41521 |
| gagtatggac taaaatgtga gtcaggcatg ccagatgggt atgttctatt gacttcaagg | 41581 |
| attcctcatg ctgtgggaag gaacctctcc agaagagaga cagagcagaa ccctctaaat | 41641 |
| gtggggcaca aagcaggagc ccctcttgct ggattcaaag ggtcatactg gaagagtgta | 41701 |
| ggttgagtct tattctcaca tcactcatat cacttacaca cttcttttat agccttagca | 41761 |
| gccatgccac aaagagaaac tcttcatgca tatcttttgg tccataagtc ataaatagtt | 41821 |
| atccttgatc ccatgtcttt tttagagcca tggacagaga gaagcaaaaa tataccaagt | 41881 |
| tcacactgag ttgtctccct tcatatctct tagcagtcac tgaaaggtta tgagactcag | 41941 |
| gctgggtttc tatcctctgt ccctgaaacg acaacgttga cctcgtgatc aaccctagaa | 42001 |
| tccagagcaa gatctcagac tgtcctctct actaccagac agcacacttt gttttggggg | 42061 |
| ctgtgtgctt gaaatattag ctatggcaaa aggctttgag tcttatgaca ccccaagtaa | 42121 |
| cttttacttt aggaatttga aatacagcct tgctgtaatg ctgtctcctt aacaaagcag | 42181 |
| tacctttgaa atatttaaca acttgaaaag gaaaccgagc ttgaattttc ctttcaggtg | 42241 |
| ctcaggaaat aatgtttcac ttctgtctga aattcaccat ctcctcagac aaagaaggct | 42301 |
| cttatggtaa aaggaatggc attttctcca caattttcga ataaaagata aagagaaaac | 42361 |
| agcactgcag cctttttgtt aggatctaac aataaagaaa taatacggtt ttgccagggg | 42421 |
| agagctctgg ttttaagctc agaatacaaa aataggctga caaaatttta caaaggaata | 42481 |
| ttctcagcta ccactctgag gatggtagaa agtgaaattt caagaaaatt atattatttg | 42541 |
| attatttgat gatgattaag ctgattggcc agtcctatgt gaaattctaa agtagaagaa | 42601 |
| atgtgatgtt gtcttttctg ctcacctctc ccctcattcc tacccccaaa tctctgcctc | 42661 |
| taccccaaac ccagcctgac ctttgggaaa ggaatggggg ctgtcacttg caccgtagct | 42721 |
| cctcctgcct gcagtactct ccccccacag tccagcctcc ctctgctcag ctaacttttc | 42781 |
| ctatagctcc ttctggctac ttctaaggaa ccttccataa tgctgcccac cctctcagta | 42841 |
| acagcccctc tactatccct tgattacatg cactgtaatt ggttagtaat tgattttatg | 42901 |
| tcctctgcca aattatactc catgagagca aaaatcatga gtattatctt taatgtttaa | 42961 |
| atctccacaa ctatccccca tataagtcta gagaataaat gaatgagtga attaatgaat | 43021 |
| agaaactcaa gccattatgt tgccactcct aggatatttg gattaacttt aactgaagag | 43081 |
| taaaaagcat ttacctgtcc taaaggagac ataaaattag tgggagagta tttggagaaa | 43141 |
| aaaaagacac tgtaatacat tcttttgtgt tgcctaccct gatgtagtca ggtgtccctg | 43201 |
| atatggggtg ggctgaggat ttgaaataaa atacctaatt tgacattgta agtggaggaa | 43261 |
| tcaggatttg aaaccaaatt ggtttgacct aaatcaagct attatgataa ggacttgcaa | 43321 |
| gaaaaaagga atccataaaa accatatgaa tagctaccaa ttagtaagca tttatatgtg | 43381 |
| tcaattacaa agccaaatgc aaatcatgct ttatttatat tagccacctt ttatagatga | 43441 |
| agaaattgag acctgaatat taaaattgcc tacttttaca tagtaagtaa aggaatcagg | 43501 |
| gtttgaaccc aaattggttt cacctaaggt agaaaaccat cccagcaagt ctcctattaa | 43561 |
| ctggaaccct attgtggtgg cctgagatat aacagtagct gtggaagcgc tgtagagtcc | 43621 |
| tggccatcct atgtgctcct gatctggtcc ctcctgccac ctgcttctgc tccctgtgcc | 43681 |
| atccacccat ctggaagtct cccagtgtcc atcttcgggg gagacactca ccagagtttc | 43741 |
| cagcttccag ccagtatgga gtgcccctgt cccacagcaa tctcaccgaa atcacagcta | 43801 |
| catctgttaa aattaggcta ccaatgagtg atagatgagg gggaaaaata ataatagtgt | 43861 |
| actaaacaaa acaaatgttt atttttctca cacataaaaa tctagaggtt gaagtccagg | 43921 |
| gctggtccag aggctccaag gatctgggat ttagactccc tctttcttgt ttttccacag | 43981 |
| catatggctt ccatttctgg ggccacattg gtccaaaatg tatgctgggg ctccagccat | 44041 |
| tgcatccata tttcagccac aggaaggagg aagtggggaa gaaaggacag gcccctaata | 44101 |
| cctgtatagt tcaagaagac tatcccgccc atacttccca accaccctta gttgaacaat | 44161 |
| gctgtcttaa ttcaagacac tcacatgtct agccaaaaat ctgaattctg ttacaaacaa | 44221 |
| ggagaataga gatgtgcgcc acctcaatac ctcatccata gctacctttt cctttgtgca | 44281 |
| gctgtggcca agtgaaagct gaaggagctg tggtaaccct tctgaaggga ggctggggcc | 44341 |
| tttcacaaga ggctgcatga ttgacattta tcctgcatgg cctgtgaagt acagagaaat | 44401 |
| attttctctt gaagccacat catagcagtg gctgctttgt agcctgattc caccattatg | 44461 |
| cctttaaagt gcctagcaat tcagccttca catcatgcaa agaggaatat ctcccagtct | 44521 |
| ttgtaagatc agcttaattc taaccacctc cttacctccc actgcactcc tacacgcaca | 44581 |
| cacaaatctt cttcactcag agcagaacca taacccaagc cctaccacct agagactgaa | 44641 |
| gaatcaggct catgattaca aatatgcaat aattttttgt gtggataatg tcaatgggga | 44701 |
| tgatggtaag agaattcctt ggtttacaca ttgaccctct tccctgtccc ttacaatcag | 44761 |
| gaaatatttg tcccaacacc ttgtttcttc tgttgcaggc tcctgtggct gtcttgtcat | 44821 |
| gatattgttt gcctctgaag tgaaaatcca tcacctctca gaaaaaattg caaattataa | 44881 |
| agaagggact tatgtctaca aaacgcaaag tgaaaaatat accacctcat tctgggtcat | 44941 |
| tttcttttgc ttttttgttc attttctgaa tgggctccta atacgacttg ctggatttca | 45001 |
| gttccctttt gcaaaatcta aagacgcaga aacaactaat gtagctgcag atctaatgta | 45061 |
| ctgaaaggca aacctttcta taattttaca agggagtaga cttgctttgg tcacttttag | 45121 |
| atgtggttaa ttttgcatat ccttttagtc tgcatatatt aaagcatcag gacccttcgt | 45181 |
| gacaatgttt acaaattacg tactaaggat acaggctgga aagtaaggga agcagaagga | 45241 |
| aggctttgaa aagttgtttt atctggtggg aaattgcttg acccaggtag tcaaaggcag | 45301 |
| ttgactagaa tcgacaaatt gttactccat atatatatat gtgtgtgtgt gtgtgtgtgt | 45361 |
| gtgtgtgtaa gatgtcttcc tatcaaaaag atatcaaagg cacatggaat atattttaat | 45421 |
| aaaaacaaat aatatctcta atatatccac acatttgttg ccagatttca gaaaactgag | 45481 |
| ctgcaatcgc tttcctaaaa cagtagtgta ttaaatgaac atctataaaa tgtatcaaca | 45541 |
| cacattttaa aaaatttgtt taaagtatac tcttaggcca ggcgtggtga ctcacacctg | 45601 |
| taattccagc acttcaggag gccaaggtgg gaagatcatt tgagttcagg agttcgagtt | 45661 |
| acagcctggg caataaagtg agaccctgtc actaacaaaa ttaaaaaata aaataaatat | 45721 |
| aaaatatagg ctttaaaaaa gcatagtctt attaaccatg tctgttggtc aaaatctgca | 45781 |
| aactctaaaa gaagaaaaga agaaaaaacc aagcttaggg tatttttcct cccgtgcctg | 45841 |
| agtcccaatt acattcacga cagtactttc aatgaacata attgttagga ccactgagga | 45901 |
| atcatgaaaa atgatctctg cttagtacat ttgatgcaaa atgacttatt aggggctgtt | 45961 |
| tttctagcta tagtgtctcg agtactaata tgcaattatg aaaattatat taaatctggg | 46021 |
| attatgacgg tatcactgta tcatcttggt cttgttctgg ctgtcaccaa gcatgaccca | 46081 |
| ggtcaacttt ttttttcccc tgaattaccc atcaaattga tctgcagctg actaaaggcc | 46141 |
| acagctgagc ctggaactga cccttccttc atcctcaacc tgctgtcctc cagaaagcac | 46201 |
| caaggaaaaa gcagagaatg acagcaaaca gatcactagg cctctgacca caggtgctga | 46261 |
| gtactcagca gccctcatat aataggtttg aaagtactcc ttaaaataaa acactgtttc | 46321 |
| cctttggaac tatttacaag gatgaaacaa ccgtatacct gagaaataac ttgctctggt | 46381 |
| gtcaattcgc tattcgccag cagacatcag aacacaccga gtttccagat gctggttttt | 46441 |
| ccccttaaat caggaaatac acctggacaa tttctagaag actacaattc agtctagcca | 46501 |
| caaaggggat tttttttttt tggtaacagg ctagagcccg gttctgtaag tctttagctg | 46561 |
| aaatggtcca gtacaaaagc actggaaatg agtgggctag gaggacaagg accgtctcct | 46621 |
| gcgtgaggag ttggttggag gtccccaagg ccaggtaccc cctgcactct tattggattc | 46681 |
| ctctctgtct tcttggagtt ttgaaaaact ccttcgaaca ccaggctttt ttctttagaa | 46741 |
| aacaagtctc caatcgttct ctgttccgta gaaagagaaa gaaaacctgg agcagctgct | 46801 |

gaaaaatcta atgaggaact aagaggcaaa cccacca
- Mouse CLRN1 Protein Isoform 1
SEQ ID NO: 10
MPSQQKKIIFCMAGVLSFLCALGVVTAVGTPLWVKATILCKTGALLVNASGKELDKFMGEMQYGLFHGEGV
RQCGLGARPFRFSSRSMKERYSLYEDKGETAVFPDLVQAIPVSIHINIILFSMILVVLTMVGTAFFMYNAF
GKPFETLHGPLGLYLVSFISGSCGCLVMILFASEVKVHRLSEKIANFKEGTYAYRTQNENYTTSFWVVFIC
FFVHF LNGLLIRLAGFQFPFTKSKETETTNVASDLMY
- Dog CLRN1 Protein
SEQ ID NO: 11
MPNQQKKVVFCTAGVLSFVCALGVVTALGTPLWIKATFLCKTGALLVNASGQELDKFMGEMQYGLFHGEGI
RQCGLGARPFRFSLFPDLLKVIPVSIHVNVILFSTILVVLTMVGTAFFMYNAFGKPFETLHGPLGLYLLSF
ISGSCGCLVMILFASEVKIHHLSEKIANYKEGTYAYKTQSEKYTTSFWVVFICFLVHLLNGLLIRLAGFQF
PFAKS KDTETTNVAADLMY
- 5′ UTR
SEQ ID NO: 12
AGGAGATACTTGAAGGCAGTTTGAAAGACTTGTTTTACAGATTCTTAGTCCAA
AGATTTCCAATTAGGGAGAAGAAGCAGCAGAAAAGGAGAAAAGCCAAGTAT
GAGTGATGATGAGGCCTTCATCTACTGACATTTAACCTGGCGAGAACCGTCG
ATGGTGAAGTTGCCTTTTCAGCTGGGAGCTGTCCGTTCAGCTTCCGTAATAAA
TGCAGTCAAAGAGGCAGTCCCTTCCCATTGCTCACAAAGGTCTTGTTTTTGAA
CCTCGCCCTCACAGAAGCCGTTTCTCATC
- 5′ ITR
SEQ ID NO: 13
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCT
- 3′ ITR
SEQ ID NO: 14
AGGAACCCCTAGTGATGGAGTTGGCCACTCCCTCTCTGCGCGCTCGCTCGCTC
ACTGAGGCCGGGCGACCAAAGGTCGCCCGACGCCCGGGCTTTGCCCGGGCGG
CCTCAGTGAGCGAGCGAGCGCGCAGCTGCCTGCAGG
- 3′ UTR
SEQ ID NO: 15
AAGGCAAACCTTTCTATAATTTTACAAGGGAGTAGACTTGCTTTGGTCACTTT
TAGATGTGGTTAATTTTGCATATCCTTTTAGTCTGCATATATTAAAGCATCAG
GACCCTTCGTGACAATGTTTACAAATTACGTACTAAGGATACAGGCTGGAAA
GTAAGGGAAGCAGAAGGAAGGCTTTGAAAAGTTGTTTTATCTGGTGGGAAAT
TGCTTGACCCAGGTAGTCAAAGGCAGTTGACTAGAATCGACAAATTGTTACT
CCATATATATATATGTGTGTGTGTGTGTGTGTGTGTGTGTGTAAGATGTCTTC
CTATCAAAAAGATATCAAAGGCACATGGAATATATTTTAATAAAAACAAATA
ATATCTCTAATATATCCACACATTTGTTGCCAGATTTCAGAAAACTGAGCTGC
AATCGCTTTCCTAAAACAGTAGTGTATTAAATGAACATCTATAAAATGTATCA
ACACACATTTTAAAAAATTTGTTTAAAGTATACTCTTAGGCCAGGCGTGGTGA
CTCACACCTGTAATTCCAGCACTTCAGGAGGCCAAGGTGGGAAGATCATTTG
AGTTCAGGAGTTCGAGTTACAGCCTGGGCAATAAAGTGAGACCCTGTCACTA
ACAAAATTAAAAAATAAAATAAATATAAAATATAGGCTTTAAAAAAGCATA
GTCTTATTAACCATGTCTGTTGGTCAAAATCTGCAAACTCTAAAAGAAGAAA
AGAAGAAAAAACCAAGCTTAGGGTATTTTTCCTCCCGTGCCTGAGTCCCAATT
ACATTCACGACAGTACTTTCAATGAACATAATTGTTAGGACCACTGAGGAAT
CATGAAAAATGATCTCTGCTTAGTACATTTGATGCAAAATGACTTATTAGGGG
CTGTTTTTCTAGCTATAGTGTCTCGAGTACTAATATGCAATTATGAAAATTAT
ATTAAATCTGGGATTATGACGGTATCACTGTATCATCTTGGTCTTGTTCTGGC
TGTCACCAAGCATGACCCAGGTCAACTTTTTTTTTCCCCTGAATTACCCATCA
AATTGATCTGCAGCTGACTAAAGGCCACAGCTGAGCCTGGAACTGACCCTTC
CTTCATCCTCAACCTGCTGTCCTCCAGAAAGCACCAAGGAAAAAGCAGAGAA
TGACAGCAAACAGATCACTAGGCCTCTGACCACAGGTGCTGAGTACTCAGCA
GCCCTCATATAATAGGTTTGAAAGTACTCCTTAAAATAAAACACTGTTTCCCT
TTGGAACTATTTACAAGGATGAAACAACCGTATACCTGAGAAATAACTTGCT
CTGGTGTCAATTCGCTATTCGCCAGCAGACATCAGAACACACCGAGTTTCCA
GATGCTGGTTTTTCCCCTTAAATCAGGAAATACACCTGGACAATTTCTAGAAG
ACTACAATTCAGTCTAGCCACAAAGGGGATTTTTTTTTTTTGGTAACAGGCTA
GAGCCCGGTTCTGTAAGTCTTTAGCTGAAATGGTCCAGTACAAAAGCACTGG
AAATGAGTGGGCTAGGAGGACAAGGACCGTCTCCTGCGTGAGGAGTTGGTTG
GAGGTCCCCAAGGCCAGGTACCCCCTGCACTCTTATTGGATTCCTCTCTGTCT
TCTTGGAGTTTTGAAAAACTCCTTCGAACACCAGGCTTTTTTCTTTAGAAAAC
AAGTCTCCAATCGTTCTCTGTTCCGTAGAAAGAGAAAGAAAACCTGGAGCAG
CTGCTGAAAAATCTAATGAGGAACTAAGAGGCAAACCCACCA
- Chimeric intron
SEQ ID NO: 16
GGAGTCGCTGCGTTGCCTTCGCCCCGTGCCCCGCTCCGCGCCGCCTCGCGCCG
CCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTCGTTTC
TTTTCTGTGGCTGCGTGAAAGCCTTAAAGGGCTCCGGGAGGGCCCTTTGTGCG
GGGGGGAGCGGCTCGGGGGGTGCGTGCGTGTGTGTGTGCGTGGGGAGCGCC
GCGTGCGGCCCGCGCTGCCCGGCGGCTGTGAGCGCTGCGGGCGCGGCGCGGG
GCTTTGTGCGCTCCGCGTGTGCGCGAGGGGAGCGCGGCCGGGGGCGGTGCCC
CGCGGTGCGGGGGGGCTGCGAGGGGAACAAAGGCTGCGTGCGGGGTGTGTG
CGTGGGGGGGTGAGCAGGGGGTGTGGGCGCGGCGGTCGGGCTGTAACCCCC
CCCTGCACCCCCCTCCCCGAGTTGCTGAGCACGGCCCGGCTTCGGGTGCGGG
GCTCCGTGCGGGGCGTGGCGCGGGGCTCGCCGTGCCGGGCGGGGGGTGGCG
GCAGGTGGGGGTGCCGGGCGGGGCGGGGCCGCCTCGGGCCGGGGAGGGCTC
GGGGGAGGGGCGCGGCGGCCCCCGGAGCGCCGGCGGCTGTCGAGGCGCGGC
GAGCCGCAGCCATTGCCTTTTATGGTAATCGTGCGAGAGGGCGCAGGGACTT
CCTTTGTCCCAAATCTGTGCGGAGCCGAAATCTGGGAGGCGCCGCCGCACCC
CCTCTAGCGGGCGCGGGGCGAAGCGGTGCGGCGCCGGCAGGAAGGAAATGG
GCGGGGAGGGCCTTCGTGCGTCGCCGCGCCGCCGTCCCCTTCTCCCTCTCCAG
CCTCGGGGCTGTCCGCGGGGGGACGGCTGCCTTCGGGGGGGACGGGGCAGG
GCGGGGTTCGGCTTCTGGCGTGTGACCGGCGGCTCTAGAGCCTCTGCTAACC
ATGTTCATGCCTTCTTCTTTTTCCTACAG
- CMV Enhancer
SEQ ID NO: 17
GACATTGATTATTGACTAGTTATTAATAGTAATCAATTACGGGGTCATTAGTT
CATAGCCCATATATGGAGTTCCGCGTTACATAACTTACGGTAAATGGCCCGCC
TGGCTGACCGCCCAACGACCCCCGCCCATTGACGTCAATAATGACGTATGTT
CCCATAGTAACGCCAATAGGGACTTTCCATTGACGTCAATGGGTGGACTATTT
ACGGTAAACTGCCCACTTGGCAGTACATCAAGTGTATCATATGCCAAGTACG
CCCCCTATTGACGTCAATGACGGTAAATGGCCCGCCTGGCATTATGCCCAGT
ACATGACCTTATGGGACTTTCCTACTTGGCAGTACATCTACGTATTAGTCATC
GCTATTACCATG
- CBA promoter
SEQ ID NO: 18
TCGAGGTGAGCCCCACGTTCTGCTTCACTCTCCCCATCTCCCCCCCCTCCCCA
CCCCCAATTTTGTATTTATTTATTTTTTAATTATTTTGTGCAGCGATGGGGGCG
GGGGGGGGGGGGGCGCGCGCCAGGCGGGGCGGGGCGGGGCGAGGGGCGGG
GCGGGGCGAGGCGGAGAGGTGCGGCGGCAGCCAATCAGAGCGGCGCGCTCC
GAAAGTTTCCTTTTATGGCGAGGCGGCGGCGGCGGCGGCCCTATAAAAAGCG
AAGCGCGCGGCGGGCG
- tGFP
SEQ ID NO: 19
MESDESGLPAMEIECRITGTLNGVEFELVGGGEGTPEQGRMTNKMKSTKGALTF
SPYLLSHVMGYGFYHFGTYPSGYENPFLHAINNGGYTNTRIEKYEDGGVLHVSFS
YRYEAGRVIGDFKVMGTGFPEDSVIFTDKIIRSNATVEHLHPMGDNDLDGSFTRT
FSLRDGGYYSSVVDSHMHFKSAIHPSILQNGGPMFAFRRVEEDHSNTELGIVEYQ
HAFKTPDADAGEE
- bovine growth hormone poly-A
SEQ ID NO: 20
CTGTGCCTTCTAGTTGCCAGCCATCTGTTGTTTGCCCCTCCCCCGTGCCTTCCT
TGACCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATG
CGGTGGGCTCTATGG
- polyadenylation signal of soluble neuropilin-1
SEQ ID NO: 21
AAATAAAATACGAAATG
- Splice donor sequence
SEQ ID NO: 22
GTAAGTATCAAGGTTACAAGACAGGTTTAAGGAGACCAATAGAAACTGGGCT
TGTCGAGACAGAGAAGACTCTTGCGTTTCT
- Splice acceptor sequence
SEQ ID NO: 23
GATAGGCACCTATTGGTCTTACTG ACATCCACTTTGCCTTTCTCTCCACAG
- 3′ITR
SEQ ID NO: 24
CCTGCAGGCAGCTGCGCGCTCGCTCGCTCACTGAGGCCGCCCGGGCAAAGCC
CGGGCGTCGGGCGACCTTTGGTCGCCCGGCCTCAGTGAGCGAGCGAGCGCGC
AGAGAGGGAGTGGCCAACTCCATCACTAGGGGTTCCT
- ITR
SEQ ID NO: 25
TTGGCCACTCCCTCTCTGCGCGCTCGCTCGCTCACTGAGGCCGCCCGGGCAAA
GCCCGGGCGTCGGGCGACCTTTGGTCGCCCGGCCTCAGTGAGCGAGCGAGCG
CGCAGAGAGGGAGTGGCCAACTCCATCACTAGGGGTTCCT
- sh-chimeric intron
SEQ ID NO: 26
GGAGTCGCTGCGTTGCCTTCGCCCCGTGCCCCGCTCCGCGCCGCCTCGCGCCG
CCCGCCCCGGCTCTGACTGACCGCGTTACTCCCACAGGTGAGCGGGCGGGAC
GGCCCTTCTCCTCCGGGCTGTAATTAGCGCTTGGTTTAATGACGGCTTGTTTCT
TTTCTGTGGCTGCGTGAAAGCCTTGAGGGGCTCCGGGAGCTAGAGCCTCTGCT
AACCATGTTCATGCCTTCTTCTTTTTCCTACAG
- 3′UTR 600A
SEQ ID NO: 27
AAGGCAAACCTTTCTATAATTTTACAAGGGAGTAGACTTGCTTTGGTCACTTT
TAGATGTGGTTAATTTTGCATATCCTTTTAGTCTGCATATATTAAAGCATCAG
GACCCTTCGTGACAATGTTTACAAATTACGTACTAAGGATACAGGCTGGAAA
GTAAGGGAAGCAGAAGGAAGGCTTTGAAAAGTTGTTTTATCTGGTGGGAAAT
TGCTTGACCCAGGTAGTCAAAGGCAGTTGACTAGAATCGACAAATTGTTACT
CCATATATATATATGTGTGTGTGTGTGTGTGTGTGTGTGTGTAAGATGTCTTC
CTATCAAAAAGATATCAAAGGCACATGGAATATATTTTAATAAAAACAAATA
ATATCTCTAATATATCCACACATTTGTTGCCAGATTTCAGAAAACTGAGCTGC
AATCGCTTTCCTAAAACAGTAGTGTATTAAATGAACATCTATAAAATGTATCA
ACACACATTTTAAAAAATTTGTTTAAAGTATACTCTTAGGCCAGGCGTGGTGA
CTCACACCTGTAATTCCAGCACTTCAGGAGGCCAAGGTGGGAAGATCATTTG
AGTTCAGGAGTTCGAGTTACAG
- 3′UTR 600B
SEQ ID NO: 28
GAAGATCATTTGAGTTCAGGAGTTCGAGTTACAGCCTGGGCAATAAAGTGAG
ACCCTGTCACTAACAAAATTAAAAAATAAAATAAATATAAAATATAGGCTTT
AAAAAAGCATAGTCTTATTAACCATGTCTGTTGGTCAAAATCTGCAAACTCTA
AAAGAAGAAAAGAAGAAAAAACCAACGTTAGGGTATTTTTCCTCCCGTGCCT
GAGTCCCAATTACATTCACGACAGTACTTTCAATGAACATAATTGTTAGGACC
ACTGAGGAATCATGAAAAATGATCTCTGCTTAGTACATTTGATGCAAAATGA
CTTATTAGGGGCTGTTTTTCTAGCTATAGTGTCTCGAGTACTAATATGCAATT
ATGAAAATTATATTAAATCTGGGATTATGACGGTATCACTGTATCATCTTGGT
CTTGTTCTGGCTGTCACCAAGCATGACCCAGGTCAACTTTTTTTTTCCCCTGAA
TTACCCATCAAATTGATCTGCAGCTGACTAAAGGCCACAGCTGAGCCTGGAA
CTGACCCTTCCTTCATCCTCAACCTGCTGTCCTCCAGAAAGCACCAAGGAAAA
AGCAGAGAATGACAGCAAACA
- 3′UTR 600C
SEQ ID NO: 29
AGGCCTCTGACCACAGGTGCTGAGTACTCAGCAGCCCTCATATAATAGGTTT
GAAAGTACTCCTTAAAATAAAACACTGTTTCCCTTTGGAACTATTTACAAGGA
TGAAACAACCGTATACCTGAGAAATAACTTGCTCTGGTGTCAATTCGCTATTC
GCCAGCAGACATCAGAACACACCGAGTTTCCAGATGCTGGTTTTTCCCCTTAA
ATCAGGAAATACACCTGGACAATTTCTAGAAGACTACAATTCAGTCTAGCCA
CAAAGGGGATTTTTTTTTTTTGGTAACAGGCTAGAGCCCGGTTCTGTAAGTCT
TTAGCTGAAATGGTCCAGTACAAAAGCACTGGAAATGAGTGGGCTAGGAGG
ACAAGGACCGTCTCCTGCGTGAGGAGTTGGTTGGAGGTCCCCAAGGCCAGGT
ACCCCCTGCACTCTTATTGGATTCCTCTCTGTCTTCTTGGAGTTTTGAAAAACT
CCTTCGAACACCAGGCTTTTTTCTTTAGAAAACAAGTCTCCAATCGTTCTCTG
TTCCGTAGAAAGAGAAAGAAAACCTGGAGCAGCTGCTGAAAAATCTAATGA
GGAACTAAGAGGCAAACCCACCA
- FP
SEQ ID NO: 30
KRKRRG
- T2A
SEQ ID NO: 31
EGRGSLLTCGDVEENPGP
- eGFP
SEQ ID NO: 32
MVSKGEELFTGVVPILVELDGDVNGHKFSVSGEGEGDATYGKLTLKFICTTGKLP
VPWPTLVTTLTYGVQCFSRYPDHMKQHDFFKSAMPEGYVQERTIFFKDDGNYK
TRAEVKFEGDTLVNRIELKGIDFKEDGNILGHKLEYNYNSHNVYIMADKQKNGI
KVNFKIRHNIEDGSVQLADHYQQNTPIGDGPVLLPDNHYLSTQSALSKDPNEKRD
HMVLLEFVTAAGITLGMDELYK
- Myc
SEQ ID NO: 33
EQKLISEEDLEQKLISEEDLEQKLISEEDL
- HA
SEQ ID NO: 34
YPYDVPDYA
- 3x FLAG tag
SEQ ID NO: 35
DYKDHDGDYKDHDIDYKDDDDK
- 3′UTR 1357
SEQ ID NO: 36
AAGGCAAACCTTTCTATAATTTTACAAGGGAGTAGACTTGCTTTGGTCACTTT
TAGATGTGGTTAATTTTGCATATCCTTTTAGTCTGCATATATTAAAGCATCAG
GACCCTTCGTGACAATGTTTACAAATTACGTACTAAGGATACAGGCTGGAAA
GTAAGGGAAGCAGAAGGAAGGCTTTGAAAAGTTGTTTTATCTGGTGGGAAAT
TGCTTGACCCAGGTAGTCAAAGGCAGTTGACTAGAATCGACAAATTGTTACT
CCATATATATATATGTGTGTGTGTGTGTGTAAGATGTCTTCCTATCAAAAAGA
TATCAAAGGCACATGGAATATATTTTAATAAAAACAAATAATATCTCTAATA
TATCCACACATTTGTTGCCAGATTTCAGAAAACTGAGCTGCAATCGCTTTCCT
AAAACAGTAGTGTATTAAATGAACATCTATAAAATGTATCAACACACATTTT
AAAAAATTTGTTTAAAGTATACTCTTAGGCCAGGCGTGGTGACTCACACCTGT
AATTCCAGCACTTCAGGAGGCCAAGGTGGGAAGATCATTTGAGTTCAGGAGT
TCGAGTTACAGTCTGGGCAATAAAGTGAGACCCTGTCACTAACAAAATTAAA
AAATAAAATAAATATAAAATATAGGCTTTAAAAAAGCATAGTCTTATTAACC
ATGTCTGTTGGTCAAAATCTGCAAACTCTAAAAGAAGAAAAGAAGAAAAAA
CCAACGTTAGGGTATTTTTCCTCCCGTGCCTGAGTCCCAATTACATTCACGAC
AGTACTTTCAATGAACATAATTGTTAGGACCACTGAGGAATCATGAAAAATG
ATCTCTGCTTAGTACATTTGATGCAAAATGACTTATTAGGGGCTGTTTTTCTA
GCTATAGTGTCTCGAGTACTAATATGCAATTATGAAAATTATATTAAATCTGG
GATTATGACGGTATCACTGTATCATCTTGGTCTTGTTCTGGCTGTCACCAAGC
ATGACCCAGGTCAACTTTTTTTTTCCCCTGAATTACCCATCAAATTGATCTGC
AGCTGACTAAAGGCCACAGCTGAGCCTGGAACTGACCCTTCCTTCATCCTCA
ACCTGCTGTCCTCCAGAAAGCACCAAGGAAAAAGCAGAGAATGACAGCAAA
CAGATCACTAGGCCTCTGACCACAGGTGCTGAGTACTCAGCAGCCCTCATAT
AATAGGTTTGAAAGTACTCCTTAAAATAAAACACTGTTTCCCTTTGGAACTAT
TTACAAGGATGAAACAACCGTATACCTGAGAAATAACTTGCTCTGGTGTCAA
TTCGCTATTCGCCAGCAGACATCAGAACACACCGAGTTTCCAGATGCT
- 3′UTR 1406
SEQ ID NO: 37
AAGGCAAACCTTTCTATAATTTTACAAGGGAGTAGACTTGCTTTGGTCACTTT
TAGATGTGGTTAATTTTGCATATCCTTTTAGTCTGCATATATTAAAGCATCAG
GACCCTTCGTGACAATGTTTACAAATTACGTACTAAGGATACAGGCTGGAAA
GTAAGGGAAGCAGAAGGAAGGCTTTGAAAAGTTGTTTTATCTGGTGGGAAAT
TGCTTGACCCAGGTAGTCAAAGGCAGTTGACTAGAATCGACAAATTGTTACT
CCATATATATATATATGTGTGTGTGTGTGTAAGATGTCTTCCTATCAAAAAGA
TATCAAAGGCACATGGAATATATTTTAATAAAAACAAATAATATCTCTAATA
TATCCACACATTTGTTGCCAGATTTCAGAAAACTGAGCTGCAATCGCTTTCCT
AAAACAGTAGTGTATTAAATGAACATCTATAAAATGTATCAACACACATTTT
AAAAAATTTGTTTAAAGTATACTCTTAGGCCAGGCGTGGTGACTCACACCTGT
AATTCCAGCACTTCAGGAGGCCAAGGTGGGAAGATCATTTGAGTTCAGGAGT
TCGAGTTACAGCCTGGGCAATAAAGTGAGACCCTGTCACTAACAAAATTAAA
AAATAAAATAAATATAAAATATAGGCTTTAAAAAAGCATAGTCTTATTAACC
ATGTCTGTTGGTCAAAATCTGCAAACTCTAAAAGAAGAAAAGAAGAAAAAA
CCAACGTTAGGGTATTTTTCCTCCCGTGCCTGAGTCCCAATTACATTCACGAC
AGTACTTTCAATGAACATAATTGTTAGGACCACTGAGGAATCATGAAAAATG
ATCTCTGCTTAGTACATTTGATGCAAAATGACTTATTAGGGGCTGTTTTTCTA
GCTATAGTGTCTCGAGTACTAATATGCAATTATGAAAATTATATTAAATCTGG
GATTATGACGGTATCACTGTATCATCTTGGTCTTGTTCTGGCTGTCACCAAGC
ATGACCCAGGTCAACTTTTTTTTTCCCCTGAATTACCCATCAAATTGATCTGC
AGCTGACTAAAGGCCACAGCTGAGCCTGGAACTGACCCTTCCTTCATCCTCA
ACCTGCTGTCCTCCAGAAAGCACCAAGGAAAAAGCAGAGAATGACAGCAAA
CAGATCACTAGGCCTCTGACCACAGGTGCTGAGTACTCAGCAGCCCTCATAT
AATAGGTTTGAAAGTACTCCTTAAAATAAAACACTGTTTCCCTTTGGAACTAT
TTACAAGGATGAAACAACCGTATACCTGAGAAATAACTTGCTCTGGTGTCAA
TTCGCTATTCGCCAGCAGACATCAGAACACACCGAGTTTCCAGATGCTGGTTT
TTCCCCTTAAATCAGGAAATACACCTGGACAATTTCTAGAAGAC
- CLRN1 antibody epitope
SEQ ID NO: 38
EKIANYKEGTYVYKTQSEKY
- CLRN-0
SEQ ID NO: 39
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCATGCCTAGCCAGCAGAAGAAAAT
CATCTTCTGCATGGCCGGCGTGTTCAGCTTCGCCTGTGCTCTGGGAGTTGTGA
CAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAG
ATGCAGTACGGCCTGTTCCATGGCGAAGGCGTCAGACAGTGTGGCCTGGGAG
CCAGACCTTTCAGATTCAGCTTCTTCCCAGACCTGCTGAAGGCTATCCCCGTG
TCCATCCACGTGAACGTGATCCTGTTCAGCGCCATCCTGATCGTGCTGACAAT
GGTCGGAACCGCCTTCTTCATGTACAACGCCTTCGGCAAGCCCTTCGAGACA
CTGCATGGACCTCTGGGCCTGTACCTGCTGAGCTTTATCAGCGGCAGCTGTGG
CTGCCTGGTCATGATTCTGTTCGCCAGCGAAGTGAAGATCCACCACCTGAGC
GAGAAGATCGCCAACTACAAAGAGGGCACCTACGTCTACAAGACCCAGTCC
GAGAAGTACACCACCAGCTTTTGGGTTATCTTCTTCTGTTTCTTCGTGCACTTC
CTGAACGGCCTGCTGATCAGACTGGCCGGCTTCCAGTTTCCATTCGCCAAGA
GCAAGGACGCCGAAACCACAAACGTGGCCGCCGATCTGATGTACTAAGAGCT
CAAGGCAAACCTTTCTATAATTTTACAAGGGAGTAGACTTGCTTTGGTCACTT
TTAGATGTGGTTAATTTTGCATATCCTTTTAGTCTGCATATATTAAAGCATCA
GGACCCTTCGTGACAATGTTTACAAATTACGTACTAAGGATACAGGCTGGAA
AGTAAGGGAAGCAGAAGGAAGGCTTTGAAAAGTTGTTTTATCTGGTGGGAAA
TTGCTTGACCCAGGTAGTCAAAGGCAGTTGACTAGAATCGACAAATTGTTAC
TCCATATATATATATGTGTGTGTGTGTGTGTGTGTGTGTGTGTAAGATGTCTTC
CTATCAAAAAGATATCAAAGGCACATGGAATATATTTTAATAAAAACAAATA
ATATCTCTAATATATCCACACATTTGTTGCCAGATTTCAGAAAACTGAGCTGC
AATCGCTTTCCTAAAACAGTAGTGTATTAAATGAACATCTATAAAATGTATCA
ACACACATTTTAAAAAATTTGTTTAAAGTATACTCTTAGGCCAGGCGTGGTGA
CTCACACCTGTAATTCCAGCACTTCAGGAGGCCAAGGTGGGAAGATCATTTG
AGTTCAGGAGTTCGAGTTACAGCCTGGGCAATAAAGTGAGACCCTGTCACTA
ACAAAATTAAAAAATAAAATAAATATAAAATATAGGCTTTAAAAAAGCATA
GTCTTATTAACCATGTCTGTTGGTCAAAATCTGCAAACTCTAAAAGAAGAAA
AGAAGAAAAAACCAACGTTAGGGTATTTTTCCTCCCGTGCCTGAGTCCCAATT
ACATTCACGACAGTACTTTCAATGAACATAATTGTTAGGACCACTGAGGAAT
CATGAAAAATGATCTCTGCTTAGTACATTTGATGCAAAATGACTTATTAGGGG
CTGTTTTTCTAGCTATAGTGTCTCGAGTACTAATATGCAATTATGAAAATTAT
ATTAAATCTGGGATTATGACGGTATCACTGTATCATCTTGGTCTTGTTCTGGC
TGTCACCAAGCATGACCCAGGTCAACTTTTTTTTTCCCCTGAATTACCCATCA
AATTGATCTGCAGCTGACTAAAGGCCACAGCTGAGCCTGGAACTGACCCTTC
CTTCATCCTCAACCTGCTGTCCTCCAGAAAGCACCAAGGAAAAAGCAGAGAA
TGACAGCAAACAGATCACTAGGCCTCTGACCACAGGTGCTGAGTACTCAGCA
GCCCTCATATAATAGGTTTGAAAGTACTCCTTAAAATAAAACACTGTTTCCCT
TTGGAACTATTTACAAGGATGAAACAACCGTATACCTGAGAAATAACTTGCT
CTGGTGTCAATTCGCTATTCGCCAGCAGACATCAGAACACACCGAGTTTCCA
GATGCTGGTTTTTCCCCTTAAATCAGGAAATACACCTGGACAATTTCTAGAAG
ACTACAATTCAGTCTAGCCACAAAGGGGATTTTTTTTTTTTGGTAACAGGCTA
GAGCCCGGTTCTGTAAGTCTTTAGCTGAAATGGTCCAGTACAAAAGCACTGG
AAATGAGTGGGCTAGGAGGACAAGGACCGTCTCCTGCGTGAGGAGTTGGTTG
GAGGTCCCCAAGGCCAGGTACCCCCTGCACTCTTATTGGATTCCTCTCTGTCT
TCTTGGAGTTTTGAAAAACTCCTTCGAACACCAGGCTTTTTTCTTTAGAAAAC
AAGTCTCCAATCGTTCTCTGTTCCGTAGAAAGAGAAAGAAAACCTGGAGCAG
CTGCTGAAAAATCTAATGAGGAACTAAGAGGCAAACCCACCACTGTGCCTTC
TAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATT
GTCTGAGTAGGTGTCATTCTATTCTGGGGGGTGGGGTGGGGCAGGACAGCAA
GGGGGAGGATTGGGAAGACAATAGCAGGCATGCTGGGGATGCGGTGGGCTC
TATGGAAGCTTGAATTCAGCTGACGTGCCTCGGACCGCTAGGAACCCCTAGT
GATGGAGTTGGCCACTCCCTCTCTGCGCGCTCGCTCGCTCACTGAGGCCGGGC
GACCAAAGGTCGCCCGACGCCCGGGCTTTGCCCGGGCGGCCTCAGTGAGCGA
GCGAGCGCGCAGCTGCCTGCAGG
- CLRN-1
SEQ ID NO: 40
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCATGCCTAGCCAGCAGAAGAAAAT
CATCTTCTGCATGGCCGGCGTGTTCAGCTTCGCCTGTGCTCTGGGAGTTGTGA
CAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAG
ATGCAGTACGGCCTGTTCCATGGCGAAGGCGTCAGACAGTGTGGCCTGGGAG
CCAGACCTTTCAGATTCAGCTTCTTCCCAGACCTGCTGAAGGCTATCCCCGTG
TCCATCCACGTGAACGTGATCCTGTTCAGCGCCATCCTGATCGTGCTGACAAT
GGTCGGAACCGCCTTCTTCATGTACAACGCCTTCGGCAAGCCCTTCGAGACA
CTGCATGGACCTCTGGGCCTGTACCTGCTGAGCTTTATCAGCGGCAGCTGTGG
CTGCCTGGTCATGATTCTGTTCGCCAGCGAAGTGAAGATCCACCACCTGAGC
GAGAAGATCGCCAACTACAAAGAGGGCACCTACGTCTACAAGACCCAGTCC
GAGAAGTACACCACCAGCTTTTGGGTTATCTTCTTCTGTTTCTTCGTGCACTTC
CTGAACGGCCTGCTGATCAGACTGGCCGGCTTCCAGTTTCCATTCGCCAAGA
GCAAGGACGCCGAAACCACAAACGTGGCCGCCGATCTGATGTACGGCAGCG
GAGAAGGCAGAGGCAGCCTGCTTACATGTGGCGACGTGGAAGAGAACCCCG
GACCTATGCAGGCTCTGCAGCAGCAGCCAGTGTTCCCCGATCTGCTGAAAGC
CATTCCTGTCAGCATCCATGTCAACGTCATCCTCTTCTCTGCCATCCTCATTGT
CCTCACTATGGTTGGAACGGCCTTTTTTATGTATAATGCCTTTGGGAAGCCGT
TTGAAACCCTGCACGGACCCCTGGGACTCTATCTCCTGAGCTTCATCTCCGGC
TCTTGCGGCTGCCTCGTGATGATCCTCTTTGCCTCTGAAGTCAAAATTCACCA
CCTGTCTGAGAAAATTGCTAATTACAAAGAAGGGACATACGTTTACAAAACG
CAGAGCGAAAAGTATACGACCAGCTTCTGGCTGACCAAGGGCCACTCTTAAG
AGCTCGCTGATCAGCCTCGACTGTGCCTTCTAGTTGCCAGCCATCTGTTGTTT
GCCCCTCCCCCGTGCCTTCCTTGACCCTGGAAGGTGCCACTCCCACTGTCCTT
TCCTAATAAAATGAGGAAATTGCATCGCATTGTCTGAGTAGGTGTCATTCTAT
TCTGGGGGGTGGGGTGGGGCAGGACAGCAAGGGGGAGGATTGGGAAGACAA
TAGCAGGCATGCTGGGGATGCGGTGGGCTCTATGGAAGCTTGAATTCAGCTG
ACGTGCCTCGGACCGCTAGGAACCCCTAGTGATGGAGTTGGCCACTCCCTCT
CTGCGCGCTCGCTCGCTCACTGAGGCCGGGCGACCAAAGGTCGCCCGACGCC
CGGGCTTTGCCCGGGCGGCCTCAGTGAGCGAGCGAGCGCGCAGCTGCCTGCA
GG
- CLRN-2eGFP
SEQ ID NO: 41
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCATGCCTAGCCAGCAGAAGAAAAT
CATCTTCTGCATGGCCGGCGTGTTCAGCTTCGCCTGTGCTCTGGGAGTTGTGA
CAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAG
ATGCAGTACGGCCTGTTCCATGGCGAAGGCGTCAGACAGTGTGGCCTGGGAG
CCAGACCTTTCAGATTCAGCTTCTTCCCAGACCTGCTGAAGGCTATCCCCGTG
TCCATCCACGTGAACGTGATCCTGTTCAGCGCCATCCTGATCGTGCTGACAAT
GGTCGGAACCGCCTTCTTCATGTACAACGCCTTCGGCAAGCCCTTCGAGACA
CTGCATGGACCTCTGGGCCTGTACCTGCTGAGCTTTATCAGCGGCAGCTGTGG
CTGCCTGGTCATGATTCTGTTCGCCAGCGAAGTGAAGATCCACCACCTGAGC
GAGAAGATCGCCAACTACAAAGAGGGCACCTACGTCTACAAGACCCAGTCC
GAGAAGTACACCACCAGCTTTTGGGTTATCTTCTTCTGTTTCTTCGTGCACTTC
CTGAACGGCCTGCTGATCAGACTGGCCGGCTTCCAGTTTCCATTCGCCAAGA
GCAAGGACGCCGAAACCACAAACGTGGCCGCCGATCTGATGTACGGCAGCG
GAGAAGGCAGAGGCAGCCTGCTTACATGTGGCGACGTGGAAGAGAACCCCG
GACCTATGCAGGCTCTGCAGCAGCAGCCAGTGTTCCCCGATCTGCTGAAAGC
CATTCCTGTCAGCATCCATGTCAACGTCATCCTCTTCTCTGCCATCCTCATTGT
CCTCACTATGGTTGGAACGGCCTTTTTTATGTATAATGCCTTTGGGAAGCCGT
TTGAAACCCTGCACGGACCCCTGGGACTCTATCTCCTGAGCTTCATCTCCGGC
TCTTGCGGCTGCCTCGTGATGATCCTCTTTGCCTCTGAAGTCAAAATTCACCA
CCTGTCTGAGAAAATTGCTAATTACAAAGAAGGGACATACGTTTACAAAACG
CAGAGCGAAAAGTATACGACCAGCTTCTGGCTGACCAAGGGCCACTCTGGAT
CAGGCGAAGGCAGGGGATCTCTGCTGACCTGCGGAGATGTCGAAGAGAATCC
TGGACCAatggtgagcaagggcgaggagctgttcaccggggtggtgcccatcctggtcgagctggacggcgacgtaaa
cggccacaagttcagcgtgtccggcgagggcgagggcgatgccacctacggcaagctgaccctgaagttcatctgcaccacc
ggcaagctgcccgtgccctggcccaccctcgtgaccaccctgacctacggcgtgcagtgcttcagccgctaccccgaccacat
gaagcagcacgacttcttcaagtccgccatgcccgaaggctacgtccaggagcgcaccatcttcttcaaggacgacggcaact
acaagacccgcgccgaggtgaagttcgagggcgacaccctggtgaaccgcatcgagctgaagggcatcgacttcaaggagg
acggcaacatcctggggcacaagctggagtacaactacaacagccacaacgtctatatcatggccgacaagcagaagaacgg
catcaaggtgaacttcaagatccgccacaacatcgaggacggcagcgtgcagctcgccgaccactaccagcagaacaccccc
atcggcgacggccccgtgctgctgcccgacaaccactacctgagcacccagtccgccctgagcaaagaccccaacgagaag
cgcgatcacatggtcctgctggagttcgtgaccgccgccgggatcactctcggcatggacgagctgtacaagTAAGAGC
TCGCTGATCAGCCTCGACTGTGCCTTCTAGTTGCCAGCCATCTGTTGTTTGCCC
CTCCCCCGTGCCTTCCTTGACCCTGGAAGGTGCCACTCCCACTGTCCTTTCCT
AATAAAATGAGGAAATTGCATCGCATTGTCTGAGTAGGTGTCATTCTATTCTG
GGGGGTGGGGTGGGGCAGGACAGCAAGGGGGAGGATTGGGAAGACAATAGC
AGGCATGCTGGGGATGCGGTGGGCTCTATGGAAGCTTGAATTCAGCTGACGT
GCCTCGGACCGCTAGGAACCCCTAGTGATGGAGTTGGCCACTCCCTCTCTGC
GCGCTCGCTCGCTCACTGAGGCCGGGCGACCAAAGGTCGCCCGACGCCCGGG
CTTTGCCCGGGCGGCCTCAGTGAGCGAGCGAGCGCGCAGCTGCCTGCAGG
- CLRN-3
SEQ ID NO: 42
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTAGGAGATACTTGAAGGCAGTTTGAAAGAC
TTGTTTTACAGATTCTTAGTCCAAAGATTTCCAATTAGGGAGAAGAAGCAGC
AGAAAAGGAGAAAAGCCAAGTATGAGTGATGATGAGGCCTTCATCTACTGAC
ATTTAACCTGGCGAGAACCGTCGATGGTGAAGTTGCCTTTTCAGCTGGGAGCT
GTCCGTTCAGCTTCCGTAATAAATGCAGTCAAAGAGGCAGTCCCTTCCCATTG
CTCACAAAGGTCTTGTTTTTGAACCTCGCCCTCACAGAAGCCGTTTCTCATCG
CCACCATGCCTAGCCAGCAGAAGAAAATCATCTTCTGCATGGCCGGCGTGTT
CAGCTTCGCCTGTGCTCTGGGAGTTGTGACAGCCCTGGGAACCCCTCTGTGGA
TCAAAGCCACAGTGCTGTGCAAGACAGGCGCCCTGCTGGTTAATGCCTCTGG
CCAAGAGCTGGACAAGTTCATGGGCGAGATGCAGTACGGCCTGTTCCATGGC
GAAGGCGTCAGACAGTGTGGCCTGGGAGCCAGACCTTTCAGATTCAGCTTCT
TCCCAGACCTGCTGAAGGCTATCCCCGTGTCCATCCACGTGAACGTGATCCTG
TTCAGCGCCATCCTGATCGTGCTGACAATGGTCGGAACCGCCTTCTTCATGTA
CAACGCCTTCGGCAAGCCCTTCGAGACACTGCATGGACCTCTGGGCCTGTAC
CTGCTGAGCTTTATCAGCGGCAGCTGTGGCTGCCTGGTCATGATTCTGTTCGC
CAGCGAAGTGAAGATCCACCACCTGAGCGAGAAGATCGCCAACTACAAAGA
GGGCACCTACGTCTACAAGACCCAGTCCGAGAAGTACACCACCAGCTTTTGG
GTTATCTTCTTCTGTTTCTTCGTGCACTTCCTGAACGGCCTGCTGATCAGACTG
GCCGGCTTCCAGTTTCCATTCGCCAAGAGCAAGGACGCCGAAACCACAAACG
TGGCCGCCGATCTGATGTACTAAGAGCTCAAGGCAAACCTTTCTATAATTTTA
CAAGGGAGTAGACTTGCTTTGGTCACTTTTAGATGTGGTTAATTTTGCATATC
CTTTTAGTCTGCATATATTAAAGCATCAGGACCCTTCGTGACAATGTTTACAA
ATTACGTACTAAGGATACAGGCTGGAAAGTAAGGGAAGCAGAAGGAAGGCT
TTGAAAAGTTGTTTTATCTGGTGGGAAATTGCTTGACCCAGGTAGTCAAAGGC
AGTTGACTAGAATCGACAAATTGTTACTCCATATATATATATGTGTGTGTGTG
TGTGTAAGATGTCTTCCTATCAAAAAGATATCAAAGGCACATGGAATATATTT
TAATAAAAACAAATAATATCTCTAATATATCCACACATTTGTTGCCAGATTTC
AGAAAACTGAGCTGCAATCGCTTTCCTAAAACAGTAGTGTATTAAATGAACA
TCTATAAAATGTATCAACACACATTTTAAAAAATTTGTTTAAAGTATACTCTT
AGGCCAGGCGTGGTGACTCACACCTGTAATTCCAGCACTTCAGGAGGCCAAG
GTGGGAAGATCATTTGAGTTCAGGAGTTCGAGTTACAGTCTGGGCAATAAAG
TGAGACCCTGTCACTAACAAAATTAAAAAATAAAATAAATATAAAATATAGG
CTTTAAAAAAGCATAGTCTTATTAACCATGTCTGTTGGTCAAAATCTGCAAAC
TCTAAAAGAAGAAAAGAAGAAAAAACCAACGTTAGGGTATTTTTCCTCCCGT
GCCTGAGTCCCAATTACATTCACGACAGTACTTTCAATGAACATAATTGTTAG
GACCACTGAGGAATCATGAAAAATGATCTCTGCTTAGTACATTTGATGCAAA
ATGACTTATTAGGGGCTGTTTTTCTAGCTATAGTGTCTCGAGTACTAATATGC
AATTATGAAAATTATATTAAATCTGGGATTATGACGGTATCACTGTATCATCT
TGGTCTTGTTCTGGCTGTCACCAAGCATGACCCAGGTCAACTTTTTTTTTCCCC
TGAATTACCCATCAAATTGATCTGCAGCTGACTAAAGGCCACAGCTGAGCCT
GGAACTGACCCTTCCTTCATCCTCAACCTGCTGTCCTCCAGAAAGCACCAAGG
AAAAAGCAGAGAATGACAGCAAACAGATCACTAGGCCTCTGACCACAGGTG
CTGAGTACTCAGCAGCCCTCATATAATAGGTTTGAAAGTACTCCTTAAAATAA
AACACTGTTTCCCTTTGGAACTATTTACAAGGATGAAACAACCGTATACCTGA
GAAATAACTTGCTCTGGTGTCAATTCGCTATTCGCCAGCAGACATCAGAACA
CACCGAGTTTCCAGATGCTCTGTGCCTTCTAGTTGCCAGCCATCTGTTGTTTGC
CCCTCCCCCGTGCCTTCCTTGACCCTGGAAGGTGCCACTCCCACTGTCCTTTC
CTAATAAAATGAGGAAATTGCATCGCATTGTCTGAGTAGGTGTCATTCTATTC
TGGGGGGTGGGGTGGGGCAGGACAGCAAGGGGGAGGATTGGGAAGACAATA
GCAGGCATGCTGGGGATGCGGTGGGCTCTATGGAAGCTTGAATTCAGCTGAC
GTGCCTCGGACCGCTAGGAACCCCTAGTGATGGAGTTGGCCACTCCCTCTCTG
CGCGCTCGCTCGCTCACTGAGGCCGGGCGACCAAAGGTCGCCCGACGCCCGG
GCTTTGCCCGGGCGGCCTCAGTGAGCGAGCGAGCGCGCAGCTGCCTGCAGG
- CLRN-4
SEQ ID NO: 43
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCATGCCTAGCCAGCAGAAGAAAAT
CATCTTCTGCATGGCCGGCGTGTTCAGCTTCGCCTGTGCTCTGGGAGTTGTGA
CAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAG
ATGCAGTACGGCCTGTTCCATGGCGAAGGCGTCAGACAGTGTGGCCTGGGAG
CCAGACCTTTCAGATTCAGCTTCTTCCCAGACCTGCTGAAGGCTATCCCCGTG
TCCATCCACGTGAACGTGATCCTGTTCAGCGCCATCCTGATCGTGCTGACAAT
GGTCGGAACCGCCTTCTTCATGTACAACGCCTTCGGCAAGCCCTTCGAGACA
CTGCATGGACCTCTGGGCTGTACCTGCTGAGCTTTATCAGCGGCAGCTGTGG
CTGCCTGGTCATGATTCTGTTCGCCAGCGAAGTGAAGATCCACCACCTGAGC
GAGAAGATCGCCAACTACAAAGAGGGCACCTACGTCTACAAGACCCAGTCC
GAGAAGTACACCACCAGCTTTTGGGTTATCTTCTTCTGTTTCTTCGTGCACTTC
CTGAACGGCCTGCTGATCAGACTGGCCGGCTTCCAGTTTCCATTCGCCAAGA
GCAAGGACGCCGAAACCACAAACGTGGCCGCCGATCTGATGTACGGCAGCG
GAGAAGGCAGAGGCAGCCTGCTTACATGTGGCGACGTGGAAGAGAACCCCG
GACCTATGCAGGCTCTGCAGCAGCAGCCAGTGTTCCCCGATCTGCTGAAAGC
CATTCCTGTCAGCATCCATGTCAACGTCATCCTCTTCTCTGCCATCCTCATTGT
CCTCACTATGGTTGGAACGGCCTTTTTTATGTATAATGCCTTTGGGAAGCCGT
TTGAAACCCTGCACGGACCCCTGGGACTCTATCTCCTGAGCTTCATCTCCGGC
TCTTGCGGCTGCCTCGTGATGATCCTCTTTGCCTCTGAAGTCAAAATTCACCA
CCTGTCTGAGAAAATTGCTAATTACAAAGAAGGGACATACGTTTACAAAACG
CAGAGCGAAAAGTATACGACCAGCTTCTGGCTGACCAAGGGCCACTCTTAAG
AGCTCAAGGCAAACCTTTCTATAATTTTACAAGGGAGTAGACTTGCTTTGGTC
ACTTTTAGATGTGGTTAATTTTGCATATCCTTTTAGTCTGCATATATTAAAGCA
TCAGGACCCTTCGTGACAATGTTTACAAATTACGTACTAAGGATACAGGCTG
GAAAGTAAGGGAAGCAGAAGGAAGGCTTTGAAAAGTTGTTTTATCTGGTGGG
AAATTGCTTGACCCAGGTAGTCAAAGGCAGTTGACTAGAATCGACAAATTGT
TACTCCATATATATATATATGTGTGTGTGTGTGTAAGATGTCTTCCTATCAAA
AAGATATCAAAGGCACATGGAATATATTTTAATAAAAACAAATAATATCTCT
AATATATCCACACATTTGTTGCCAGATTTCAGAAAACTGAGCTGCAATCGCTT
TCCTAAAACAGTAGTGTATTAAATGAACATCTATAAAATGTATCAACACACA
TTTTAAAAAATTTGTTTAAAGTATACTCTTAGGCCAGGCGTGGTGACTCACAC
CTGTAATTCCAGCACTTCAGGAGGCCAAGGTGGGAAGATCATTTGAGTTCAG
GAGTTCGAGTTACAGCCTGGGCAATAAAGTGAGACCCTGTCACTAACAAAAT
TAAAAAATAAAATAAATATAAAATATAGGCTTTAAAAAAGCATAGTCTTATT
AACCATGTCTGTTGGTCAAAATCTGCAAACTCTAAAAGAAGAAAAGAAGAAA
AAACCAACGTTAGGGTATTTTTCCTCCCGTGCCTGAGTCCCAATTACATTCAC
GACAGTACTTTCAATGAACATAATTGTTAGGACCACTGAGGAATCATGAAAA
ATGATCTCTGCTTAGTACATTTGATGCAAAATGACTTATTAGGGGCTGTTTTT
CTAGCTATAGTGTCTCGAGTACTAATATGCAATTATGAAAATTATATTAAATC
TGGGATTATGACGGTATCACTGTATCATCTTGGTCTTGTTCTGGCTGTCACCA
AGCATGACCCAGGTCAACTTTTTTTTTCCCCTGAATTACCCATCAAATTGATC
TGCAGCTGACTAAAGGCCACAGCTGAGCCTGGAACTGACCCTTCCTTCATCCT
CAACCTGCTGTCCTCCAGAAAGCACCAAGGAAAAAGCAGAGAATGACAGCA
AACAGATCACTAGGCCTCTGACCACAGGTGCTGAGTACTCAGCAGCCCTCAT
ATAATAGGTTTGAAAGTACTCCTTAAAATAAAACACTGTTTCCCTTTGGAACT
ATTTACAAGGATGAAACAACCGTATACCTGAGAAATAACTTGCTCTGGTGTC
AATTCGCTATTCGCCAGCAGACATCAGAACACACCGAGTTTCCAGATGCTGG
TTTTTCCCCTTAAATCAGGAAATACACCTGGACAATTTCTAGAAGACAGGCCT
CTGTGCCTTCTAGTTGCCAGCCATCTGTTGTTTGCCCCTCCCCCGTGCCTTCCT
TGACCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATG
CGGTGGGCTCTATGGAAGCTTGAATTCAGCTGACGTGCCTCGGACCGCTAGG
AACCCCTAGTGATGGAGTTGGCCACTCCCTCTCTGCGCGCTCGCTCGCTCACT
GAGGCCGGGCGACCAAAGGTCGCCCGACGCCCGGGCTTTGCCCGGGCGGCCT
CAGTGAGCGAGCGAGCGCGCAGCTGCCTGCAGG
- CLRN-6eGFP
SEQ ID NO: 44
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCatggtgagcaagggcgaggagctgttcaccggggtg
gtgcccatcctggtcgagctggacggcgacgtaaacggccacaagttcagcgtgtccggcgagggcgagggcgatgccacc
tacggcaagctgaccctgaagttcatctgcaccaccggcaagctgcccgtgccctggcccaccctcgtgaccaccctgacctac
ggcgtgcagtgcttcagccgctaccccgaccacatgaagcagcacgacttcttcaagtccgccatgcccgaaggctacgtcca
ggagcgcaccatcttcttcaaggacgacggcaactacaagacccgcgccgaggtgaagttcgagggcgacaccctggtgaac
cgcatcgagctgaagggcatcgacttcaaggaggacggcaacatcctggggcacaagctggagtacaactacaacagccac
aacgtctatatcatggccgacaagcagaagaacggcatcaaggtgaacttcaagatccgccacaacatcgaggacggcagcg
tgcagctcgccgaccactaccagcagaacacccccatcggcgacggccccgtgctgctgcccgacaaccactacctgagcac
ccagtccgccctgagcaaagaccccaacgagaagcgcgatcacatggtcctgctggagttcgtgaccgccgccgggatcact
ctcggcatggacgagctgtacaagTAATAAGAGCTCAAGGCAAACCTTTCTATAATTTTAC
AAGGGAGTAGACTTGCTTTGGTCACTTTTAGATGTGGTTAATTTTGCATATCC
TTTTAGTCTGCATATATTAAAGCATCAGGACCCTTCGTGACAATGTTTACAAA
TTACGTACTAAGGATACAGGCTGGAAAGTAAGGGAAGCAGAAGGAAGGCTT
TGAAAAGTTGTTTTATCTGGTGGGAAATTGCTTGACCCAGGTAGTCAAAGGC
AGTTGACTAGAATCGACAAATTGTTACTCCATATATATATATGTGTGTGTGTG
TGTGTGTGTGTGTGTGTAAGATGTCTTCCTATCAAAAAGATATCAAAGGCACA
TGGAATATATTTTAATAAAAACAAATAATATCTCTAATATATCCACACATTTG
TTGCCAGATTTCAGAAAACTGAGCTGCAATCGCTTTCCTAAAACAGTAGTGTA
TTAAATGAACATCTATAAAATGTATCAACACACATTTTAAAAAATTTGTTTAA
AGTATACTCTTAGGCCAGGCGTGGTGACTCACACCTGTAATTCCAGCACTTCA
GGAGGCCAAGGTGGGAAGATCATTTGAGTTCAGGAGTTCGAGTTACAGCCTG
GGCAATAAAGTGAGACCCTGTCACTAACAAAATTAAAAAATAAAATAAATAT
AAAATATAGGCTTTAAAAAAGCATAGTCTTATTAACCATGTCTGTTGGTCAAA
ATCTGCAAACTCTAAAAGAAGAAAAGAAGAAAAAACCAACGTTAGGGTATT
TTTCCTCCCGTGCCTGAGTCCCAATTACATTCACGACAGTACTTTCAATGAAC
ATAATTGTTAGGACCACTGAGGAATCATGAAAAATGATCTCTGCTTAGTACA
TTTGATGCAAAATGACTTATTAGGGGCTGTTTTTCTAGCTATAGTGTCTCGAG
TACTAATATGCAATTATGAAAATTATATTAAATCTGGGATTATGACGGTATCA
CTGTATCATCTTGGTCTTGTTCTGGCTGTCACCAAGCATGACCCAGGTCAACT
TTTTTTTTCCCCTGAATTACCCATCAAATTGATCTGCAGCTGACTAAAGGCCA
CAGCTGAGCCTGGAACTGACCCTTCCTTCATCCTCAACCTGCTGTCCTCCAGA
AAGCACCAAGGAAAAAGCAGAGAATGACAGCAAACAGATCACTAGGCCTCT
GACCACAGGTGCTGAGTACTCAGCAGCCCTCATATAATAGGTTTGAAAGTAC
TCCTTAAAATAAAACACTGTTTCCCTTTGGAACTATTTACAAGGATGAAACAA
CCGTATACCTGAGAAATAACTTGCTCTGGTGTCAATTCGCTATTCGCCAGCAG
ACATCAGAACACACCGAGTTTCCAGATGCTGGTTTTTCCCCTTAAATCAGGAA
ATACACCTGGACAATTTCTAGAAGACTACAATTCAGTCTAGCCACAAAGGGG
ATTTTTTTTTTTTGGTAACAGGCTAGAGCCCGGTTCTGTAAGTCTTTAGCTGAA
ATGGTCCAGTACAAAAGCACTGGAAATGAGTGGGCTAGGAGGACAAGGACC
GTCTCCTGCGTGAGGAGTTGGTTGGAGGTCCCCAAGGCCAGGTACCCCCTGC
ACTCTTATTGGATTCCTCTCTGTCTTCTTGGAGTTTTGAAAAACTCCTTCGAAC
ACCAGGCTTTTTTCTTTAGAAAACAAGTCTCCAATCGTTCTCTGTTCCGTAGA
AAGAGAAAGAAAACCTGGAGCAGCTGCTGAAAAATCTAATGAGGAACTAAG
AGGCAAACCCACCACTGTGCCTTCTAGTTGCCAGCCATCTGTTGTTTGCCCCT
CCCCCGTGCCTTCCTTGACCCTGGAAGGTGCCACTCCCACTGTCCTTTCCTAA
TAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAG
GCATGCTGGGGATGCGGTGGGCTCTATGGAAGCTTGAATTCAGCTGACGTGC
CTCGGACCGCTAGGAACCCCTAGTGATGGAGTTGGCCACTCCCTCTCTGCGC
GCTCGCTCGCTCACTGAGGCCGGGCGACCAAAGGTCGCCCGACGCCCGGGCT
TTGCCCGGGCGGCCTCAGTGAGCGAGCGAGCGCGCAGCTGCCTGCAGG
- CLRN-7eGFP
SEQ ID NO: 45
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCatggtgagcaagggcgaggagctgttcaccggggtg
gtgcccatcctggtcgagctggacggcgacgtaaacggccacaagttcagcgtgtccggcgagggcgagggcgatgccacc
tacggcaagctgaccctgaagttcatctgcaccaccggcaagctgcccgtgccctggcccaccctcgtgaccaccctgacctac
ggcgtgcagtgcttcagccgctaccccgaccacatgaagcagcacgacttcttcaagtccgccatgcccgaaggctacgtcca
ggagcgcaccatcttcttcaaggacgacggcaactacaagacccgcgccgaggtgaagttcgagggcgacaccctggtgaac
cgcatcgagctgaagggcatcgacttcaaggaggacggcaacatcctggggcacaagctggagtacaactacaacagccac
aacgtctatatcatggccgacaagcagaagaacggcatcaaggtgaacttcaagatccgccacaacatcgaggacggcagcg
tgcagctcgccgaccactaccagcagaacacccccatcggcgacggccccgtgctgctgcccgacaaccactacctgagcac
ccagtccgccctgagcaaagaccccaacgagaagcgcgatcacatggtcctgctggagttcgtgaccgccgccgggatcact
ctcggcatggacgagctgtacaagTAAGAGCTCAAGGCAAACCTTTCTATAATTTTACAAG
GGAGTAGACTTGCTTTGGTCACTTTTAGATGTGGTTAATTTTGCATATCCTTTT
AGTCTGCATATATTAAAGCATCAGGACCCTTCGTGACAATGTTTACAAATTAC
GTACTAAGGATACAGGCTGGAAAGTAAGGGAAGCAGAAGGAAGGCTTTGAA
AAGTTGTTTTATCTGGTGGGAAATTGCTTGACCCAGGTAGTCAAAGGCAGTTG
ACTAGAATCGACAAATTGTTACTCCATATATATATATGTGTGTGTGTGTGTGT
GTGTGTGTGTGTAAGATGTCTTCCTATCAAAAAGATATCAAAGGCACATGGA
ATATATTTTAATAAAAACAAATAATATCTCTAATATATCCACACATTTGTTGC
CAGATTTCAGAAAACTGAGCTGCAATCGCTTTCCTAAAACAGTAGTGTATTA
AATGAACATCTATAAAATGTATCAACACACATTTTAAAAAATTTGTTTAAAGT
ATACTCTTAGGCCAGGCGTGGTGACTCACACCTGTAATTCCAGCACTTCAGGA
GGCCAAGGTGGGAAGATCATTTGAGTTCAGGAGTTCGAGTTACAGCTGTGCC
TTCTAGTTGCCAGCCATCTGTTGTTTGCCCCTCCCCCGTGCCTTCCTTGACCCT
GGAAGGTGCCACTCCCACTGTCCTTTCCTAATAAAATGAGGAAATTGCATCG
CATTGTCTGAGTAGGTGTCATTCTATTCTGGGGGGTGGGGTGGGGCAGGACA
GCAAGGGGGAGGATTGGGAAGACAATAGCAGGCATGCTGGGGATGCGGTGG
GCTCTATGGAAGCTTGAATTCAGCTGACGTGCCTCGGACCGCTAGGAACCCC
TAGTGATGGAGTTGGCCACTCCCTCTCTGCGCGCTCGCTCGCTCACTGAGGCC
GGGCGACCAAAGGTCGCCCGACGCCCGGGCTTTGCCCGGGCGGCCTCAGTGA
GCGAGCGAGCGCGCAGCTGCCTGCAGG
- CLRN-8
SEQ ID NO: 46
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCATGCCTAGCCAGCAGAAGAAAAT
CATCTTCTGCATGGCCGGCGTGTTCAGCTTCGCCTGTGCTCTGGGAGTTGTGA
CAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAG
ATGCAGTACGGCCTGTTCCATGGCGAAGGCGTCAGACAGTGTGGCCTGGGAG
CCAGACCTTTCAGATTCAGCTTCTTCCCAGACCTGCTGAAGGCTATCCCCGTG
TCCATCCACGTGAACGTGATCCTGTTCAGCGCCATCCTGATCGTGCTGACAAT
GGTCGGAACCGCCTTCTTCATGTACAACGCCTTCGGCAAGCCCTTCGAGACA
CTGCATGGACCTCTGGGCCTGTACCTGCTGAGCTTTATCAGCGGCAGCTGTGG
CTGCCTGGTCATGATTCTGTTCGCCAGCGAAGTGAAGATCCACCACCTGAGC
GAGAAGATCGCCAACTACAAAGAGGGCACCTACGTCTACAAGACCCAGTCC
GAGAAGTACACCACCAGCTTTTGGGTTATCTTCTTCTGTTTCTTCGTGCACTTC
CTGAACGGCCTGCTGATCAGACTGGCCGGCTTCCAGTTTCCATTCGCCAAGA
GCAAGGACGCCGAAACCACAAACGTGGCCGCCGATCTGATGTACGGCAGCG
GAGAAGGCAGAGGCAGCCTGCTTACATGTGGCGACGTGGAAGAGAACCCCG
GACCTATGCAGGCTCTGCAGCAGCAGCCAGTGTTCCCCGATCTGCTGAAAGC
CATTCCTGTCAGCATCCATGTCAACGTCATCCTCTTCTCTGCCATCCTCATTGT
CCTCACTATGGTTGGAACGGCCTTTTTTATGTATAATGCCTTTGGGAAGCCGT
TTGAAACCCTGCACGGACCCCTGGGACTCTATCTCCTGAGCTTCATCTCCGGC
TCTTGCGGCTGCCTCGTGATGATCCTCTTTGCCTCTGAAGTCAAAATTCACCA
CCTGTCTGAGAAAATTGCTAATTACAAAGAAGGGACATACGTTTACAAAACG
CAGAGCGAAAAGTATACGACCAGCTTCTGGCTGACCAAGGGCCACTCTTAAG
AGCTCGAAGATCATTTGAGTTCAGGAGTTCGAGTTACAGCCTGGGCAATAAA
GTGAGACCCTGTCACTAACAAAATTAAAAAATAAAATAAATATAAAATATAG
GCTTTAAAAAAGCATAGTCTTATTAACCATGTCTGTTGGTCAAAATCTGCAAA
CTCTAAAAGAAGAAAAGAAGAAAAAACCAACGTTAGGGTATTTTTCCTCCCG
TGCCTGAGTCCCAATTACATTCACGACAGTACTTTCAATGAACATAATTGTTA
GGACCACTGAGGAATCATGAAAAATGATCTCTGCTTAGTACATTTGATGCAA
AATGACTTATTAGGGGCTGTTTTTCTAGCTATAGTGTCTCGAGTACTAATATG
CAATTATGAAAATTATATTAAATCTGGGATTATGACGGTATCACTGTATCATC
TTGGTCTTGTTCTGGCTGTCACCAAGCATGACCCAGGTCAACTTTTTTTTTCCC
CTGAATTACCCATCAAATTGATCTGCAGCTGACTAAAGGCCACAGCTGAGCC
TGGAACTGACCCTTCCTTCATCCTCAACCTGCTGTCCTCCAGAAAGCACCAAG
GAAAAAGCAGAGAATGACAGCAAACACTGTGCCTTCTAGTTGCCAGCCATCT
GTTGTTTGCCCCTCCCCCGTGCCTTCCTTGACCCTGGAAGGTGCCACTCCCAC
TGTCCTTTCCTAATAAAATGAGGAAATTGCATCGCATTGTCTGAGTAGGTGTC
ATTCTATTCTGGGGGGTGGGGTGGGGCAGGACAGCAAGGGGGAGGATTGGG
AAGACAATAGCAGGCATGCTGGGGATGCGGTGGGCTCTATGGAAGCTTGAAT
TCAGCTGACGTGCCTCGGACCGCTAGGAACCCCTAGTGATGGAGTTGGCCAC
TCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGCT
GCCTGCAGG
- CLRN-9
SEQ ID NO: 47
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCATGCCTAGCCAGCAGAAGAAAAT
CATCTTCTGCATGGCCGGCGTGTTCAGCTTCGCCTGTGCTCTGGGAGTTGTGA
CAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAG
ATGCAGTACGGCCTGTTCCATGGCGAAGGCGTCAGACAGTGTGGCCTGGGAG
CCAGACCTTTCAGATTCAGCTTCTTCCCAGACCTGCTGAAGGCTATCCCCGTG
TCCATCCACGTGAACGTGATCCTGTTCAGCGCCATCCTGATCGTGCTGACAAT
GGTCGGAACCGCCTTCTTCATGTACAACGCCTTCGGCAAGCCCTTCGAGACA
CTGCATGGACCTCTGGGCCTGTACCTGCTGAGCTTTATCAGCGGCAGCTGTGG
CTGCCTGGTCATGATTCTGTTCGCCAGCGAAGTGAAGATCCACCACCTGAGC
GAGAAGATCGCCAACTACAAAGAGGGCACCTACGTCTACAAGACCCAGTCC
GAGAAGTACACCACCAGCTTTTGGGTTATCTTCTTCTGTTTCTTCGTGCACTTC
CTGAACGGCCTGCTGATCAGACTGGCCGGCTTCCAGTTTCCATTCGCCAAGA
GCAAGGACGCCGAAACCACAAACGTGGCCGCCGATCTGATGTACGGCAGCG
GAGAAGGCAGAGGCAGCCTGCTTACATGTGGCGACGTGGAAGAGAACCCCG
GACCTATGCAGGCTCTGCAGCAGCAGCCAGTGTTCCCCGATCTGCTGAAAGC
CATTCCTGTCAGCATCCATGTCAACGTCATCCTCTTCTCTGCCATCCTCATTGT
CCTCACTATGGTTGGAACGGCCTTTTTTATGTATAATGCCTTTGGGAAGCCGT
TTGAAACCCTGCACGGACCCCTGGGACTCTATCTCCTGAGCTTCATCTCCGGC
TCTTGCGGCTGCCTCGTGATGATCCTCTTTGCCTCTGAAGTCAAAATTCACCA
CCTGTCTGAGAAAATTGCTAATTACAAAGAAGGGACATACGTTTACAAAACG
CAGAGCGAAAAGTATACGACCAGCTTCTGGCTGACCAAGGGCCACTCTTAAG
AGCTCAGGCCTCTGACCACAGGTGCTGAGTACTCAGCAGCCCTCATATAATA
GGTTTGAAAGTACTCCTTAAAATAAAACACTGTTTCCCTTTGGAACTATTTAC
AAGGATGAAACAACCGTATACCTGAGAAATAACTTGCTCTGGTGTCAATTCG
CTATTCGCCAGCAGACATCAGAACACACCGAGTTTCCAGATGCTGGTTTTTCC
CCTTAAATCAGGAAATACACCTGGACAATTTCTAGAAGACTACAATTCAGTC
TAGCCACAAAGGGGATTTTTTTTTTTTGGTAACAGGCTAGAGCCCGGTTCTGT
AAGTCTTTAGCTGAAATGGTCCAGTACAAAAGCACTGGAAATGAGTGGGCTA
GGAGGACAAGGACCGTCTCCTGCGTGAGGAGTTGGTTGGAGGTCCCCAAGGC
CAGGTACCCCCTGCACTCTTATTGGATTCCTCTCTGTCTTCTTGGAGTTTTGAA
AAACTCCTTCGAACACCAGGCTTTTTTCTTTAGAAAACAAGTCTCCAATCGTT
CTCTGTTCCGTAGAAAGAGAAAGAAAACCTGGAGCAGCTGCTGAAAAATCTA
ATGAGGAACTAAGAGGCAAACCCACCACTGTGCCTTCTAGTTGCCAGCCATC
TGTTGTTTGCCCCTCCCCCGTGCCTTCCTTGACCCTGGAAGGTGCCACTCCCA
CTGTCCTTTCCTAATAAAATGAGGAAATTGCATCGCATTGTCTGAGTAGGTGT
CATTCTATTCTGGGGGGTGGGGTGGGGCAGGACAGCAAGGGGGAGGATTGG
GAAGACAATAGCAGGCATGCTGGGGATGCGGTGGGCTCTATGGAAGCTTGAA
TTCAGCTGACGTGCCTCGGACCGCTAGGAACCCCTAGTGATGGAGTTGGCCA
CTCCCTCTCTGCGCGCTCGCTCGCTCACTGAGGCCGGGCGACCAAAGGTCGC
CCGACGCCCGGGCTTTGCCCGGGCGGCCTCAGTGAGCGAGCGAGCGCGCAGC
TGCCTGCAGG
- CLRN-10
SEQ ID NO: 48
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCATGCCTAGCCAGCAGAAGAAAAT
CATCTTCTGCATGGCCGGCGTGTTCAGCTTCGCCTGTGCTCTGGGAGTTGTGA
CAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAG
ATGCAGTACGGCCTGTTCCATGGCGAAGGCGTCAGACAGTGTGGCCTGGGAG
CCAGACCTTTCAGATTCAGCTTCTTCCCAGACCTGCTGAAGGCTATCCCCGTG
TCCATCCACGTGAACGTGATCCTGTTCAGCGCCATCCTGATCGTGCTGACAAT
GGTCGGAACCGCCTTCTTCATGTACAACGCCTTCGGCAAGCCCTTCGAGACA
CTGCATGGACCTCTGGGCCTGTACCTGCTGAGCTTTATCAGCGGCAGCTGTGG
CTGCCTGGTCATGATTCTGTTCGCCAGCGAAGTGAAGATCCACCACCTGAGC
GAGAAGATCGCCAACTACAAAGAGGGCACCTACGTCTACAAGACCCAGTCC
GAGAAGTACACCACCAGCTTTTGGGTTATCTTCTTCTGTTTCTTCGTGCACTTC
CTGAACGGCCTGCTGATCAGACTGGCCGGCTTCCAGTTTCCATTCGCCAAGA
GCAAGGACGCCGAAACCACAAACGTGGCCGCCGATCTGATGTACGGATCCTA
TCCCTATGATGTGCCAGACTATGCTAAGCGGAAGAGAAGAGGCGAAGGCAG
AGGCAGCCTGCTTACATGTGGCGACGTGGAAGAGAACCCCGGACCTATGCAG
GCTCTGCAGCAGCAGCCAGTGTTCCCCGATCTGCTGAAAGCCATTCCTGTCAG
CATCCATGTCAACGTCATCCTCTTCTCTGCCATCCTCATTGTCCTCACTATGGT
TGGAACGGCCTTTTTTATGTATAATGCCTTTGGGAAGCCGTTTGAAACCCTGC
ACGGACCCCTGGGACTCTATCTCCTGAGCTTCATCTCCGGCTCTTGCGGCTGC
CTCGTGATGATCCTCTTTGCCTCTGAAGTCAAAATTCACCACCTGTCTGAGAA
AATTGCTAATTACAAAGAAGGGACATACGTTTACAAAACGCAGAGCGAAAA
GTATACGACCAGCTTCTGGCTGACCAAGGGCCACTCTGGATCCGACTACAAA
GACCATGACGGTGATTATAAAGATCATGACATCGATTACAAGGATGACGATG
ACAAGTAAGAGCTCGCTGATCAGCCTCGACTGTGCCTTCTAGTTGCCAGCCAT
CTGTTGTTTGCCCCTCCCCCGTGCCTTCCTTGACCCTGGAAGGTGCCACTCCC
ACTGTCCTTTCCTAATAAAATGAGGAAATTGCATCGCATTGTCTGAGTAGGTG
TCATTCTATTCTGGGGGGTGGGGTGGGGCAGGACAGCAAGGGGGAGGATTGG
GAAGACAATAGCAGGCATGCTGGGGATGCGGTGGGCTCTATGGAAGCTTGAA
TTCAGCTGACGTGCCTCGGACCGCTAGGAACCCCTAGTGATGGAGTTGGCCA
CTCCCTCTCTGCGCGCTCGCTCGCTCACTGAGGCCGGGCGACCAAAGGTCGC
CCGACGCCCGGGCTTTGCCCGGGCGGCCTCAGTGAGCGAGCGAGCGCGCAGC
TGCCTGCAGG
- CLRN-10myc
SEQ ID NO: 49
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCATGCCTAGCCAGCAGAAGAAAAT
CATCTTCTGCATGGCCGGCGTGTTCAGCTTCGCCTGTGCTCTGGGAGTTGTGA
CAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAG
ATGCAGTACGGCCTGTTCCATGGCGAAGGCGTCAGACAGTGTGGCCTGGGAG
CCAGACCTTTCAGATTCAGCTTCTTCCCAGACCTGCTGAAGGCTATCCCCGTG
TCCATCCACGTGAACGTGATCCTGTTCAGCGCCATCCTGATCGTGCTGACAAT
GGTCGGAACCGCCTTCTTCATGTACAACGCCTTCGGCAAGCCCTTCGAGACA
CTGCATGGACCTCTGGGCCTGTACCTGCTGAGCTTTATCAGCGGCAGCTGTGG
CTGCCTGGTCATGATTCTGTTCGCCAGCGAAGTGAAGATCCACCACCTGAGC
GAGAAGATCGCCAACTACAAAGAGGGCACCTACGTCTACAAGACCCAGTCC
GAGAAGTACACCACCAGCTTTTGGGTTATCTTCTTCTGTTTCTTCGTGCACTTC
CTGAACGGCCTGCTGATCAGACTGGCCGGCTTCCAGTTTCCATTCGCCAAGA
GCAAGGACGCCGAAACCACAAACGTGGCCGCCGATCTGATGTACGGATCCG
AGCAGAAACTCATCTCTGAAGAAGATCTGGAACAAAAGTTGATTTCAGAAGA
AGATCTGGAACAGAAGCTCATCTCTGAGGAAGATCTGAAGCGGAAGAGAAG
AGGCGAAGGCAGAGGCAGCCTGCTTACATGTGGCGACGTGGAAGAGAACCC
CGGACCTATGCAGGCTCTGCAGCAGCAGCCAGTGTTCCCCGATCTGCTGAAA
GCCATTCCTGTCAGCATCCATGTCAACGTCATCCTCTTCTCTGCCATCCTCATT
GTCCTCACTATGGTTGGAACGGCCTTTTTTATGTATAATGCCTTTGGGAAGCC
GTTTGAAACCCTGCACGGACCCCTGGGACTCTATCTCCTGAGCTTCATCTCCG
GCTCTTGCGGCTGCCTCGTGATGATCCTCTTTGCCTCTGAAGTCAAAATTCAC
CACCTGTCTGAGAAAATTGCTAATTACAAAGAAGGGACATACGTTTACAAAA
CGCAGAGCGAAAAGTATACGACCAGCTTCTGGCTGACCAAGGGCCACTCTGG
ATCCGACTACAAAGACCATGACGGTGATTATAAAGATCATGACATCGATTAC
AAGGATGACGATGACAAGTAAGAGCTCGCTGATCAGCCTCGACTGTGCCTTC
TAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATT
GTCTGAGTAGGTGTCATTCTATTCTGGGGGGTGGGGTGGGGCAGGACAGCAA
GGGGGAGGATTGGGAAGACAATAGCAGGCATGCTGGGGATGCGGTGGGCTC
TATGGAAGCTTGAATTCAGCTGACGTGCCTCGGACCGCTAGGAACCCCTAGT
GATGGAGTTGGCCACTCCCTCTCTGCGCGCTCGCTCGCTCACTGAGGCCGGGC
GACCAAAGGTCGCCCGACGCCCGGGCTTTGCCCGGGCGGCCTCAGTGAGCGA
GCGAGCGCGCAGCTGCCTGCAGG
- CLRN-10NF
SEQ ID NO: 50
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCATGCCTAGCCAGCAGAAGAAAAT
CATCTTCTGCATGGCCGGCGTGTTCAGCTTCGCCTGTGCTCTGGGAGTTGTGA
CAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAG
ATGCAGTACGGCCTGTTCCATGGCGAAGGCGTCAGACAGTGTGGCCTGGGAG
CCAGACCTTTCAGATTCAGCTTCTTCCCAGACCTGCTGAAGGCTATCCCCGTG
TCCATCCACGTGAACGTGATCCTGTTCAGCGCCATCCTGATCGTGCTGACAAT
GGTCGGAACCGCCTTCTTCATGTACAACGCCTTCGGCAAGCCCTTCGAGACA
CTGCATGGACCTCTGGGCCTGTACCTGCTGAGCTTTATCAGCGGCAGCTGTGG
CTGCCTGGTCATGATTCTGTTCGCCAGCGAAGTGAAGATCCACCACCTGAGC
GAGAAGATCGCCAACTACAAAGAGGGCACCTACGTCTACAAGACCCAGTCC
GAGAAGTACACCACCAGCTTTTGGGTTATCTTCTTCTGTTTCTTCGTGCACTTC
CTGAACGGCCTGCTGATCAGACTGGCCGGCTTCCAGTTTCCATTCGCCAAGA
GCAAGGACGCCGAAACCACAAACGTGGCCGCCGATCTGATGTACGGATCCTA
TCCCTATGATGTGCCAGACTATGCTAAGGGCGAAGGCAGAGGCAGCCTGCTT
ACATGTGGCGACGTGGAAGAGAACCCCGGACCTATGCAGGCTCTGCAGCAGC
AGCCAGTGTTCCCCGATCTGCTGAAAGCCATTCCTGTCAGCATCCATGTCAAC
GTCATCCTCTTCTCTGCCATCCTCATTGTCCTCACTATGGTTGGAACGGCCTTT
TTTATGTATAATGCCTTTGGGAAGCCGTTTGAAACCCTGCACGGACCCCTGGG
ACTCTATCTCCTGAGCTTCATCTCCGGCTCTTGCGGCTGCCTCGTGATGATCCT
CTTTGCCTCTGAAGTCAAAATTCACCACCTGTCTGAGAAAATTGCTAATTACA
AAGAAGGGACATACGTTTACAAAACGCAGAGCGAAAAGTATACGACCAGCT
TCTGGCTGACCAAGGGCCACTCTGGATCCGACTACAAAGACCATGACGGTGA
TTATAAAGATCATGACATCGATTACAAGGATGACGATGACAAGTAAGAGCTC
GCTGATCAGCCTCGACTGTGCCTTCTAGTTGCCAGCCATCTGTTGTTTGCCCCT
CCCCCGTGCCTTCCTTGACCCTGGAAGGTGCCACTCCCACTGTCCTTTCCTAA
TAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAG
GCATGCTGGGGATGCGGTGGGCTCTATGGAAGCTTGAATTCAGCTGACGTGC
CTCGGACCGCTAGGAACCCCTAGTGATGGAGTTGGCCACTCCCTCTCTGCGC
GCTCGCTCGCTCACTGAGGCCGGGCGACCAAAGGTCGCCCGACGCCCGGGCT
TTGCCCGGGCGGCCTCAGTGAGCGAGCGAGCGCGCAGCTGCCTGCAGG
- CLRN-11
SEQ ID NO: 51
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCATGCCTAGCCAGCAGAAGAAAAT
CATCTTCTGCATGGCCGGCGTGTTCAGCTTCGCCTGTGCTCTGGGAGTTGTGA
CAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAG
ATGCAGTACGGCCTGTTCCATGGCGAAGGCGTCAGACAGTGTGGCCTGGGAG
CCAGACCTTTCAGATTCAGCTTCTTCCCAGACCTGCTGAAGGCTATCCCCGTG
TCCATCCACGTGAACGTGATCCTGTTCAGCGCCATCCTGATCGTGCTGACAAT
GGTCGGAACCGCCTTCTTCATGTACAACGCCTTCGGCAAGCCCTTCGAGACA
CTGCATGGACCTCTGGGCCTGTACCTGCTGAGCTTTATCAGCGGCAGCTGTGG
CTGCCTGGTCATGATTCTGTTCGCCAGCGAAGTGAAGATCCACCACCTGAGC
GAGAAGATCGCCAACTACAAAGAGGGCACCTACGTCTACAAGACCCAGTCC
GAGAAGTACACCACCAGCTTTTGGGTTATCTTCTTCTGTTTCTTCGTGCACTTC
CTGAACGGCCTGCTGATCAGACTGGCCGGCTTCCAGTTTCCATTCGCCAAGA
GCAAGGACGCCGAAACCACAAACGTGGCCGCCGATCTGATGTACGGATCCTA
TCCCTATGATGTGCCAGACTATGCTAAGCGGAAGAGAAGAGGCGAAGGCAG
AGGCAGCCTGCTTACATGTGGCGACGTGGAAGAGAACCCCGGACCTATGCAG
GCTCTGCAGCAGCAGCCAGTGTTCCCCGATCTGCTGAAAGCCATTCCTGTCAG
CATCCATGTCAACGTCATCCTCTTCTCTGCCATCCTCATTGTCCTCACTATGGT
TGGAACGGCCTTTTTTATGTATAATGCCTTTGGGAAGCCGTTTGAAACCCTGC
ACGGACCCCTGGGACTCTATCTCCTGAGCTTCATCTCCGGCTCTTGCGGCTGC
CTCGTGATGATCCTCTTTGCCTCTGAAGTCAAAATTCACCACCTGTCTGAGAA
AATTGCTAATTACAAAGAAGGGACATACGTTTACAAAACGCAGAGCGAAAA
GTATACGACCAGCTTCTGGCTGACCAAGGGCCACTCTGGATCCGACTACAAA
GACCATGACGGTGATTATAAAGATCATGACATCGATTACAAGGATGACGATG
ACAAGTAAGAGCTCAAGGCAAACCTTTCTATAATTTTACAAGGGAGTAGACT
TGCTTTGGTCACTTTTAGATGTGGTTAATTTTGCATATCCTTTTAGTCTGCATA
TATTAAAGCATCAGGACCCTTCGTGACAATGTTTACAAATTACGTACTAAGG
ATACAGGCTGGAAAGTAAGGGAAGCAGAAGGAAGGCTTTGAAAAGTTGTTTT
ATCTGGTGGGAAATTGCTTGACCCAGGTAGTCAAAGGCAGTTGACTAGAATC
GACAAATTGTTACTCCATATATATATATATGTGTGTGTGTGTGTAAGATGTCT
TCCTATCAAAAAGATATCAAAGGCACATGGAATATATTTTAATAAAAACAAA
TAATATCTCTAATATATCCACACATTTGTTGCCAGATTTCAGAAAACTGAGCT
GCAATCGCTTTCCTAAAACAGTAGTGTATTAAATGAACATCTATAAAATGTAT
CAACACACATTTTAAAAAATTTGTTTAAAGTATACTCTTAGGCCAGGCGTGGT
GACTCACACCTGTAATTCCAGCACTTCAGGAGGCCAAGGTGGGAAGATCATT
TGAGTTCAGGAGTTCGAGTTACAGCCTGGGCAATAAAGTGAGACCCTGTCAC
TAACAAAATTAAAAAATAAAATAAATATAAAATATAGGCTTTAAAAAAGCAT
AGTCTTATTAACCATGTCTGTTGGTCAAAATCTGCAAACTCTAAAAGAAGAA
AAGAAGAAAAAACCAACGTTAGGGTATTTTTCCTCCCGTGCCTGAGTCCCAA
TTACATTCACGACAGTACTTTCAATGAACATAATTGTTAGGACCACTGAGGA
ATCATGAAAAATGATCTCTGCTTAGTACATTTGATGCAAAATGACTTATTAGG
GGCTGTTTTTCTAGCTATAGTGTCTCGAGTACTAATATGCAATTATGAAAATT
ATATTAAATCTGGGATTATGACGGTATCACTGTATCATCTTGGTCTTGTTCTG
GCTGTCACCAAGCATGACCCAGGTCAACTTTTTTTTTCCCCTGAATTACCCAT
CAAATTGATCTGCAGCTGACTAAAGGCCACAGCTGAGCCTGGAACTGACCCT
TCCTTCATCCTCAACCTGCTGTCCTCCAGAAAGCACCAAGGAAAAAGCAGAG
AATGACAGCAAACAGATCACTAGGCCTCTGACCACAGGTGCTGAGTACTCAG
CAGCCCTCATATAATAGGTTTGAAAGTACTCCTTAAAATAAAACACTGTTTCC
CTTTGGAACTATTTACAAGGATGAAACAACCGTATACCTGAGAAATAACTTG
CTCTGGTGTCAATTCGCTATTCGCCAGCAGACATCAGAACACACCGAGTTTCC
AGATGCTGGTTTTTCCCCTTAAATCAGGAAATACACCTGGACAATTTCTAGAA
GACAGGCCTCTGTGCCTTCTAGTTGCCAGCCATCTGTTGTTTGCCCCTCCCCC
GTGCCTTCCTTGACCCTGGAAGGTGCCACTCCCACTGTCCTTTCCTAATAAAA
TGAGGAAATTGCATCGCATTGTCTGAGTAGGTGTCATTCTATTCTGGGGGGTG
GGGTGGGGCAGGACAGCAAGGGGGAGGATTGGGAAGACAATAGCAGGCATG
CTGGGGATGCGGTGGGCTCTATGGAAGCTTGAATTCAGCTGACGTGCCTCGG
ACCGCTAGGAACCCCTAGTGATGGAGTTGGCCACTCCCTCTCTGCGCGCTCGC
TCGCTCACTGAGGCCGGGCGACCAAAGGTCGCCCGACGCCCGGGCTTTGCCC
GGGCGGCCTCAGTGAGCGAGCGAGCGCGCAGCTGCCTGCAGG
- CLRN-11myc
SEQ ID NO: 52
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCATGCCTAGCCAGCAGAAGAAAAT
CATCTTCTGCATGGCCGGCGTGTTCAGCTTCGCCTGTGCTCTGGGAGTTGTGA
CAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAG
ATGCAGTACGGCCTGTTCCATGGCGAAGGCGTCAGACAGTGTGGCCTGGGAG
CCAGACCTTTCAGATTCAGCTTCTTCCCAGACCTGCTGAAGGCTATCCCCGTG
TCCATCCACGTGAACGTGATCCTGTTCAGCGCCATCCTGATCGTGCTGACAAT
GGTCGGAACCGCCTTCTTCATGTACAACGCCTTCGGCAAGCCCTTCGAGACA
CTGCATGGACCTCTGGGCCTGTACCTGCTGAGCTTTATCAGCGGCAGCTGTGG
CTGCCTGGTCATGATTCTGTTCGCCAGCGAAGTGAAGATCCACCACCTGAGC
GAGAAGATCGCCAACTACAAAGAGGGCACCTACGTCTACAAGACCCAGTCC
GAGAAGTACACCACCAGCTTTTGGGTTATCTTCTTCTGTTTCTTCGTGCACTTC
CTGAACGGCCTGCTGATCAGACTGGCCGGCTTCCAGTTTCCATTCGCCAAGA
GCAAGGACGCCGAAACCACAAACGTGGCCGCCGATCTGATGTACGGATCCG
AGCAGAAACTCATCTCTGAAGAAGATCTGGAACAAAAGTTGATTTCAGAAGA
AGATCTGGAACAGAAGCTCATCTCTGAGGAAGATCTGAAGCGGAAGAGAAG
AGGCGAAGGCAGAGGCAGCCTGCTTACATGTGGCGACGTGGAAGAGAACCC
CGGACCTATGCAGGCTCTGCAGCAGCAGCCAGTGTTCCCCGATCTGCTGAAA
GCCATTCCTGTCAGCATCCATGTCAACGTCATCCTCTTCTCTGCCATCCTCATT
GTCCTCACTATGGTTGGAACGGCCTTTTTTATGTATAATGCCTTTGGGAAGCC
GTTTGAAACCCTGCACGGACCCCTGGGACTCTATCTCCTGAGCTTCATCTCCG
GCTCTTGCGGCTGCCTCGTGATGATCCTCTTTGCCTCTGAAGTCAAAATTCAC
CACCTGTCTGAGAAAATTGCTAATTACAAAGAAGGGACATACGTTTACAAAA
CGCAGAGCGAAAAGTATACGACCAGCTTCTGGCTGACCAAGGGCCACTCTGG
ATCCGACTACAAAGACCATGACGGTGATTATAAAGATCATGACATCGATTAC
AAGGATGACGATGACAAGTAAGAGCTCAAGGCAAACCTTTCTATAATTTTAC
AAGGGAGTAGACTTGCTTTGGTCACTTTTAGATGTGGTTAATTTTGCATATCC
TTTTAGTCTGCATATATTAAAGCATCAGGACCCTTCGTGACAATGTTTACAAA
TTACGTACTAAGGATACAGGCTGGAAAGTAAGGGAAGCAGAAGGAAGGCTT
TGAAAAGTTGTTTTATCTGGTGGGAAATTGCTTGACCCAGGTAGTCAAAGGC
AGTTGACTAGAATCGACAAATTGTTACTCCATATATATATATATGTGTGTGTG
TGTGTAAGATGTCTTCCTATCAAAAAGATATCAAAGGCACATGGAATATATTT
TAATAAAAACAAATAATATCTCTAATATATCCACACATTTGTTGCCAGATTTC
AGAAAACTGAGCTGCAATCGCTTTCCTAAAACAGTAGTGTATTAAATGAACA
TCTATAAAATGTATCAACACACATTTTAAAAAATTTGTTTAAAGTATACTCTT
AGGCCAGGCGTGGTGACTCACACCTGTAATTCCAGCACTTCAGGAGGCCAAG
GTGGGAAGATCATTTGAGTTCAGGAGTTCGAGTTACAGCCTGGGCAATAAAG
TGAGACCCTGTCACTAACAAAATTAAAAAATAAAATAAATATAAAATATAGG
CTTTAAAAAAGCATAGTCTTATTAACCATGTCTGTTGGTCAAAATCTGCAAAC
TCTAAAAGAAGAAAAGAAGAAAAAACCAACGTTAGGGTATTTTTCCTCCCGT
GCCTGAGTCCCAATTACATTCACGACAGTACTTTCAATGAACATAATTGTTAG
GACCACTGAGGAATCATGAAAAATGATCTCTGCTTAGTACATTTGATGCAAA
ATGACTTATTAGGGGCTGTTTTTCTAGCTATAGTGTCTCGAGTACTAATATGC
AATTATGAAAATTATATTAAATCTGGGATTATGACGGTATCACTGTATCATCT
TGGTCTTGTTCTGGCTGTCACCAAGCATGACCCAGGTCAACTTTTTTTTTCCCC
TGAATTACCCATCAAATTGATCTGCAGCTGACTAAAGGCCACAGCTGAGCCT
GGAACTGACCCTTCCTTCATCCTCAACCTGCTGTCCTCCAGAAAGCACCAAGG
AAAAAGCAGAGAATGACAGCAAACAGATCACTAGGCCTCTGACCACAGGTG
CTGAGTACTCAGCAGCCCTCATATAATAGGTTTGAAAGTACTCCTTAAAATAA
AACACTGTTTCCCTTTGGAACTATTTACAAGGATGAAACAACCGTATACCTGA
GAAATAACTTGCTCTGGTGTCAATTCGCTATTCGCCAGCAGACATCAGAACA
CACCGAGTTTCCAGATGCTGGTTTTTCCCCTTAAATCAGGAAATACACCTGGA
CAATTTCTAGAAGACAGGCCTCTGTGCCTTCTAGTTGCCAGCCATCTGTTGTT
TGCCCCTCCCCCGTGCCTTCCTTGACCCTGGAAGGTGCCACTCCCACTGTCCT
TTCCTAATAAAATGAGGAAATTGCATCGCATTGTCTGAGTAGGTGTCATTCTA
TTCTGGGGGGTGGGGTGGGGCAGGACAGCAAGGGGGAGGATTGGGAAGACA
ATAGCAGGCATGCTGGGGATGCGGTGGGCTCTATGGAAGCTTGAATTCAGCT
GACGTGCCTCGGACCGCTAGGAACCCCTAGTGATGGAGTTGGCCACTCCCTC
TCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGCAGCTGCCTGC
AGG
- CLRN-11NF
SEQ ID NO: 53
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCATGCCTAGCCAGCAGAAGAAAAT
CATCTTCTGCATGGCCGGCGTGTTCAGCTTCGCCTGTGCTCTGGGAGTTGTGA
CAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAG
ATGCAGTACGGCCTGTTCCATGGCGAAGGCGTCAGACAGTGTGGCCTGGGAG
CCAGACCTTTCAGATTCAGCTTCTTCCCAGACCTGCTGAAGGCTATCCCCGTG
TCCATCCACGTGAACGTGATCCTGTTCAGCGCCATCCTGATCGTGCTGACAAT
GGTCGGAACCGCCTTCTTCATGTACAACGCCTTCGGCAAGCCCTTCGAGACA
CTGCATGGACCTCTGGGCCTGTACCTGCTGAGCTTTATCAGCGGCAGCTGTGG
CTGCCTGGTCATGATTCTGTTCGCCAGCGAAGTGAAGATCCACCACCTGAGC
GAGAAGATCGCCAACTACAAAGAGGGCACCTACGTCTACAAGACCCAGTCC
GAGAAGTACACCACCAGCTTTTGGGTTATCTTCTTCTGTTTCTTCGTGCACTTC
CTGAACGGCCTGCTGATCAGACTGGCCGGCTTCCAGTTTCCATTCGCCAAGA
GCAAGGACGCCGAAACCACAAACGTGGCCGCCGATCTGATGTACGGATCCTA
TCCCTATGATGTGCCAGACTATGCTAAGGGCGAAGGCAGAGGCAGCCTGCTT
ACATGTGGCGACGTGGAAGAGAACCCCGGACCTATGCAGGCTCTGCAGCAGC
AGCCAGTGTTCCCCGATCTGCTGAAAGCCATTCCTGTCAGCATCCATGTCAAC
GTCATCCTCTTCTCTGCCATCCTCATTGTCCTCACTATGGTTGGAACGGCCTTT
TTTATGTATAATGCCTTTGGGAAGCCGTTTGAAACCCTGCACGGACCCCTGGG
ACTCTATCTCCTGAGCTTCATCTCCGGCTCTTGCGGCTGCCTCGTGATGATCCT
CTTTGCCTCTGAAGTCAAAATTCACCACCTGTCTGAGAAAATTGCTAATTACA
AAGAAGGGACATACGTTTACAAAACGCAGAGCGAAAAGTATACGACCAGCT
TCTGGCTGACCAAGGGCCACTCTGGATCCGACTACAAAGACCATGACGGTGA
TTATAAAGATCATGACATCGATTACAAGGATGACGATGACAAGTAAGAGCTC
AAGGCAAACCTTTCTATAATTTTACAAGGGAGTAGACTTGCTTTGGTCACTTT
TAGATGTGGTTAATTTTGCATATCCTTTTAGTCTGCATATATTAAAGCATCAG
GACCCTTCGTGACAATGTTTACAAATTACGTACTAAGGATACAGGCTGGAAA
GTAAGGGAAGCAGAAGGAAGGCTTTGAAAAGTTGTTTTATCTGGTGGGAAAT
TGCTTGACCCAGGTAGTCAAAGGCAGTTGACTAGAATCGACAAATTGTTACT
CCATATATATATATATGTGTGTGTGTGTGTAAGATGTCTTCCTATCAAAAAGA
TATCAAAGGCACATGGAATATATTTTAATAAAAACAAATAATATCTCTAATA
TATCCACACATTTGTTGCCAGATTTCAGAAAACTGAGCTGCAATCGCTTTCCT
AAAACAGTAGTGTATTAAATGAACATCTATAAAATGTATCAACACACATTTT
AAAAAATTTGTTTAAAGTATACTCTTAGGCCAGGCGTGGTGACTCACACCTGT
AATTCCAGCACTTCAGGAGGCCAAGGTGGGAAGATCATTTGAGTTCAGGAGT
TCGAGTTACAGCCTGGGCAATAAAGTGAGACCCTGTCACTAACAAAATTAAA
AAATAAAATAAATATAAAATATAGGCTTTAAAAAAGCATAGTCTTATTAACC
ATGTCTGTTGGTCAAAATCTGCAAACTCTAAAAGAAGAAAAGAAGAAAAAA
CCAACGTTAGGGTATTTTTCCTCCCGTGCCTGAGTCCCAATTACATTCACGAC
AGTACTTTCAATGAACATAATTGTTAGGACCACTGAGGAATCATGAAAAATG
ATCTCTGCTTAGTACATTTGATGCAAAATGACTTATTAGGGGCTGTTTTTCTA
GCTATAGTGTCTCGAGTACTAATATGCAATTATGAAAATTATATTAAATCTGG
GATTATGACGGTATCACTGTATCATCTTGGTCTTGTTCTGGCTGTCACCAAGC
ATGACCCAGGTCAACTTTTTTTTTCCCCTGAATTACCCATCAAATTGATCTGC
AGCTGACTAAAGGCCACAGCTGAGCCTGGAACTGACCCTTCCTTCATCCTCA
ACCTGCTGTCCTCCAGAAAGCACCAAGGAAAAAGCAGAGAATGACAGCAAA
CAGATCACTAGGCCTCTGACCACAGGTGCTGAGTACTCAGCAGCCCTCATAT
AATAGGTTTGAAAGTACTCCTTAAAATAAAACACTGTTTCCCTTTGGAACTAT
TTACAAGGATGAAACAACCGTATACCTGAGAAATAACTTGCTCTGGTGTCAA
TTCGCTATTCGCCAGCAGACATCAGAACACACCGAGTTTCCAGATGCTGGTTT
TTCCCCTTAAATCAGGAAATACACCTGGACAATTTCTAGAAGACAGGCCTCT
GTGCCTTCTAGTTGCCAGCCATCTGTTGTTTGCCCCTCCCCCGTGCCTTCCTTG
ACCCTGGAAGGTGCCACTCCCACTGTCCTTTCCTAATAAAATGAGGAAATTGC
ATCGCATTGTCTGAGTAGGTGTCATTCTATTCTGGGGGGTGGGGTGGGGCAG
GACAGCAAGGGGGAGGATTGGGAAGACAATAGCAGGCATGCTGGGGATGCG
GTGGGCTCTATGGAAGCTTGAATTCAGCTGACGTGCCTCGGACCGCTAGGAA
CCCCTAGTGATGGAGTTGGCCACTCCCTCTCTGCGCGCTCGCTCGCTCACTGA
GGCCGGGCGACCAAAGGTCGCCCGACGCCCGGGCTTTGCCCGGGCGGCCTCA
GTGAGCGAGCGAGCGCGCAGCTGCCTGCAGG
- CLRN-12
SEQ ID NO: 54
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTAGGAGATACTTGAAGGCAGTTTGAAAGAC
TTGTTTTACAGATTCTTAGTCCAAAGATTTCCAATTAGGGAGAAGAAGCAGC
AGAAAAGGAGAAAAGCCAAGTATGAGTGATGATGAGGCCTTCATCTACTGAC
ATTTAACCTGGCGAGAACCGTCGATGGTGAAGTTGCCTTTTCAGCTGGGAGCT
GTCCGTTCAGCTTCCGTAATAAATGCAGTCAAAGAGGCAGTCCCTTCCCATTG
CTCACAAAGGTCTTGTTTTTGAACCTCGCCCTCACAGAAGCCGTTTCTCATCG
CCACCATGCCTAGCCAGCAGAAGAAAATCATCTTCTGCATGGCCGGCGTGTT
CAGCTTCGCCTGTGCTCTGGGAGTTGTGACAGCCCTGGGAACCCCTCTGTGGA
TCAAAGCCACAGTGCTGTGCAAGACAGGCGCCCTGCTGGTTAATGCCTCTGG
CCAAGAGCTGGACAAGTTCATGGGCGAGATGCAGTACGGCCTGTTCCATGGC
GAAGGCGTCAGACAGTGTGGCCTGGGAGCCAGACCTTTCAGATTCAGCTTCT
TCCCAGACCTGCTGAAGGCTATCCCCGTGTCCATCCACGTGAACGTGATCCTG
TTCAGCGCCATCCTGATCGTGCTGACAATGGTCGGAACCGCCTTCTTCATGTA
CAACGCCTTCGGCAAGCCCTTCGAGACACTGCATGGACCTCTGGGCCTGTAC
CTGCTGAGCTTTATCAGCGGCAGCTGTGGCTGCCTGGTCATGATTCTGTTCGC
CAGCGAAGTGAAGATCCACCACCTGAGCGAGAAGATCGCCAACTACAAAGA
GGGCACCTACGTCTACAAGACCCAGTCCGAGAAGTACACCACCAGCTTTTGG
GTTATCTTCTTCTGTTTCTTCGTGCACTTCCTGAACGGCCTGCTGATCAGACTG
GCCGGCTTCCAGTTTCCATTCGCCAAGAGCAAGGACGCCGAAACCACAAACG
TGGCCGCCGATCTGATGTACGGATCCTATCCCTATGATGTGCCAGACTATGCT
TAAGAGCTCAAGGCAAACCTTTCTATAATTTTACAAGGGAGTAGACTTGCTTT
GGTCACTTTTAGATGTGGTTAATTTTGCATATCCTTTTAGTCTGCATATATTAA
AGCATCAGGACCCTTCGTGACAATGTTTACAAATTACGTACTAAGGATACAG
GCTGGAAAGTAAGGGAAGCAGAAGGAAGGCTTTGAAAAGTTGTTTTATCTGG
TGGGAAATTGCTTGACCCAGGTAGTCAAAGGCAGTTGACTAGAATCGACAAA
TTGTTACTCCATATATATATATGTGTGTGTGTGTGTGTAAGATGTCTTCCTATC
AAAAAGATATCAAAGGCACATGGAATATATTTTAATAAAAACAAATAATATC
TCTAATATATCCACACATTTGTTGCCAGATTTCAGAAAACTGAGCTGCAATCG
CTTTCCTAAAACAGTAGTGTATTAAATGAACATCTATAAAATGTATCAACACA
CATTTTAAAAAATTTGTTTAAAGTATACTCTTAGGCCAGGCGTGGTGACTCAC
ACCTGTAATTCCAGCACTTCAGGAGGCCAAGGTGGGAAGATCATTTGAGTTC
AGGAGTTCGAGTTACAGTCTGGGCAATAAAGTGAGACCCTGTCACTAACAAA
ATTAAAAAATAAAATAAATATAAAATATAGGCTTTAAAAAAGCATAGTCTTA
TTAACCATGTCTGTTGGTCAAAATCTGCAAACTCTAAAAGAAGAAAAGAAGA
AAAAACCAACGTTAGGGTATTTTTCCTCCCGTGCCTGAGTCCCAATTACATTC
ACGACAGTACTTTCAATGAACATAATTGTTAGGACCACTGAGGAATCATGAA
AAATGATCTCTGCTTAGTACATTTGATGCAAAATGACTTATTAGGGGCTGTTT
TTCTAGCTATAGTGTCTCGAGTACTAATATGCAATTATGAAAATTATATTAAA
TCTGGGATTATGACGGTATCACTGTATCATCTTGGTCTTGTTCTGGCTGTCACC
AAGCATGACCCAGGTCAACTTTTTTTTTCCCCTGAATTACCCATCAAATTGAT
CTGCAGCTGACTAAAGGCCACAGCTGAGCCTGGAACTGACCCTTCCTTCATC
CTCAACCTGCTGTCCTCCAGAAAGCACCAAGGAAAAAGCAGAGAATGACAG
CAAACAGATCACTAGGCCTCTGACCACAGGTGCTGAGTACTCAGCAGCCCTC
ATATAATAGGTTTGAAAGTACTCCTTAAAATAAAACACTGTTTCCCTTTGGAA
CTATTTACAAGGATGAAACAACCGTATACCTGAGAAATAACTTGCTCTGGTG
TCAATTCGCTATTCGCCAGCAGACATCAGAACACACCGAGTTTCCAGATGCT
CTGTGCCTTCTAGTTGCCAGCCATCTGTTGTTTGCCCCTCCCCCGTGCCTTCCT
TGACCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATG
CGGTGGGCTCTATGGAAGCTTGAATTCAGCTGACGTGCCTCGGACCGCTAGG
AACCCCTAGTGATGGAGTTGGCCACTCCCTCTCTGCGCGCTCGCTCGCTCACT
GAGGCCGGGCGACCAAAGGTCGCCCGACGCCCGGGCTTTGCCCGGGCGGCCT
CAGTGAGCGAGCGAGCGCGCAGCTGCCTGCAGG
- CLRN-13
SEQ ID NO: 55
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCATGCCTAGCCAGCAGAAGAAAAT
CATCTTCTGCATGGCCGGCGTGTTCAGCTTCGCCTGTGCTCTGGGAGTTGTGA
CAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAG
ATGCAGTACGGCCTGTTCCATGGCGAAGGCGTCAGACAGTGTGGCCTGGGAG
CCAGACCTTTCCGGTTCAGCTGCTACTTTCTGGACCCCTTCATGGGCCTGCCT
ACCGGCGTTCCACATCTGCTGTCTCTGCCTTGCAGCACCAGCTGCAGAAGAG
AGCACACCAGCGAGAGAGTGCAAGAGCCTGCCGGCTGTTTTTCTGCCGTGCG
GTCTAAACTGCACGCCGGACCTGCTGCCGCCACCAGCTTTTCTAGATTCGCCC
AGAGCAACCCCAGCGAGCACCCTAGACAGTGTCACAGCCTGCTGTGTCACCC
CTACTGTGTGAATCACGGCGGCGATTCTCTGCTGCATGTGCAGTGCTTTTGGA
AAACCTTCGGCAGCGACTACAAGGACCACGACGGCGATTATAAGGATCACG
ATATCGATTACAAGGACGATGACGACAAGGGCGAAGGCAGAGGCTCCCTGCT
GACATGCGGAGATGTCGAAGAGAACCCCGGACCTATGCCTTCTCAGCAGAAA
AAGATTATTTTCTGTATGGCTGGGGTGTTCTCCTTCGCTTGCGCCCTGGGTGTT
GTTACCGCTCTCGGAACACCACTGTGGATTAAGGCTACCGTCCTGTGTAAAAC
CGGCGCTCTGCTCGTGAATGCCAGCGGACAAGAACTGGATAAGTTTATGGGA
GAAATGCAATATGGGCTCTTTCACGGCGAGGGTGTTAGACAGTGCGGACTCG
GCGCTAGACCCTTCAGATTCAGCTTCTTCCCAGACCTGCTGAAGGCTATCCCC
GTGTCCATCCACGTGAACGTGATCCTGTTCAGCGCCATCCTGATCGTGCTGAC
AATGGTCGGAACCGCCTTCTTCATGTACAACGCCTTCGGCAAGCCCTTCGAG
ACACTGCATGGACCTCTGGGCCTGTACCTGCTGAGCTTTATCAGCGGCAGCTG
TGGCTGCCTGGTCATGATTCTGTTCGCCAGCGAAGTGAAGATCCACCACCTGA
GCGAGAAGATCGCCAACTACAAAGAGGGCACCTACGTCTACAAGACCCAGT
CCGAGAAGTACACCACAAGCTTTTGGGTTATCTTCTTCTGTTTCTTCGTGCACT
TCCTGAACGGCCTGCTGATCAGACTGGCCGGCTTCCAGTTTCCATTCGCCAAG
TCCAAGGACGCCGAAACCACAAACGTGGCCGCCGACCTGATGTACAGCGGCT
ACCCCTACGACGTGCCAGATTATGCATAAGAGCTCAAGGCAAACCTTTCTAT
AATTTTACAAGGGAGTAGACTTGCTTTGGTCACTTTTAGATGTGGTTAATTTT
GCATATCCTTTTAGTCTGCATATATTAAAGCATCAGGACCCTTCGTGACAATG
TTTACAAATTACGTACTAAGGATACAGGCTGGAAAGTAAGGGAAGCAGAAG
GAAGGCTTTGAAAAGTTGTTTTATCTGGTGGGAAATTGCTTGACCCAGGTAGT
CAAAGGCAGTTGACTAGAATCGACAAATTGTTACTCCATATATATATATGTGT
GTGTGTGTGTGTGTGTGTGTGTGTAAGATGTCTTCCTATCAAAAAGATATCAA
AGGCACATGGAATATATTTTAATAAAAACAAATAATATCTCTAATATATCCA
CACATTTGTTGCCAGATTTCAGAAAACTGAGCTGCAATCGCTTTCCTAAAACA
GTAGTGTATTAAATGAACATCTATAAAATGTATCAACACACATTTTAAAAAA
TTTGTTTAAAGTATACTCTTAGGCCAGGCGTGGTGACTCACACCTGTAATTCC
AGCACTTCAGGAGGCCAAGGTGGGAAGATCATTTGAGTTCAGGAGTTCGAGT
TACAGGCTGATCAGCCTCGACTGTGCCTTCTAGTTGCCAGCCATCTGTTGTTT
GCCCCTCCCCCGTGCCTTCCTTGACCCTGGAAGGTGCCACTCCCACTGTCCTT
TCCTAATAAAATGAGGAAATTGCATCGCATTGTCTGAGTAGGTGTCATTCTAT
TCTGGGGGGTGGGGTGGGGCAGGACAGCAAGGGGGAGGATTGGGAAGACAA
TAGCAGGCATGCTGGGGATGCGGTGGGCTCTATGGAAGCTTGAATTCAGCTG
ACGTGCCTCGGACCGCTAGGAACCCCTAGTGATGGAGTTGGCCACTCCCTCT
CTGCGCGCTCGCTCGCTCACTGAGGCCGGGCGACCAAAGGTCGCCCGACGCC
CGGGCTTTGCCCGGGCGGCCTCAGTGAGCGAGCGAGCGCGCAGCTGCCTGCA
GG
- CLRN-14
SEQ ID NO: 56
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCATGCCTAGCCAGCAGAAGAAAAT
CATCTTCTGCATGGCCGGCGTGTTCAGCTTCGCCTGTGCTCTGGGAGTTGTGA
CAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAG
ATGCAGTACGGCCTGTTCCATGGCGAAGGCGTCAGACAGTGTGGCCTGGGAG
CCAGACCTTTCCGGTTCAGCTGCTACTTTCTGGACCCCTTCATGGGCCTGCCT
ACCGGCGTTCCACATCTGCTGTCTCTGCCTTGCAGCACCAGCTGCAGAAGAG
AGCACACCAGCGAGAGAGTGCAAGAGCCTGCCGGCTGTTTTTCTGCCGTGCG
GTCTAAACTGCACGCCGGACCTGCTGCCGCCACCAGCTTTTCTAGATTCGCCC
AGAGCAACCCCAGCGAGCACCCTAGACAGTGTCACAGCCTGCTGTGTCACCC
CTACTGTGTGAATCACGGCGGCGATTCTCTGCTGCATGTGCAGTGCTTTTGGA
AAACCTTCGGCAGCGGCGAAGGCAGAGGCTCCCTGCTGACATGCGGAGATGT
CGAAGAGAACCCCGGACCTATGCCTTCTCAGCAGAAAAAGATTATTTTCTGT
ATGGCTGGGGTGTTCTCCTTCGCTTGCGCCCTGGGTGTTGTTACCGCTCTCGG
AACACCACTGTGGATTAAGGCTACCGTCCTGTGTAAAACCGGCGCTCTGCTC
GTGAATGCCAGCGGACAAGAACTGGATAAGTTTATGGGAGAAATGCAATATG
GGCTCTTTCACGGCGAGGGTGTTAGACAGTGCGGACTCGGCGCTAGACCCTT
CAGATTCAGCTTCTTCCCAGACCTGCTGAAGGCTATCCCCGTGTCCATCCACG
TGAACGTGATCCTGTTCAGCGCCATCCTGATCGTGCTGACAATGGTCGGAACC
GCCTTCTTCATGTACAACGCCTTCGGCAAGCCCTTCGAGACACTGCATGGACC
TCTGGGCCTGTACCTGCTGAGCTTTATCAGCGGCAGCTGTGGCTGCCTGGTCA
TGATTCTGTTCGCCAGCGAAGTGAAGATCCACCACCTGAGCGAGAAGATCGC
CAACTACAAAGAGGGCACCTACGTCTACAAGACCCAGTCCGAGAAGTACACC
ACAAGCTTTTGGGTTATCTTCTTCTGTTTCTTCGTGCACTTCCTGAACGGCCTG
CTGATCAGACTGGCCGGCTTCCAGTTTCCATTCGCCAAGTCCAAGGACGCCG
AAACCACAAACGTGGCCGCCGACCTGATGTACTAAGAGCTCAAGGCAAACCT
TTCTATAATTTTACAAGGGAGTAGACTTGCTTTGGTCACTTTTAGATGTGGTT
AATTTTGCATATCCTTTTAGTCTGCATATATTAAAGCATCAGGACCCTTCGTG
ACAATGTTTACAAATTACGTACTAAGGATACAGGCTGGAAAGTAAGGGAAGC
AGAAGGAAGGCTTTGAAAAGTTGTTTTATCTGGTGGGAAATTGCTTGACCCA
GGTAGTCAAAGGCAGTTGACTAGAATCGACAAATTGTTACTCCATATATATA
TATGTGTGTGTGTGTGTGTGTGTGTGTGTGTAAGATGTCTTCCTATCAAAAAG
ATATCAAAGGCACATGGAATATATTTTAATAAAAACAAATAATATCTCTAAT
ATATCCACACATTTGTTGCCAGATTTCAGAAAACTGAGCTGCAATCGCTTTCC
TAAAACAGTAGTGTATTAAATGAACATCTATAAAATGTATCAACACACATTTT
AAAAAATTTGTTTAAAGTATACTCTTAGGCCAGGCGTGGTGACTCACACCTGT
AATTCCAGCACTTCAGGAGGCCAAGGTGGGAAGATCATTTGAGTTCAGGAGT
TCGAGTTACAGgctgatcagcctcgaCTGTGCCTTCTAGTTGCCAGCCATCTGTTGTTT
GCCCCTCCCCCGTGCCTTCCTTGACCCTGGAAGGTGCCACTCCCACTGTCCTT
TCCTAATAAAATGAGGAAATTGCATCGCATTGTCTGAGTAGGTGTCATTCTAT
TCTGGGGGGTGGGGTGGGGCAGGACAGCAAGGGGGAGGATTGGGAAGACAA
TAGCAGGCATGCTGGGGATGCGGTGGGCTCTATGGAAGCTTGAATTCAGCTG
ACGTGCCTCGGACCGCTAGGAACCCCTAGTGATGGAGTTGGCCACTCCCTCT
CTGCGCGCTCGCTCGCTCACTGAGGCCGGGCGACCAAAGGTCGCCCGACGCC
CGGGCTTTGCCCGGGCGGCCTCAGTGAGCGAGCGAGCGCGCAGCTGCCTGCA
GG
- CLRN-15
SEQ ID NO: 57
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCATGCCTAGCCAGCAGAAGAAAAT
CATCTTCTGCATGGCCGGCGTGTTCAGCTTCGCCTGTGCTCTGGGAGTTGTGA
CAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAG
ATGCAGTACGGCCTGTTCCATGGCGAAGGCGTCAGACAGTGTGGCCTGGGAG
CCAGACCTTTCCGGTTCAGCTGCTACTTTCTGGACCCCTTCATGGGCCTGCCT
ACCGGCGTTCCACATCTGCTGTCTCTGCCTTGCAGCACCAGCTGCAGAAGAG
AGCACACCAGCGAGAGAGTGCAAGAGCCTGCCGGCTGTTTTTCTGCCGTGCG
GTCTAAACTGCACGCCGGACCTGCTGCCGCCACCAGCTTTTCTAGATTCGCCC
AGAGCAACCCCAGCGAGCACCCTAGACAGTGTCACAGCCTGCTGTGTCACCC
CTACTGTGTGAATCACGGCGGCGATTCTCTGCTGCATGTGCAGTGCTTTTGGA
AAACCTTCGGCAGCGACTACAAGGACCACGACGGCGATTATAAGGATCACG
ATATCGATTACAAGGACGATGACGACAAGTAAGAGCTCAAGGCAAACCTTTC
TATAATTTTACAAGGGAGTAGACTTGCTTTGGTCACTTTTAGATGTGGTTAAT
TTTGCATATCCTTTTAGTCTGCATATATTAAAGCATCAGGACCCTTCGTGACA
ATGTTTACAAATTACGTACTAAGGATACAGGCTGGAAAGTAAGGGAAGCAGA
AGGAAGGCTTTGAAAAGTTGTTTTATCTGGTGGGAAATTGCTTGACCCAGGT
AGTCAAAGGCAGTTGACTAGAATCGACAAATTGTTACTCCATATATATATAT
GTGTGTGTGTGTGTGTGTGTGTGTGTGTAAGATGTCTTCCTATCAAAAAGATA
TCAAAGGCACATGGAATATATTTTAATAAAAACAAATAATATCTCTAATATA
TCCACACATTTGTTGCCAGATTTCAGAAAACTGAGCTGCAATCGCTTTCCTAA
AACAGTAGTGTATTAAATGAACATCTATAAAATGTATCAACACACATTTTAA
AAAATTTGTTTAAAGTATACTCTTAGGCCAGGCGTGGTGACTCACACCTGTAA
TTCCAGCACTTCAGGAGGCCAAGGTGGGAAGATCATTTGAGTTCAGGAGTTC
GAGTTACAGgctgatcagcctcgaCTGTGCCTTCTAGTTGCCAGCCATCTGTTGTTTGC
CCCTCCCCCGTGCCTTCCTTGACCCTGGAAGGTGCCACTCCCACTGTCCTTTC
CTAATAAAATGAGGAAATTGCATCGCATTGTCTGAGTAGGTGTCATTCTATTC
TGGGGGGTGGGGTGGGGCAGGACAGCAAGGGGGAGGATTGGGAAGACAATA
GCAGGCATGCTGGGGATGCGGTGGGCTCTATGGAAGCTTGAATTCAGCTGAC
GTGCCTCGGACCGCTAGGAACCCCTAGTGATGGAGTTGGCCACTCCCTCTCTG
CGCGCTCGCTCGCTCACTGAGGCCGGGCGACCAAAGGTCGCCCGACGCCCGG
GCTTTGCCCGGGCGGCCTCAGTGAGCGAGCGAGCGCGCAGCTGCCTGCAGG
- CLRN-16
SEQ ID NO: 58
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGT
TTAATGACGGCTCGTTTCTTTTCTGTGGCTGCGTGAAAGCCTTAAAGGGCTCC
GGGAGGGCCCTTTGTGCGGGGGGGAGCGGCTCGGGGGGTGCGTGCGTGTGTG
TGTGCGTGGGGAGCGCCGCGTGCGGCCCGCGCTGCCCGGCGGCTGTGAGCGC
TGCGGGCGCGGCGCGGGGCTTTGTGCGCTCCGCGTGTGCGCGAGGGGAGCGC
GGCCGGGGGCGGTGCCCCGCGGTGCGGGGGGGCTGCGAGGGGAACAAAGGC
TGCGTGCGGGGTGTGTGCGTGGGGGGGTGAGCAGGGGGTGTGGGCGCGGCG
GTCGGGCTGTAACCCCCCCCTGCACCCCCCTCCCCGAGTTGCTGAGCACGGCC
CGGCTTCGGGTGCGGGGCTCCGTGCGGGGCGTGGCGCGGGGCTCGCCGTGCC
GGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGG
CTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGA
GGCGCCGCCGCACCCCCTCTAGCGGGCGCGGGGCGAAGCGGTGCGGCGCCG
GCAGGAAGGAAATGGGCGGGGAGGGCCTTCGTGCGTCGCCGCGCCGCCGTC
CCCTTCTCCCTCTCCAGCCTCGGGGCTGTCCGCGGGGGGACGGCTGCCTTCGG
GGGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGGGCA
ACGTGCTGGTTATTGTGACCGGTGCCACCATGCCTAGCCAGCAGAAGAAAAT
CATCTTCTGCATGGCCGGCGTGTTCAGCTTCGCCTGTGCTCTGGGAGTTGTGA
CAGCCCTGGGAACCCCTCTGTGGATCAAAGCCACAGTGCTGTGCAAGACAGG
CGCCCTGCTGGTTAATGCCTCTGGCCAAGAGCTGGACAAGTTCATGGGCGAG
ATGCAGTACGGCCTGTTCCATGGCGAAGGCGTCAGACAGTGTGGCCTGGGAG
CCAGACCTTTCCGGTTCAGCTGCTACTTTCTGGACCCCTTCATGGGCCTGCCT
ACCGGCGTTCCACATCTGCTGTCTCTGCCTTGCAGCACCAGCTGCAGAAGAG
AGCACACCAGCGAGAGAGTGCAAGAGCCTGCCGGCTGTTTTTCTGCCGTGCG
GTCTAAACTGCACGCCGGACCTGCTGCCGCCACCAGCTTTTCTAGATTCGCCC
AGAGCAACCCCAGCGAGCACCCTAGACAGTGTCACAGCCTGCTGTGTCACCC
CTACTGTGTGAATCACGGCGGCGATTCTCTGCTGCATGTGCAGTGCTTTTGGA
AAACCTTCGGCAGCTAAGAGCTCAAGGCAAACCTTTCTATAATTTTACAAGG
GAGTAGACTTGCTTTGGTCACTTTTAGATGTGGTTAATTTTGCATATCCTTTTA
GTCTGCATATATTAAAGCATCAGGACCCTTCGTGACAATGTTTACAAATTACG
TACTAAGGATACAGGCTGGAAAGTAAGGGAAGCAGAAGGAAGGCTTTGAAA
AGTTGTTTTATCTGGTGGGAAATTGCTTGACCCAGGTAGTCAAAGGCAGTTGA
CTAGAATCGACAAATTGTTACTCCATATATATATATGTGTGTGTGTGTGTGTG
TGTGTGTGTGTAAGATGTCTTCCTATCAAAAAGATATCAAAGGCACATGGAA
TATATTTTAATAAAAACAAATAATATCTCTAATATATCCACACATTTGTTGCC
AGATTTCAGAAAACTGAGCTGCAATCGCTTTCCTAAAACAGTAGTGTATTAA
ATGAACATCTATAAAATGTATCAACACACATTTTAAAAAATTTGTTTAAAGTA
TACTCTTAGGCCAGGCGTGGTGACTCACACCTGTAATTCCAGCACTTCAGGAG
GCCAAGGTGGGAAGATCATTTGAGTTCAGGAGTTCGAGTTACAGgctgatcagcctc
gaCTGTGCCTTCTAGTTGCCAGCCATCTGTTGTTTGCCCCTCCCCCGTGCCTTC
CTTGACCCTGGAAGGTGCCACTCCCACTGTCCTTTCCTAATAAAATGAGGAAA
TTGCATCGCATTGTCTGAGTAGGTGTCATTCTATTCTGGGGGGTGGGGTGGGG
CAGGACAGCAAGGGGGAGGATTGGGAAGACAATAGCAGGCATGCTGGGGAT
GCGGTGGGCTCTATGGAAGCTTGAATTCAGCTGACGTGCCTCGGACCGCTAG
GAACCCCTAGTGATGGAGTTGGCCACTCCCTCTCTGCGCGCTCGCTCGCTCAC
TGAGGCCGGGCGACCAAAGGTCGCCCGACGCCCGGGCTTTGCCCGGGCGGCC
TCAGTGAGCGAGCGAGCGCGCAGCTGCCTGCAGG
- CLRN-17
SEQ ID NO: 59
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCT
- CLRN-18
SEQ ID NO: 60
CCTGCAGGCAGCTGCGCGCTCGCTCGCTCACTGAGGCCGCCCGGGCGTCGGG
CGACCTTTGGTCGCCCGGCCTCAGTGAGCGAGCGAGCGCGCAGAGAGGGAGT
GGCCAACTCCATCACTAGGGGTTCCTGCGGCCGCACGCGTGACATTGATTATT
GACTAGTTATTAATAGTAATCAATTACGGGGTCATTAGTTCATAGCCCATATA
TGGAGTTCCGCGTTACATAACTTACGGTAAATGGCCCGCCTGGCTGACCGCC
CAACGACCCCCGCCCATTGACGTCAATAATGACGTATGTTCCCATAGTAACG
CCAATAGGGACTTTCCATTGACGTCAATGGGTGGACTATTTACGGTAAACTGC
CCACTTGGCAGTACATCAAGTGTATCATATGCCAAGTACGCCCCCTATTGACG
TCAATGACGGTAAATGGCCCGCCTGGCATTATGCCCAGTACATGACCTTATG
GGACTTTCCTACTTGGCAGTACATCTACGTATTAGTCATCGCTATTACCATGG
GTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGC
GGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGC
GAAGCGCGCGGCGGGCGGGAGTCGCTGCGTTGCCTTCGCCCCGTGCCCCGCT
CCGCGCCGCCTCGCGCCGCCCGCCCCGGCTCTGACTGACCGCGTTACTCCCAC
AGGTGAGCGGGCGGGACGGCCCTTCTCCTCCGGGCTGTAATTagcgcttggtttaatgac
ggcttgtttcttttctgtggctgcgtgaaagccttgaggggctccgggagctagagcctctgctaaccatgttcatgccttc
ttattttcctacagctcctgggcaacgtgctggttattgtgctgtctcatcattttggcaaagAGGAGATACTTGAAGGCAG
TTTGAAAGACTTGTTTTACAGATTCTTAGTCCAAAGATTTCCAATTAGGGAGA
AGAAGCAGCAGAAAAGGAGAAAAGCCAAGTATGAGTGATGATGAGGCCTTC
ATCTACTGACATTTAACCTGGCGAGAACCGTCGATGGTGAAGTTGCCTTTTCA
GCTGGGAGCTGTCCGTTCAGCTTCCGTAATAAATGCAGTCAAAGAGGCAGTC
CCTTCCCATTGCTCACAAAGGTCTTGTTTTTGAACCTCGCCCTCACAGAAGCC
GTTTCTCATCACCGGTGCCACCATGCCTAGCCAGCAGAAGAAAATCATCTTCT
GCATGGCCGGCGTGTTCAGCTTCGCCTGTGCTCTGGGAGTTGTGACAGCCCTG
GGAACCCCTCTGTGGATCAAAGCCACAGTGCTGTGCAAGACAGGCGCCCTGC
TGGTTAATGCCTCTGGCCAAGAGCTGGACAAGTTCATGGGCGAGATGCAGTA
CGGCCTGTTCCATGGCGAAGGCGTCAGACAGTGTGGCCTGGGAGCCAGACCT
TTCAGATTCAGCTTCTTCCCAGACCTGCTGAAGGCTATCCCCGTGTCCATCCA
CGTGAACGTGATCCTGTTCAGCGCCATCCTGATCGTGCTGACAATGGTCGGA
ACCGCCTTCTTCATGTACAACGCCTTCGGCAAGCCCTTCGAGACACTGCATGG
ACCTCTGGGCCTGTACCTGCTGAGCTTTATCAGCGGCAGCTGTGGCTGCCTGG
TCATGATTCTGTTCGCCAGCGAAGTGAAGATCCACCACCTGAGCGAGAAGAT
CGCCAACTACAAAGAGGGCACCTACGTCTACAAGACCCAGTCCGAGAAGTAC
ACCACCAGCTTTTGGGTTATCTTCTTCTGTTTCTTCGTGCACTTCCTGAACGGC
CTGCTGATCAGACTGGCCGGCTTCCAGTTTCCATTCGCCAAGAGCAAGGACG
CCGAAACCACAAACGTGGCCGCCGATCTGATGTACTAAGAGCTCAAGGCAAA
CCTTTCTATAATTTTACAAGGGAGTAGACTTGCTTTGGTCACTTTTAGATGTG
GTTAATTTTGCATATCCTTTTAGTCTGCATATATTAAAGCATCAGGACCCTTC
GTGACAATGTTTACAAATTACGTACTAAGGATACAGGCTGGAAAGTAAGGGA
AGCAGAAGGAAGGCTTTGAAAAGTTGTTTTATCTGGTGGGAAATTGCTTGAC
CCAGGTAGTCAAAGGCAGTTGACTAGAATCGACAAATTGTTACTCCATATAT
ATATATGTGTGTGTGTGTGTGTGTGTGTGTGTGTAAGATGTCTTCCTATCAAA
AAGATATCAAAGGCACATGGAATATATTTTAATAAAAACAAATAATATCTCT
AATATATCCACACATTTGTTGCCAGATTTCAGAAAACTGAGCTGCAATCGCTT
TCCTAAAACAGTAGTGTATTAAATGAACATCTATAAAATGTATCAACACACA
TTTTAAAAAATTTGTTTAAAGTATACTCTTAGGCCAGGCGTGGTGACTCACAC
CTGTAATTCCAGCACTTCAGGAGGCCAAGGTGGGAAGATCATTTGAGTTCAG
GAGTTCGAGTTACAGCCTGGGCAATAAAGTGAGACCCTGTCACTAACAAAAT
TAAAAAATAAAATAAATATAAAATATAGGCTTTAAAAAAGCATAGTCTTATT
AACCATGTCTGTTGGTCAAAATCTGCAAACTCTAAAAGAAGAAAAGAAGAAA
AAACCAACGTTAGGGTATTTTTCCTCCCGTGCCTGAGTCCCAATTACATTCAC
GACAGTACTTTCAATGAACATAATTGTTAGGACCACTGAGGAATCATGAAAA
ATGATCTCTGCTTAGTACATTTGATGCAAAATGACTTATTAGGGGCTGTTTTT
CTAGCTATAGTGTCTCGAGTACTAATATGCAATTATGAAAATTATATTAAATC
TGGGATTATGACGGTATCACTGTATCATCTTGGTCTTGTTCTGGCTGTCACCA
AGCATGACCCAGGTCAACTTTTTTTTTCCCCTGAATTACCCATCAAATTGATC
TGCAGCTGACTAAAGGCCACAGCTGAGCCTGGAACTGACCCTTCCTTCATCCT
CAACCTGCTGTCCTCCAGAAAGCACCAAGGAAAAAGCAGAGAATGACAGCA
AACAGATCACTAGGCCTCTGACCACAGGTGCTGAGTACTCAGCAGCCCTCAT
ATAATAGGTTTGAAAGTACTCCTTAAAATAAAACACTGTTTCCCTTTGGAACT
ATTTACAAGGATGAAACAACCGTATACCTGAGAAATAACTTGCTCTGGTGTC
AATTCGCTATTCGCCAGCAGACATCAGAACACACCGAGTTTCCAGATGCTGG
TTTTTCCCCTTAAATCAGGAAATACACCTGGACAATTTCTAGAAGACTACAAT
TCAGTCTAGCCACAAAGGGGATTTTTTTTTTTTGGTAACAGGCTAGAGCCCGG
TTCTGTAAGTCTTTAGCTGAAATGGTCCAGTACAAAAGCACTGGAAATGAGT
GGGCTAGGAGGACAAGGACCGTCTCCTGCGTGAGGAGTTGGTTGGAGGTCCC
CAAGGCCAGGTACCCCCTGCACTCTTATTGGATTCCTCTCTGTCTTCTTGGAG
TTTTGAAAAACTCCTTCGAACACCAGGCTTTTTTCTTTAGAAAACAAGTCTCC
AATCGTTCTCTGTTCCGTAGAAAGAGAAAGAAAACCTGGAGCAGCTGCTGAA
AAATCTAATGAGGAACTAAGAGGCAAACCCACCACTGTGCCTTCTAGTTGCC
AGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAG
TAGGTGTCATTCTATTCTGGGGGGTGGGGTGGGGCAGGACAGCAAGGGGGAG
GATTGGGAAGACAATAGCAGGCATGCTGGGGATGCGGTGGGCTCTATGGAA
GCTTGAATTCAGCTGACGTGCCTCGGACCGCTAGGAACCCCTAGTGATGGAG
TTGGCCACTCCCTCTCTGCGCGCTCGCTCGCTCACTGAGGCCGGGCGACCAAA
GGTCGCCCGACGCCCGGGCTTTGCCCGGGCGGCCTCAGTGAGCGAGCGAGCG
CGCAGCTGCCTGCAGG

1.-228. (canceled)

229. A method comprising:
introducing into a cochlea of a mammal a therapeutically effective amount of a composition comprising a single nucleic acid vector, wherein the vector comprises a first coding sequence encoding a first isoform of CLRN1 protein,
wherein the first coding sequence comprises a nucleotide sequence spanning two consecutive exons of a CLRN1 genomic DNA, and lacking an intronic sequence between the two consecutive introns.

230. The method of claim 229, wherein the mammal is a human.

231. The method of claim 230, wherein the mammal has been previously identified as having a defective endogenous CLRN1 gene.

232. A composition comprising a single nucleic acid vector, wherein the vector comprises:
a first coding sequence encoding a first isoform of CLRN1 protein,
wherein the first coding sequence comprises a nucleotide sequence spanning two consecutive exons of a CLRN1 genomic DNA, and lacking an intronic sequence between the two consecutive introns.

233. The composition of claim 232, wherein the first isoform of the CLRN1 protein comprises a sequence that is at least 95% identical to SEQ ID NO: 3.

234. The composition of claim 232, wherein the first isoform of the CLRN1 protein comprises SEQ ID NO: 3.

235. The composition of claim 232, wherein the single nucleic acid vector further comprises a 5′ untranslated region (UTR), a 3′ UTR, or both.

236. The composition of claim 235, wherein the 5′ UTR comprises at least 10 contiguous nucleotides from SEQ ID NO: 12.

237. The composition of claim 236, wherein the 5′ UTR comprises at least 20 contiguous nucleotides from SEQ ID NO: 12.

238. The composition of claim 235, wherein the 3′ UTR comprises at least 10 contiguous nucleotides from SEQ ID NO: 36.

239. The composition of claim 238, wherein the 3′ UTR comprises at least 20 contiguous nucleotides from SEQ ID NO: 36.

240. The composition of claim 232, wherein the single nucleic acid vector is a plasmid, a transposon, a cosmid, an artificial chromosome, or a viral vector.

241. The composition of claim 232, wherein the single nucleic acid vector is a human artificial chromosome (HAC), yeast artificial chromosome (YAC), bacterial artificial chromosome (BAC), or a P1-derived artificial chromosome (PAC).

242. The composition of claim 232, wherein the single nucleic acid vector is a viral vector selected from an adeno-associated virus (AAV) vector, an adenovirus vector, a lentivirus vector, or a retrovirus vector.

243. The composition of claim 242, wherein the single nucleic acid vector is an AAV vector.

244. The composition of claim 232, wherein the single nucleic acid vector further comprises one or both of a promoter and a Kozak sequence.

245. The composition of claim 244, wherein the single nucleic acid vector comprises a promoter that is an inducible promoter, a constitutive promoter, or a tissue-specific promoter.

246. The composition of claim 244, wherein the single nucleic acid vector further comprises a polyadenylation signal sequence.

247. The composition of claim 232, further comprising a pharmaceutically acceptable excipient.

248. A kit comprising a composition of claim 232.

249. A method of increasing expression of a full-length CLRN1 protein in a mammalian cell, the method comprising introducing the composition of claim 232 into the mammalian cell.

250. A method of increasing expression of a full-length CLRN1 protein in an inner hair cell, an outer hair cell, or both, in a cochlea of a mammal, the method comprising:
introducing into the cochlea of the mammal a therapeutically effective amount of the composition of claim 232.

251. A method of increasing expression of a full-length CLRN1 protein in an eye of a mammal, the method comprising:
intraocularly administering to the eye of the mammal a therapeutically effective amount of the composition of claim 232.

252. A method of treating hearing loss in a subject identified as having a defective CLRN1 gene, the method comprising:
administering a therapeutically effective amount of a composition of claim 232 into the cochlea of the subject.

253. A method of treating vision loss in a subject identified as having a defective CLRN1 gene, the method comprising:
administering a therapeutically effective amount of a composition of claim 232 into the eye of the subject.
